(19) United States
(12) Patent Application Publication
(10) Pub. No.: US 2022/0267838 A1
(43) Pub. Date: Aug. 25, 2022

(54) Sensitive and Accurate Genome-wide Profiling of RNA Structure In Vivo

(71) Applicant: The Penn State Research Foundation, University Park, PA (US)

(72) Inventors: Philip C. Bevilacqua, State College, PA (US); Sarah M. Assmann, State College, PA (US); Zhao Su, State College, PA (US); Laura Ritchey, Martinsburg, PA (US); David Mitchell, State College, PA (US)

(21) Appl. No.: 16/762,820

(22) PCT Filed: Nov. 13, 2018
(86) PCT No.: PCT/US18/60660

§ 371 Date: May 8, 2020

Related U.S. Application Data
(60) Provisional application No. 62/585,011, filed on Nov. 13, 2017.

(51) Int. Cl. C12Q 1/6869; C12Q 1/6806

(52) U.S. Cl. CPC C12Q 1/6869; C12Q 1/6806

(57) ABSTRACT

The invention provides improved methods for determining the structure of RNA molecules with increased sensitivity, improved data quality, reduced ligation bias, and improved read coverage, incorporating the removal of undesired bi-products and ligation using a fast, efficient, and low-sequence bias hybridization-ligation method.

### CROSS-REFERENCE TO RELATED APPLICATIONS

This application is a U.S. national phase application filed under 35 U.S.C. § 371 claiming benefit to International Patent Application No. PCT/US2018/060660, filed Nov. 13, 2018, which is entitled to priority under 35 U.S.C. § 119(e) to U.S. Provisional Application No. 62/585,011, filed Nov. 13, 2017, each of which application is hereby incorporated herein by reference in its entirety.

### STATEMENT REGARDING FEDERALLY SPONSORED RESEARCH OR DEVELOPMENT

This invention was made with government support under IOS1339282 awarded by the National Science Foundation (NSF). The government has certain rights in the invention.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in the ASCII text file; 206032-0076-00US_SubstituteSequenceListing; created on May 3, 2021, and having a size of 16,005 bytes, is hereby incorporated by reference.

### BACKGROUND OF THE INVENTION

Unlike DNA, RNA is single stranded, can leave the nucleus of a cell, and is relatively unstable. RNA structure can be described in terms of its primary (sequence), secondary (hairpins, bulges and internal loops), tertiary (A-minor motif, 3-way junction, pseudoknot, etc.) and quaternary structure (supermolecular organization), also known as the RNA structure hierarchy.

For quite some time, RNA was considered merely an intermediate between DNA and protein. However, research has now shown that RNA itself can be functional. In fact, the complex structures are responsible for RNAs biological activity, such as catalyzing reactions, regulating gene expression, encoding proteins, and other essential cellular and biological roles. As RNA is now appreciated to serve numerous cellular roles, the understanding of RNA structure is important for understanding the mechanism of action (how RNA folds to produce the various functions). The study of functional and structural aspects of RNA across all the RNA molecules in a cell or system is called transcriptomics research.

In order to advance transcriptomics research to better understand RNA, structure prediction and determination technologies have been developed. The experimental methods for measuring RNA 3D structure include, but are not limited to, X-ray crystallography, NMR spectroscopy, computational algorithms & modeling, and high throughput RNA sequencing (RNA-seq) technologies. RNA sequencing can measure the expression levels of thousands of genes simultaneously and provide insight into functional pathways and regulation in biological processes.

Many of the experimental methods for measuring RNA structure are in vitro. However, RNA structures in vivo often differ from in vitro structures and, moreover, change dramatically in vivo because they are remodeled in response to changes in the prevailing physico-chemical environment of the cell, as well as by inter-molecular base pairing and interactions with RNA binding proteins.

Traditional methods for RNA structure determination include X-ray crystallography, NMR, cryo-electron microscopy, spectroscopy, gel electrophoresis (PAGE) and capillary electrophoresis. Many of these classical methods utilize chemical and enzymatic (RNase) probing of one RNA at a time and can only provide information on approximately 150-500 nucleotides of one given transcript at a time. Therefore, these traditional approaches are low throughput, tedious for studying long RNAs, and difficult to scale. DMS was first used in the 1980s as a reagent to probe single RNA sequences. These methods have limitations to determine stereo-chemical structure due to the rapid degradation of RNA, limitations in the length of the probed RNA, and limitations in analyzing only one single RNA per experiment.

A major limitation to RNase methods is that the RNA must be extracted from the cell because the enzymes used cannot easily penetrate the cell membrane, making them limited to in vitro applications. In addition, this technique strips away RNA-binding proteins, which can dramatically alter the structure, enzyme digestion can be nonspecific, digestion conditions must be carefully controlled, RNA can be overdigested, and the large physical size of RNases can restrict their ability to detect RNA structural fingerprints.

Determination of RNA secondary and tertiary structures still remains a challenging problem, particularly studying co-transcriptional folding on a genome-wide scale. The probing pattern obtained is from an average of structures and the structure of RNA as it is being transcribed is likely different from the fully folded structure.

RNA serves many functions in biology such as splicing, temperature sensing, and innate immunity. These functions are often determined by the structure of RNA. There is thus a pressing need to understand RNA structure and how it changes during diverse biological processes both in vivo and genome-wide. Many of these can be informed via a global RNA structurome and thus genome-wide information on RNA structure is highly valuable. High-throughput methods provide an efficient, cost-effective alternative to classical one-off gene-specific, typically gel-based studies of RNA structure, Recently, several high-throughput RNA structural methods have been developed (Bevilacqua et al., 2016, Annu Rev Genet, 50:235-266; Kwok et al., 2015, Trends Biochem Sci. 40:221-232; Strobel et al., 2016. Curr Opin Biotechnol, 39:182-191; Kubota et al, 2015, Nat Chem Biol, 11:933-941). Among these methods, Structure-seq (Ding et al., 2015, Nat Protoc, 10:1050-1066; Ding et al., 2014, Nature, 505:696-700), has some advantages in experimental and computational pipelines. Most importantly, because Structure-seq relies on chemical modification rather than nuclease cleavage, it can be performed in vivo, which is significant as in vivo and in vitro structures often differ (Leamy et al, 2016, Q Rev Biophys, 49:e10). The experimental approach of Structure-seq has an advantage over other protocols in that reverse transcription (RT) is conducted immediately after RNA purification to minimize RNA degradation. Structure-seq also provides a powerful, user-friendly computational pipeline called StructureFold (Tang et al., 2015, Bioinformatics, 31:2668-26751.

In the original Structure-seq method (Ding et al., 2014, Nature, 505:696-700), RNA is probed in vivo with dimethyl sulfate (DMS), under single-hit kinetics conditions, which covalently modifies unprotected adenines and cytosines. After RNA extraction and mRNA enrichment, reverse transcription (RT) with a random hexamer-containing primer is performed, which stops at the nucleotide before the modified nucleotide. After adaptor ligation to the cDNA Y end, the product is PCR-amplified and sequenced. The RT stop signal of a minus DMS sample is subtracted from that of the plus DMS sample and reactivities are calculated which can be used as restraints to predict RNA structures genome-wide (Reuter and Mathers, 2010, BMC Bioinformatics, 11:129), While Structure-seq is powerful, there are steps that can be improved to provide competitive advantages in time, labor, technological benefits, and cost.

Thus, there is a need in the art for an improved method for obtaining nucleotide-resolution RNA structural information in vivo and genome-wide with increased sensitivity, improved data quality, reduced ligation bias, more rigorous structure prediction, and improved read coverage. The present invention satisfies this unmet need.

### SUMMARY OF THE INVENTION

In one embodiment, the invention relates to a method of obtaining nucleotide-resolution RNA structural information in vivo comprising the ordered steps of: a) treating an RNA molecule in vivo with an agent which covalently modifies unprotected nucleobases, b) performing reverse transcription (RT) with a random hexamer-containing primer to generate a cDNA molecule, c) ligating a hairpin donor molecule to the 3′ end of the cDNA molecule, d) performing PCR amplification of the ligated construct and e) sequencing the amplified products.

In one embodiment the agent is dimethyl sulfate (DMS), glyoxal, methylglyoxal, phenylglyoxal, 1-cyclohexyl-3-(2-morpholinoethyl)-carbodiimide methyl-p-toluenesulfonate (CMCT), nicotinoyl azide (NAz) or 1-ethyl-3-(3-dimethylaminopropyl)carbodiimide (EDC), and SHAPE (Selective Hydroxyl Acylation analyzed by Primer Extension) reagents that react with the 2′ hydroxyl, including, but not limited to, 1M7 (1-methyl-7-nitroisatoic anhydride), 1M6 (1-methyl-6-nitroisatoic anhydride), NMIA (N-methyl-isatoic anhydride), FAT (2-methyl-3-furoic acid imidazolide), NAI (2-methylnicotinic acid imidazolide), and NAI-N3 (2-(azidomethyl)nicotinic acid acyl imidazole).

In one embodiment, the random hexamer-containing primer of step b) comprises a nucleotide sequence of SEQ ID NO:6.

In one embodiment, the ligation in step c) comprises ligating a hairpin donor molecule comprising SEQ ID NO:1 to the 3′ end of the cDNA molecule.

In one embodiment, the ligation is performed using T4 DNA ligase.

In one embodiment, the PCR amplification in step d) comprises contacting the ligated construct with a forward primer having a sequence as set forth in SEQ ID NO:3 and a reverse primer having a sequence as set forth in SEQ ID NO:4.

In one embodiment, the sequencing in step e) is performed using a sequencing primer as set forth in SEQ ID NO:5.

In one embodiment, the method further comprises at least one purification step. In one embodiment, the method further comprises at least one purification step after step b) and before step c). In one embodiment, the method further comprises at least one purification step after step c) and before step d). In one embodiment, the method further comprises at least one purification step after step d) and before step e).

In one embodiment, at least one purification step comprises polyacrylamide gel (PAGE) purification.

In one embodiment, at least one purification step comprises affinity purification. In one embodiment, the affinity purification comprises biotin/streptavidin affinity purification.

In one embodiment, the method comprises three purification steps.

In one embodiment, the method comprises a first purification step after step b) and before step c), a second purification step after step c) and before step d), and a third purification step after step d) and before step e).

In one embodiment, the invention relates to a nucleic acid molecule comprising a sequence selected from the group consisting of SEQ ID NO:3, SEQ ID NO:4, SEQ 1D NO:5 and SEQ ID NO:6.

In one embodiment, the invention relates to a kit comprising a nucleic acid molecule comprising a sequence selected from the group consisting of SEQ ID NO:3, SEQ ID NO:4, SEQ ID NO:5 SEQ ID NO:6 and a combination thereof.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A and FIG. 1B, depicts schematic diagrams showing exemplary methods of use of the improved Structure-seq methods (Structure-seq2) used to produce high quality data. In Structure-seq2, RNA is first modified by DMS or another chemical that can be read-out through reverse transcription. The RNA is then prepared for Illumina NGS sequencing by conversion to cDNA (Step 1A/1B), ligating an adaptor (Step 3A/3B), and amplifying the products while incorporating TruSeq primer sequences (Step 5A/5B). In order to increase library quality, numerous improvements were made to the original Structure-seq protocol (boxed). These include performing the ligation with a hairpin adaptor and T4 DNA ligase (Step 3A/3B), and adding various purification steps to remove a deleterious by-product (FIG. 3A and FIG. 3B). FIG. 1A depicts purification using polyacrylamide gel (PAGE) purification. In the PAGE purification method, an additional PAGE purification step is added after reverse transcription (Step 2A). FIG. 1B depicts a biotin-streptavidin pull down. In the biotin-streptavidin pull down method, biotinylated dNTPs are incorporated into the extended product during reverse transcription (Step 1B) and are purified via a magnetic streptavidin pull down after reverse transcription (Step 2B) and after ligation (Step 4B). There is also a common, final PAGE purification step following amplification (Step 5A/5B). Finally, a custom sequencing primer is used during sequencing (Step 7A/7B) to further provide high quality data.

FIG. 2, comprising FIG. 2A through FIG. 2F, depicts exemplary experimental results demonstrating that library replicates have good correlation. FIG. 2A through FIG. 2D depict exemplary experimental results demonstrating the RT stop counts between individual replicates for −DMS and +DMS conditions prepared using either the PAGE method or the biotin method are all well correlated. FIG. 2E and FIG. 2F depict exemplary experimental results demonstrating the RT stop counts between PAGE variation and biotin variation are also well correlated in both −DMS and +DMS libraries.

FIG. 3, comprising FIG. 3A and FIG. 3B, depicts exemplary experimental results demonstrating that Structure-seq2 leads to a lower ligation bias and overall mismatch rate in rice (Oryza sativa). FIG. 3A depicts exemplary experimental results demonstrating that after reverse transcription (FIG. 1, step 1A/1B), excess of the 27 nt primer (top, right) is still present in the solution. During ligation (FIG. 1, step 3A/3B), this primer can also ligate to the 40 nt hairpin adaptor to form an unwanted 67 nt by-product which has no insert and so results in sequencing reads with no utility. FIG. 3B depicts exemplary experimental results demonstrating that the complement of the first nucleotide after the adaptor sequence read during sequencing is the nucleotide that ligated to the adaptor. The T4 DNA ligase-based method (−DMS and +DMS)(see U.S. Pat. Pub. No. 2014/0193860 A1, incorporated herein by reference), substantially decreases ligation bias as compared to the previous Circligase-based method. Percentages equaling the transcriptomic distribution of the four nucleotides are ideal.

FIG. 4 depicts exemplary experimental results demonstrating that the by-product formed from the ligation of the reverse transcription primer to the hairpin adaptor (dashed boxed region, see FIG. 3) can readily be amplified to produce a 149/151 by product. The two sizes are due to different sizes of the barcodes (6-8 nt) incorporated in the primers,

FIG. 5 depicts exemplary experimental results demonstrating that the by-product is formed from ligation of the reverse transcription (RT) primer and the ligation hairpin adaptor. The T4 DNA ligation reaction is performed with various components present. The RT primer can ligate to the ligation adaptor (FIG. 3) to form the 67 nt by-product, indicated with an arrow, if both are present in the ligation reaction (lane 4). The RT primer is 27 nt (lane 2) and the ligation adaptor is 40 nt (lane 3). If there is no enzyme present in the reaction (lane 1), no product is formed. Lane M1 is a GeneRuler Low Range DNA Ladder, and Lanes M2 are a mixture of ssDNA oligonucleotides (67 nt and 91 nt) to allow for proper identification of the by-product (67 mt) and the cut site (90 nt). The 10% acrylamide-8.3 M urea PAGE gel is stained with SybrGold for visualization.

FIG. 6 depicts exemplary experimental results demonstrating that post-reverse transcription PAGE purification is necessary to obtain sufficient library sample from 500 ng of RNA. Bioanalyzer traces of samples without (top) and with (bottom) a post-reverse transcription PAGE purification step (FIG. 1, step 2A/2B). These samples were otherwise treated identically. The addition of the PAGE purification step improves the efficiency of the subsequent steps, which produce a product between 300 and 600 bp.

FIG. 7 depicts exemplary experimental results demonstrating that bioanalyzer traces can reveal the presence of by-product prior to sequencing. Bioanalyzer traces show the presence of by-product. Markers of 35 bp and 10,380 bp are provided. Additionally, the extent to which the Illumina MiSeq instrument returns a read as a stretch of 35 N's (% N35) correlates with the amount of by-product seen on the Bioanalyzer. It was noted that the by-product runs at ˜172 bp, rather than at its true length of 1491151 bp. This is likely due to the third denaturing PAGE gel that caused the by-product to become single-stranded, prior to Bioanalyzer analysis.

FIG. 8, comprising FIG. 8A and FIG. 88, depicts exemplary experimental results demonstrating that biotin does not affect nucleotide composition or read depth. FIG. 8A depicts exemplary experimental results demonstrating that adding biotin during reverse transcription does not alter the distribution of nucleotide reads. Addition of dCTP as the only biotinylated dNTP during reverse transcription does not affect the nucleotide composition of the reads. “Structure-seq2” and “Structure-seq2 with biotin” refer to samples prepared via the methods described in FIG. 1. “Biotin” refers to a sample prepared with biotinylated-dCTP incorporated during RT, but purified via PAGE gels. FIG. 8B depicts exemplary experimental results demonstrating that the read depth on 25S rRNA is similar regardless of whether samples are purified via the PAGE variation or biotin variation. In fact, in some instances, the biotin variation provides a higher read depth than the PAGE variation. The read depth here is shown as lines to directly compare the two methods.

FIG. 9, comprising FIG. 9A through FIG. 9D, depicts exemplary experimental results demonstrating that biotin does not affect the read profiles of the transcripts, FIG. 9A and FIG. 9B depicts exemplary experimental results demonstrating that the read profiles between +DMS and −DMS are well correlated using both the biotin and the PAGE variations. FIG. 9C and FIG. 90 depicts exemplary experimental results demonstrating that the read profiles between the PAGE and biotin variations are also well correlated for both the +DMS and the −DMS treatments. The ten transcripts with the highest G content, and the ten transcripts with the lowest G content are dispersed throughout the read profiles.

FIG. 10, comprising FIG. 10A and FIG. 10B, depicts the results of exemplary experiments demonstrating Structure-seq2 identifies a previously unreported m¹A in 25S rRNA. FIG. 10A depicts exemplary experimental results demonstrating that using the original Structure-seq method for reverse transcription denaturation (65° C. with no monovalent salt), there are regions that receive no reads (denoted with arrows). FIG. 10B depicts exemplary experimental results demonstrating that increasing the denaturation conditions (90° C. with monovalent salt) allows these regions to be and narrows regions of low read depth. Total number of reads is similar in FIG. 10A and FIG. 10B. Reads continue to decrease until they go to zero at nucleotide 539. The region between nucleotides 432 and 644 is 79% GC-rich with a read depth less than 100 on each nucleotide. FIG. 10C depicts exemplary experimental results demonstrating that this site corresponds to a high reverse transcription stop count at the precise location in the −DMS data.

FIG. 11 depicts exemplary experimental results demonstrating that Structure-seq2 DMS reactivity correlates well with traditional gel-based reactivity of 5.8S rRNA. After DIS treatment, a traditional 5.8S rRNA gene-specific gel-based chemical probing analysis was completed. Using ImageQuant software, a vertical line was drawn through the appropriate portion of the PAGE gel for the manual footprinting of 5.8S rRNA and integrated. The integrated data for the manual footprinting (line) was aligned with the Structure-seq2 data (bars), with small accommodations to account for the logarithmic nature of PAGE.

FIG. 12 depicts exemplary experimental results demonstrating DMS reactivity of rRNA in Bacillus subtilis. Gel-based probing reveals that in vivo DMS treatment selectively modifies adenosine and cytosine residues in solvent-accessible regions. This includes bases that are unpaired and on the surface of the structure. Left structures show the gel-based reactivity mapped onto the secondary structure of 23S, 16S and 5S rRNA (from top). The panels on the right show the reactivities mapped onto a crystal structure of B. subtilis (39W) (Sohmen et al. 2015, Nat Commun. 6:6941). Reactivities were calculated using a 2%-8% normalization. High reactivity (>0.6); medium reactivity (0.3-0.6); low reactivity (<0.3).

FIG. 13, comprising FIG. 13A through FIG. 13C, depicts exemplary experimental results demonstrating that Structure-seq2 can be benchmarked on rRNA. FIG. 13A depicts exemplary experimental results demonstrating that by mapping the reactivities generated from Structure-seq2 onto the completely conserved, ancient peptidyl transferase center of 25S rRNA, nucleotides with high reactivity map onto single-stranded regions of the rRNA, (dark grey: DMS reactivity ≥0.6; light grey: DMS reactivity 0.346; medium grey: DMS reactivity ≤0.3 or no data). FIG. 13B depicts exemplary experimental results demonstrating that, when comparing the reactivity values obtained between the original Structure-seq method in Arabidopsis and Structure-seq2 in rice, there is overlap in reactivity position. FIG. 13C depicts exemplary experimental results demonstrating that there is a good correlation of reactivities between the species (r=0.7738).

FIG. 14 depicts exemplary experimental results demonstrating the reactivity pattern of an aligned conserved region compared between rice and Arabidopsis. The region of the mRNA with the highest reactivity coverage in the MiSeq data generated herein, OS121T0274700-02, aligns well with AT5G38420.1. The alignment is shown with reactivities plotted on the individual nucleotides (high >0.6 (dark grey); medium, 0.3-0.6 (light grey); low <0.3 (medium grey)). Only reactivities corresponding to nucleotides that were an A or a C in both organisms were considered for the correlation or the alignment. Using the continuous reactivities calculated through StructureFold, there was a good correlation (r=0.4239) on the orthologous transcripts between these two species, indicating that structure as well as sequence may be conserved.

FIG. 15 depicts multiple RNA structure diagrams demonstrating that the location of the large drop in reads downstream of the single region in 25S that remains absent of reads corresponds to a site known to contain a m¹A in yeast, human, and H. marismortui (Cannone et al., 2002, BMC Bioinformatics, 3:2; Piekna-Przybylska et al, 2008, Nucleic Acids Res, 36:D178-183),

FIG. 16 depicts close ups of the m¹A containing regions of the multiple RNA structure diagrams of FIG. 15.

FIG. 17 depicts exemplary experimental results demonstrating that structure-seq2 demonstrates the presence of two hidden breaks in chloroplast rRNA. At the two locations known to harbor hidden breaks in chloroplast rRNA, the −DMS RT stop count data spike. The spike at the first hidden break differs by one nucleotide from the published break site in spinach and Arabidopsis (Bieri et al., 2017, EMBO J, 36:475-486; Liu et al., 2015, Plant Physiol, 168:205-221), which could be due to the slight sequence variation between species (Arabidopsis: 5′-GGGAGUGAAA*UAGAACA-3′ (SEQ ID NO:21), Rice: 5′-GGGUAGUGAAAU*AGAACG-3′(SEQ ID NO:22), where indicates the proposed break site). The spike at the second hidden break occurs precisely at the published cleavage site for spinach and Arabidopsis (Dieri et al, 2017, EMBO J, 36:475-486; Liu et al., 2015, Plant Physiol, 168:205-221).

FIG. 18 depicts a schematic diagram of the workflow of temperature treatment and rice library construction using Structure-seq2. Two-week-old rice shoots were treated with DMS (+DMS sample) for 10 min at 22° C. or 42° C. DMS covalently modifies single-stranded As and Cs. These modifications cause reverse transcription to stop one nucleotide before the modification; occasional native RNA modifications or strong in vitro RNA structure can also cause stops, which are accounted for using control (−DMS) libraries. Random hexamers (N6) with a TruSeq adaptor were employed for reverse transcription. DNA ligation was performed using T4 DNA ligase, which can ligate a hairpin DNA linker donor to the 3′end of cDNAs. Library amplicons were then generated by PCR using Q5 high fidelity polymerase. Urea polyacrylamide gel electrophoresis (Urea-PAGE) was used for all DNA purifications. Illumina MiSeq sequencing was used for library quality determination and Illumina HiSeq sequencing was used for final data generation. DMS reactivity at nucleotide resolution was generated using the StructureFold program. Boxes indicate steps in the current Structure-seq2 protocol (3) that are improvements from the original Structure-seq method (Ding et al., 2015, Nat Protoc, 10:1050-1066; Ding et al., 2014, Nature, 505:696-700).

FIG. 19, comprising FIG. 19A through FIG. 19D, depicts the experimental results demonstrating Experimental design and Structure-seq library statistics. FIG. 19A depicts the timeline of Structure-seq, RNA-seq, and Ribo-seq experiments. [Scale bar for rice seedlings, 4 cm.] FIG. 19B depicts the overlap of mRNAs with sufficient structure-probing coverage between 22° C. and 42° C. FIG. 19C depicts heat stress-induced structural reactivity changes across the rice mRNA structurome. Each horizontal line represents a different mRNA. Reactivity information is obtained at single nucleotide resolution (inset). Vertical line marks start codon. FIG. 19D depicts the average DMS reactivity is significantly greater at 42° C. than 22° C. (whole transcripts; P=5.27×10⁻⁷⁷; r=0.82). [Scale bar (gradient), numbers of RNAs.] In the analyses of FIG. 19C and FIG. 19D, only transcripts with sufficient Structure-seq coverage under both temperature conditions are shown and used.

FIG. 20, comprising FIG. 20A through FIG. 20F, depicts experiments demonstrating the high correlation of single nucleotide reverse transcription (RT) stop counts between replicates in +DMS libraries. FIG. 20A through FIG. 20C depicts the correlation between 3 biological replicates at 22° C. FIG. 20D through FIG. 20F depicts the Correlation between 3 biological replicates at 42° C. All of the biological replicates at each temperature are highly correlated.

FIG. 21, comprising FIG. 21A through FIG. 21I, depicts experiments demonstrating that the majority of Structure-seq reads are from mRNAs. FIG. 21A depicts a −DMS library at 22° C. (136,504,440 total mapped reads). FIG. 21B depicts a +DMS library at 22° C. (152,310,815 total mapped reads). FIG. 21C depicts a −DMS library at 42° C. (125,305,132 total mapped reads). FIG. 21D depicts a +DMS library at 42° C. (141,636,436 total mapped reads). FIG. 21E through FIG. 21H depicts experiments demonstrating that nucleotide modifications in the +DMS libraries are specific to As and Cs. FIG. 21E depicts a −DMS library at 22° C. FIG. 21F depicts a +DMS library at 22° C. FIG. 21G depicts a −DMS library at 42° C. FIG. 21H depicts a +DMS library at 42° C. FIG. 21I depicts an analysis demonstrating that +DMS libraries show greater modification of A and C than of U and G.

FIG. 22, comprising FIG. 22A through FIG. 22D, depicts the distribution of structure-probing coverage, and 3′UTRs show greatest heat-induced change in DMS reactivity (42° C.-22° C.). FIG. 22A depicts the distribution of coverage of all transcripts in Structure-seq datasets at 22° C. Structure-seq provided structural information at nucleotide resolution on 16,411 RNAs with coverage over 1 at 22° C. FIG. 22B depicts the distribution of coverage of all transcripts in Structure-seq datasets at 42° C. Structure-seq provided structural information at nucleotide resolution on 14,738 RNAs with coverage over 1 at 42° C. Lengths of regions (5′ UTR, CDS, 3′UTR) on each mRNA were normalized and aligned for plotting. Red indicates 5′UTR, black indicates CDS, and blue indicates 3′UTR (Zero value is included for clarity, as indicated). FIG. 22C depicts the distribution of the 2,000 spots with the most elevated DMS reactivity at 42° C. as compared to 22° C. (change in DMS reactivity; left axis). A ‘spot’ is defined as average reactivity in a 100 nt window. The 2.000 spots were identified solely based on reactivity change, independent of location on the mRNA. Distribution shows enrichment of the hot spots in 3′UTRs. Line shows the distribution of the total number of spots (spot density; right axis) along each normalized region, for the 1,170 mRNAs harboring the 2.000 spots. FIG. 22D depicts the distribution of the 2,000 spots with the most reduced DMS reactivity at 42° C. as compared to 22° C. Line shows the distribution of the total number of spots along each normalized region, for the 982 mRNAs harboring the 2,000 spots.

FIG. 23, comprising FIG. 23A through FIG. 23J, depicts exemplary experiments demonstrating that the average DMS reactivity is higher on all mRNA regions at elevated temperature. Average DMS reactivity is significantly greater at 42° C. for all mRNA subregions. (FIG. 23A) 5′UTR (P:=4.00×10⁻¹⁸; r=0.74). (FIG. 23B) CDS (P=8.08×10^{31 12}; r=0.83). (FIG. 23C) 3′UTR (P 2.24×10⁻⁸⁹; r=0.87). DMS reactivities on whole transcripts were cross-normalized between temperatures to correct for the higher chemical reactivity of DMS at higher temperature (SI Appendix. Materials and Methods). [Scale bars (gradient) in FIG. 23A-FIG. 23C, numbers of mRNAs.](FIG. 23D) Average AU content is significantly greater in 3′UTRs than in 5′UTRs or CDS, especially at the 3′ end (last 100 nt). (FIG. 23E and FIG. 23F) Mean of the average DMS reactivity at 22° C. (FIG. 23E) and 42° C. (FIG. 23F) in the 5′ UTR, CDS, 3′UTR regions. (FIG. 23G) Change in average DMS reactivity (42° C.-22° C.) in the 5′UTR. CDS, and 3′UTR regions. (H and 1) Mean of single strandedness at 22° C. (FIG. 23H) and 42° C. (FIG. 23I) in the 5′UTR, CDS, 3′UTR regions, Here, single-strandedness is the percentage of single-stranded nucleotides in the RNA structure predicted with in vivo restraints. (FIG. 23J) Change in average single strandedness (42° C.-22° C.) in the 5′UTR, CDS, 3′UTR regions. In the analyses of A-J, only transcripts with sufficient Structure-seq coverage under both temperature conditions were used. In FIG. 23E-FIG. 23J, *P<0.01; *P<10⁻¹⁰; ***P<10⁻⁵⁰, respectively.

FIG. 24, comprising FIG. 24A through FIG. 24D, depicts correlations between U and AU content at the 3′ends of 3′UTRs and heat-induced DMS reactivity changes. FIG. 24A depicts the U content of the last 10 nt at the 3′end of the 5% of mRNAs with most elevated (Top 5%) or reduced (Bottom 5%) DMS reactivity at 42° C. as compared to 22° C. FIG. 24B depicts transcripts with high U content (≥8) in the last 10 nt of the 3′UTR showed significantly higher heat-induced change in average DMS reactivity of the entire 3′UTR than the ones with low U content (≤3) in the last 10 nt of 3′UTR (P=0.03), FIG. 24C depicts the single nucleotide frequency (left y-axis) and FIG. 24D depicts the dinucleotide frequency (left y-axis) and DMS reactivity change (42° C.-22° C.; right y-axis) along the 3′UTRs. Nucleotide frequencies and DMS reactivities are binned into 40 bins (10 nt per bin). The UTR region depicted excludes the very 3′ end where DMS reactivity data do not meet the minimum coverage requirement. The five most common dinucleotides near the 3′ end are UU, GU, AU, UA, and UG (annotated), suggesting that melting of AU and GU base pairing may contribute to enhanced DMS reactivity under heat.

FIG. 25, comprising FIG. 25A through FIG. 25D, depicts exemplary experiments demonstrating Ribo-seq data statistics and the absence of correlations between temperature induced changes in DMS reactivity and in the translatome. (FIG. 25A) Distribution of sequence read length of Ribo-seq data, peaking at 30-32 nucleotides, as expected for ribosome footprinting. (FIG. 25B) Percentage of mRNA-mapped Riboseq reads that map to the CDS. (FIG. 25C) Distribution of sequence read count around start codon and stop codon. Shown are 32-nt reads as the example: reading frames are shown in red (first position), blue (second position), and green (third position), and UTRs are highlighted in pink and gray. (FIG. 25D and FIG. 25E) High correlation of transcript abundance between replicates of Ribo-seq libraries. Transcript abundance was calculated as TPM (transcripts per million). (FIG. 25G) 22° C. (FIG. 25E) 42° C. (FIG. 25F-FIG. 25H) No correlation detected between the change in average DMS reactivity (42° C.-22° C.) and change in Ribo-seq signal (42° C.-22° C.) for (FIG. 25F) all transcripts (n=14,197). (FIG. 25G) 5′UTR (n:=9,895), (FIG. 25H) start codon region (˜50 nt to +50 nt; n=8,726). n, number of candidates with both sufficient coverage in Structure-seq and presence in Ribo-seq datasets.

FIG. 26, comprising FIG. 26A through FIG. 26D, depicts experiments demonstrating a negative correlation between DMS reactivity and mRNA abundance change as measured from DMS Structure-seq libraries, and high correlation of mRNA abundance between Structure-seq and RNA-seq libraries. FIG. 26A and FIG. 26B depict a negative correlation between change of average DMS reactivity (42° C.-22° C.) and RNA abundance change (42° C.-22° C.), measured from Structure-seq libraries as log 2(TPM) at 22° C. and 42° C. for the 14,292 mRNAs with coverage above 1 in Structure-seq analysis. Colors indicate numbers of mRNAs. FIG. 26A depicts −DMS libraries. FIG. 26B depicts+DMS libraries. FIG. 26C and FIG. 26D depict a strong positive correlation between mRNA abundance as calculated from Structure-seq−DMS libraries and mRNA abundance as calculated from RNA-seq 10 min libraries at 22° C. (FIG. 26C) and 42° C. (FIG. 26D).

FIG. 27 depicts the hierarchical clustering of RNA-seq datasets indicates the relationships of the samples and the recovery of the transcriptome following 10 minutes of 42° C. heat shock. C=control, H=heat shock for 10 minutes. HR=heat recovery. Scale indicates transcriptome percent similarity between samples. The tree was generated using MEN software (mev.tm4.org). TPM-based RNA-seq timecourse datasets were analyzed using hierarchical clustering to show the relationship between the samples.

FIG. 28, comprising FIG. 28A through FIG. 28C, depicts experiments demonstrating that no correlation was detected between heat shock induced change in Ribo-seq signal (42° C.-22° C.) and mRNA abundance change between 42° C. and 22° C. at 10 minutes (=end of 42° C. treatment). FIG. 28A depicts the correlation of abundance change with Ribo-seq signal change for the whole transcripts, FIG. 288 depicts the correlation of the transcripts with 1.5 fold decrease in Ribo-seq signal (log 2(ribo-seq signal)<−0.58)(zoom-in of lefthand portion of FIG. 28A). FIG. 28C depicts the Correlation of the transcripts with 1.5 fold increase in Ribo-seq signal (log 2(riboseq signal)>0.58)(zoom-in of right-hand portion of FIG. 28A).

FIG. 29, comprising FIG. 29A through FIG. 29H, depicts exemplary experiments demonstrating Strong negative correlation between heat-shock-induced DMS reactivity change and heat-shock-induced mRNA abundance (TPM) change that gradually dissipates after heat shock. (FIG. 29A-FIG. 29E) Change of average DMS reactivity (42° C.-22° C.) from Structure-seq (all 10 min) vs. fold change (log 2) in mRNA abundance (42° C.-22° C.) from RNA-seq (see FIG. 19A for time course), calculated on all mRNAs with sufficient Structure-seq coverage. (FIG. 29A) 10 minutes (=end of 42° C. treatment), (FIG. 29B) 20 minutes, (FIG. 29C) 1 hour, (FIG. 29D) 2 hours, (FIG. 29E) 10 hours. (FIG. 29F) Distribution of change in average DMS reactivity of all transcripts with sufficient Structure-seq coverage within the top 5% of mRNAs with increased abundance and the bottom 5% of mRNAs with decreased abundance. (FIG. 29G and FIG. 29H) The abundance of degradome fragments of the top/bottom 5% most/least DMS reactive transcripts at (FIG. 29G) 42° C. and (FIG. 29H) 22° C. is compared, showing that more reactive transcripts have a higher mean number of degradome fragments.

FIG. 30, comprising FIG. 30A through FIG. 30D, depicts exemplary experiments demonstrating that the Y-end+A15 polyA tail RNA unfold in the range of heat treatment and the mRNAs of T2 and T3 decay faster under heat. FIG. 30A depicts raw melts of four candidate RNAs from the top 5% that lose abundance under heat treatment. Sloping baselines are likely due to the 15 A's unstacking, given the tendency of polyA to stack. FIG. 30B depicts derivatives of the optical melting data from T2 and T3, which show appreciable sigmoidal characteristic in FIG. 30A. FIG. 30C depicts the fraction folded of T2 and T3. Fraction folded is calculated from the equation Fraction Folded=(A−Au)(Af−Au), where A is the absorbance at a given temperature, Au is the absorbance of the unfolded RNA which is calculated from the linear fit of the upper baseline, and Af is the absorbance of the folded RNA which is calculated from the linear fit of the lower baseline. Sequences were derived from the following genes: T1 (OS06T0105350-00) Similar to Scarecrow-like 6; T2 (OS02T0662100-01) Similar to Tfm5 protein; T3 (OS03T0159900-02) Hypothetical conserved gene: T4 (OS02T0769100-01) Auxin responsive SAUR protein family protein. See Materials and Methods for specific sequences and methodological details. FIG. 30D depicts the RNA decay rate analysis of T2 and T3 under two temperature conditions (42° C. vs 22° C.) in the presence of cordycepin shows accelerated decay at 42° C.

FIG. 31, comprising FIG. 31A through FIG. 31H, depicts AU content and U content at the 5′ end are significantly different between top 5% and bottom 5% of mRNAs; XRN targets show significantly higher 5′UTR AU content and DMS reactivity change (42° C.-22° C.) than non-XRN targets and decay rapidly under heat. FIG. 31A depicts the AU content of the first 10 nt at the 5′end of the 5% mRNAs with most elevated (Top 5%) or reduced (Bottom 5%) DMS reactivity at 42° C. as compared to 22° C. FIG. 32A depicts the AU content of the 5′UTRs of the 5% of mRNAs with most elevated (Top 5%) and reduced (Bottom 5%) DMS reactivity at 42° C. as compared to 22° C. FIG. 31C depicts the higher AU content of the 5′UTRs of rice orthologs (derived from the MSU Rice Genome Annotation Project; rice.plantbiology.msu,edu/index.shtml) of mRNAs subject to heat-induced XRN4-mediated decay vs. XRN4 non-responsive mRNAs from published datasets (Merret et al., 2015, Nucleic Acids Res. 43(8):4121-4132). P values are from Chisquared tests. FIG. 32D depicts the distribution of change in DMS reactivity of rice orthologs of XRN targets identified from (Merret et al., 2015) at 42° C. as compared to 22° C. The average change in DMS reactivity (42° C. compared to 22° C.) of rice orthologs of XRN target mRNAs is significantly higher than that of mRNAs which are not XRN target orthologs. (p=0.02, two sample t-test). FIG. 31E through FIG. 31H depict the mRNA decay rate analysis of XRN target transcripts under two temperature conditions (42° C. vs 22° C.) in the presence of cordycepin shows accelerated decay at 42° C.

FIG. 32, comprising FIG. 32A through FIG. 32D, depicts exemplary experiments demonstrating that gene ontology analysis uncovers enrichment of transcription factors in mRNAs with the greatest heat-induced DMS reactivity increases. (FIG. 32A) Enrichment of gene ontology functional categories in the 5% of mRNAs with most elevated DMS reactivity at 42 T. (FIG. 32B) DMS reactivity profiles for four transcription factors in the “regulation of transcription” category; these show dramatic heat-induced increase in DMS reactivity. For visualization, reactivity differences (42° C.-22° C.) on all nucleotides in a transcript were placed into 100 bins and averaged within each bin. Green and black arrowheads point to the end of 5′UTR and the start of 3′UTR, respectively. (FIG. 32C) Heat-promoted mRNA decay. Loss in mRNA abundance at 10 minutes in the presence of cordycepin (42° C.-22° C.). (FIG. 32D) Transcription factors in the top 5% of transcripts with elevated mRNA DMS reactivity after 10 minutes of 42° C. heat shock (H10m) show decreased abundance ater 10 minutes heat shock (RNA-seq analysis). FIG. 33 provides the corresponding RNA-seq beat map at other points).

FIG. 33, comprising FIG. 33A through FIG. 33B, depicts mRNAs of transcription factors with increased DMS reactivity present in the top 5% group show decreased abundance post-heat shock, as compared to the control, and show accelerated heat-induced decay. FIG. 33A depicts mRNAs of transcription factors present in the top 5% of transcripts with increased DMS reactivity aRer heat shock show obvious heat shock-induced decreases in abundance over the time-course, especially at 10 and 20 minute (H10 min and HR20 min), as compared to their abundance in the control (C10 min and C 20 min). Each expression value (Log 2(TPM)) was normalized by the average value of each row (i.e. the average expression value of that mRNA). In the heat map, blue represents low relative expression values ((Log 2TPM)actual −(Log 2TPM)average≤0) and yellow represents high relative expression ((Log 2TPM)actual (Log 2TPM)average ≥1) “HR” denotes recovery after heat shock. FIG. 33B depicts mRNA decay analysis of transcription factors that showed increased DMS reactivity under heat. In the presence of cordycepin, these transcription factors show an accelerated decrease in mRNA abundance at 10 minutes after 42° C. treatment as compared to 22° C.

FIG. 34, comprising FIG. 34A through FIG. 34B, depicts exemplary experiments demonstrating that in vitro modification of rice 5.8S rRNA by EDC analyzed by denaturing page of cDNAs after reverse transcription. (FIG. 34A) Reactions with the indicated EDC concentrations for 5 minutes. Dideoxy sequencing lanes, a control reaction lacking EDC, and reactions with EDC are shown. Blue text to the left indicates nucleotides within the sequence of the examined range of GS3 to C143. (FIG. 34B) Reactive nucleotides in either 57 mM or 85 mM EDC mapped as hexagons and circles, respectively, onto the relevant portion of the rice 5.8S rRNA comparative structure. Colors indicate the level of modification for nucleotides exceeding the calculated significance value for which a base is considered modified after normalization and scaling such that all values fall between 0 and 1.

FIG. 35 depicts in vitro modification of rice 5.8S rRNA by EDC, for a 2 minute reaction duration, and analyzed by denaturing page of cDNAs after reverse transcription. Reactions with the indicated EDC concentrations. A control reaction lacking EDC and reactions with 5.7 mM to 113 mM EDC are shown. Text to the left indicates the sequence of the examined range of G53 to C143.

FIG. 36 depicts a reaction scheme for base modification by EDC, shown in red. In the first step. EDC abstracts a proton from the endocyclic N3 of U. The resulting anionic lone pair on the nucleobase attacks the cationic carbodiimide moiety, leading to neutralization and covalent attachment of the EDC adduct to the base. EDC reacts with the endocyclic N1 of G in a similar fashion.

FIG. 37 depicts in vitro EDC modification of rice 5.8S rRNA in vitro at various pH and EDC concentrations. Denaturing PAGE analysis of cDNAs generated after reverse transcription. Reaction conditions at pH 6, pH 7, and pH 8 are shown along with dideoxy sequencing lanes.

FIG. 38, comprising FIG. 38A through FIG. 388, depicts in vitro EDC modification of rice 5.8S rRNA at various pH and EDC concentrations. (FIG. 38A) Denaturing PAGE analysis of cDNAs generated after reverse transcription. Reaction conditions at pH 7, pH 8, and pH 9.2 are shown along with dideoxy sequencing lanes. (FIG. 38B) Comparison of band intensities for all Us and Gs within the examined range of G55 to G138; reactions at 113 mM EDC are excluded due to excessive modification of the RNA. Shaded boxes represent U or G modification above the calculated significance value (S); shaded boxes represent S to 3 S; >3 S to 6 S; >6 S to 10 S: and >10 S. White boxes represent Us or Gs that are not significantly modified by EDC.

FIG. 39 depicts a cryo-EM structure of Saccharomyces cerevisiae 60S subunit (PDB: 5GAK), a homolog of rice 60S subunit, is used here as no rice ribosome structure currently exists. Shown exclusively is 5.8S rRNA. The long-range helix at left shows A45 to A48 and U104 to G107. Note that G107 is in a sheared base pair and U106 forms a wobble pair. The stem-loop from G ill to G119 is shown, with the splayed out U117 and A113. This stem-loop has an identical sequence in rice. The remainder of 5.8S rRNA is shown in transparent white.

FIG. 40, comprising FIG. 40A through FIG. 40C, depicts in vivo EDC modification of rice 5.8S rRNA analyzed by denaturing PAGE of cDNAs after reverse transcription. (FIG. 40A) Reaction conditions at buffer pH 8 with 113 mM, 283 mM, and 565 mM EDC are shown along with dideoxy sequencing lanes. (FIG. 40B) Reaction conditions at buffer pH from 6 to 9.2 and at 113 mM or 283 mM EDC are shown along with dideoxy sequencing lanes. Reactions with 113 mM EDC at buffer pH 9.2 are shown twice, in lanes 12 and 13. (FIG. 40C) Reaction conditions at buffer pH 7 and 283 mM EDC with 2 minutes, 5 minutes, and 10 minutes durations are shown along with dideoxy sequencing lanes. The sequencing lanes were run on a different portion of the same gel as the experimental lanes, as indicated by the grey brackets.

FIG. 41, comprising FIG. 41A through FIG. 41C, depicts in vitro probing of rice 5.8S rRNA by EDC to test quench conditions. (FIG. 41A) Tests of DTT and sodium acetate reaction quenches analyzed by denaturing PAGE of cDNAs after reverse transcription. The dideoxy sequencing lanes at left were run on a different part of the same gel, and the transposition of these lanes is indicated by the grey brackets. Four different quench compositions were examined: water (Q1), 2.5 mM DTT (Q2), 1 M sodium acetate. pH 5 (Q3), and a combination of 1.3 M DTT and 1 M sodium acetate, pH 5 (Q4). Times are when the quench solution was added, with 0 minutes indicating addition of the quench before adding 113 mM EDC and 5 minutes indicating addition of the quench 5 minutes after reacting total rice RNA with EDC. (FIG. 41B) Plot of normalized nucleotide reactivities against reaction time for EDC-modified nucleotides between U102 and U131. Lines represent linear fits. The bold line indicates the fit to the average reactivity for all examined nucleotides. (FIG. 41C) Test of lysis buffer composition analyzed by denaturing page of cDNAs after reverse transcription. The sequencing lanes are for ATP aptamer RNA, an RNA sequence not found in rice total RNA, which was doped into lysis buffer before RNA extraction for lanes 6, 8, 11, and 13. Lanes 5, 7, 10, and 12 do not contain ATP aptamer RNA. Lane 9, labeled NT, contains untreated ATP aptamer RNA not added to lysis buffer for which reverse transcription was done separately. Less RNA was added to the RT reaction for NT, which accounts for the lower band intensity in lane 9 compared to lanes 6, 8, 11, and 13.

FIG. 42, comprising FIG. 42A through FIG. 42D, depicts a comparison of in vivo EDC and phenylglyoxal modification of rice 5.8S and 28S rRNAs analyzed by denaturing PAGE of cDNAs after reverse transcription. (FIG. 42A) Comparison of EDC and phenylglyoxal (PG) modification of rice 5.8S rRNA under conditions where either a water wash (W) or 1 g of DTT (D) was used as a reaction quench, along with dideoxy sequencing lanes. Rice tissue not treated with reagent nor subjected to quenching is shown as NRT in lane 11. The three Gs modified by phenylglyoxal are G82, G89 and G99, while the remaining Gs were modified by both EDC and phenylglyoxal. The section from C122 to C133 was run on a different portion of the same gel. (FIG. 42B) Nucleotides reactive with phenylglyoxal or EDC mapped as hexagons or circles, respectively, onto the relevant portion of rice 5.8S rRNA comparative structure. Colors indicate the level of modification after normalization and scaling such that all values fall between 0 and 1. The quench composition (water wash or DTT; see Supplemental Information) had no effect on observed EDC reactivity. (FIG. 42C) Comparison of EDC and phenylglyoxal modification of rice 28S rRNA. Conditions are the same as in FIG. 42A. (FIG. 42D) Nucleotides reactive with EDC or phenylglyoxal mapped onto the relevant portion of rice 28S rRNA comparative structure. Red discs indicate nucleotides modified solely by EDC while cyan discs indicate nucleotides modified by both EDC and phenylglyoxal. Data between 280 and 270 are omitted as too close to the primer, which ends at 280.

FIG. 43 depicts a comparison of in vivo EDC and phenylglyoxal modification of rice 28S rRNA analyzed by denaturing PAGE of cDNAs after reverse transcription. Specified here is the range from A150 to C270. EDC and phenylglyoxal (PG) modifications under conditions where either a water wash (W) or 1 g of DTT (D) was used as a reaction quench are shown, along with dideoxy sequencing lanes. The dideoxy sequencing reactions were performed separately and run on a separate gel, as indicated by the grey brackets and asterisk in the text next to Sequencing Lanes. Rice tissue not treated with reagent nor subjected to quenching is shown as NRT. Text at left indicates the sequence of 28S rRNA. Text at right indicate nucleotides modified by EDC. G260 was modified by both EDC and phenylglyoxal. Asterisks indicate natural reverse transcription stops.

FIG. 44, comprising FIG. 44A through FIG. 44D, depicts in vivo EDC modification of E. coli 16S rRNA. (FIG. 44A) EDC concentration assays. Denaturing PAGE analysis of cDNAs generated after reverse transcription. Reactions in EDC from 28 mM to 85 mM are shown along with sequencing lanes. Text inset in the gel shows the true position of the sequence in relation to the experimental lanes, as part of the sequencing lanes were shifted by a crease in the gel. (FIG. 44B) Agarose gel analysis of rRNA extracted from E. coli after treatment with 28 mM to 113 mM EDC. (FIG. 44C) Lower EDC concentration trials. Denaturing PAGE analysis of cDNAs after reverse transcription. Reactions in EDC from 6 mM to 28 mM are shown along with sequencing lanes. Red text indicates modified nucleotides. (FIG. 44D) Nucleotides reactive with EDC mapped onto the relevant portion of E. coli 16S rRNA comparative structure. Arrows pointing to the reactive nucleotides show reactions in 17 mM, 23 mM, and 28 mM EDC in separate segments, with the 17 mM EDC segment located closest to the arrow head. The shading within each segment indicates the relative extent of modification above the significance value (S).

FIG. 45, comprising FIG. 45A through FIG. 45E, depicts a crystal structure of the Escherichia coli 70S ribosome (PDB: 4V9D) to show uracils (U) and guanines (G) within the examined range for EDC reactivity. Lack of reactivity of some Gs and Us can be explained by solvent inaccessibility and hydrogen bonding, while others can be explained by hydrogen bonding alone. (FIG. 45A) Comparison of EDC-modified and EDC-unmodified Gs and Us within 16S rRNA. In this and all subsequent panels, the examined range (1-90) within 16S rRNA is dark, the remainder of 16S rRNA is pale, Us and Gs modified by EDC (see FIG. 44) are G39, U56, G62, U84, U85 and G86, Us and Gs unmodified by EDC are G31, G38, U49 and G64. (FIG. 45B) G31 is partially buried and in position to form a hydrogen bond between its N1 and the bridging O5′ of C48. The N1G or N3U is shown as a sphere in this and subsequent panels. (FIG. 45C) U49 is also buried within the ribosome. A slice of the ribosome structure is removed to allow easy viewing. U49 forms a sugar edge interaction with G362. (FIG. 45D) G38 is in position to form a hydrogen bond between N1 and a non-bridging phosphate oxygen of A397. (FIG. 45E) G64 forms a Hoogsteen base pair with G68, which in turn forms a sheared pair with A101 (not shown),

FIG. 46 depicts an RNA structure model of the ROSE element.

FIG. 47 depicts predicted RNA structures at 22° C. and 42° C. in silico and in vivo (with DMS reactivities as restraints) of ROSE element candidates in Oryza saliva. The squares mark the SD sequence region. Structures were predicted using RNA structure.

FIG. 48 depicts an RNA structure model of the four U element.

FIG. 49 depicts predicted RNA structures at 22° C. and 42° C. in silico and in vivo (with DMS reactivities as restraints) of four U element candidates in Oryza sativa. The squares mark the SD sequence region.

FIG. 50 depicts an RNA structure model of the UCCU element.

FIG. 51 depicts predicted RNA structures at 22° C. and 42° C. in silico and in vivo (with DMS reactivities as restraints) of UCCU element candidates in Oryza saliva. The squares mark the SD sequence region.

FIG. 52 depicts RNA structure models of prfA (left) and cssA (right) RNATs. The elongated nucleotide hairpin with internal loops and bulges of the prfA RNAT is drawn schematically. The symbols at the tops of the structures represent non-obligatory parts of the RNAT.

FIG. 53 depicts predicted in silico and in vivo RNA secondary structures of the 50 nt upstream of start codon of atpH at 22° C. and 42° C. The squares mark the SD sequence.

FIG. 54, comprising FIG. 54A through FIG. 54D, depicts the distribution of free energy per nucleotide within the entire 5′UTR of HSP mRNAs and other mRNAs in the Structure-seq dataset. FIG. 54A depicts the distribution of the free energy per nucleotide within the 5′UTRs of all Oryza sativa HSP mRNAs with sufficient coverage from Strature-seq (n: 93), based on RNA structure prediction using DMS reactivities as restraints FIG. 54B depicts the distribution of the free energy per nucleotide within the 5UTRs of all HSP mRNAs (n w 168), based on structures predicted in silico. FIG. 54C depicts a comparison of the distribution of the free energy per nucleotide of the 5′UTRs of all HSP mRNAs (n=93) and all other mRNAs (n=9,875) with 5′UTR annotation and with sufficient coverage from Structure-seq. In FIG. 54A and FIG. 54B, the data for HSP90 mRNA arm marked with a purple horizontal line. In the violin plots of panels A-C, green indicates the distribution of free energy per nt of the 5′UTR of all HSP mRNAs at 22° C. dark yellow indicates the distribution of free energy per nt of the 5′UTR of all HSP mRNAs at 42° C.; blue indicates the distribution of free energy per nt of the 5′UTR of all mRNAs other than HSPs with 5′UTR annotation and with sufficient coverage from Structure-seq (n=9,875); red indicates the distribution of free energy per nt of the 5′UTR of all mRNAs other than HSPs at 42° C. with 5′UTR annotation and with sufficient coverage from Structure-seq (n 9,875). FIG. 54D depicts the predicted RNA structure of the 5′UTR of rice HSP90 in silico or with DMS reactivities as restraints at 22° C. and 42° C.

FIG. 55, comprising FIG. 35A through FIG. 55F, depicts that there was a lack of correlation between change of DMS reactivity on Kozak sequences and mRNA abundance changes (log 2) at 22° C. and 42° C. at different time points (FIG. 55A through FIG. 55E), and Ribo-seq signal change at 22° C. and 42° C. (FIG. 55F), (FIG. 55A) 10 min (FIG. 55B) 20 min (FIG. 55C) 1 hr (FIG. 55D) 2 hrs (FIG. 55E) 10 hrs (FIG. 55F) Ribo-seq 10 min.

FIG. 56, comprising FIG. 56A through FIG. 560, depicts the overrepresented sequence motifs in different mRNA classes. Overrepresented sequence motifs in the 50 nucleotides upstream of the start codon within (FIG. 56A) top group (FIG. 56B) bottom group (FIG. 56C) all mRNAs with elevated Ribo-seq signal at 42° C. based on Ribo-seq data and with 5′UTR length ≥50 nt (FIG. 56D) all mRNAs with S48 sufficient coverage from Structure-seq and with 5′UTR length ≥50 nt. Here, motifs are ranked according to the significance of overrepresentation.

FIG. 57 depicts a table demonstrating the change of DMS reactivity, mRNA abundance, and Ribo-seq signal of the identified ROSE element candidates in Oryza sativa. Reactivity difference is the difference in average DMS reactivity between 22° C. and 42° C. (from Structure-seq data); RNA abundance fold change is the fold change of mRNA abundance between 22° C. and 42° C. at each time point (from time-series RNA-seq data); Ribo-seq difference is the difference in average Ribo-seq signal between 22° C. and 42° C. (from Ribo-seq data). SD stands for the Shine-Dalgamo sequence (AGGA) and the table shows the average reactivity of the four nucleotide. “Whole” stands for the whole transcript and the table shows the average reactivity of the whole transcript. NA indicates data not available in the dataset. Asterisks mark statistically significant changes of abundance (t-test, p value <0.05).

FIG. 58 depicts a table demonstrating the change of DMS reactivity, mRNA abundance, and Ribo-seq signal of the identified candidates in Oryza sativa with four U elements. Reactivity difference is the difference in average DMS reactivity between 22° C. and 42° C. (from Structure-seq data); RNA abundance fold change is the fold change of mRNA abundance between 22° C. and 42° C. at each time point (from time-series RNA-seq data); Ribo-seq difference is the difference in average Ribo-seq signal between 22° C. and 42° C. (from Ribo-seq data). SD stands for the Shine-Dalgarno sequence (AGGA) and the table shows the average reactivity of the four nucleotides. “Whole” stands for the whole transcript and the table shows the average reactivity of the whole transcript, inf indicates infinite value (division by 0). Asterisks mark statistically significant changes of abundance (t-test, p value <0.05).

FIG. 59 depicts a table demonstrating the change of DMS reactivity, mRNA abundance, and Ribo-seq signal of identified UCCU element candidates in Oryza sativa. Reactivity difference is the difference in average DMS reactivity between 22° C. and 42° C. (from Structure-seq data); RNA abundance fold change is the fold change of mRNA abundance between 22° C. and 42° C. at each time point (from time-series RNA-seq data); Ribo-seq difference is the difference in average Ribo-seq signal between 22° C. and 42° C. (from Ribo-seq data). SD stands for the Shine-Dalgarno sequence (AGGA) and the table shows the average reactivity of the four nucleotides. “Whole” stands for the whole transcript and the table shows the average reactivity of the whole transcript. Asterisks mark statistically significant changes of abundance (t-test, p value<0.05).

### DETAILED DESCRIPTION

The present invention is based, in part, on the development of an improved method for obtaining nucleotide-resolution RNA structural information in vivo and genome-wide with increased sensitivity, improved data quality, reduced ligation bias, and improved read coverage. Accordingly, the invention provides methods of purifying and ligating nucleic acids that overcomes the nucleotide bias and inefficiencies associated with currently used protocols. In one embodiment, the methods reduce the generation of deleterious by-products. In one embodiment, the methods reduce the time and cost associated with obtaining nucleotide-resolution RNA structural information in vivo as compared to other methods in the art.

In one embodiment, the method comprises the steps, in order, of a) treating an RNA molecule in vivo with an agent which covalently modifies unprotected nucleobases, b) performing reverse transcription (RT) with a random hexamer-containing primer to generate a cDNA molecule, c) ligating a sequencing adaptor to the 3′end of the cDNA using a hairpin donor molecule, d) performing PCR amplification of the ligated construct and e) sequencing the amplified products.

In one embodiment, the method comprises the steps, in order, of a) treating an RNA molecule in vivo with dimethyl sulfate (DMS), which covalently modifies unprotected adenines and cytosines, b) performing reverse transcription (RT) with a random hexamer-containing primer to generate a cDNA molecule, c) ligating a sequencing adaptor to the 3′ end of the cDNA using a hairpin donor molecule, d) performing PCR amplification of the ligated construct and e) sequencing the amplified products.

In one embodiment, the method comprises the steps, in order, of a) treating an RNA molecule in vivo with 1-ethyl-3-(3-dimethylaminopropyl)carbodiimide (EDC), which covalently modifies unprotected uracils and guanines, b) performing reverse transcription (RT) with a random hexamer-containing primer to generate a cDNA molecule, c) ligating a sequencing adaptor to the 3′end of the cDNA using a hairpin donor molecule, d) performing PCR amplification of the ligated construct and e) sequencing the amplified products.

In one embodiment, the step of reverse transcription (step b) comprises contacting an RNA molecule with a random hexamer primer to form a RNA:primer complex, and contacting the RNA:primer complex with a reverse transcriptase and a pool of nucleotides. In one embodiment, the pool of nucleotides comprises a modified nucleotide. In one embodiment a modified nucleotide is modified to allow specific recognition or binding of the modified nucleotide after incorporation into a nucleic acid molecule. For example, in one embodiment, a nucleotide is biotinylated to allow for binding of the nucleotide to streptavidin after incorporation into a nucleic acid molecule.

In one embodiment, the method further comprises at least one purification steps. In one embodiment, a purification step is performed after reverse transcription (step b) and before ligation (step c). In one embodiment, a purification step is performed after ssDNA ligation (step c) and before performing PCR amplification (step d). In one embodiment, a purification step is performed after PCR amplification (step d) and before sequencing (step e).

In one embodiment at least one purification step comprises purifying a product using PAGE extraction. In one embodiment, the method comprises at least one, at least two, or at least three PAGE extractions. In one embodiment, the method comprises three PAGE purification steps.

In one embodiment at least one purification step comprises purifying a product using streptavidin pull down. In one embodiment, the method comprises at least one or at least two streptavidin pull down purification steps.

In one embodiment, the method comprises two streptavidin pull down purification steps and at least one PAGE purification step. In one embodiment, a streptavidin pull down purification is performed after reverse transcription (step b) and before ligation (step c), a streptavidin pull down purification is performed after ssDNA ligation (step c) and before performing PCR amplification (step d), and PAGE purification is performed after PCR amplification (step d) and before sequencing (step e).

In one embodiment, the step of ssDNA ligation (step c) comprises ligating a donor nucleic acid molecule to a purified cDNA molecule. In one embodiment, the donor molecule comprises a hairpin structure and a 3-overhang comprising a random hexamer sequence. In one embodiment, the donor molecule comprises a sequence as set forth in SEQ ID NO:1. In one embodiment, the ligation between the cDNA molecule and the donor molecule is accomplished through the actions of a ligase. In one embodiment, the ligase is a T4 DNA ligase. Generally, the donor molecule hybridizes with a cDNA 3′-end to yield the desired ligation product (e.g., a hybrid molecule comprising the cDNA and donor molecule).

In one embodiment, the step of PCR amplification (step d) is performed using a) a forward primer comprising at least one of a sequence for use as a sequencing adapter and a sequence complementary to the sequence of the hairpin region of the donor molecule, and b) a reverse primer comprising a sequence for use as sequencing barcode and a sequence complementary to a sequence of the random hexamer primer used for step b. In one embodiment, the forward primer has a sequence as set forth in SEQ ID NO:3, and the reverse primer has a sequence as set forth in SEQ ID NO:4.

In one embodiment, the step of sequencing (step e) is performed using a sequencing primer having a 3′ end which is complementary to the 5′ end of the donor molecule, such that the primer abuts the unique region of the cDNA molecule to be sequenced. In one embodiment the sequencing primer has a sequence of

(SEQ ID NO: 5)
TCTTCCGATCTTGAACAGCGACTAGGCTCTTCA.

In one embodiment the invention relates to kits for use in the methods of the invention. For example, in one embodiment, the kit comprises at least one of a random hexamer RT primer, a hairpin donor molecule, a forward and reverse PCR primer, and a custom sequencing primer for use in the methods of the invention.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Ampliftication” refers to any means by which a polynucleotide sequence is copied and thus expanded into a larger number of polynucleotide molecules, e.g., by reverse transcription, polymerase chain reaction, and ligase chain reaction, among others. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

Herein, the term “barcode” refers to a sequence that can or will be used to group nucleic acid molecules. The present invention provides for attaching a barcode sequence to a nucleic acid of interest, such as a naturally occurring or a synthetically derived nucleic acids. For example, sequences that undergo randomly primed synthesis in the proximity of a particular surface can or will be physically attached to the sequence of a barcode or to the sequences of a barcode set, as defined below.

The term “barcode set” refers to one or more barcodes that contain sequence features that distinguish them as distinct from other barcode sets. A barcode set can contain unrelated sequences, or sequences that are in some manner related, such as sequences in which there are errors or intentional differences introduced during their synthesis. As a non-limiting example, each barcode in a barcode set can have a sequence such as XRRXXX, in which X indicates a defined nucleotide, such as guanine (G), adenine (A), thymine (T), cytosine (C), uracil (U), and inosine (I), or other nucleotide, and R indicates any purine nucleotide. These nucleotides will be referred to by their single letter codes, G, A, T, C, U, and I, throughout.

“Binding” is used herein to mean that a first moiety interacts with a second moiety.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Denaturing” or “denaturation of” a complex comprising two polynucleotides (such as a first primer extension product and a second primer extension product) refers to dissociation of two hybridized polynucleotide sequences in the complex. The dissociation may involve a portion or the whole of each polynucleotide. Thus, denaturing or denaturation of a complex comprising two polynucleotides can result in complete dissociation (thus generating two single stranded polynucleotides), or partial dissociation (thus generating a mixture of single stranded and hybridized portions in a previously double stranded region of the complex).

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Identical” or “identity” as used herein, refer to comparisons among amino acid and nucleic acid sequences. When referring to nucleic acid molecules, “identity,” or “percent identical” refers to the percent of the nucleotides of the subject nucleic acid sequence that have been matched to identical nucleotides by a sequence analysis program. Identity can be readily calculated by known methods. Nucleic acid sequences and amino acid sequences can be compared using computer programs that align the similar sequences of the nucleic or amino acids and thus define the differences. In preferred methodologies, the BLAST programs (NCBI) and parameters used therein are employed, and the ExPaSy is used to align sequence fragments of genomic DNA sequences. However, equivalent alignment assessments can be obtained through the use of any standard alignment software.

“Hybridization probes” are oligonucleotides capable of binding in a base-specific manner to a complementary strand of nucleic acid. Such probes include peptide nucleic acids, as described in Nielsen et al., 1991, Science 254, 1497.1500, and other nucleic acid analogs and nucleic acid mimetics. See U.S. Pat. No. 6,156,501.

The term “hybridization” refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example, two complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature and the concentration of salt in the reaction. Hybridizations are usually performed under stringent conditions, for example, at a salt concentration or no more than 1 M and a temperature of at least 25° C. For example, conditions of 5×SSPE (750 mM NaCl, 50 mM Na Phosphate, 5 mM EDTA, pH 7.4) or 100 mM MES, 1 M Na, 20 mM EDTA, 0.01% Tween-20 and a temperature of 25-50° C. are suitable for allele-specific probe hybridizations. In a particularly preferred embodiment, hybridizations are performed at 40-50° C. Acetylated BSA and herring sperm DNA may be added to hybridization reactions. Hybridization conditions suitable for microarrays are described in the Gene Expression Technical Manual and the GeneChip Mapping Assay Manual available from Affymetrix (Santa Clara, Calif.).

A first oligonucleotide anneals with a second oligonucleotide with “high stringency” if the two oligonucleotides anneal under conditions whereby only oligonucleotides which are at least about 75%, and preferably at least about 90% or at least about 95%, complementary anneal with one another. The stringency of conditions used to anneal two oligonucleotides is a function of, among other factors, temperature, ionic strength of the annealing medium, the incubation period, the length of the oligonucleotides, the G-C content of the oligonucleotides, and the expected degree of non-homology between the two oligonucleotides, if known. Methods of adjusting the stringency of annealing conditions are known (see, e.g. Sambrook et al., 2012, Molecular Cloning: A Laboratory Manual, Cold Spring Harbor Laboratory Press. Cold Spring Harbor, N.Y.).

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

An “isolated nucleic acid” refers to a nucleic acid (or a segment or fragment thereof) which has been separated from sequences which flank it in a naturally occurring state, e.g., a RNA fragment which has been removed from the sequences which are normally adjacent to the fragment. The term also applies to nucleic acids which have been substantially purified from other components which naturally accompany the nucleic acid, e.g., RNA or DNA or proteins, which naturally accompany it in the cell. The term therefore includes, for example, purified genomic or transcriptomic cellular content.

The term “label” as used herein refers to a luminescent label, a light scattering label or a radioactive label. Fluorescent labels include, but are not limited to, the commercially available fluorescein phosphoramidites such as Fluoreprime (Pharmacia), Fluoredite (Millipore) and FAM (ABI). See U.S. Pat. No. 6,287,778.

As used herein, the term “ligation agent” can comprise any number of enzymatic or non-enzymatic reagents. For example, ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Afu ligase. Taq ligase, Tfl ligase, Tth ligase. Tth HB8 ligase, Thermus species AK16D ligase and Pfu ligase (see for example Published P.C.T. Application WO00/26381, Wu et al., Gene, 76(2):245-254. (1989), Luo et al., Nucleic Acids Research, 24(15): 3071-3078 (1996). The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits. Further, reversibly inactivated enzymes (see for example U.S. Pat. No. 5,773,258) can be employed in some embodiments of the present teachings. Chemical ligation agents include, without limitation, activating, condensing, and reducing agents, such as carbodiimide, cyanogen bromide (BrCN), N-cyanoimidazole, imidazole, 1-methylimidazole/carbodiimidelcystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09(1992); Sievers and von Kiedrowski, Nature 369:221-24 (1994); Liu and Taylor, Nucleic Acids Res. 26:3300-04 (1999); Wang and Koot, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al FEBS Letters 232:153-55 (1988); Naylor and Gilham, Biochemisty 5:2722-28 (1966); and U.S. Pat. No. 5,476,930.

As used herein, the term “nucleic acid” refers to both naturally-occurring molecules such as DNA and RNA, but also various derivatives and analogs, Generally, the probes, hairpin linkers, and target polynucleotides of the present teachings are nucleic acids, and typically comprise DNA. Additional derivatives and analogs can be employed as will be appreciated by one having ordinary skill in the art.

The term “nucleotide base”, as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6 delta 2-isopentenyladenine (6iA), N6-delta 2-isopentenyl-2-methylthioadenine (2 ms6iA) N2-dimethylguanine (dmG), 7methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-rhiouracil, 06-methylguanine, N6-methyladenine, 04-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology. pp. 385-394, CRC Press, Boca Raton, Fla., and the references cited therein.

The term “nucleotide”, as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR2 or halogen groups, where each R is independently H, C1-C6 alkyl or C5-C14 aryl. Exemplary riboses include, but are not limited to, 2′4C1-C6)alkoxyribose, 2′-(C5-C14)aryloxyribose, 2′,3′-didehydroribose, 2′-deoxy-3′-haloribose, 2′-deoxy-3-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4% anomeric nucleotides, 1′-anomeric nucleotides, 2′-4′- and 3′-4′-linked and other “locked” or “LNA”, bicyclic sugar modifications (see, e.g., PCT published application nos. WO 98/22489, WO 98/39352; and WO 99/14226). The term “nucleic acid” typically refers to large polynucleotides.

The term “oligonucleotide” typically refers to short polynucleotides, generally, no greater than about S nucleotides. It will be understood that when a nucleotide sequence is represented by a DNA sequence (i.e., A, T. G, C), this also includes an RNA sequence (i.e., A, U, G, C) in which “U” replaces “T.”

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning and amplification technology, and the like, and by synthetic means. An “oligonucleotide” as used herein refers to a short polynucleotide, typically less than 100 bases in length.

Conventional notation is used herein to describe polynucleotide sequences: the left-hand end of a single-stranded polynucleotide sequence is the 5′-end. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.” In the sequences described herein:

A=adenine,

G=guanine,

T=thymine,

C=cytosine,

U=uracil,

H=A, C or T/U,

R=A or G,

M=A or C,

K=G or T/U,

S=G or C,

Y=C or T/U,

W=A or T/U,

B=G or C or T/U,

D=A or G, or T/U,

V=A or G or C.

N=A or G or C or TAU.

The skilled artisan will understand that all nucleic acid sequences set forth herein throughout in their forward orientation, are also useful in the compositions and methods of the invention in their reverse orientation, as well as in their forward and reverse complementary orientation, and are described herein as well as if they were explicitly set forth herein.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, e.g., in the presence of nucleotides, a complementary polynucleotide template, and an agent for polymerization such as DNA polymerase. A primer is typically single-stranded, but may be double-stranded, Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with a detectable label, e.g., chromogenic, radioactive, or fluorescent moieties and used as detectable moieties. Examples of fluorescent moieties include, but are not limited to, rare earth chelates (europium chelates), Texas Red. rhodamine, fluorescein, dansyl, phycocrytherin, phycocyanin, spectrum orange, spectrum green, and/or derivatives of any one or more of the above. Other detectable moieties include digoxigenin and biotin.

A “random primer,” as used herein, is a primer that comprises a sequence that is designed not necessarily based on a particular or specific sequence in a sample, but rather is based on a statistical expectation (or an empirical observation) that the sequence of the random primer is hybridizable (under a given set of conditions) to one or more sequences in the sample. The sequence of a random primer (or its complement) may or may not be naturally-occurring, or may or may not be present in a pool of sequences in a sample of interest. The amplification of a plurality of nucleic acid species in a single reaction mixture would generally, but not necessarily, employ a multiplicity of random primers. As is well understood in the art, a “random primer” can also refer to a primer that is a member of a population of primers (a plurality of random primers) which collectively are designed to hybridize to a desired and/or a significant number of target sequences. A random primer may hybridize at a plurality of sites on a nucleic acid sequence. The use of random primers provides a method for generating primer extension products complementary to a target polynucleotide which does not require prior knowledge of the exact sequence of the target. Conventional notation is used herein to describe polynucleotide sequences: the left-hand end of a single-stranded polynucleotide sequence is the 5-end; the left-hand direction of a double-stranded polynucleotide sequence is referred to as the 5′-direction.

A “restriction site” is a portion of a double-stranded nucleic acid which is recognized by a restriction endonuclease. A portion of a double-stranded nucleic acid is “recognized” by a restriction endonuclease if the endonuclease is capable of cleaving both strands of the nucleic acid at a specific location in the portion when the nucleic acid and the endonuclease are contacted. Restriction endonucleases, their cognate recognition sites and cleavage sites are well known in the art. See, for instance, Roberts et al., 2005, Nucleic Acids Research 33:D230-D232.

A “sequence read” corresponds to a determination of the nucleotides in a target nucleic acid molecule in the order in which they occur and can or will include only a part of the target molecule, and can or will exclude other parts of the target molecule. The sequencing read in this context does not necessarily correspond to a fixed length. Current sequencing methods can produce reads of various lengths. Some sequencing methods, including but not limited to those that use physical separation of molecules of different sizes, can or will produce sequence reads ranging from one nucleotide to more than a thousand nucleotides. Alternatively, some sequencing methods produce shorter reads consisting of 1 to 50 nucleotides, 1 to 100 nucleotides, 1 to 200 nucleotides and longer, and the possible lengths may increase as technology improves.

The term “sequence” refers to the sequential order of nucleotides in a nucleic acid molecule, or, depending on context, refers to a molecule or part of a molecule in which a particular sequential order of nucleotides exists.

The term “transcript” refers to a length of RNA or DNA that has been transcribed respectively from a DNA or RNA template.

“Transcriptomics” as used herein refers to the study of any transcript molecule, which includes all types of RNA such as messenger RNA, ribosomal RNA, transfer RNA, and non-coding RNAs present in a sample, cell, or population of cells.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention is based, in part, on the development of improved methods for investigating RNA structure in vivo. RNA molecules that can be investigated using the methods of the invention include, but are not limited to mRNA, rRNA, noncodingRNA (ncRNA), large ncRNA (lncRNA), small nuclear RNA (snRNA), small cytoplasmic RNA (scRNA), small nucleolar RNA (snoRNA), small interfering RNA (siRNA) and microRNA (miRNA) molecules. The RNA molecules can be naturally occurring (e.g., transcriptomic RNA molecules), synthetic RNA molecules (e.g., recombinant RNA molecules), or transcripts made from naturally occurring or recombinant DNA molecules.

In one embodiment, the method comprises the steps, in order, of a) treating an RNA molecule in vivo with an agent, which covalently modifies unprotected nucleobases, b) performing reverse transcription (RT) with a random hexamer-containing primer to generate a cDNA molecule, c) ligating a sequencing adaptor to the 3′ end of the cDNA using a hairpin donor molecule, d) performing PCR amplification of the ligated construct and e) sequencing the amplified products.

Agents which covalently modify unprotected nucleobases include, but are not limited to, dimethyl sulfate (DMS), glyoxal, methylglyoxal, phenylglyoxal, i-cyclohexyl-3-(2-morpholinoethyl)-carbodiimide methyl-p-toluenesulfonate (CMCT), nicotinoyl azide (NAz) or 1-ethyl-3-(3-dimethylaminopropyl)carbodiimide (EDC), and SHAPE (Selective Hydroxyl Acylation analyzed by Primer Extension) reagents that react with the 2′ hydroxyl, including, but not limited to, 1M7 (1-methyl-7-nitroisatoic anhydride), 1M6 (1-methyl-6-nitroisatoic anhydride), NMIA (N-methyl-isatoic anhydride), FAI (2-methyl-3-furoic acid imidazolide), NAI (2-methylnicotinic acid imidazolide), and NA1-N3 (2-(azidomethyl)nicotinic acid acyl imidazole).

In one embodiment, the method comprises the steps, in order, of a) treating an RNA molecule in vivo with DMS, which covalently modifies unprotected adenines and cytosines, b) performing reverse transcription (RT) with a random hexamer-containing primer to generate a cDNA molecule, c) ligating a sequencing adaptor to the 3′end of the cDNA using a hairpin donor molecule, d) performing PCR amplification of the ligated construct and e) sequencing the amplified products.

In one embodiment, the method comprises the steps, in order, of a) treating an RNA molecule in vivo with EDC, which covalently modifies unprotected uracils and guanines, b) performing reverse transcription (RT) with a random hexamer-containing primer to generate a cDNA molecule, c) ligating a sequencing adaptor to the 3′ end of the cDNA using a hairpin donor molecule, d) performing PCR amplification of the ligated construct and e) sequencing the amplified products.

### Treatment of RNA

In one embodiment, the RNA molecules for investigation, or a portion of the RNA molecules for investigation, using the methods of the invention are treated prior to analysis. In one embodiment, the treatment comprises treatment with dimethyl sulfate (DMS). Such a treatment is useful, for example, for modification of impaired adenosine and cytidine nucleotides for structural analysis of RNA molecules. Alternatively, in one embodiment, the method is useful for structural analysis of an RNA-protein complex. Therefore, in one embodiment, the method of the invention comprises obtaining an RNA sample, treating at least a portion of the sample with DMS, and analyzing both the treated and untreated samples using the methods of the invention, and determining the structure of the RNA molecule based on the comparison of the sequence of the treated RNA to that of the untreated RNA.

Generation of cDNA

The method of the invention includes a step of generating a cDNA molecule from an RNA molecule. Methods for generating cDNA from RNA are generally known in the art in one embodiment, the method includes hybridizing a DNA primer to a target RNA molecule and extending the primer using a reverse transcription (RT) polymerase. In one embodiment, the method comprises hybridizing a mixed population of DNA primers wherein the DNA primers comprise a random hexamer sequence, to a pool of multiple RNA molecules. In one embodiment, a random hexamer primer has a sequence ofCAGACGTGTGCTCTTCCGATCNNNNNN (SEQ ID NO:6). Such an embodiment allows reverse transcription of multiple RNA molecules in a single reaction.

RT according to the present invention may be performed by contacting the target nucleic acid with an RT solution comprising all the necessary reagents for RT. Then, RT may be accomplished by exposing the mixture to any suitable denaturing, polymerase annealing and polymerase extension regimen known in the art. In one embodiment, the RT solution comprises at least one modified nucleotide, such that a modified nucleotide is incorporated into the cDNA product formed from RT of the target RNA molecule(s). For example, in one embodiment, the modified nucleotide is biotinylated, allowing for capture and purification of the cDNA molecules using streptavidin affinity purification methods.

### Ligation

The method of the invention includes a step of ligating single stranded nucleic acids. “Ligation” refers to the joining of a 5′-phosphorylated end of one nucleic acid molecule to a 3′-hydroxyl end of the same or another nucleic acid molecule by an enzyme called a “ligase.” Alternatively, in some embodiments of the invention, ligation is effected by a type I topoisomerase moiety attached to one end of a nucleic acid (see U.S. Pat. No. 5,766,891, incorporated herein by reference). The terms “ligating,” “ligation,” and “ligase” are often used in a general sense herein and are meant to comprise any suitable method and composition for joining a 5′-end of one nucleic acid to a 3′-end of the same or another nucleic acid.

In addition, ligation can be mediated by chemical agents. Chemical ligation agents include, without limitation, activating, condensing, and reducing agents, such as carbodiimide, cyanogen bromide (BrCN), N-cyanoimidazole, imidazole, I-methylimidazole/carbodiimide/cystamine, dithiothreitol (DTT) and ultraviolet light, Autoligation, i.e., spontaneous ligation in the absence of a ligating agent, is also within the scope of the teachings herein. Detailed protocols for chemical ligation methods and descriptions of appropriate reactive groups can be found in, among other places, Xu et al, Nucleic Acid Res., 27:875-81 (1999); Gryaznov and Letsinger, Nucleic Acid Res. 21:1403-08 (1993); Gryaznov et al., Nucleic Acid Res. 22:2366-69(1994); Kanaya and Yanagawa, Biochemistry 25:7423-30 (1986); Luebke and Dervan, Nucleic Acids Res. 20:3005-09(1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Punmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

In general, if a nucleic acid to be ligated comprises RNA, a ligase such as, but not limited to, T4 RNA ligase, a ribozyme or deoxyribozyme ligase, Tsc RNA Ligase (Prokaria Ltd., Reykjavik. Iceland), or another ligase can be used for non-homologous joining of the ends. T4 DNA ligase can be used to ligate DNA molecules, and can also be used to ligate RNA molecules when a 5′-phosphoryl end is adjacent to a 3′-hydroxyl end annealed to a complementary sequence (e.g., see U.S. Pat. No. 5,807,674 of Tyagi).

If the nucleic acids to be joined comprise DNA and the 5′-phosphorylated and the 3′-hydroxyl ends are ligated when the ends are annealed to a complementary DNA so that the ends are adjacent (such as, when a “ligation splint” is used), then enzymes such as, but not limited to, T4 DNA ligase, Ampligase™ DNA Ligase (Epicentre Technologies. Madison, Wis. USA), Tth DNA ligase, T DNA ligase, or Tsc DNA Ligase (Prokaria Ltd., Reykjavik, Iceland) can be used. However, the invention is not limited to the use of a particular ligase and any suitable ligase can be used. Still further, Faruqui discloses in U.S. Pat. No. 6,368,801 that T4 RNA ligase can efficiently ligate DNA ends of nucleic acids that are adjacent to each other when hybridized to an RNA strand. Thus, T4 RNA ligase is a suitable ligase of the invention in embodiments in which DNA ends are ligated on a ligation splint oligonucleotide comprising RNA or modified RNA, such as, but not limited to modified RNA that contains 2′-F-dCTP and 2′-F-dUTP made using the DuraScribe™ T7 Transcription Kit (Epicentre Technologies, Madison. Wis. USA) or the N4 mini-vRNAP Y678F mutant enzyme described herein. With respect to ligation on a homologous ligation template, especially ligation using a “ligation splint” or a “ligation splint oligonucleotide” (as discussed elsewhere herein), a region, portion, or sequence that is “adjacent” to another sequence directly abuts that region, portion, or sequence.

In some embodiments, a gap of at least one nucleotide is present in the unligated hybrid molecule of the invention that comprises a donor molecule and an acceptor molecule. In some embodiments, the gap is filled in by a polymerase, and the resulting product ligated. Several modifying enzymes are utilized for the nick repair step, including but not limited to polymerases, ligases, and kinases. DNA polymerases that can be used in the methods of the invention include, for example, E. coli DNA polymerase I, Thermoanaerobacter themohydrosrulfuricus polymerase 1, and bacteriophage phi 29. In a preferred embodiment, the ligase is T4 DNA ligase and the kinase is T4 polynucleotide kinase.

In one embodiment, ligation of the donor and acceptor molecule involves contacting the hybridized molecules with a ligase under conditions that allow for ligation between any two terminal regions of the molecules whose 3′ and 5′ ends after hybridization are positioned in a way that ligation may occur.

Any DNA ligase is suitable for use in the ligation step. Preferred ligases are those that preferentially form phosphodiester bonds at nicks in double-stranded DNA. That is, ligases that fail to ligate the free ends of free single-stranded DNA at a significant rate are preferred. In some instances, thermostable ligases can be used. In other instances, thermosensitive ligases are preferred because the ligase can be heat inactivated. Many suitable ligases are known, such as T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE™ (Kalin et al. Mutat. Res., 283(2): 119-123 (1992); Winn-Deen et al., Mol Cell Probes (England) 7(3):179-186(1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothernius marima DNA ligase (Thorbjarnardottir et al., Gene 151:177-180 (1995)). T4 DNA ligase is preferred for ligations involving RNA target sequences due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill., Nov. 3-7, 1995)).

In one embodiment, the ligation method comprises: a) contacting a single stranded acceptor nucleic acid molecule with a donor nucleic acid molecule wherein the donor nucleic acid molecule comprises one or more nucleic acids having a double stranded region and a single stranded 3′ terminal region; b) hybridizing the single stranded 3′ terminal region of the donor nucleic acid molecule to the acceptor molecule thereby forming an acceptor-donor hybrid molecule comprising a nick or gap between the acceptor nucleic acid and donor nucleic acid molecule; c) and ligating one 5′ end of the donor nucleic acid molecule to the 3′ end of the acceptor nucleic acid molecule.

The present invention makes use of a hybridization-based strategy whereby a donor hairpin oligonucleotide is used to hybridize with an acceptor molecule (e.g., a cDNA molecule) that is fast, efficient, and has a low-sequence bias. In one embodiment, the acceptor molecule can be a cDNA molecule generated through RT, whereas the donor molecule is designed to form a hairpin structure and further produces a single stranded 3′-overhang region such that the overhang on the donor molecule is able to hybridize to nucleotides present in the 3′ end of the acceptor molecule. In one embodiment, the hairpin donor molecule comprises a random hexamer region in the 3% overhang region such that random hexamers are positioned immediately adjacent to the hairpin-forming sequence. In one embodiment, the donor molecule comprises a sequence as set forth in SEQ ID NO:1.

In one embodiment, the acceptor molecule comprises a hydroxyl group at its 3′-terminus and the donor molecule comprises a phosphate at its 5′-end. In this manner, the 5′-end of the donor molecule ligates with the 3′-terminal nucleotide of the acceptor molecule to yield the desired ligation product.

In one embodiment, the donor molecule of the invention comprises a double stranded region and a single stranded region. In one embodiment, the single stranded region is found at the 3′ end of the donor molecule. In one embodiment, the random hexamer sequence of the single stranded region is at least partially complementary to a sequence found on an acceptor molecule of the invention. This complementary sequence found in the donor molecule allows for the hybridization between the acceptor and donor molecules of the invention.

3′ Overhang

In one embodiment, the 3′-overhang region of the donor molecule comprises nucleotides that hybridize to nucleotides found in the 3′ end of the acceptor molecule such that the hybridization between the acceptor molecule and the donor molecule forms a complex that can be ligated by either enzymatic or chemical means.

In one embodiment, the 3′-overhang region comprises at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 20 nucleotides, at least 25 nucleotides, at least 30 nucleotides, at least 35 nucleotides, or at least 40 nucleotides that are complementary to sequences found in the acceptor molecule when the acceptor and donor molecules are hybridized to one another. In this manner, the 3′-overhang region of the donor molecule is considered as the region of the donor molecule that binds to the 3′ region of the acceptor molecule.

In various embodiments, the 3′-overhang region comprises at least 1 nucleotide, preferably at least 2 nucleotides, preferably at least 3 nucleotides, preferably at least 4 nucleotides, and preferably at least 5 nucleotides that are mismatched with nucleotides found in the acceptor molecule when the acceptor and donor molecules are hybridized to one another.

In one embodiment, the hybridization between the acceptor molecule and the donor molecule forms a structure that comprises a “nick” wherein the nick can be ligated by either enzymatic or chemical means. A nick in a strand is a break in the phosphodiester bond between two nucleotides in the backbone in one of the strands of a duplex between a sense and an antisense strand.

In another embodiment, the hybridization between the acceptor molecule and the donor molecule forms a structure that comprises a “gap” wherein the gap can be ligated by either enzymatic or chemical means. A gap in a strand is a break between two nucleotides in the single strand.

In one embodiment, the hybridization between the acceptor molecule and the donor molecule forms a structure that is stable at temperatures that is as high as 35° C., as high as 40° C. as high as 45° C., as high as 50° C., as high as 55° C., as high as 60° C., as high as 65° C., as high as 70° C. as high as 75°, as high as 80° C., as high as 85° C., or more.

### Amplification

In one embodiment, the method of the invention comprises at least one amplification step wherein the copy number of a target or template nucleic acid molecule is increased. In one embodiment, the target or template nucleic acid molecule is a ligation product. The ligation product or otherwise the template nucleic acid may be amplified by any suitable method. Such methods include, but are not limited to polymerase chain reaction (PCR), reverse transcription, ligase chain reaction, loop mediated isothermal amplification, multiple displacement amplification, and nucleic acid sequence based amplification. In one embodiment, an amplification product is generated during sequencing, for example by a polymerase enzyme during single-molecule sequencing.

In one embodiment, DNA amplification is performed by PCR. To briefly summarize PCR, nucleic acid primer, complementary to opposite strands of a nucleic acid amplification target sequence, are permitted to anneal to the target. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag. In one embodiment, the detection of the duplex is done using at least one primer directed to the target nucleic acid. In yet another embodiment of PCR, the detection of the hybridized duplex comprises electrophoretic gel separation followed by dye-based visualization.

Nucleic acid amplification procedures by PCR are well known and are described in U.S. Pat. No. 4,683,202, Briefly, the primers anneal to the target nucleic acid at sites distinct from one another and in an opposite orientation. A primer annealed to the target sequence is extended by the enzymatic action of a heat stable polymerase. The extension product is then denatured from the target sequence by heating, and the process is repeated. Successive cycling of this procedure on both strands provides exponential amplification of the region flanked by the primers.

PCR according to the present invention may be performed by contacting the target nucleic acid with a PCR solution comprising all the necessary reagents for PCR. Then, PCR may be accomplished by exposing the mixture to any suitable thermocycling regimen known in the art. In a preferred embodiment, 30 to 50 cycles, preferably about 40 cycles, of amplification are performed. It is desirable, but not necessary, that following the amplification procedure there be one or more hybridization and extension cycles following the cycles of amplification. In a preferred embodiment, 10 to 30 cycles, preferably about 25 cycles, of hybridization and extension are performed (e.g., as described in the examples).

In particular embodiments of the invention the polymerase used for PCR is a polymerase from a thermophile organism or a thermostable polymerase or is selected from the group consisting of Thermus thermophilus (Tth) DNA polymerase, Thermus aquaticus (Taq) DNA polymerase, Thermotoga maritima (Tma) DNA polymerase, Thermococcus litoralis (Tli) DNA polymerase, Pyrococcus furiosus (Pfu) DNA polymerase, Pyrococcus woesei (Pwo) DNA polymerase, Pyrococcus kodakaraensis KOD DNA polymerase, Thermus filiformis (Tfl) DNA polymerase, Sulfolobus solfataricus Dpo4 DNA polymerase, Thermus pacificus (Tpac) DNA polymerase, Thermus eggerissonii (Teg) DNA polymerase, Thermus brockianus (Tbr) and Thermus flavus (Tfl) DNA polymerase. In one embodiment, the polymerase used for PCR is a modified polymerase designed to have increased fidelity as compared to its unmodified counterpart. High-fidelity polymerases that may be used in the methods of the invention include, but are not limited to, Q5®, Phusion®, PrimeSTAR® GXL, Platinum™ Taq, and MyTaq™ DNA polymerases.

In one embodiment, a target or template nucleic acid molecule is isolated or amplified using primers having a sequence that is capable of hybridizing to the template. In one embodiment, the template nucleic acid molecule is a ligated product formed from ligation of a donor hairpin molecule to a cDNA molecule. In one embodiment, the primers comprise a sequence that is capable of hybridizing to the hairpin forming region of the hairpin forming region of the donor molecule. In one embodiment, one or more primers further comprise an additional sequence that does not hybridize to the target molecule to be amplified (e.g., a sequence to be used as an adaptor for sequencing or a barcode). In one embodiment, the amplification is performed using a forward and reverse primer as set forth in SEQ ID NO:3 and SEQ ID NO:4 respectively.

In one embodiment, amplification using primers containing a random hexamer sequence results in the primers hybridizing together and amplification of the primer pair to form an undesired primer dimer product. In one embodiment the products that result from the PCR amplification process are purified to remove primer dimer products. In one embodiment, the purification is performed using PAGE extraction. In one exemplary embodiment, products in the range of 220 nt to 600 nt are extracted using PAGE extraction to purify the amplified template away from primer dimers formed from during amplification using the primers as set forth in SEQ ID NO:3 and SEQ ID NO:4.

### Sequencing

In some embodiments, the methods of the invention include methods of sequencing an isolated nucleic acid. In one embodiment, the nucleic acid may be prepared (e.g., library preparation) for massively parallel sequencing in any manner as would be understood by those having ordinary skill in the art. Current methods for library preparation attempt to uniformly sample all sequences across every nucleic acid molecule, optimally with sufficient overlap to allow reassembly of the sequences from which they derive, or alternatively, to allow inference of the sequence by alignment with reference sequences. These methods are generally known in the art and generally relate to generating multiple copies of (amplifying) the complementary sequence of the nucleic acid sequences of interest. These standard methods have in common that the libraries of sequences that they contain correspond to the sequences of genes, or in various embodiments, from the messenger RNAs (i.e., mRNAs) transcribed from genes. In one embodiment, the libraries include RNA sequences from DNA regions that are not necessarily considered to be genes, including but not limited to microRNAs, short interfering RNAs, long non-coding RNAs, and others.

While there are many variations of library preparation, the purpose is to construct nucleic acid fragments of a suitable size for a sequencing instrument and to modify the ends of the sample nucleic acid to work with the chemistry of a selected sequencing process. Depending on application, nucleic acid fragments may be generated having a length of about 100-1000 bases. It should be appreciated that the present invention can accommodate any nucleic acid fragment size range that can be generated by a sequencer. This can be achieved by capping the ends of the fragments with nucleic acid adapters. These adapters have multiple roles: first to allow attachment of the specimen strands to a substrate (bead or slide) and second have nucleic acid sequence that can be used to initiate the sequencing reaction (priming). In many cases, these adapters also contain unique sequences (bar-coding) that allow for identification of individual samples in a multiplexed run. The key component of this attachment process is that only one nucleic acid fragment is attached to a bead or location on a slide. This single fragment can then be amplified, such as by a PCR reaction, to generate hundreds of identical copies of itself in a clustered region (bead or slide location).

One aspect of the present invention provides for methods to attach barcodes to nucleic acid molecules by primed synthesis in which the barcode is attached to the randomized or partially randomized primer, and the subsequent preparation of the resulting barcoded nucleic acid molecules for sequencing. The invention provides in part for grouping the nucleic acid molecules with attached barcodes and inferring or deducing the sequences of the single sample from which they derive.

In one embodiment, clusters of identical nucleic acid molecules form a product that is sequenced. The sequencing can be performed using any standard sequencing method or platform, as would be understood by those having ordinary skill in the an. Representative sequencing methods that can be used in the method of the invention include, but are not limited to direct manual sequencing (Church and Gilbert, 1988, Proc Nat Acad Sci U.S.A, 81:1991-1995; Sanger et al., 1977, Proc Natl Acad Sci U.S.A., 74:5463-5467; Beavis et al. U.S. Pat. No. 5,288,644): automated fluorescent sequencing: single-stranded conformation polymorphism assays (SSCP); clamped denaturing gel electrophoresis (CDGE); denaturing gradient gel electrophoresis (DGGE) (Sheffield et al., 1981, Proc Nat Acad Sci U.S.A., 86:232-236), mobility shift analysis (Orita et al., 1989, Proc Natl Acad Sci U.S.A., 86:2766.2770; Rosenbaum and Reissner, 1987, Biophys. Chem, 265:1275; Keen et al., 1991, Trends Genet, 7:5); RNase protection assays (Myers, et al., 1985, Science, 230:1242); Luminex xMAP™ technology; HTS (Gundry and Vijg, 2011, Mutat Res, doi:10.1016/j.mrfmmm.2011.10.001); NGS (Voelkerding et al., 2009, Clinical Chemistry, 55:641-658; Su et al., 2011, Expert Rev Mol Diagn, 11:333-343; Ji and Myllykangas, 2011, Biotechnol Genet Eng Rev, 27:135-158); and/or ion semiconductor sequencing (Rusk, 2011. Nature Methods, doi:10.1038/nmeth.f.330; Rothberg et al., 2011, Nature, 475:348-352). Next-gen sequencing platforms including, but not limited to, Illumina HiSeq, Illumina MiSeq, Life Technologies PGM, Pacific biosciences RSII and Helicos Heliscope can be used in the method of the invention for sequencing the nucleic acid molecules. These and other methods, alone or in combination, can be used to detect and quantify at least one nucleic acid molecule of interest.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ⁼P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

The invention also provides methods which employ (usually, analyze) the products of the methods of the invention, such as preparation of libraries (including cDNA and differential expression libraries); sequencing, detection of sequence alteration(s) (e.g., genotyping or nucleic acid mutation detection); determining presence or absence of a sequence of interest; gene expression profiling; differential amplification; preparation of an immobilized nucleic acid (which can be a nucleic acid immobilized on a microarray), and characterizing (including detecting and/or quantifying) mutations in nucleic acid products generated by the methods of the invention.

Methods of analyzing the sequencing reads may include the use of bioinformatics methods for filtering, aligning, and characterizing sequencing reads. Such bioinformatics methods may include, but are not limited to, filtering of sequencing reads for unique sequences, trimming of sequencing reads (e.g., to remove sequencing adaptor sequences or low quality bases), filtering of sequencing reads for reads greater than a minimum length, generation of contigs and alignment of sequencing reads to a reference genome.

### Purification

The methods of the present invention include at least one, at least 2, or at least 3 purification steps to improve the yield of desired product and remove unwanted bi-products that can accumulate at different stages. One or more purification steps can be performed, for example, after reverse transcription and before ligation to remove excess RT primers. One or more purification steps can be performed, for example, after ssDNA ligation and before performing PCR amplification to remove excess hairpin donor molecules. One or more purification steps can be performed, for example, after PCR amplification and before sequencing to remove primer dimers.

Multiple methods of purification and size selection of nucleic acid molecules are known in the art and are appropriate for use in the method of the invention, including, but not limited to, PAGE extraction, SPRIselect, Select-a-Size DNA Clean & Concentrator™, Pippin Prep and affinity purification.

### Applications

The methods of the invention are useful for efficiently generating RNA structural information, while minimizing generation of a deleterious by-product. Further, the methods can be used to generate sequencing data having a more uniform read-depth, therefore having overall higher quality. The method of the present invention may be used in a wide variety of protocols and technologies. For example, in certain embodiments, the methods can be used to determine the structure of naturally occurring RNA molecules, artificially generated RNA molecules, disease-associated RNA molecules, regulatory RNA molecules, RNA:protein interactions and the like. In one embodiment, the method may be used for revealing known and novel regulatory pathways. That is, the methods may be used in any technology that may require or benefit from analysis of the structure of at least one RNA molecule. In one embodiment, the method of the invention is applicable to DMS/SHAPE-LMPCR and Structure-Seq, and DMS-seq. These technologies are described, for example, in Kwok et al, (Kwok et al., 2013, Anal Biochem, 435:181-186), Ding et at (Ding et at, 2014, Nature, 505:696-700), and Rouskin et al. (Rouskin et al., 2014, Nature, 505(7485):701-705), respectively, the contents of which are incorporated by reference herein in their entirety.

In one embodiment, the method of the invention can be used in a DMS/SHAPE-LMPCR method to determine RNA structure in vin and in vitro in low-abundance transcripts.

In another embodiment, the method of the invention can be used in Structure-Seq, a method that allows for genome-wide profiling of RNA secondary structure, both in vivo and in vitro, for any organism, cell, tissue or virus.

In another embodiment, the method of the invention can be used in DMS-Seq, another method that allows genome-wide probing of RNA secondary structure, both in vivo and in vitro, in any organism, cell, tissue or virus.

In another embodiment, a detailed understanding of the RNA content of an organism, cell, tissue or virus may provide invaluable understanding for differential expression in normal and disease processes (i.e. elucidation of disease processes) for human, animal and/or agricultural applications.

In yet another embodiment, the method of the invention may be used in drug development, especially for identification of drugs that can alter or effect RNA secondary structure.

### Kits

The present invention also includes kits useful in the methods of the invention. Such kits comprise components useful in any of the methods described herein, including for example, primers, hairpin donor molecules, means for amplification of a subject's nucleic acids, means for reverse transcribing a subject's RNA, means for analyzing a subject's nucleic acid sequence, and instructional materials. For example, in one embodiment, the kit comprises components useful for one or more of the generation, detection and quantification of at least one nucleic acid molecule. In various embodiments, at least one control nucleic acid molecule is contained in the kit, such as a positive control, a negative control, or a nucleic acid molecule useful for assessing the quality of a sequencing run.

In one embodiment, the kit additionally comprises a ligase. In another embodiment, the kit additionally comprises a polymerase. The kit may additionally also comprise a nucleotide mixture and (a) reaction buffer(s) and/or a set of primers and optionally a probe for the amplification and detection of the ligation product between an acceptor and donor molecule.

In some embodiments, one or more of the components are premixed in the same reaction container.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Structure-Seq: Sensitive and Accurate Genome-Wide Profiling of RNA Structure In Vivo

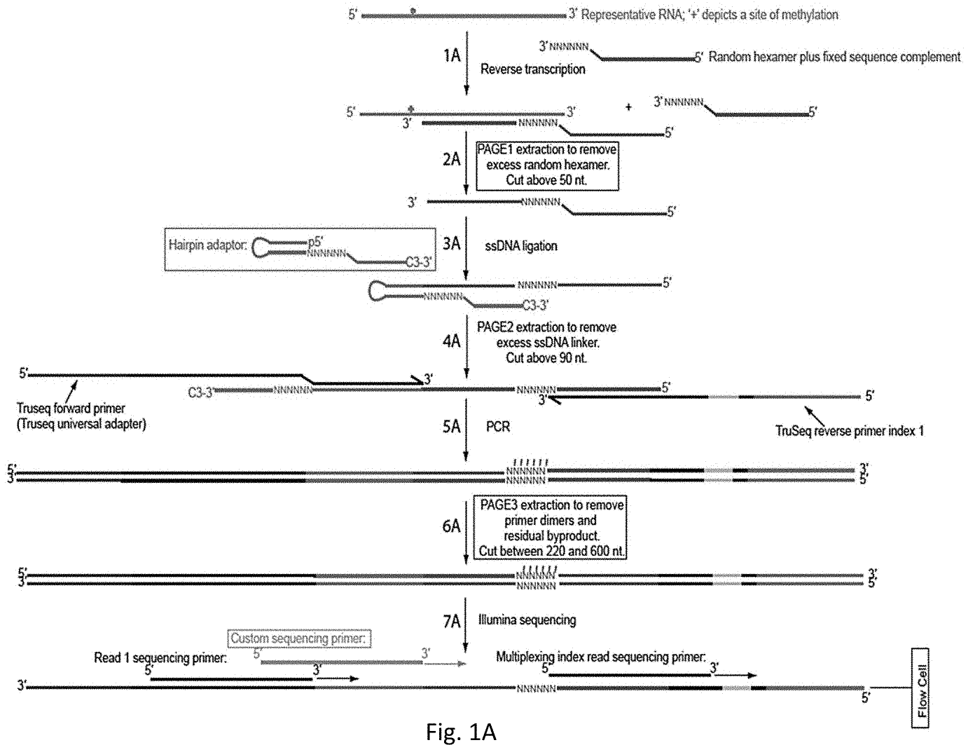

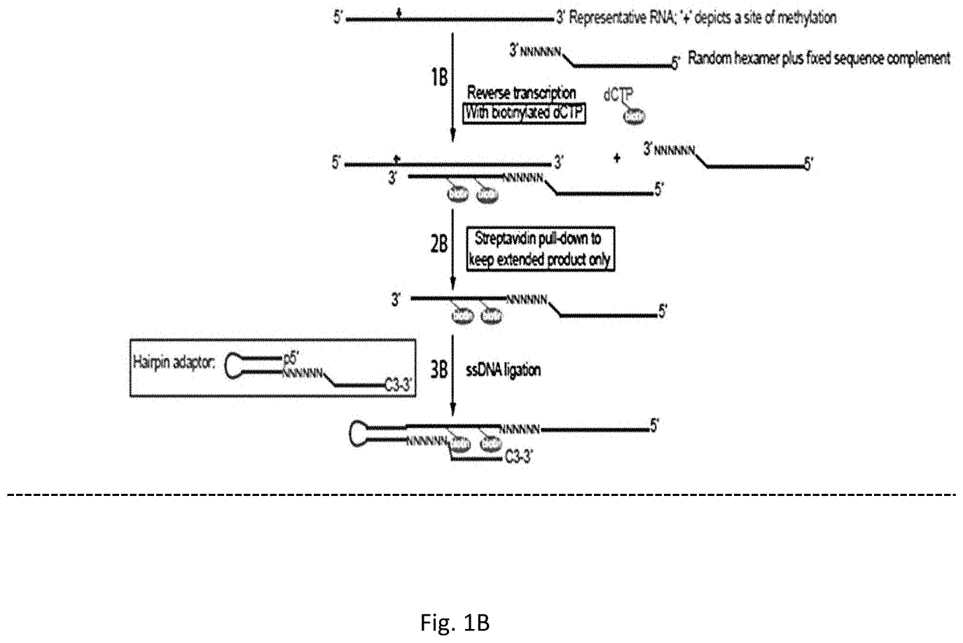

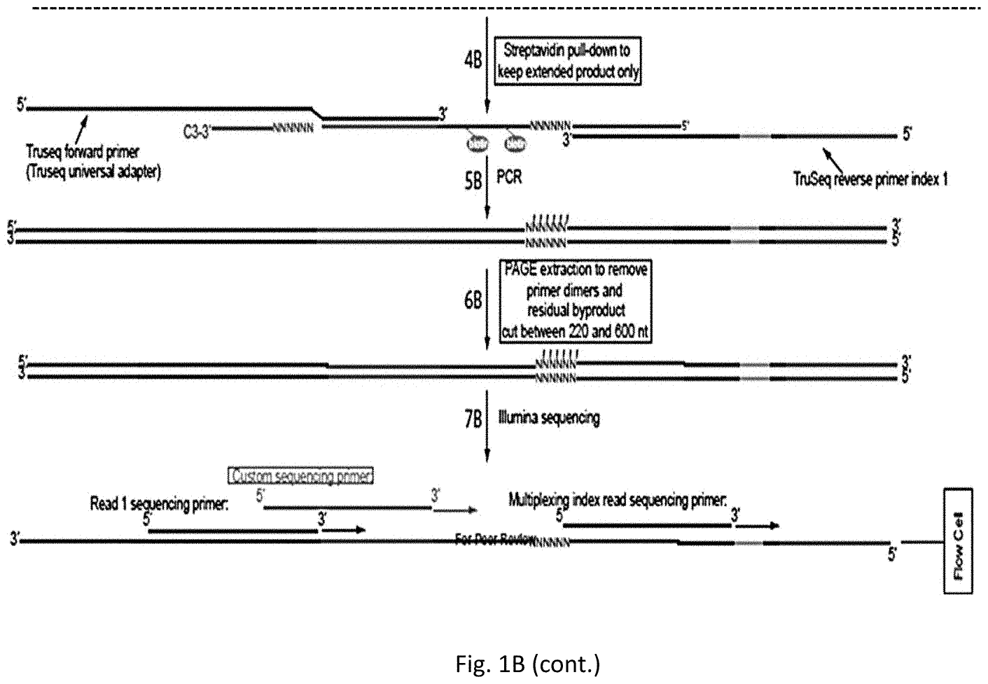

Herein, an improved method for genome-wide profiling of RNA (referred to herein as Structure-seq2) is described (FIG. 1), and its applicability is demonstrated using a new species of rice (Oryza sativa). In Structure-seq2, the amount of starting material needed is reduced from 2,000 to 300-500 ng poly(A)-selected RNA, a different ligation method is used, and two additional denaturing PAGE gels are introduced (FIG. 1). To circumvent the time and cost of these gels, a variation that utilizes streptavidin pulldown of biotinylateddCTP incorporated during RT, which streamlines the protocol.

Structure-seq2 provides a sensitive and accurate method for profiling RNA structure in vivo. While Structure-seq is a powerful tool for determining genome-wide structural information, Structure-seq2 overcomes several limitations of the original Structure-seq protocol (Ding et al., 2015, Nat Protoc, 10:1050-1066). First, a deleterious by-product was found to form between excess RT primer and the ligation adaptor. Removing this by-product significantly increases the quality of the sequenced libraries. Structure-seq2 provides two orthogonal methods to remove this by-product and thus can be tuned to the user's preferences. One of these methods purifies the desired product from the by-product by a total of three PAGE purifications, while the other saves time and material by purifying biotin-containing extension products via a streptavidin purification protocol thus circumventing two of the three PAGE gels. Thereby lowering end-user costs in terms of time and labor and materials costs; thus opening up potentially more applications that are cost-sensitive.

The materials and methods employed in these experiments are now described.

Plant Growth

Wild-type rice (Oryza sativa ssp. japonica cv. Nipponbare) was used in this study. Rice seeds were sown on wet filter paper in a petri dish for germination in a greenhouse with a 16 hour/8 hour day/night photoperiod. Light intensity was 500 μmol m⁻² s⁻¹ with daytime temperatures of 28-32° C. and nighttime temperatures of 25-28° C. After 4-5 days, the rice seedlings were transferred to 6×6 inch nursery pots with water saturated soil (Metro Mix 360 growing medium, Sun Gro Horticulture, Bellevue, Wash.). Five plants were grown per pot. The plants were watered one additionally time, a week after transferring to pots. The shoot tissue of two-week-old plants were used for in vivo DMS probing.

In Vivo DMS Treatment

Rice shoots (1 g total) were excised at the soil line and immersed in 20 mL DMS reaction buffer (100 mM KCl, 40 mM HEPES (pH 7.5), and 0.5 mM MgCl2) in a 50 mL Falcon centrifuge tube. For DMS treatment, 150 μL DMS was added (final concentration 0.75% or ˜75 mM) to the solution, and the DMS reaction was allowed to proceed for 10 minutes with intermittent inversion and mixing. To quench the reaction, 1.5 g of DTT was added to the solution (final concentration of 0.5 M). Vigorous vortexing was applied for 2 minutes. The solution was decanted from the centrifuge tube, and 50 mL of distilled deionized water was added to wash the samples. The wash step was repeated once, then the material was patted dry and immediately frozen in liquid nitrogen. A control treatment (−DMS) was performed as described, but without the addition of DMS.

RNA Extraction and Purification

All RNA extraction steps were done in a chemical fume hood with strong airflow (>250 fpm). Total RNA was extracted using the NucleoSpin RNA Plant kit (Macherey-Nagel, Germany) following the manufacturer's protocol. 500 μg total RNA comprised the starting material for one-round of poly(A) selection using the Poly(A) purist Kit (Thermo Fisher Scientific). To obtain proportionally more reads from mRNA, an additional round of poly(A) selection can be included.

Library Construction

Fifteen different libraries were prepared to determine the outcomes of various modifications to the original Structure-seq method. Table 1 through Table 4 highlights these changes. Two biological replicates each of Structure-seq2−/+DMS without (Libraries 1-4) and with (Libraries 6-9) the biotin variation were made. Each of the other libraries converted one step of the protocol (FIG. 1) back to what was performed in the original version of Structure-seq (Ding et al., 2015. Nat Protoc, 10:1050-1066: Ding et al., 2014, Nature, 505:696-700).

Reverse Transcription

For each sample, two 20 μL reverse transcription (RT)(FIG. 1, Step 1A) reactions were performed in two separate tubes each containing 250 ng (half of the total amount) of poly(A)-selected RNA. To increase coverage of primer annealing, the denaturation and annealing steps of the SuperScript III First-Strand Synthesis kit (Invitrogen) were adjusted. Namely, in the Structure-seq2 samples, the mRNA, random hexamer fused with an Illumina TruSeq Adapter, the 10× RT buffer, and the dNTP mix, were denatured at 90° C. for 1 minute then cooled on ice for 1 minute before adding MgCl2 and DTT to a final concentration of 5 mM each. The samples were then preheated to 55° C. for 1 minute and the SuperScript III was added and the reaction allowed to proceed for 50 minutes. Each reaction contained 250 ng poly(A) RNA, 5 μM RT primer, 20 mM Tris-HCl (pH 8.4), 50 mM KCl, 0.5 mM dNTP (each), 5 mM MgCl2, 5 mM DTT, and 200 U SuperScript III The reaction was terminated by heating to 85° C. for 5 minutes. Residual RNA was cleaved by adding 5U of RNase H and incubating at 37° C. for 20 minutes. Library 12 used the RT denaturation conditions from the original Structure-seq method; the RNA, and the dNTP mix were denatured at 65° C. for 5 minutes then cooled on ice for 1 minute before adding the 10× RT buffer, MgCl2 and DTT to the same final concentrations as in Structure-seq2, Library 13 tested the RT reaction temperature of the original Structure-seq method in which the RT reaction was conducted at 50° C. rather than 55° C. to monitor mutation rates during RT.

For the biotin variation of Structure-seq2 (libraries 6-9) and library 5, which was a control library to test the addition of biotin only (without streptavidin purification). RT was performed as in Structure-seq2, except with Biotin-16-Aminoallyl-2′-deoxycytidine-5′-Triphosphate (TriLink BioTechnologies) doped into the reaction mixture (FIG. 1, Step 18). The final reactions contained 20 mM Tris-HCl (pH 8.0), 50 mM KCl, 5% DMSO, 0.5 mM dNTP (each), and 0.125 mM biotin-dCTP.

PAGE Purification

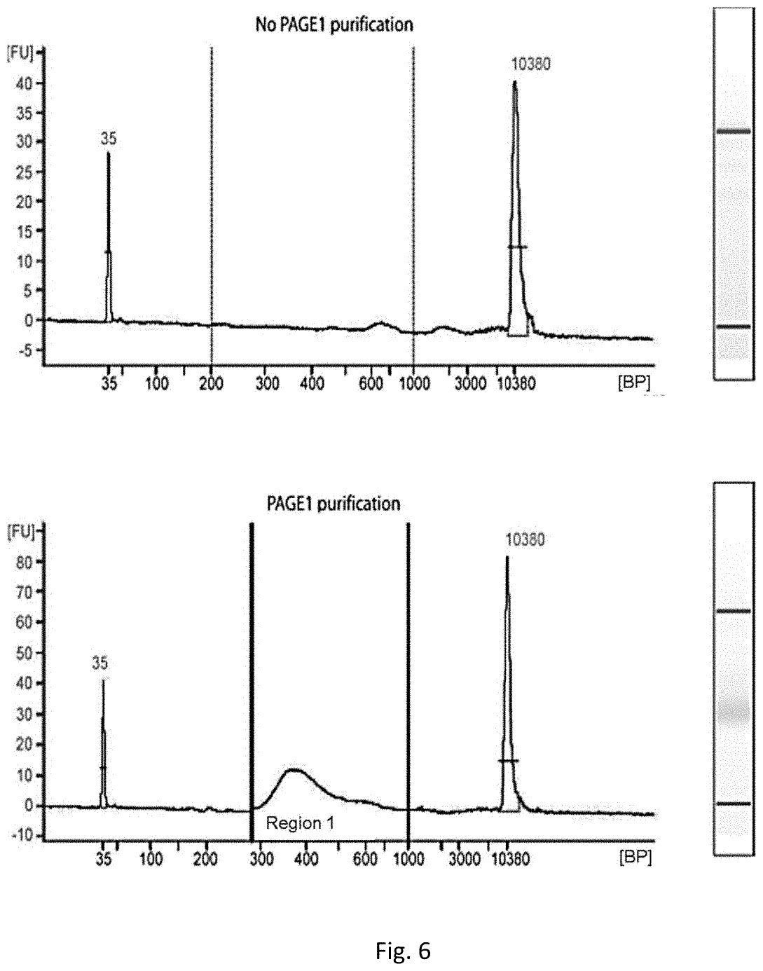

The two separate reaction tubes of each sample were combined for all samples and fractionated on a denaturing PAGE gel containing 10% acrylamide and 8.3 M urea. The gel containing the product was excised above 50 nt, according to a GeneRuler Low Range size ladder (ThermoFisher), to avoid excess RT primer (27 at) (FIG. 1, Step 2A). The excised gel piece was placed in a 50 mL Falcon tube, crushed to fine pieces, and weighed. A volume of TEN250 at least twice as much in mL as the mass of the gel piece in grams was used to submerge the gel pieces. The tube was then placed in a shaker/incubator at 37° C. overnight. Ethanol precipitation was performed by first using a 0.12 μm syringe filter (PALL Scientific) to remove gel fragments and expel the buffer into a new 50 mL Falcon tube, then adding 2.5-3× the volume of 100% ice cold ethanol and 0.5 μL of GlycoBlue, and placing the tube on dry ice for at least 1 hour. The sample was spun down at 12,000 g for 30 minutes before decanting the liquid and re-suspending the pellet with 1-2 mL 70% ice cold ethanol. The sample was spun down at 12,000 g for 5 minutes, the liquid was decanted, and the sample spun down for 1 minute before removing the last bit of liquid with a pipette. The pellet was dried to completion in a 37° C. incubator and then dissolved in 100 μL of water and transferred to a 1.7 mL Eppendorf tube. The sample was then concentrated to the proper volume for the subsequent reactions. The above RT-PAGE purification step was excluded for library 15 which tested the necessity of this gel (FIG. 6).

Streptavidin Purification

For the biotin variation, the two separate RT reaction tubes of each sample were combined and diluted to 100 μL. Phenol:chloroform extraction was performed as described in the original Structure-seq (Ding et al., 2015. Nat Protoc, 10:1050-1066). The final extraction product was purified with an illustra MicroSpinG-50 column (GE Healthcare) to remove excess dNTP and biotin-dCTP. Ethanol precipitation was performed as described previously (Ding et al., 2015, Nat Protoc, 10:1050-1066) and the cDNA was dissolved in 50 μL of 1× Wash/Binding Buffer (0.5 M NaCl, 20 mM Tris-HCl (pH 7.5), 1 mM EDTA).

During the final ethanol precipitation step, 25 μL of magnetic hydrophilic streptavidin beads were washed with 50 μL of 1× Wash/Binding Buffer in a 1.7 mL microcentrifuge tube. A magnet was applied to pull the beads to the side of the tube, and the supernatant was pipetted off. The beads were washed two more times with 50 μL of 1× Wash/Binding Buffer. After the final wash was discarded, the cDNA in 50 μL of 1× Wash/Binding buffer was added to the beads, and the beads were suspended by vortexing. The sample was incubated at room temperature for 10 minutes with occasional agitation by hand. A magnet was applied, and the supernatant was discarded. The beads were washed twice with 100 μL of 1× Wash/Binding buffer, and twice with 100 μL warm (40° C.) Low Salt Buffer (0.15 M NaCl, 20 mM Tris-HCl (pH 7.5), 1 mM EDTA). Each wash included vortexing to suspend the beads, pulse spinning to pull the solution to the bottom of the tube, applying a magnet, and pipetting off the supernatant. To elute the product from the beads, 25 μL of Formamide Buffer (95% formamide, 10 mM EDTA) was added to the beads, the tubes were vortexed and incubated at 95° C. for 2 minutes, a magnet was applied, and the supernatant was transferred to a clean 1.7 microcentrifuge tube. The elution was repeated with another 25 μL of Formamide Buffer, and the supernatant added to the first elution. The solution was diluted to 200 μL with RNase-free water, and ethanol precipitation was performed (FIG. 1, Step 28).

T4 DNA Ligation

The ligation method was performed with T4 DNA ligase (FIG. 1, Step 3A/3B)(Kwok et al., 2013, Anal Biochem, 435:181-186). After renaturing the purified cDNA with betaine, polyethylene glycol 8000 (PEG 8000), and hairpin donor (5′-pTGAAGAGCCTAGTCGCTGTTCANNNNNNCTGCCCATAGAG-3′-Spacer (SEQ ID NO:1), where ‘5-p’ is a 5′ phosphate and 3′-Spacer is a 3-carbon linker), 10×T4 DNA ligase buffer and T4 DNA ligase (NEB) were added to give a final 10 μL reaction mixture containing 500 mM Betaine, 20% PEG 8000, 10 μM hairpin donor, 1×T4 DNA ligase buffer, and 400 U T4 DNA ligase. The reaction proceeded at 16° C. for 6 hours, followed by 30° C. for 6 hours, and was stopped by incubating at 65° C. for 15 minutes. Library 11 tested the ligation method of the original Structure-seq. A 20 μL reaction containing the cDNA, 5 μM ssDNA unstructured linker (5′-pNNNAGATCGGAAGAGCGTCGTGTAG-3′-Spacer)(SEQ ID NO:2), Ix Circligase reaction buffer, 50 μM ATP, 2.5 mM MnCl2, and 100 U Circligase was incubated at 65′C for 12 hours and was stopped by incubating at 85° C. for 15 minutes.

The ligated cDNA was fractionated on a denaturing PAGE gel containing 10% acrylamide and 8.3M urea. The gel containing the product was excised above 90 nt to avoid excess hairpin donor (40 nt) and by-product (67 nt), according to GeneRuler low range DNA size ladder and custom ssDNA oligonucleotides of 67 nt and 91 nt (FIG. 1, Step 4A). For the biotin variation, streptavidin purification was performed as described above (FIG. 1, Step 48).

Library Amplification by PCR

PCR amplification (FIG. 1, Step 5A/5B) was performed using Q5 High Fidelity DNA polymerase (NEB) and Illumina TruSeq primers (Illumina TruSeq forward primer, 5′-AATGATACGGCGACCACCGAGATCTACACTCTTTCCCTACACGACGCTCTTCC GATCTTGAACAGCGACTAGGCTCTTCA-3′(SEQ ID NO:3); Illumina TruSeq reverseprimers, 5′-CAAGCAGAAGACGGCATACGAGAT(N)₆₋₈GTGACTGGAGTTCAGACGTTGCTCTTCCGA′TC-3′(SEQ ID NO:4) where (N)₆₋₈ refers to the unique 6-8 nt Illumina barcode for each sample. Reactions (25 μL) contained 1× Q5 reaction buffer, 0.2 mM dNTPs (each), 0.4 μM forward primer, and 0.4 μM reverse primer and 0.5 U Q5 DNA polymerase. The samples were initially denatured at 98° C. for 1 minutes, cycled through a denaturation step of 98° C. for 8 seconds and an extension step of 72° C. for 45 seconds, then subjected to a final extension step at 72° C. for 10 minutes. Library 10 used the original Structure-seq protocol for amplification; the 25 IL reaction contained 1× Ex Taq buffer, 0.2 mM dNTPs (each), 0.2 μM forward primer, and 0.2 μM reverse primer and 0.1 U Ex Taq DNA polymerase. After a PCR cycle test to determine the minimum number of cycles needed to obtain sufficient product, the amplification was completed at the selected cycle number, and the PCR product was purified via a 10% acrylamide 8.3 M urea denaturing PAGE gel to remove the by-product and obtain products between 220-600 nt according to a ss100 DNA ladder from Simplex Sciences (FIG. 1, Step 6A/6B). Note that it is important that this gel have even heating across the entire glass plate to avoid slower migration of the DNA at the outer edges of the plate, often referred to as ‘smiling’, as this can lead to imprecise excision of the desired DNA and carry over of the by-product into sequencing. Library 14 tested this final purification using the original version of Structure-seq: the sample was extracted from three successive agarose gels instead of extracting from a PAGE gel.

Illumina Sequencing

The quality of the purified libraries was evaluated by analysis on an Agilent Bioanalyzer system to evaluate the relative amounts of desired product vs. by-product, and by qPCR to quantify the concentration of each library and balance between them in order to achieve even sequencing output from the various libraries. Libraries were sequenced using a MiSeq desktop sequencer (Illumina) with single-end reads of 150 bp. Approximately 20 at are the minimum needed for accurate read mapping to the rice transcriptome, although this value may vary for other organisms, and this is the basis for cutting no closer than 20 at above the primer.

Sequence Generation, Processing, and Mapping

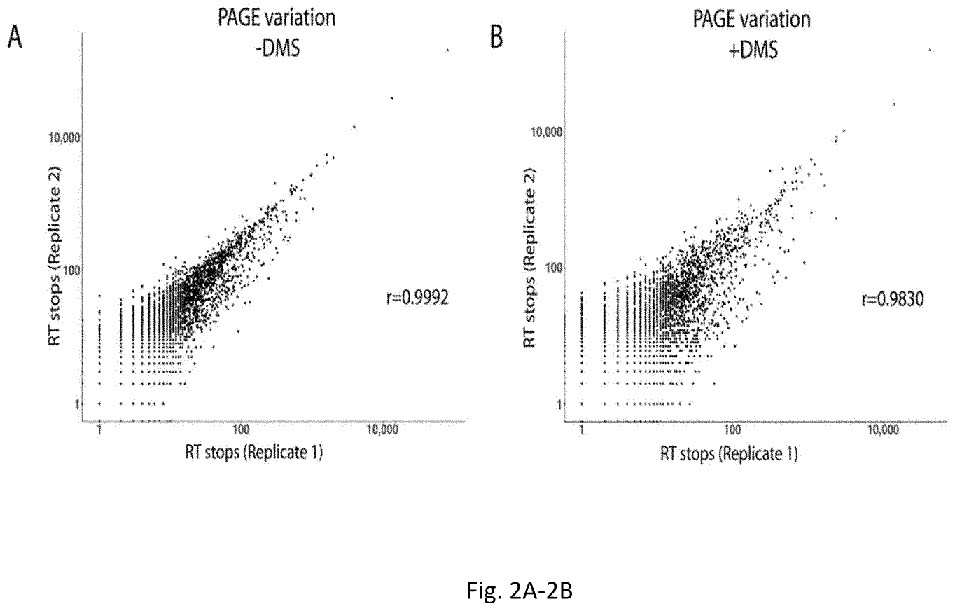

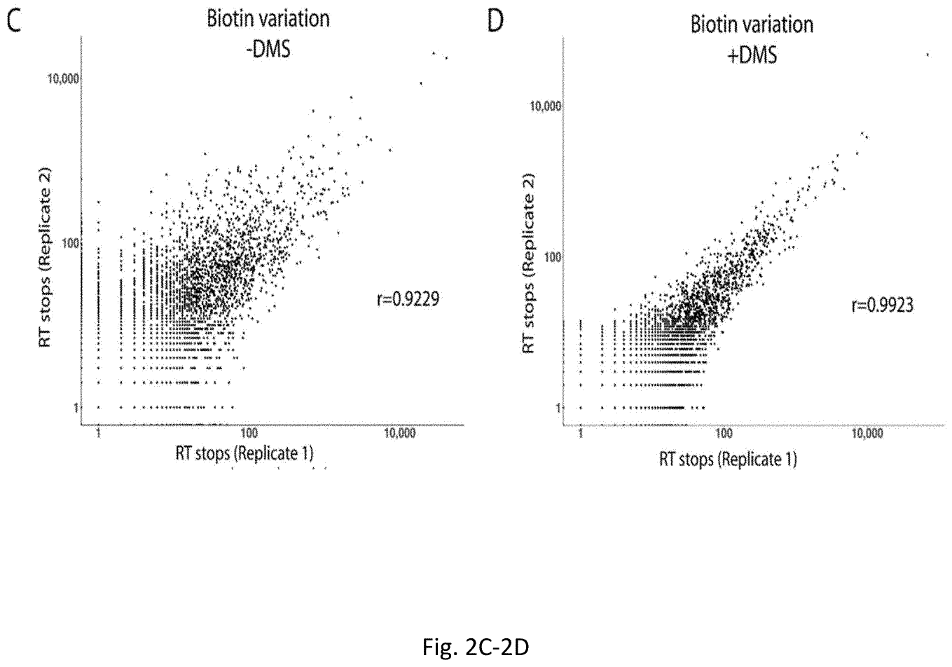

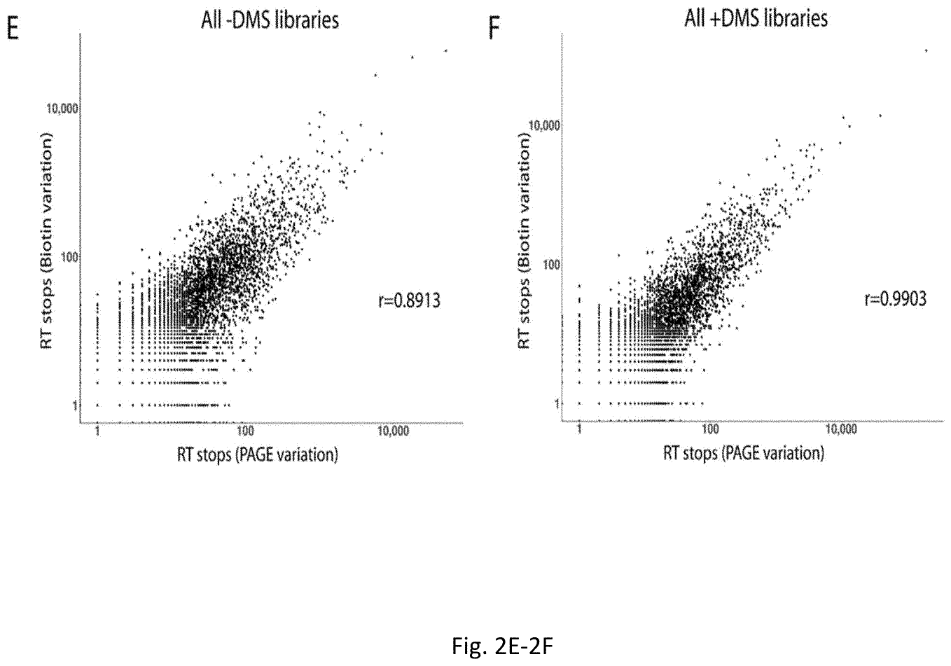

Sequenced reads (150 nt) were obtained with an Illumina MiSeq. For Strucure-seq2, adapters were removed computationally and reads were filtered for a quality score of >30 and a length of >20 using cutdapt (Martin, 2011, EMBnet.Journal, 17:10-12), whereas Structure-seq used iterative mapping. Filtered reads were mapped to the rice reference cDNA and rRNA libraries using Bowtie2 (Langmead and Salzberg, 2012, Nature methods, 9:357-359)(as compared to iterative Bowtie mapping in Structure-seq). Reads with a mismatch on the first 5′ nucleotide were discarded in Structure-seq2. Biological replicates were combined after validating correlation (PAGE−DMS libraries r=0.999; PAGE+DMS libraries r=0.983; biotin −DMS libraries r=0.923; biotin+DMS libraries r=0992) (FIG. 2A through FIG. 2D, respectively). When analyzing biological aspects rather than technical improvements, PAGE and biotin libraries were combined (−DMS r=0.891; +DMS r=0.990) (FIG. 2E through FIG. 2F). Raw DMS reactivities were derived using the same computational pipeline as for Structure-seq, except that 2-8% normalization was performed at the transcript level rather than at the global level as in Structure-seq (Tang et al., 2015, Bioinformatics, 31:2668-2675),

The Results of the Experiments are Now Described.

The Structure-seq2 method is summarized in FIG. 1. Key improvements of Structure-seq2 are removal of a by-product, reduction of ligation bias, leveling out of read depth, lowering of mutation rate, and improvement of sequencing quality. Structure-seq2 is then benchmarked with rRNA and mRNA structure,

Removal of the Deleterious by-Product

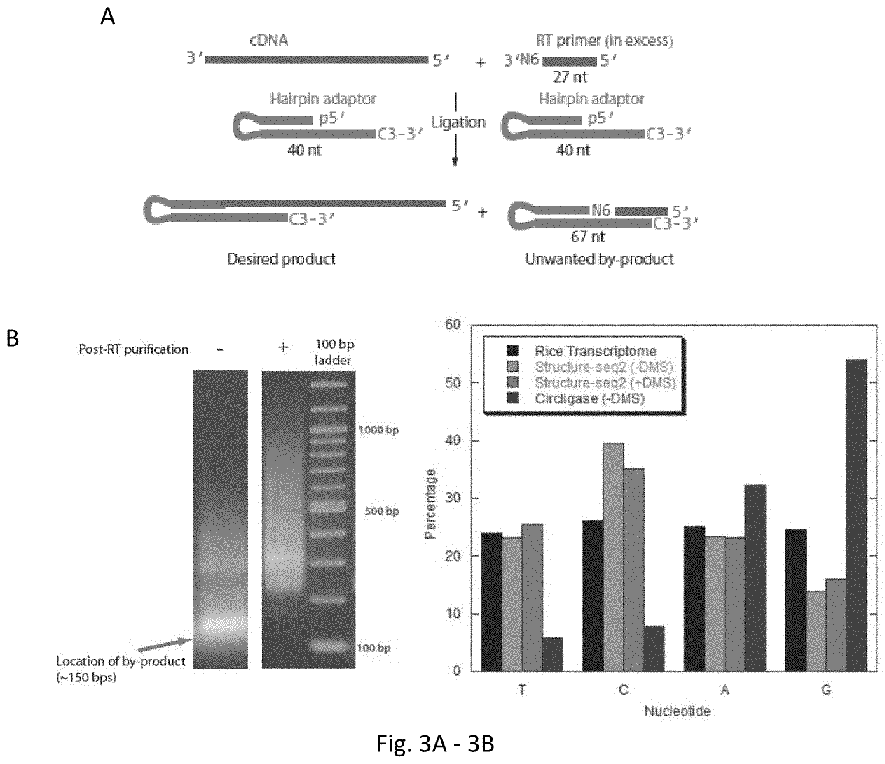

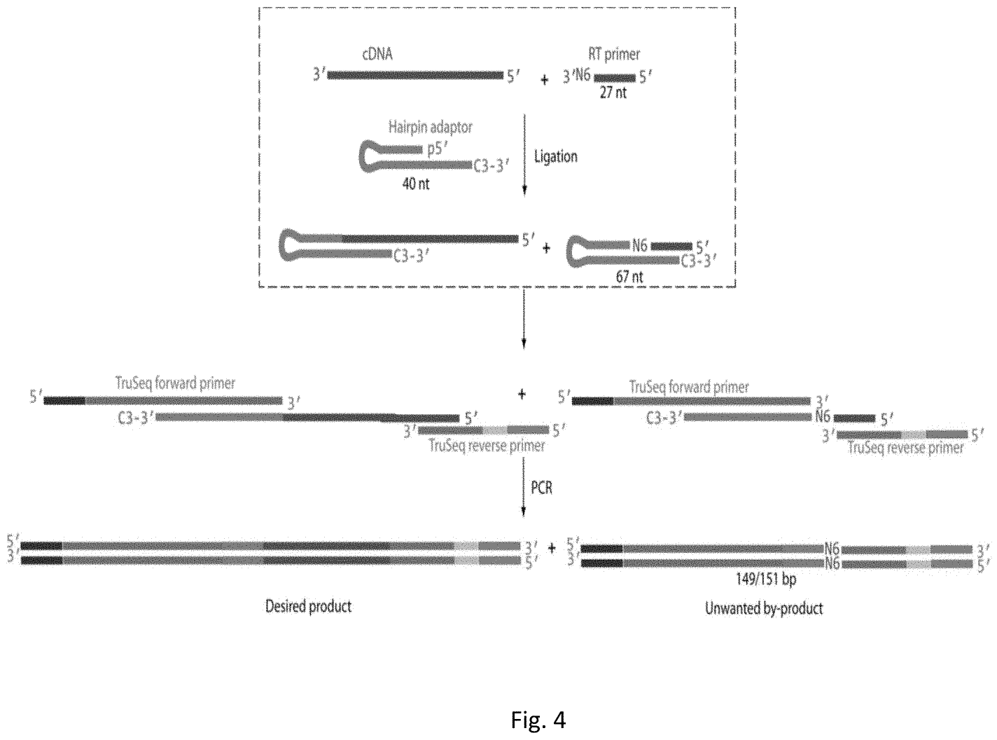

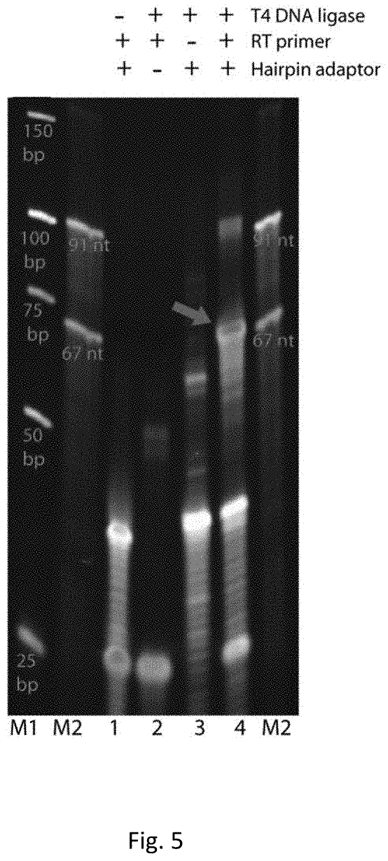

The original Structure-seq method leads to formation of an undesired by-product between the RT primer and ligation adaptor (FIG. 3A, FIG. 4 and FIG. 5). Because the by-product is shorter than a ligated extension product, it amplifies readily in PCR making it especially problematic. Presence of the by-product in the libraries reduces the proportion of useful reads. Previous runs with the original Structure-seq often became poisoned with the by-product such that either the desired library could not be prepared at all or such that effective read rates were as low as 10% to 50%. However, Structure-seq2 unexpectedly produces results with effective read rates around 90% (Table 1-Table 5). To minimize formation of this by-product, three single nucleotide-resolution PAGE purifications were performed.

TABLE 1
RT of libraries

| Library | | RT | RT | Biotin | RT |
|---|---|---|---|---|---|
| number | Library | denaturation | reaction | added | Purification |
| 1 | Structure-seq2 (−DMS) | 90° C. with salt | 55° C. | no | PAGE |
| 2 | Structure-seq2 (−DMS) | 90° C. with salt | 55° C. | no | PAGE |
| 3 | Structure-seq2 (+DMS) | 90° C. with salt | 55° C. | no | PAGE |
| 4 | Structure-seq2 (+DMS) | 90° C. with salt | 55° C. | no | PAGE |
| 5 | Biotin only | 90° C. with salt | 55° C. | yes | PAGE |
| 6 | Structure-seq2 Biotin | 90° C. with salt | 55° C. | yes | streptavidin |
| | variation (−DMS) | | | | |
| 7 | Structure-seq2 Biotin | 90° C. with salt | 55° C. | yes | streptavidin |
| | variation (−DMS) | | | | |
| 8 | Structure-seq2 Biotin | 90° C. with salt | 55° C. | yes | streptavidin |
| | variation (+DMS) | | | | |
| 9 | Structure-seq2 Biotin | 90° C. with salt | 55° C. | yes | streptavidin |
| | variation (+DMS) | | | | |
| 10 | Ex Taq DNA polymerase | 90° C. with salt | 55° C. | no | PAGE |
| 11 | Circligase | 90° C. with salt | 55° C. | no | PAGE |
| 12 | Low RT denaturation | 65° C. without salt | 55° C. | no | PAGE |
| 13 | Low RT reaction | 90° C. with salt | 50° C. | no | PAGE |
| 14 | Agarose purification | 90° C. with salt | 55° C. | no | PAGE |
| 15 | No RT purification | 90° C. with salt | 55° C. | no | none |

TABLE 2
Ligation of libraries

| Library | | Ligation | Ligation | PCR | Final |
|---|---|---|---|---|---|
| number | Library | method | purification | enzyme | purification |
| 1 | Structure-seq2 (−DMS) | T4 DNA ligase | PAGE | Q5 | PAGE |
| | | | | | (lower cut) |
| 2 | Structure-seq2 (−DMS) | T4 DNA ligase | PAGE | Q5 | PAGE |
| 3 | Structure-seq2 (+DMS) | T4 DNA ligase | PAGE | Q5 | PAGE |
| | | | | | (lower cut) |
| 4 | Structure-seq2 (+DMS) | T4 DNA ligase | PAGE | Q5 | PAGE |
| 5 | Biotin only | T4 DNA ligase | PAGE | Q5 | PAGE |
| 6 | Structure-seq2 Biotin | T4 DNA ligase | streptavidin | Q5 | PAGE |
| | variation (−DMS) | | | | (lower cut) |
| 7 | Structure-seq2 Biotin | T4 DNA ligase | streptavidin | Q5 | PAGE |
| | variation (−DMS) | | | | |
| 8 | Structure-seq2 Biotin | T4 DNA ligase | streptavidin | Q5 | PAGE |
| | variation (+DMS) | | | | |
| 9 | Structure-seq2 Biotin | T4 DNA ligase | streptavidin | Q5 | PAGE |
| | variation (+DMS) | | | | (lower cut) |
| 10 | Ex Taq DNA polymerase | T4 DNA ligase | PAGE | Ex Taq | PAGE |
| 11 | Circligase | Circligase | PAGE | Q5 | PAGE |
| 12 | Low RT denaturation | T4 DNA ligase | PAGE | Q5 | PAGE |
| 13 | Low RT reaction | T4 DNA ligase | PAGE | Q5 | PAGE |
| 14 | Agarose purification | T4 DNA ligase | PAGE | Q5 | triple |
| | | | | | agarose |
| 15 | No RT purification | T4 DNA ligase | PAGE | Q5 | PAGE |

TABLE 3
Sequencing of libraries using standard primer

| | | | % of | Effective | | Mapped | |
|---|---|---|---|---|---|---|---|
| | Total | | total | read (ER) | % of | read(MR) | % of |
| Library | Sequence | N35 | (a) | (b) | total | to genome | ER |
| 1 | 915043 | 125386 | 13.70% | 635387 | 69.44% | 367037 | 57.77% |
| 2 | 1212755 | 30379 | 2.50% | 1E+06 | 86.74% | 817151 | 77.68% |
| 3 | 813143 | 172595 | 21.23% | 500967 | 61.61% | 357985 | 71.46% |
| 4 | 1157912 | 19392 | 1.67% | 1E+06 | 88.05% | 802951 | 78.76% |
| 5 | 1151725 | 58489 | 5.08% | 986329 | 85.64% | 786103 | 79.70% |
| 6 | 706321 | 222154 | 31.45% | 415429 | 58.82% | 324307 | 78.07% |
| 7 | 960949 | 129236 | 13.45% | 671466 | 69.88% | 525981 | 78.33% |
| 8 | 824538 | 97191 | 11.79% | 599229 | 72.67% | 477638 | 79.71% |
| 9 | 738228 | 257135 | 34.83% | 364596 | 49.39% | 278036 | 76.26% |
| 10 | 1241920 | 59467 | 4.79% | 1E+06 | 85.26% | 844233 | 79.73% |
| 11 | 1062036 | 20674 | 1.95% | 1E+06 | 97.79% | 949711 | 91.45% |
| 12 | 1143535 | 5091 | 0.45% | 1E+06 | 89.72% | 677884 | 66.07% |
| 13 | 1065001 | 7115 | 0.67% | 946937 | 88.91% | 766115 | 80.90% |
| 14 | 345119 | 96715 | 28.02% | 209646 | 60.75% | 160073 | 76.35% |
| 15 | (c) | (c) | (c) | (c) | (c) | (c) | (c) |

(a) Although the percentage of by-product slightly increases in Structure-seq2 when using biotin, this may be overcome by more stringent washing during streptavidin pulldown.
(b) Effective reads are high quality reads that are longer than 20 nucleotides.
(c) Sample was not of high enough quality to sequence.

TABLE 4
Sequencing of libraries using custom primer

| | | | % of | Effective | | Mapped | |
|---|---|---|---|---|---|---|---|
| | Total | | total | read (ER) | % of | read(MR) | % of |
| Library | Sequence | N35 | (a) | (b) | total | to genome | ER |
| 1 | 1384644 | 683118 | 49.34% | 666535 | 48.14% | 629632 | 94.46% |
| 2 | 1761816 | 147304 | 8.36% | 2E+06 | 90.45% | 1520059 | 95.39% |
| 3 | 1329765 | 540034 | 40.61% | 713822 | 53.68% | 658151 | 92.20% |
| 4 | 1630005 | 95652 | 5.87% | 2E+06 | 93.30% | 1459111 | 95.94% |
| 5 | 1771983 | 159908 | 9.02% | 2E+06 | 90.18% | 1476263 | 92.38% |
| 6 | 1100795 | 481475 | 43.74% | 606799 | 55.12% | 594456 | 97.97% |
| 7 | 1434134 | 438145 | 30.55% | 964624 | 67.26% | 935031 | 96.93% |
| 8 | 1259329 | 360519 | 28.63% | 879375 | 69.83% | 852302 | 96.92% |
| 9 | 1243245 | 711201 | 57.21% | 496010 | 39.90% | 471881 | 95.14% |
| 10 | 1814731 | 152908 | 8.43% | 2E+06 | 91.04% | 1535705 | 92.95% |
| 11 | NA | NA | NA | NA | NA | NA | NA |
| 12 | 1496609 | 11643 | 0.78% | 1E+06 | 98.91% | 1251124 | 84.52% |
| 13 | 1631502 | 16490 | 1.01% | 2E+06 | 98.74% | 1524688 | 94.64% |
| 14 | 1067259 | 472010 | 44.23% | 554160 | 51.92% | 533232 | 96.22% |
| 15 | NA | NA | NA | NA | NA | NA | NA |

TABLE 5
Mismatch rates

| | | Over all | | Over all | |
|---|---|---|---|---|---|
| | Se- | mismatch | Se- | mismatch | Mutation |
| Library | quencing | rate per | quencing | rate per | rate at 25S- |
| number | primer | nucleotide | primer | nucleotide | A648 (a) |
| 1 | standard | 0.96% | custom | 0.89% | 14.89% |
| 2 | standard | 0.89% | custom | 0.82% | 19.70% |
| 3 | standard | 1.06% | custom | 0.99% | 17.65% |
| 4 | standard | 0.94% | custom | 0.89% | 13.96% |
| 5 | standard | 1.17% | custom | 1.10% | 5.26% |
| 6 | standard | 0.82% | custom | 0.74% | 0.78% |
| 7 | standard | 0.83% | custom | 0.78% | 10.69% |
| 8 | standard | 0.85% | custom | 0.80% | 8.77% |
| 9 | standard | 0.88% | custom | 0.83% | 0.00% |
| 10 | standard | 1.15% | custom | 1.07% | 6.17% |
| 11 | standard | 0.82% | NA | NA | 12.57% |
| 12 | standard | 1.06% | custom | 0.99% | 13.68% |
| 13 | standard | 0.97% | custom | 0.88% | 16.26% |
| 14 | standard | 1.06% | custom | 0.89% | 12.77% |
| 15 | (b) | (b) | NA | NA | NA |

(a) Mutation rate at 25S-A648 is calculated by combining all data from both sequencing runs (with and without custom primer)
(b) Sample was not of high enough quality to sequence.

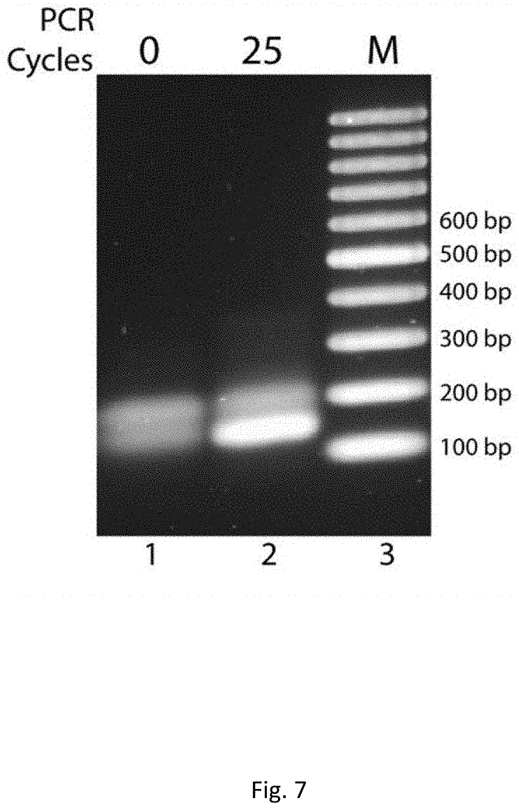

In the first gel (FIG. 1, Step 2A), excess RT primer is removed. The RT product smear is fractionated by denaturing PAGE and the gel is excised above 50 nt, which is ˜20 nt above the 27 nt RT primer. This significantly reduces by-product formation. Without the reduction in by-product afforded by this new Step 2A, the lower amount of starting RNA yields insufficient PCR product for library preparation and sequencing (FIG. 6). The next PAGE gel (FIG. 1, Step 4A), which was also present in the original Structure-seq, removes excess ligation adaptor as well as any residual by-product by excising above 90 nt, which is ˜20 nt above the by-product (67 nt, which comes from the 27 nt RT primer and the 40 nt ligation adaptor). The third PAGE gel, representing the second new PAGE gel, removes any residual by-product amplified during PCR, as well as PCR primers and any primer dimers (FIG. 1, Steps 6A, 6B), This PAGE gel replaces three consecutive native agarose gels used in Structure-seq. Native agarose gels are potentially problematic because they do not offer single nucleotide resolution; moreover, single-stranded nucleic acids in this protocol do not migrate true to size on lower-resolution native agarose gels (FIG. 7). Given these limitations, native agarose gel purifications have been entirely removed from Structure-seq2. Proper size selection on the third PAGE gel is 220-600 nt, which avoids the 149/151 bp by-product (FIG. 4 and FIG. 7). Imprecise cutting at this third PAGE gel step may result in a lower effective sequencing rate due to the fact that PCR has already occurred, and so any carryover of by-product has been amplified (FIG. 7 and Table 1 through Table 5).

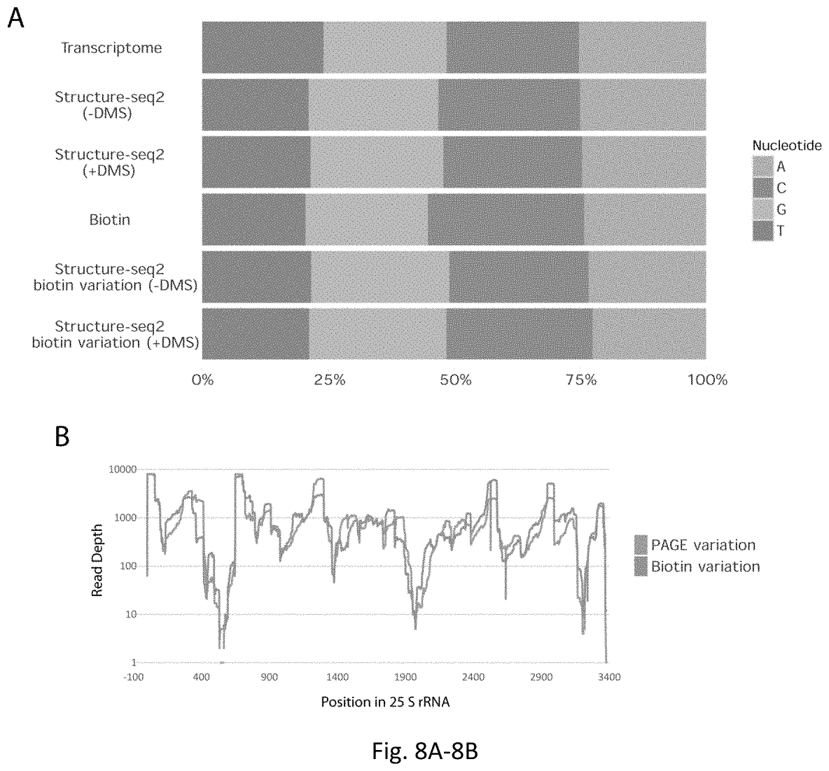

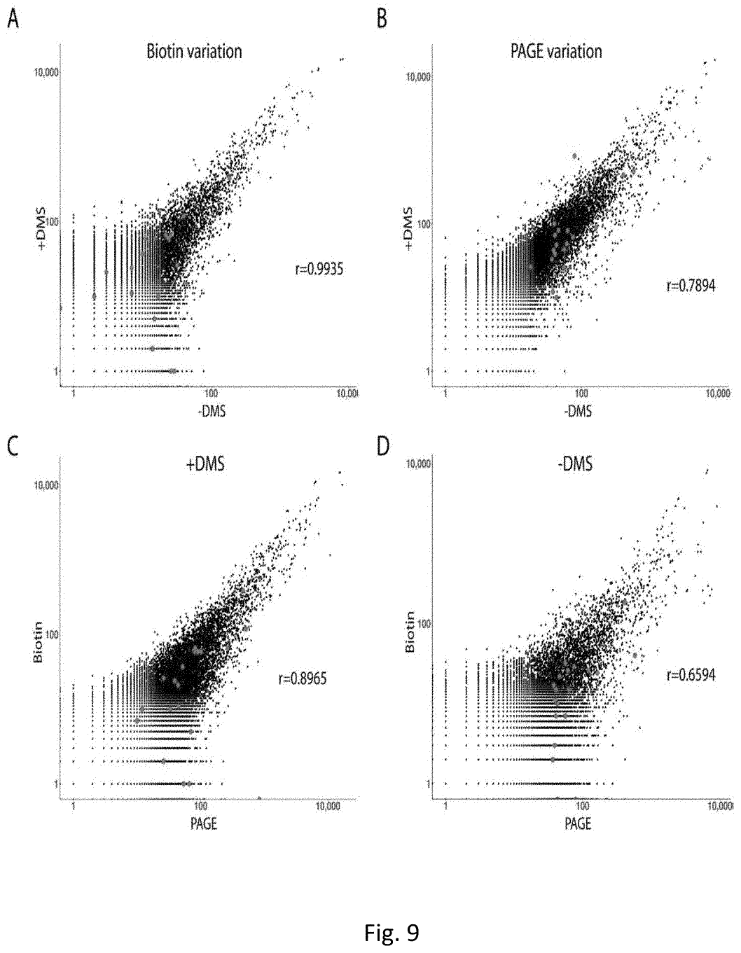

While Structure-seq2 removes the by-product, running three PAGE gels is labor intensive. In practice, it takes approximately a day for each PAGE gel step in the protocol. Accordingly, a facile variation was devised that incorporates biotinylated dNTPs into the RT extension product (Sterling et al., 2015, Nucleic Acids Res, 43:e1) (FIG. 1, Step 1B), allowing the extension product to be separated from the RT primer and by-product by two pull-downs with streptavidin-coated magnetic beads (FIG. 1, Steps 2B,4B). Each of these steps takes only ˜30 minutes. This variation of Structure-seq2 supplants two PAGE gels (Steps 2A, 4A) and thus is more efficient, reducing the library preparation time from over a week to 2.5 days. Importantly, adding biotin-dCTP during RT does not alter the distribution of nucleotide reads (FIG. 8), increase the overall mutation rate during RT or PCR (Table 6), or change the read profiles (FIG. 9).

TABLE 6
Higher mismatch rate with Ex Taq DNA polymerase and
a lower reverse transcription reaction temperature

| | RT reaction | PCR | Mismatch rate per |
|---|---|---|---|
| Library | temperature | polymerase | nucleotide (a) |
| Structure-seq2 (−DMS) | 55° C. | Q5 | 0.89% |
| Structure-seq2 biotin | 55° C. | Q5 | 0.83% |
| variation (−DMS) | | | |
| Ex Taq DNA polymerase | 55° C. | Ex Taq | 1.15% |
| Lower RT reaction | 50° C. | Q5 | 0.97% |
| temperature | | | |

(a) Reads with more than two mismatches are not included as they cannot be confidently mapped

Ligation-Bias Reduction.

The original Structure-seq used Circligase to ligate an adaptor onto the 3′ end of the cDNA, but Circligase has a known nucleotide bias (Kwok et al., 2013, Anal Biochem, 435:181-186; Poulsen et al., 2015, RNA, 21:1042-1052). A ssDNA ligation method was utilized that overcomes this bias (Kwok et al., 2013, Anal Biochem, 435:181-186). A hairpin adaptor is used that base pairs with the 3′end of the cDNA, which is then ligated by T4 DNA ligase. When comparing libraries prepared using T4 DNA ligase and the hairpin adaptor to a library prepared using the Circligase ligation, nucleotide ratios are much closer to transcriptome ratios, demonstrating reduced bias (FIG. 3B). For example, when using Circligase the percentage of T nucleotides at the ligation junction is 6%, while the percentage of G nucleotides is 54%. However, when using T4 DNA ligation, the percentages of T and G residues improve to 23% and 14%, respectively, much closer to the genomic values of 24% and 25%, respectively (FIG. 3B).

More Even Read Depth

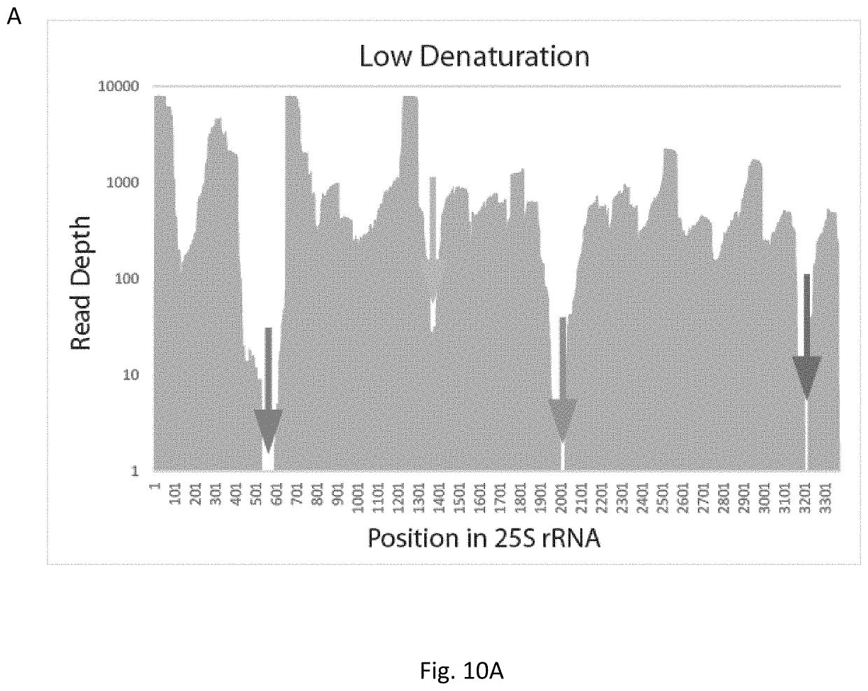

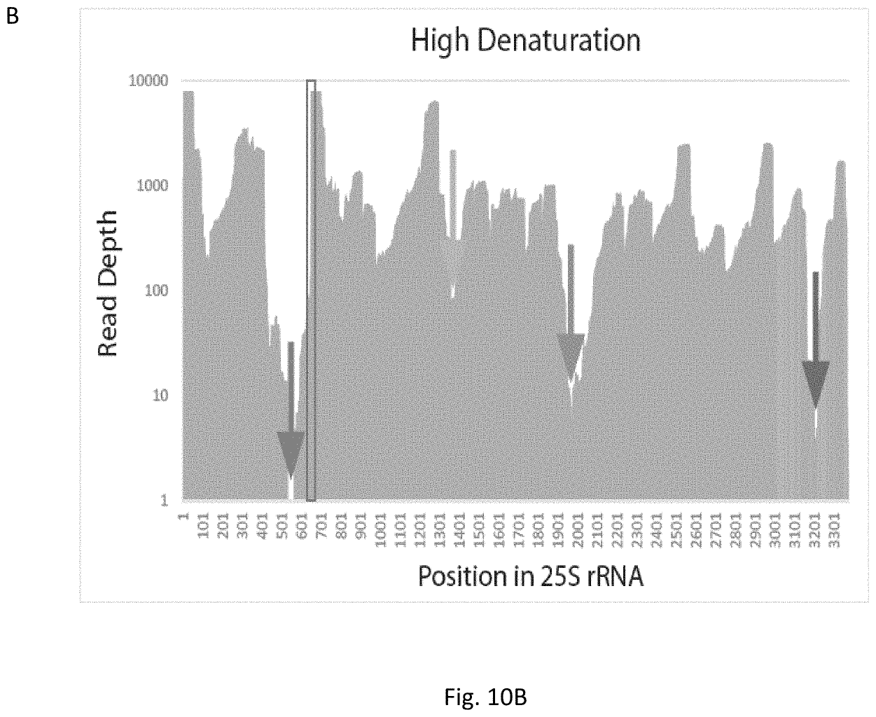

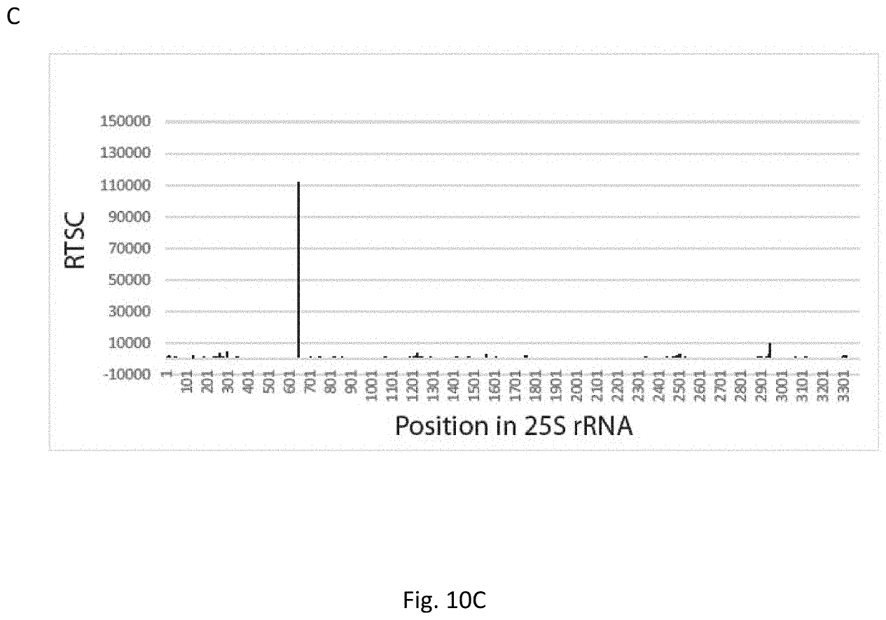

Structure-seq uses a random hexamer during RT to allow hybridization along the entire length of each RNA. Although each transcript should be covered evenly, certain regions are not read as deeply as others and some regions have no reads (FIG. 10A). Regions of low/no coverage could be due to RNA structure interfering with RT primer binding. To address this possibility, two features of the original Structure-seq method were altered. The temperature of the RT annealing step was increased to favor RNA denaturation, and 50 mM KCl was added to favor DNA-RNA annealing. These changes increased read depth at sites of low or no reads. For example, regions in 25S rRNA that had just 27, 1 and 0 reads improved to 83, 6, and 4 reads (FIG. 10A and FIG. 10B); moreover, the width of these three poor read regions narrowed almost two-fold. Certain other positions still had no reads but these also narrowed. For example, there were no reads between 533 and 582, but this region narrowed to 534-539. The cause of these low read regions is likely in vitro RNA self-structure. Specifically, the three regions in 25S rRNA that have less than 10 reads (FIG. 10B, arrows) have GC contents of 83%, 77%, and 94%, compared to an overall GC content of 59% for 25S rRNA.

Lower Mutation Rate and Higher Quality Sequencing Rates

Mutations lower the number of reads that can be reliably mapped to the transcriptome. Without wishing to be bound by theory, it was reasoned that increasing the R.T temperature and changing to a higher fidelity polymerase during PCR might decrease the number of mismatches (Table 6). Upon increasing the RT temperature from 50° C. to 55° C., the mismatch rate per nucleotide decreased from 0.97% to 0.89% (an 8% decrease). When comparing Ex Taq DNA polymerase to the higher fidelity Q5 DNA polymerase, the mismatch rate per nucleotide decreased from 1.15% to 0.89% (a 23% decrease). Thus both elevated RT temperature and high fidelity Q5 polymerase are used in Structure-seq2.

In Structure-seq2, the first 22 nt sequenced are identical for all reads (FIG. 1). Such low diversity can lead to poor sequencing quality by reducing the fidelity of cluster identification during Illumina sequencing (Krueger et al., 2011, PLoS One, 6:e16607), To address this, a custom sequencing primer was designed that abuts the unique region (FIG. 1). Using this custom primer, the mapping rate of effective reads in Structure-seq2, averaged over all libraries, increased sharply from 75% to 94% (Table 3 and Table 4). This custom primer was used in Structure-seq2.

Benchmarking Structure-Seq2

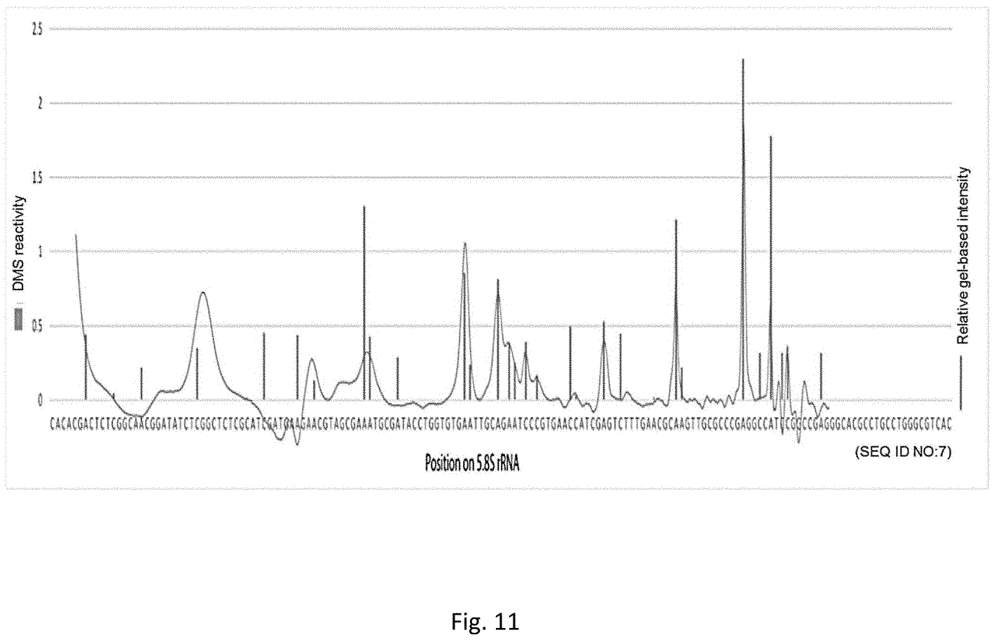

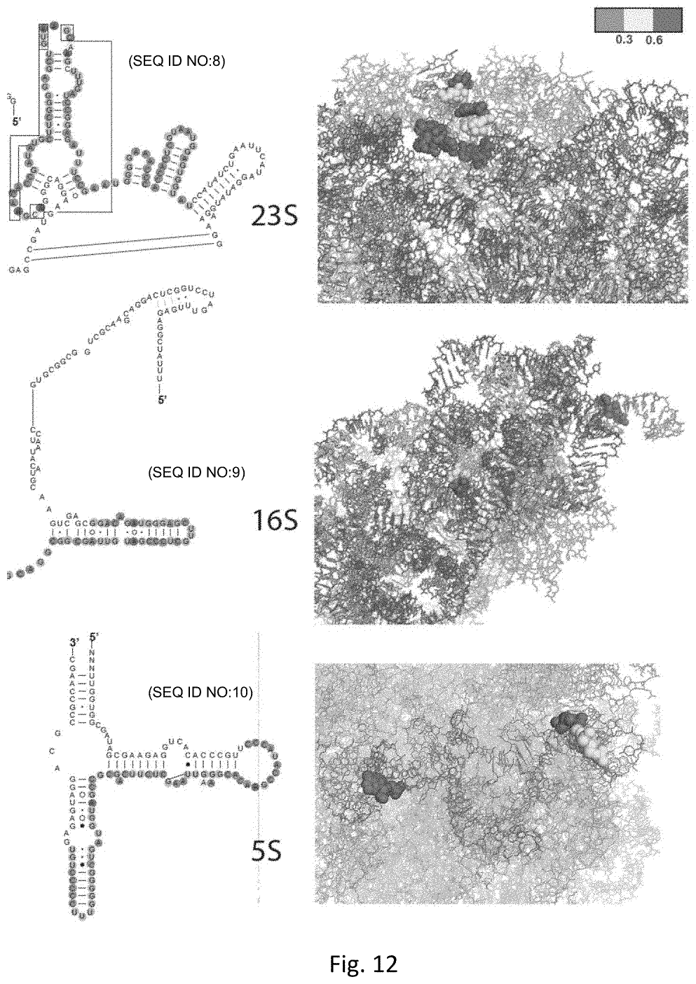

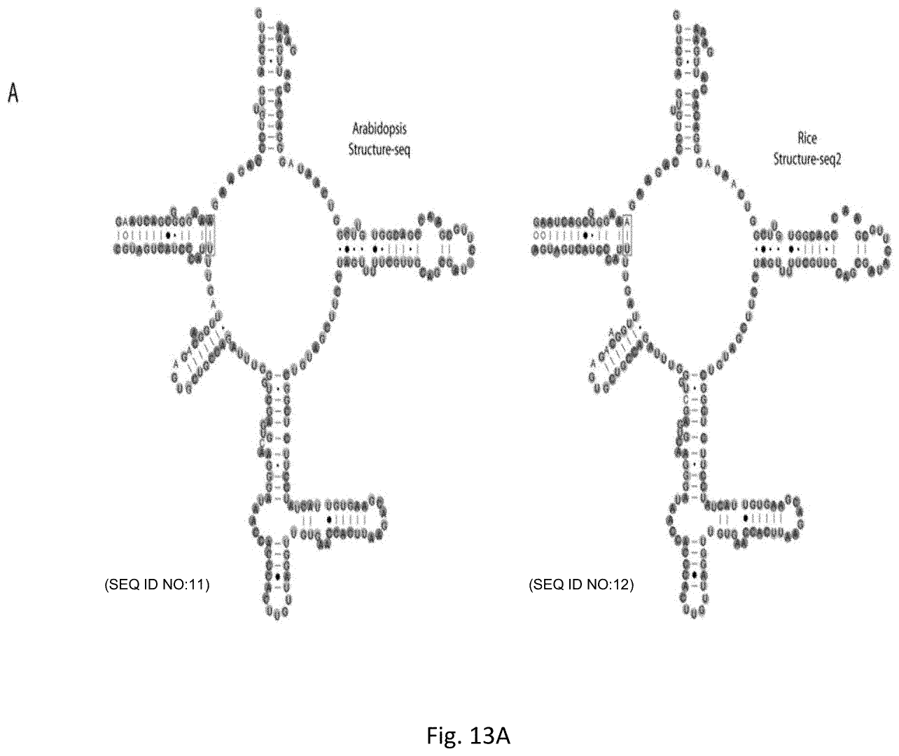

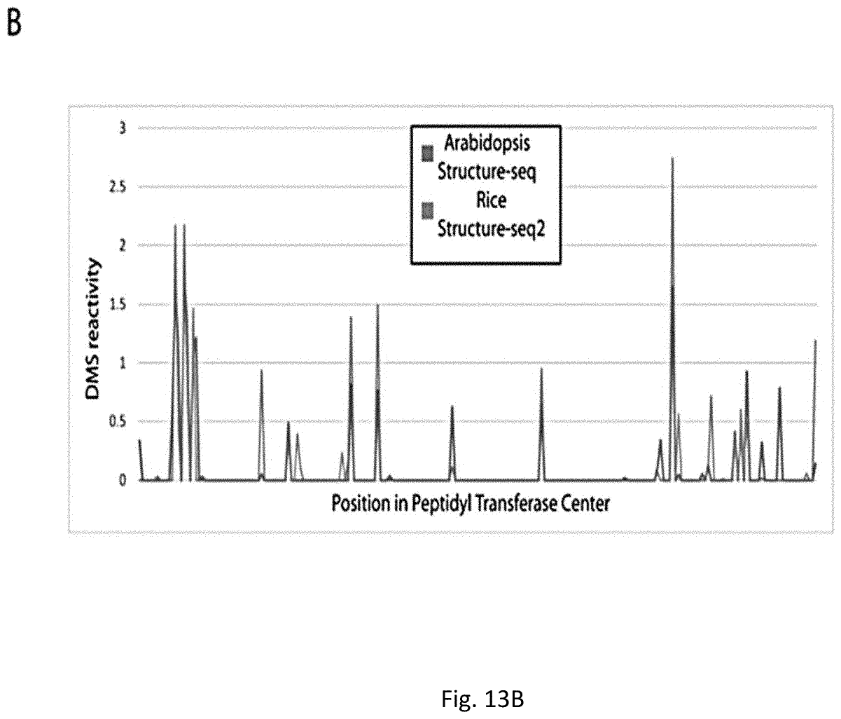

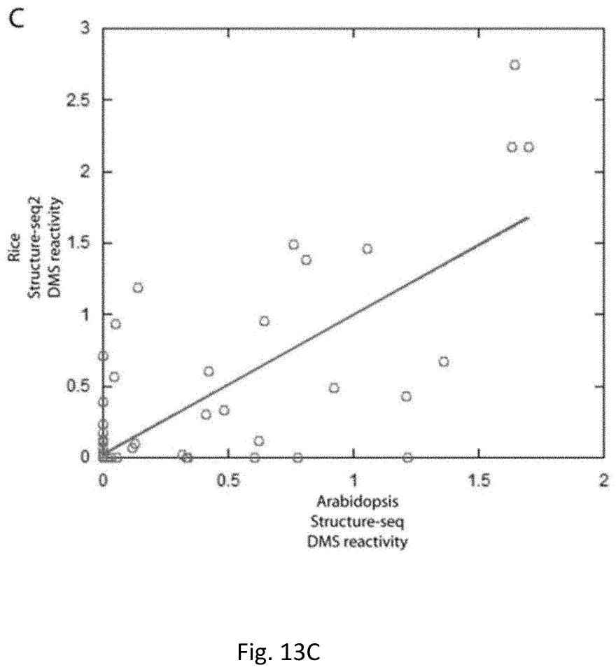

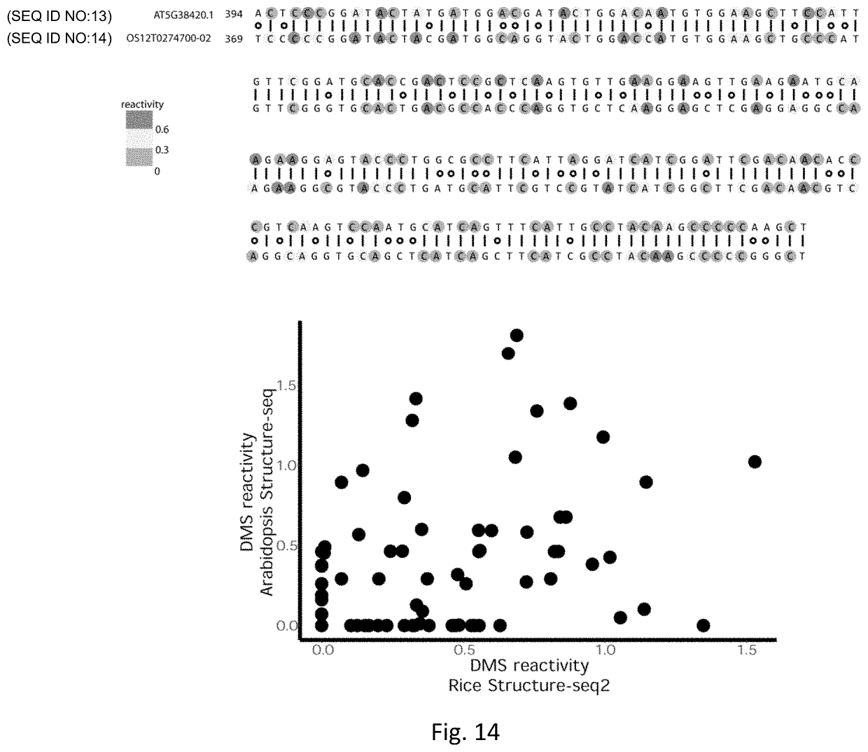

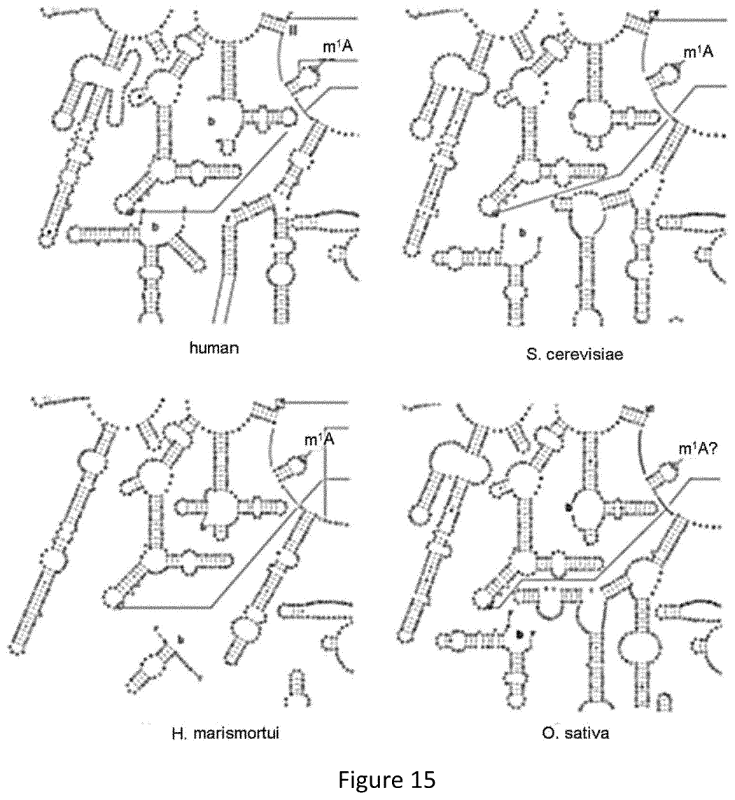

To assure that Structure-seq2 reliably reports on RNA structure, it was benchmarked in three different ways. First, reactivity was compared between Structure-seq2 and gel-based probing, which was done on 5.8S rRNA. As shown (FIG. 11), there is excellent agreement between the two methods. Second, reactivity data was mapped onto 25S rRNA. As shown in FIG. 12, the reactivities agree with 25S rRNA secondary structure known from comparative analysis, confirming the ability of Structure-seq2 to report on the structure of the ribosome (Cannone et al, 2002. BMC Bioinformatics, 3:2). Third, Structure-seq2 was compared to the original Structure-seq performed on Arabidopsis by assessing the continuous reactivity on the completely conserved ancient peptidyl transferase center in rice and Arabidopsis (FIG. 13A), There is a strong correlation (r=0.7738) between continuous reactivity values in the two methods. In addition, reactivity between a region of the orthologous transcripts of RUBISCO SMALL SUBUNIT 2B in OS12T0274700-02 (rice) and ATSG38420.1 (Arabidopsis) (149/196 bp, identity 76%) (FIG. 14)(Proost et al., 2015, Nucleic Acids Res, 43:1974-981), The result shows a similar pattern of continuous reactivity (r=0.4239; p-value=3.9e^{-0.5}) between rice and Arabidopsis on this mRNA, implying both fidelity between both Structure-seq methods and partial conservation of RNA secondary structure.

Using Structure-Seq2 to Identify Novel Biological Features

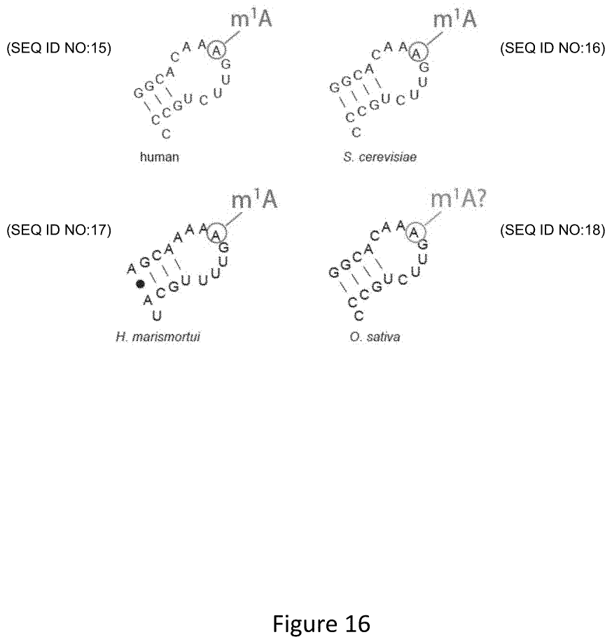

Without wishing to be bound by theory, it was hypothesized that Structure-seq2 could lead to novel insight into biological systems. Ribosomal RNAs are known to be methylated at the N1 position of A648 (rice numbering) of the large ribosomal subunit in human, S. cerevisiae, and H. marismortui (Piekna-Przybylska et al., 2008, Nucleic Acids Res, 36:D178-183), This region is likely to be methylated in rice given the conserved secondary structures and sequences (FIG. 15 and FIG. 16). In fact, the −DMS data in Structure-seq2 provides a very strong RT stop count at this position (FIG. 10C). Intriguingly, there is a very sharp decrease in reads at this site (FIG. 10B, box). Specifically, the read depth is ˜8,000 before A648 and ˜300 at and after it. For the reads that do extend through A648, the mutation rate at this site is elevated to ˜19% as compared to an overall mutation rate of just 0.89% on each nucleotide (Table 5). Importantly, read depth adjacent to this site is improved in the high denaturation condition (FIG. 10A and FIG. 10B, arrows). Structure-seq2 is thus able to identify positions of natural methylation, without fragmenting the RNA as was required for other methods (Hauenschild et al., 2016, Biomolecules, 6:42; Hauenschild et al., 2015, Nucleic Acids Res, 43:9950-9964).

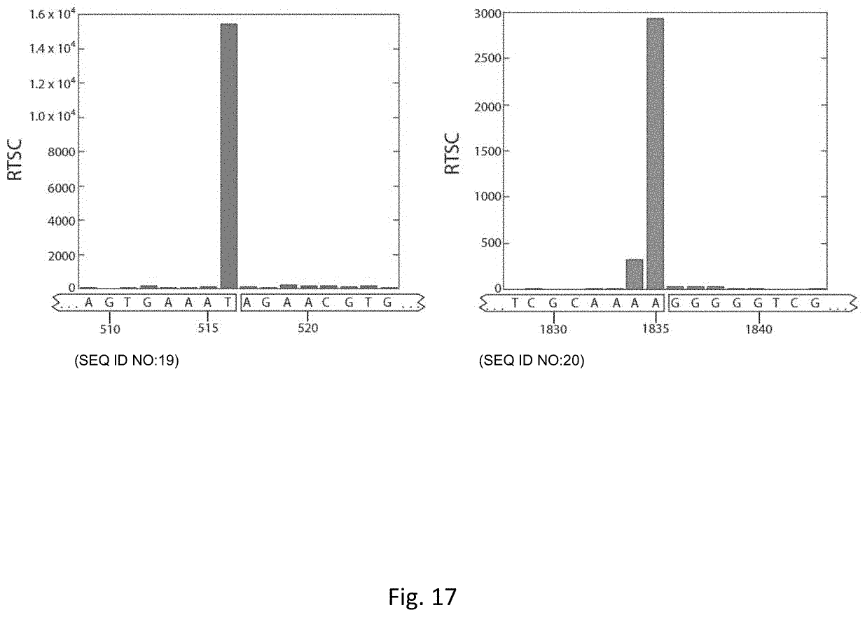

Photosynthetic plant cells are unique in that they harbor chloroplasts, which have their own ribosomes. An unusual feature of chloroplast 23S rRNA is that it has two hidden breaks, which are specific nuclease-mediated covalent breaks in the backbone of a hairpin that are necessary for efficient translation (Bieri et al., 2017, EMBO J, 36:475-486; Leaver, 1973, Biochemn J, 135:237-240). The Structure-seq2 data correctly identify the location of these breaks by a strong signal in the −DMS RT stop data (FIG. 17). Notably, these breaks would not be detectable by RNA-seq, in which the RNA is fragmented before analysis.

In addition to increasing library quality through by-product removal. Structure-seq2 implements optimizations that reduce ligation bias, improve read depth coverage, lower the overall mutation rate, and increase mapping rate. Using T4 DNA ligase with a hairpin ligation adaptor reduces ligation bias. Performing the RT denaturation and annealing steps with conditions that disfavor RNA self-structure (higher heat) and favor RNA-DNA hybridization (50 mM KCl) leads to an improved read depth coverage. Increasing the RT reaction temperature and using a higher fidelity PCR polymerase lowers the overall mutation rate. Using a custom sequencing primer to minimize low-diversity sequencing reads dramatically increases the mapping rate. Through the incorporation of these improvements, the starting material needed for adequate read counts was lowered by over four-fold while also reducing the number of PCR cycles. These improvements are important for cases where RNA samples are limited, significantly reducing the cost of preparing the input poly(A) mRNA, and minimizing mutations arising from DNA amplification.

The high-resolution data obtained from Structure-seq2 applied to rice suggest that a previously unreported m¹A is present in 25S rRNA of rice. Additionally, Structure-seq2 data contain reads closer to this natural modification than data obtained using the RT denaturation conditions found in the original version of Structure-seq. Further, hidden breaks are detectable in chloroplast 23S rRNA using Structure-seq2. While the improvements are applied here to Structure-seq, they can be extended to other genome-wide RNA structure methods including SHAPE-seq, SHAPES, CIRS-seq, HRF-seq, MAP-seq, and ChemModSeq (Poulsen et al., 2015, RNA, 21:1042-1052; Incarnato et al., 2014. Genome Biol, 15:491; Kielpinski and Vinther, 2014. Nucleic Acids Res, 42:e70; Seetin et al, 2014, Methods Mol Biol. 1086:95-117; Hector et al, 2014, Nucleic Acids Res, 42:12138-12154; Loughrey et al. 2014, Nucleic Acids Res, 42:e165).

### Example 2: Genome-Wide RNA Structurome Reprogramming by Acute Heat Shock Globally Regulates mRNA Abundance

Heat stress can have dramatic effects on organisms. After exposure to high temperatures, severe cellular damage occurs in many living systems, including in crop species such as rice (Oryza sativa L.), the staple food for almost half the human population (1). Increasing temperatures and climate variability seriously threaten crop production levels and food security (2), and vulnerability to heat stress results in direct negative effects on yield (3, 4).

A variety of regulatory reprogramming mechanisms occur in organisms in response to high temperature stress, including changes in the transcriptome, proteome, and metabolorne (5-7). RNA secondary and tertiary structure are known to influence numerous processes related to gene expression (8), including transcription (9), RNA maturation (10), translation initiation (11), and transcript degradation (12). However, how heat stress affects RNA structure on a genome-wide scale in vivo is an important yet missing piece of the puzzle concerning temperature based gene regulation.

The combination of RNA structure probing methods and high-throughput sequencing has made it possible to obtain genome-wide RNA structural information at nucleotide resolution in one assay, essentially overcoming many of the limitations of length and abundance of RNA molecules that arise in gel probing of individual RNA species. In yeast, melting temperatures have been obtained for RNA structures genomewide in vitro by probing with V1 nuclease, which cleaves at double-stranded regions (13). In the bacterium Yersinia pseudotuberculosis, in vitro RNA structuromes were mapped at different temperatures using both V1 and the single-stranded nuclease S1 (14). In several other bacterial species, temperature-induced changes in the structures of individual RNA thermometers, as assessed in vitro, have been documented to modulate mRNA translation efficiency (15).

However, in contrast to the above in vitro data, the extent to which temperature stress functionally alters the RNA structurome in living cells is not understood, despite the advent of methods to probe RNA structure genome-wide in vivo (16-20) and extensive evidence that in vivo structure of an RNA molecule can differ dramatically from its in vitro or in silico structures (16, 18). Moreover, in vivo, RNA structures can be altered by numerous endogenous factors that are not present in the test tube, including cellular solutes, proteins, and endogenous crowding agents (21), leading to significant biological consequences. Here, a genome-wide investigation of how elevated temperatures regulate the in vivo structurome was performed by applying Structure-seq2 methodology (19) to profile in vivo RNA structure in the crop plant rice (O. sativa L). Structural data was obtained on 14,292 transcripts and assessed with respect to possible RNA thermometers of the type described in prokaryotes. RNA structurome data was combined with Ribo-seq analyses to identify mRNAs undergoing translation, as well as RNA-seq time courses to quantify post-heatshock transcriptomes. An analysis of relationships among the structure, translation, and abundance of thousands of individual mRNAs identifies a heretofore unappreciated structural basis for the dynamic regulation of mRNA abundance after heat shock.

The materials and methods employed in these experiments are now described.

Preparation of RNA structurome and Ribo-seq libraries followed the procedures of Ritchey et at. (19) and Juntawong et at. (39), respectively, with some modifications. RNA-seq library preparation followed the standard Illumina TruSeq RNA Library preparation pipeline.

Plant Material and Growth Conditions

Seeds of rice (Oryza sativa ssp. japonica cv. Nipponbare) were sown on wet filter paper in a petri dish and geminated for five days in a greenhouse with 16 hour/8 hour day/night photoperiod, with light intensity ˜500 μmol m⁻² s⁻¹ supplied by natural daylight supplemented with 1000 W metal halide lamps (Philips Lighting Co). The temperature was 28-32° C. during the day and 25-28° C. during the night. The rice seedlings were then transferred to 6×6 inch nursery pots filled with water-saturated soil (Metro Mix 360 growing medium, Sun Gro Horticulture, Bellevue, Wash.). Nine plants were grown per pot and were watered once a week after transferring the seedlings to the pots. Shoot tissue of two-week-old plants was used for in vivo DMS probing. All tissue collection started at ˜4 p.m. for all genome-wide experiments to minimize circadian effects.

In Vivo DMS Probing Under Two Temperature Conditions

All manipulations using DMS were conducted with proper safety equipment including lab coats and double gloves. All disposables were disposed of as hazardous waste. DMS treatment was applied in a chemical fume hood with strong airflow (>200 fpm). For the 22° C. treatment, non-DMS-treated (−DMS) and DMS-treated (+DMS) samples were prepared. One g of shoot tissue was excised from the plant immediately before each treatment. For the 4DMS sample, the material was immersed in 20 mL DMS reaction buffer (40 mM HEPES (pH 7.5), 100 mM KCl, and 0.5 mM MgCl2) in a 50 mL conical centrifuge tube. Then 150 μl DMS (D186309, Sigma-Aldrich) was immediately added to the solution to a final concentration of 0.75%(˜75 mM), followed by 10 minutes of gentle inversion and mixing for every 30 seconds. Next, to quench DMS in the reaction (1), dithiothreitol (DTT) at a final concentration of 0.5 M was supplied by adding 1.5 g DTT powder into the solution. After vigorous vortexing to dissolve the DTT, the quench proceeded for 2 minutes. The solution was decanted, and each sample was washed twice with distilled deionized water. Residual water was removed by inverting the tube onto paper towels, and the tissue was immediately frozen in liquid nitrogen. The −DMS sample was processed through the same procedure without addition of DMS. Three biological replicates were prepared for each −/+DMS sample for a total of six samples.

For the heat shock treatment. −DMS and +DMS samples were similarly prepared. For the DMS treatment, 1 g of shoot was excised and placed into 20 mL of 42° C. pre-warmed DMS reaction buffer for 30 seconds in a 50 mL centrifuge tube for temperature equilibration of the tissue. Then 150 μl DMS was added, followed by 10 min of intermittent inversion and mixing in a 42° C. water bath to maintain the temperature. Then 1.5 g of DTT powder was added into the reaction solution for a final DTT concentration of 0.5 M to quench the DMS with the tube immersed in the 42° C. water bath for 2 minutes. The solution was decanted, and samples were washed twice and immediately frozen in liquid nitrogen. The −DMS 42° C. samples were processed through the same procedure, without DMS addition. Three biological replicates were prepared for each sample, for a total of six additional samples.

Structure-Seq Library Generation

Library generation followed a previous library construction pipeline (Ding et al., 2014, Nature 505(7485):696-700; Ritchey et al., 2017, Nucleic Acids Res. 45(14):e135) with some optimization. Total RNA for the 12 individual biological samples was obtained in a chemical fume hood using the NucleoSpin RNA Plant kit (Cat #740949, Macherey-Nagel, Germany) following the manufacturer's protocol. For each sample, 300 μg total RNA comprised the starting material for two rounds of poly(A) selection using the Poly(A) Purist MAG Kit (Cat #AM1922. ThermoFisher), which provided high purity mRNA for library construction, poly(A) purified mRNA (500 ng) was used as the input for Structure-seq library construction following the Structure-seq2 protocol (Ritchey et al., 2017, Nucleic Acids Res. 45(14):e135). Reverse transcription was performed using SuperScript III First-Strand Synthesis System kit (Cat #18080051, ThermoFisher) using the same RT primer as previously used (Ding et al., 2015, Nat. Protoc. 10(7):1050-1066): 5′CAGACGTGTGCTCTTCCGATCNNNNNN3′ (SEQ ID NO:6) which is a fusion of a random hexamer and an Illumina TruSeq Adapter. The first-strand cDNA was size-selected above 52 nt on a 8M urea 10% polyacrylamide gel to remove excess RT primer and increase the ligation efficiency in the next step. After recovering cDNA using the crush-soak method, the cDNA was dissolved in 5 pt. RNase-free water. Ligation was performed using T4 DNA ligase (Cat #M0202, New England Biolabs) which ligated the 3′ end of the cDNA to a low bias single stranded DNA linker (Kwok et al., 2013, Anal. Biochem. 435(2):181-186)/5Phos/TGAAGAGCCTAGTCGCTGTTCANNNNNNCTGCCCATAGAG/3SpC3/(SEQ ID NO:1) where the underlined sequence can form a hairpin structure and the random hexamer can then hybridize to any cDNA fragment (Kwok et al, 2013, Anal. Biochem. 435(2):181-186). Reagents were added into the cDNA solution as follows: 2 μL 10× buffer, 2 μL SM betaine, 2 μL 100 μM linker DNA, 8 μL 50% PEG8000, 1 μL T4 DNA ligase (400 U/μL). The ligation was performed at 16° C. for 6 hours and then 30° C. for 6 hours, and the ligase was then deactivated at 65° C. for 15 minutes. The ligation product was size selected above 90 nt on 8M urea 10% polyacrylamide gels to remove extra single stranded linker DNA and a 67 nt ligation byproduct, consisting of one copy of the hexamer and one copy of the linker DNA. After recovery using the crush-soak method, the purified ligation product was dissolved in 10 μL RNase-free water. PCR amplification (20 cycles) was performed using a primer specific to the single stranded linker DNA and fused with an Illumina TruSeq Universal Adapter: 5′AATGATACGGCGACCACCGAGATCTACACTCTTCCCTACACGACGCTCTT CCGATCTTCAACAGCGACTAGGCTCTTCA3′ (SEQ ID NO:39)(the sequence to prime single-stranded linker DNA is underlined and also needs to be trimmed from sequencing reads), and 12 different Illumina TruSeq Index Adapter reverse complementary primers (SEQ ID NO: 40 through SEQ ID NO:5).

The product was run on an 8M urea 10% polyacrylamide gel for DNA size separation to remove primer dimers and further eliminate byproduct contamination. DNA between 200 bp and 600 bp was collected by reference to both an Ultra Low Range DNA Ladder (Cat #SM1213. ThermoFisher) and a Low Range DNA Ladder (Cat #SMI 193, ThermoFisher). Library DNA size distribution and consistency between biological replicates was assessed by Agilent 2100 Bioanalyzer (Agilent Technologies). After qPCR to quantify the library molarity, a pool of all libraries at equal molarity was made, and libraries were subjected to next-generation sequencing on an Illumina HiSeq 2500 at the Genomics Core Facility of the Penn State University to generate 150 nt single end reads. The Strucutre-seq2 raw sequencing reads are available at the Gene Expression Omnibus (GEO) at the National Center for Biotechnology Information (NCBI) with the series entry GSE100714.

RNA-Seq Library Preparation and Sequencing

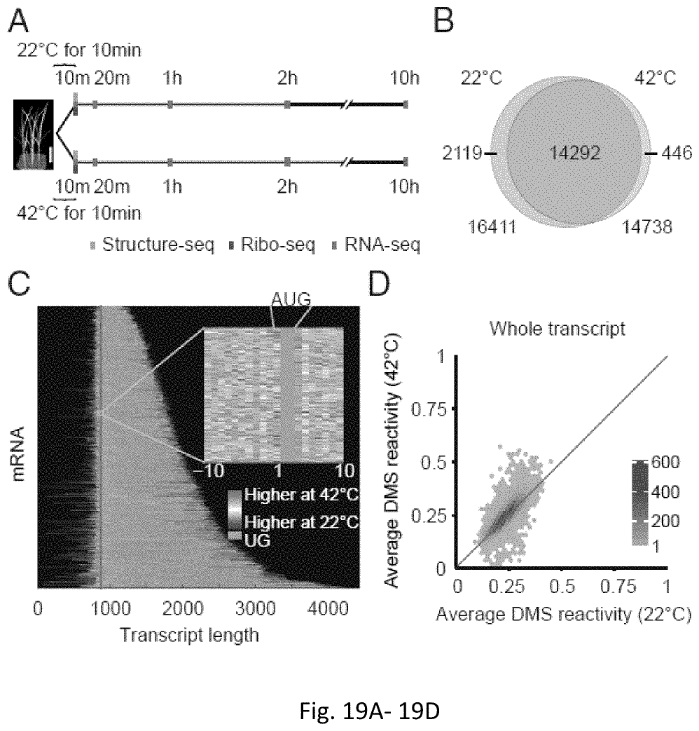

To impose the same heat shock as in the Structure-seq experiment, two week old rice plants in pots were inverted and the shoots were immersed in a water bath at 22° C. or 42° C. for a 10 minute treatment, and the plants were then transferred to a growth chamber set at the same temperature as in the greenhouse (30° C.) for ease of sampling during the recovery period. Three rice shoots comprised one biological replicate, and two biological replicates were obtained for each treatment and time-point, as indicated in FIG. 19A. Total RNA was extracted from each sample using the NucleoSpin RNA plant kit (Cat #740949, Macherey-nagel). After examination of the quantity and quality of these RNA samples by NanoDrop 2000 (Thermo Fisher Scientific, USA) and Bioanalyzer 2100 (Agilent Genomics, USA), total RNA samples were sent to the Genomics Core Facility at Penn State University for RNA-seq library preparation and next generation sequencing (Hiseq 2500, Illumina). Approximately 40-50 million 150 bp single-end sequencing reads were obtained for each library.

Ribosome Profiling Library Preparation and Sequencing

To test the effect of heat on ribosome footprinting, two-week-old rice plants were grown under the same conditions as described for Structure-seq probing. Ten shoots were harvested at 10 minutes as described above for the RNA-seq time course experiment, Isolation of RPFs (ribosome protected fragments) and library construction were performed as described in the literature (Juntawong et al., 2014, Proc. Natl Acad. Sci. USA, 111 (1):E203-212) with some major changes. Rice shoots were ground into powder with liquid nitrogen. For each sample, two mL of tissue powder was dissolved and homogenized in 10 mL polysome extraction buffer on ice. The buffer contains 200 mM Tris-Cl (pH 8.0), 100 mM KCl, 25 mM MgCl2, 5 mM DTT, 1 mM PMSF, 100 μg/mL cycloheximide, 1% Brj-35, 1% TritonX-100, 1% Igepal CA630, 1% Tween-20, 1% polyoxyethylene 10 tridecyl ether. After centrifugation at 16 000 g for 10 minutes at 4° C., the supernatant was collected. The supernatant was then layered on top of an 8 mL sucrose cushion (1.75 M sucrose in 200 mM Tris (pH 8.0), 100 mM KCl, 25 mM MgCl2, 5 mM DTT, 100 μg/mL cycloheximide), and centrifuged at 170 000 g at 4° C. for 3 h. The pellet was resuspended in 400 μL RNase I digestion buffer (50 mM Tris-Cl (pH 8.0), 100 mM KCl, 20 mM MgCl2, 1 mM DTT and 100 μg/mL cycloheximide). After adding 20 μL RNase 1 (Cat #AM2294, Thermo Fisher), RNase digestion was performed at room temperature with rotation for 2 hours. TRIzol reagent (Cat #15596026, Thermo Fisher) was used to extract the RPFs followed by fragment size selection using a NucleoSpin miRNA kit (Cat #740971, Macherey Nagel) to collect the fragments smaller than 200 nt. A Urea-PAGE gel (10%) was then applied to size select 28-32 nt fragments. After dephosphorylation using PNK (Cat #M0201S, NEB), the RPFs were ligated to AIR adenylated RNA linker (Cat #510201, BIOO Scientific). The ligation products were then subjected to reverse transcription using SuperScript III (Cat #18080093, Thermo Fisher) and circularization using Circligase II (Cat #CL9021K, Illumina). Sequence libraries were ultimately obtained through PCR amplification by Q5 polymerase (Cat #M0491S, NEB). The resultant ribosome profiling libraries were sequenced at the Genomics Core Facility at Penn State University to generate single-end 100 nt reads.

Sequence Mapping and Treatment

FastQC (bioinformatics.babraham.ac.uk/projectstfastqc/) software was used to check the quality of the sequencing reads. To remove the adapters at both ends of the reads, cutadapt (Martin, 2011, EMBnet.journal 17(1):10-12) was employed. Any reads shorter than 20 nt or with a quality score <30 (−q flag of cutadapt) were discarded. Reads were then mapped to rice reference cDNA and rRNA libraries using Bowtie2 (Langmead and Salzberg, 2012, Nat. Methods, 9(4):357-359). Reads with more than 3 mismatches or a mismatch on the first nucleotide at the 5′ end were discarded. A high correlation was obtained between the three biological replicates in each condition, replicates were combined for further analysis.

Determination of DMS Reactivity

The method employed to derive DMS reactivity on each nucleotide was similar to that used in previous studies (Ding et al., 2015, Nat. Protoc. 10(7):1050-1066; Ding et al., 2014, Nature 505(7485):696-700; Tang et al., 2015, Bioinformatics 31(16):2668-2675; Tack et al. 2018. Methods. 143:12-15) with additional steps of normalization between the different temperature conditions. The steps to calculate DMS reactivity from (−) DMS and (+) DMS libraries are as follows: Step 1. Normalization of RT stop counts. For each transcript, the RT stop counts on each nucleotide are incremented by 1 and then the natural log (in) is taken, followed by normalization by the transcript's abundance and length (Equation 1 and 2).

$$\begin{matrix}P ( i ) = \frac{ln [ {P}_{r} ( i ) + 1 ]}{\{ {\sum }_{i = 0}^{l} ln [ {P}_{r} ( i ) + 1 ] \} / l} & ( 1 ) \\ M ( i ) = \frac{ln [ {M}_{r} ( i ) + 1 ]}{\{ {\sum }_{i = 0}^{l} ln [ {M}_{r} ( i ) + 1 ] \} / l} & ( 2 )\end{matrix}$$

Here, Pr(i) and Mr(i) are the raw r numbers of RT stops mapped to nucleotide i (all four nucleotides are included) on the transcript in the plus (P) and minus (M) reagent libraries, respectively, and l is the length of the transcript. Pr(0) and Mr(0) are the raw numbers of 5′-runoff RT reads. Step 2. Calculation of DMS reactivity. The raw DMS reactivity is calculated by subtracting the normalized RT stop counts between (+) DMS and (−) DMS libraries with all negative values set to 0. For each nucleotide 1, the DMS reactivity is calculated as follows:

θ(i)=max[P(i)−M(i),0] (3)

Step 3. Normalize the raw DMS reactivity θ(i) of all the nucleotides on all the transcripts to obtain the derived DMS reactivity of each nucleotide as described below. In order to make account for the greater intrinsic reactivity of the DMS at 42° C., the normalization process is performed differently on the two conditions.

a. 22° C.

Perform 2%/8% normalization (Low and Weeks, 2010, Methods 52(2):150-158) on the raw DMS reactivity θ(i) of all the nucleotides on all the transcripts to obtain the derived DMS reactivity of each nucleotide, with the normalization scale derived from the 2%/8% normalization of each transcript. Here, the normalization scale is the average of the bottom four-fifths (80%) of the top 10% of the nucleotide reactivity values on each transcript.

b. 42° C.

Perform normalization on the raw DMS reactivity θ(i) of all the nucleotides on all the transcripts using the normalization scale from the 22° C. condition of each transcript to obtain the final DMS reactivity of each nucleotide. The normalized reactivity is capped at 7 (Kertesz et al., 2010, Nature 467(7311):103-107).

Step 4 Normalize DMS reactivities between conditions to obtain the final reactivity. Suppose θheat(i) and θrt(i) are reactivities at 42° C. and 22° C. for nucleotide i after step 3. Final reactivities are derived as follows:

$$\begin{matrix}{θ}_{final ( heat )} ( i ) = {θ}_{heat} ( i ) \cdot {c}_{heat} & ( 4 ) \\ {θ}_{final ( rt )} ( i ) = {θ}_{rt} ( i ) \cdot {c}_{rt} & ( 5 ) \\ Here , & \\ {c}_{heat} = \frac{( {θ}_{rt} + {θ}_{heat} )}{2 {θ}_{heat}} , {c}_{rt} = \frac{( {θ}_{rt} + {θ}_{heat} )}{2 {θ}_{rt}} , & \\ {θ}_{rt} = {\sum }_{i ϵ S}^{} {θ}_{rt} ( i ) , {θ}_{heat} = {\sum }_{i ϵ S}^{} {θ}_{heat} ( i ) & \end{matrix}$$

S is the set of all nucleotides on all RNAs with coverage ≥1 at 22° C. and 42° C.

RNA-Seq Library Data Analysis

After sequencing, adapter contamination was computationally removed from the libraries and adapter sequences were trimmed from the 3′ ends of the raw reads using cutadapt (Martin, 2011, EMBnet.journal 17(1):10-12). Low-quality bases (Q<30) were also trimmed from both the 5′ and 3′ ends of the reads. Next, reads from each of the four libraries were mapped independently to the rice genome (IRGSP-1.0) using STAR (Dobin et al., 2013, Bioinformatics 29(1):15-21), with a GTF (Gene Transfer File) annotation file supplied as an argument. Mapping information is provided in Table 8. Transcript abundance and differential gene expression were calculated using DEseq2 (Love et al., 2014. Genome Biol. 15(12):550). TPM (transcripts per million)-based gene expression levels were generated for downstream analysis. The RNA-seq raw sequencing reads are available at the Gene Expression Omnibus (GEO) at the National Center for Biotechnology Information (NCBI) with the series entry GSE100713.

Analysis of the Degradome Dataset

The supplementary file from GEO accession GSM1040649, rice degradome data under 28° C. from ZH11 WT plants. GSM1040649_ZH11.fa.gz was downloaded. The fragment sequences were mapped to the rice transcriptome (Oryza sativa.IRGSP-1.0.30.cdna.all.fa) using Bowtie2, and a custom Python script was used to combine the mapping results (.sam) with the fragment counts, generating a combined count of all degradome fragments per transcript. The degradome data of each transcript were merged with the calculated average reactivity data and imported into R. The correlation function (cor( )) was used to test correlation between number of normalized fragments (log 2(#fragments), transcript length) and transcript reactivity at both temperatures. The quantile function was used to subset the data into the 5% highest and 5% lowest average transcript reactivity groups and then the mean number of fragments in each of these groups was compared using two-tailed Student's t-test. To compare the shape of the distribution from each group (abundance increases or decreases) the Matching package (Sekhon, 2011, J. Stat. Softw. 42:1-52) was used to run a bootstrapped KS test (boot.ks, nboots=4000) between the increased and decreased distributions at each respective time point.

Motif Analysis

Sequences and reactivity values for 3′UTR regions of transcripts were extracted from the whole transcript sequence and reactivity data. All instances of the UUAG motif within the 3′UTR of transcripts with coverage over one were identified and the reactivity change was cataloged within the UUAG motif via the react_static_motif.py (SF2) module (Tack et al, 2018, Methods, 143:12-15). The 3′UTR regions of transcripts with coverage over one were then subdivided via a sliding window analysis into windows of 50 nt by 20 nt steps and ranked by total increase and decrease of reactivity via the react_windows.py (SF2) module (Tack et al., 2018, Methods, 143:12-1S). Fasta formatted files corresponding to the top and bottom 1% of reactivity increases and decreases among these windows were used as the input to MEME suite analysis. The discovered enriched motifs were compared to the protein-binding motifs published in Gosai et al., 2015, Mol. Cell 57(2):376-388).

Ribosome Profiling Data Analysis

To calculate ribosome association and its modulation by temperature, the adapter 5′-ACTGTAGGCACCATCAAT-3′(SEQ ID NO:52) at the 3′ end of the reads was first removed using cutadapt. Any reads shorter than 20 nt or longer than 40 nt or with a quality score <30 (−q flag of cutadapt) were discarded. Reads were then mapped to the rice reference genome and cDNA libraries using Howtie2. Since we obtained a high correlation between the 2 biological replicates in each condition, replicates were combined for further analysis. Ribosome association in each condition was derived using the resultant ribosome profiling library, with the RNA-seq library at 10 min as the control library. Read depth of each nucleotide on each RNA was normalized by the total number of reads in each library and then the natural log (In) was taken on the normalized read depth. The Ribo-seq signal of each nucleotide was calculated by subtracting the natural log of the normalized read depth of each nucleotide in the RNA-seq library from that in the ribosome profiling library. The Ribo-seq signal per transcript is the average of the value of all nucleotides in the transcript. The change in Ribo-seq signal was calculated by subtracting the average Ribo-seq signal in heat (42° C.) from that in the control condition (22° C.), The Ribo-seq raw sequencing reads are available at the Gene Expression Omnibus (GEO) at the National Center for Biotechnology Information (NCBI) with the series entry GSE102216.

Optical Melting

As is standard for analyses of optical melting, RNA was denatured at 95° C. for 90 seconds in water and then allowed to refold at 4° C. for 90 seconds, then room temperature for 5 minutes. After the 5 minutes, the buffer was adjusted to 40 mM HEPES pH 7.5, 100 mM KCl, and 0.5 mM Mg2+, and allowed to equilibrate at room temperature for 10 minutes. Samples were spun down at 14,000 rpm for 5 minutes at room temperature to remove air bubbles and particulates, then transferred to a quartz cuvette. Final sample concentrations were 1.1 μM RNA. The transitions for T2 and T3 were confirmed to be independent over a range of concentrations from 0.55 μM to 5.5 μM, supporting that transition is from the hairpin rather than duplex state. T1: OS06T0105350-00, Similar to Scarecrow-like 6 (SEQ ID NO:53); T2: OS02T0662100-01, Similar to Tfm5 protein (SEQ ID NO:54); T3: OS03T059900-02, Hypothetical conserved gene (SEQ ID NO:55); T4: OS02T0769100-01, Auxin responsive SAUR protein family protein (SEQ ID NO:56).

Thermal denaturation experiments were performed on an HP 8452 diode-array refurbished by OLIS, Inc. with a data point collected every 0.5° C. with absorbance detection from 200-600 nm. Data at 260 nm were converted to fraction folded assuming linear baselines.

mRNA Decay Analysis

mRNA decay rate determination was performed by following a previously described method (Park et al., 2014, Plant Physiol. 159(3): 1111-1124) with modifications. The conditions of rice seedling growth were the same as for our other genome-wide assays. After 13 days of growth, rice seedlings were gently removed from the soil, carefully washed to remove dirt from the root tissue and transferred to tap water to recover for 1 day, similar to the method of Park et al. (2012). Cordycepin solution with a final concentration of 1 mM was prepared in tap water and equilibrated at the prevailing temperature in the greenhouse (30° C.) before treatment. Rice seedlings were then transferred to cordycepin solution, with the roots immersed, and pretreated for 30 min before the start of temperature treatment. For temperature treatment, 1 mM cordycepin solutions were prepared before use and equilibrated in a water bath for 42° C. treatment and on the lab bench for 22° C. treatment. After the 30 minute pretreatment, seedlings were quickly transferred to either 42C cordycepin solution for heat treatment or 22° C. solution for room temperature (control) treatment, for 10 minutes. This protocol followed the identical protocol as used to obtain the Structure-seq and ribo-seq 10 minute datasets (FIG. 19A). The seedlings were then transferred back to the 30° C. cordycepin solution and placed in a 30° C. growth chamber for recovery, identical to the recovery protocol used for the RNA-seq timecourse (FIG. 19A). Plant materials were sampled at the end of the cordycepin pretreatment as control sample (C0), then immediately after 10 minutes of the two temperature treatments (H10m and C10m), then after 50 minutes of “heat recovery” (HR) in the growth chamber (HR1h and C1h). Three biological replicates were prepared for each sample. After RNA extraction using the RNA Plant kit (Cat #740949, Macherey-nagel), cDNA was synthesized using the SuperScript III first-strand synthesis system (Cat #: 18080051, ThermoFisher), qRT-PCR analysis was performed using a Bio-Rad real-time PCR detection systems with SYBR Green Supermix (Cat. #. 1708880, Bio-Rad). qRT-PCR was performed using the following protocol: 95° C. for 5 minutes, followed by 49 cycles of 95° C. for 20 s, 53° C. for 20 s, and 72° C. for 30 s, and then melting curve analysis (60° C.-95° C. at a heating rate of 0.1C/S). qRT-PCR was performed in triplicate for each cDNA sample. Using rice Ubiquitin 1(Ubi1, Os06g0681400) as the internal control, the relative abundance of each transcript at each time point was normalized by Ubi1 abundance within the same sample. Relative decay post temperature treatment was then normalized by comparison to the relative abundance at the 0 min time point. Relative decay was plotted as a line graph to show the trend of change in remaining mRNA abundance. To identify candidate rice XRN targets in rice, we first used the most reliable XRN targets list in Arabidopsis, designated “Class II.” by (Merret et al., 2015), then we consulted the PLAZA database to identify the best BLAST hits from A. thaliana to O. sativa. Every O. sativa ortholog of each A. thaliana XRN responsive gene was convened from MSU format to Ensemble format before use in our data analyses.

The results of the experiments are now described.

Structure-Seq Reveals Heat-Induced Unfolding of the in Vivo Eukaryotic Transcriptome

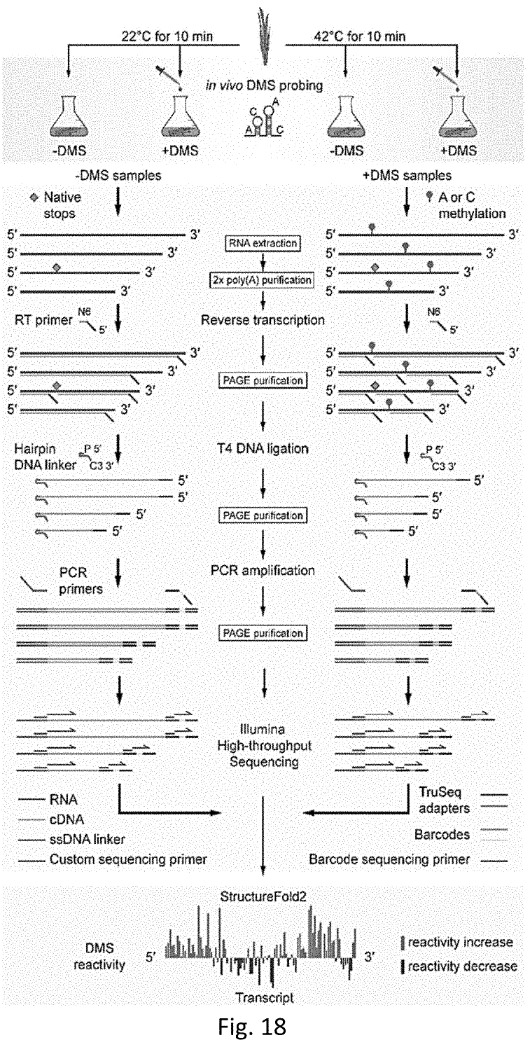

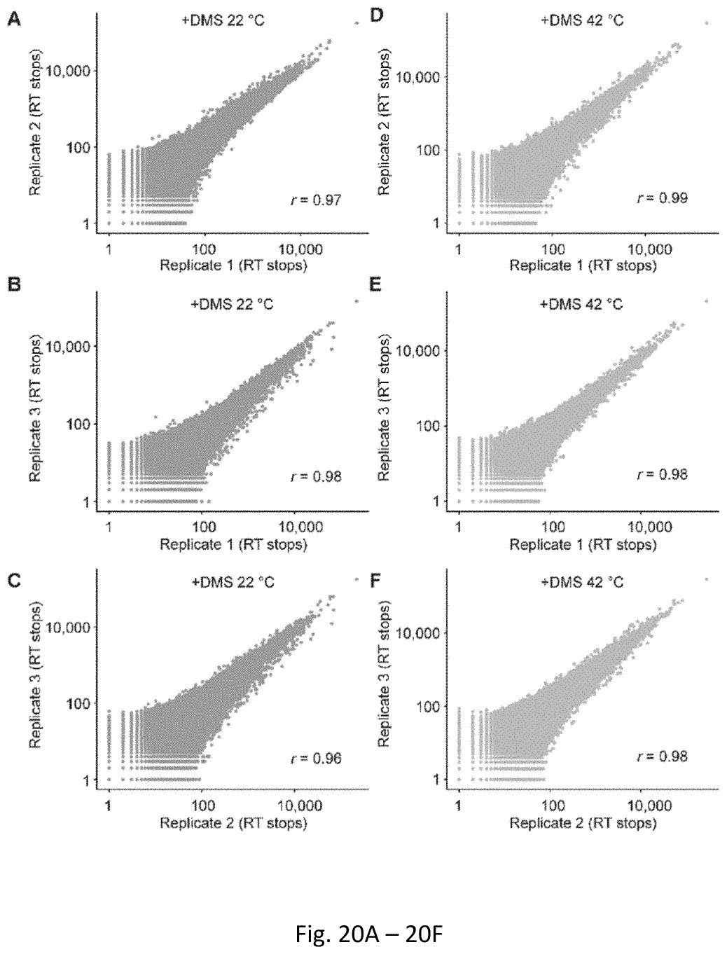

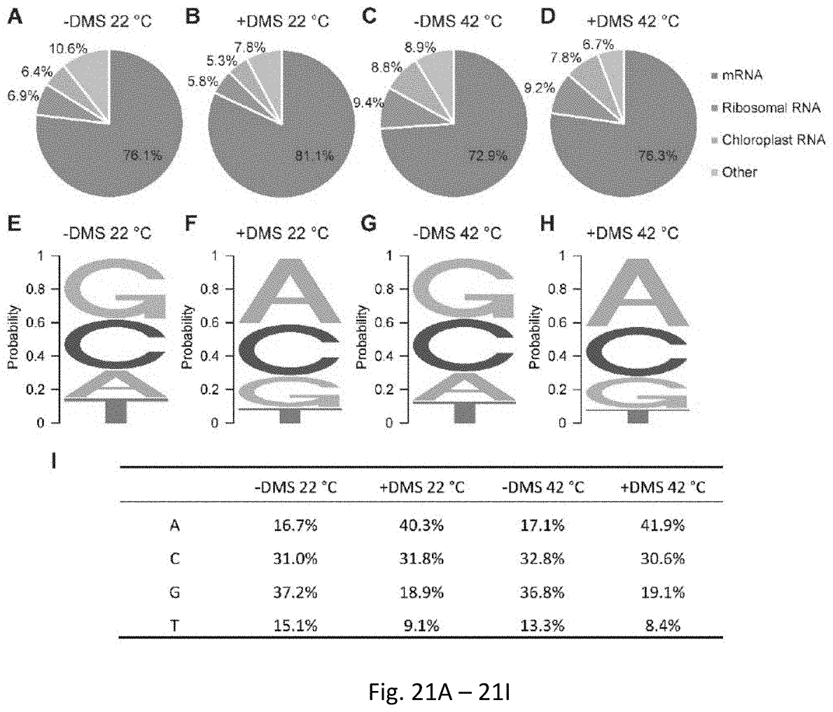

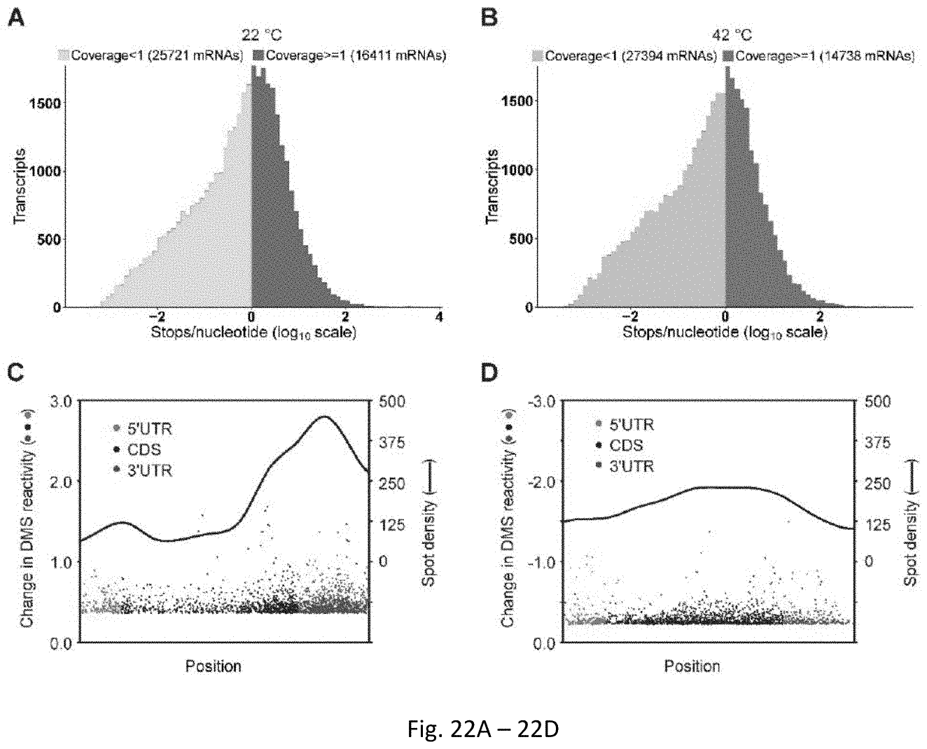

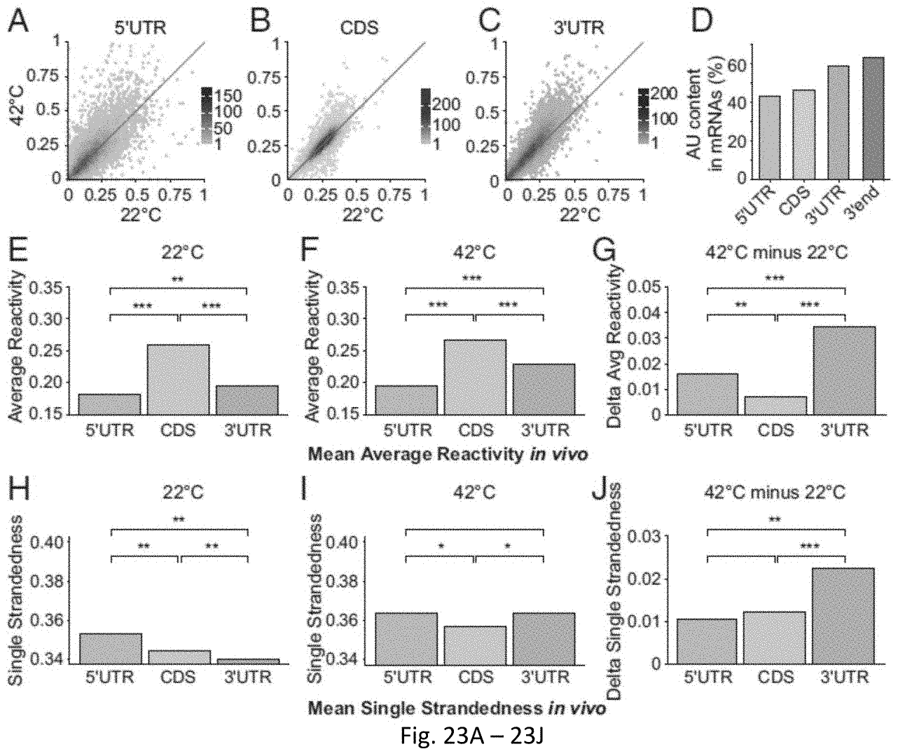

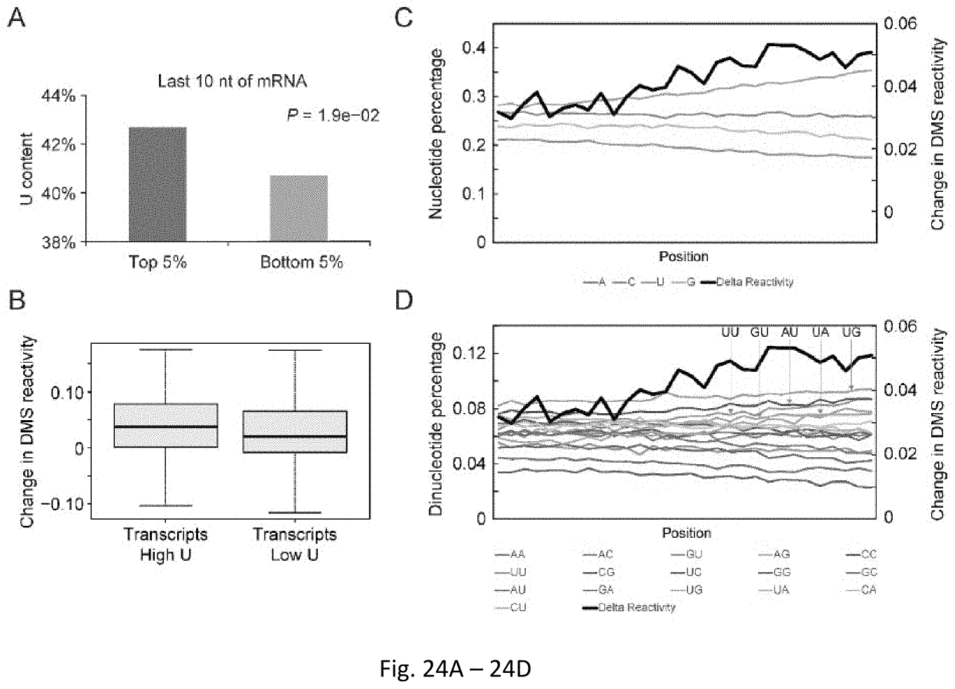

The optimized Structure-seq2 methodology (19) employs structure probing with dimethyl sulfate (DMS), which methylates adenines and cytosines on their Watson-Crick face (N1 of A and N3 of C) when they are not base-paired or otherwise protected. This methylation results in termination of reverse transcription, thus providing a read-out of the position of the modified, non-base-paired nucleotide FIG. 18, Structure-seq libraries Table 7 were generated from 14-day-old rice shoot tissue after a brief (10 minute) treatment at 22° C. (control) or 42° C. (heat shock) with or without DMS (FIG. 19A). The data show high reproducibility between biological replicates FIG. 20 and the majority of the reads map to mRNAs (FIG. 21A through FIG. 21ID). The data demonstrate the expected specificity for modification of A and C in DMS-treated samples (FIG. 21E through FIG. 21I). A short, 10-minute heat shock was used, both to optimize study of direct temperature effects on the RNA structurome, which should be rapid, and because such acute events are commonplace in crop and forest canopies because of transient heating from sunflecks (22). Sufficient structural coverage was obtained at both temperatures for 14,292 mRNAs (FIG. 19B, FIG. 19C, FIG. 22A and FIG. 22B). After normalization for chemical reactivity differences between temperatures (see Example 4), a global trend of significantly elevated average DMS reactivity at 42° C. compared with 22° C. was observed for entire transcripts (FIG. 19D), as well as for subregions (FIG. 23A through FIG. 23C). Because RNA secondary structures can melt anywhere between 1° C. and 99° C. (23), these results suggest that secondary structures of many mRNAs in rice have evolved to melt in vivo over this biologically relevant temperature range. The 3′UTRs showed the most significant increase in average DMS reactivity under heat (P=2.24×10⁻⁸⁹; (FIG. 23C, FIG. 22C and FIG. 22D). Interestingly, rice 3′UTRs harbor a higher AU content (FIG. 23D); given the weaker base-pairing of AU versus GC, this provides a mechanistic basis for melting of this region of the mRNA (FIG. 24). After folding of whole transcripts, using the in vivo restraints, that 3′UTRs are predicted to be more structured than 5S′UTRs or CDSs at 22° C., as has also been reported for mammalian 3′UTRs (24), yet show the greatest gain in predicted single-strandedness at 42° C., consistent with the marked increase in reactivity of 3′UTRs at 42° C. (FIG. 23E through FIG. 23I). These results suggest that rice 3′UTRs have increased susceptibility versus other regions of the transcript to melting out on acute heat shock (FIG. 23H through FIG. 23J).

TABLE 7
Mapping statistics of Structure-seq libraries,
generated using the Structure-seq2 protocol

| | | | Genome | | cDNA | |
|---|---|---|---|---|---|---|
| | Library | | mapped reads | % of | mapped reads | % of |
| Condition | replicate | All reads^{a,b} | (GMR) | All | (CMR) | GMR |
| −DMS | 1 | 58,686,867 | 51,393,158 | 87.6% | 39,320,200 | 76.5% |
| 22° C. | 2 | 54,050,775 | 46,705,657 | 86.4% | 35,187,054 | 75.3% |
| | 3 | 43,985,174 | 38,405,625 | 87.3% | 29,206,155 | 76.1% |
| | total | 156,722,816 | 136,504,440 | 87.1% | 103,713,409 | 76.0% |
| +DMS | 1 | 67,866,993 | 60,199,941 | 88.7% | 49,203,569 | 81.7% |
| 22° C. | 2 | 44,979,760 | 39,058,142 | 86.8% | 31,845,670 | 81.5% |
| | 3 | 59,668,373 | 53,052,732 | 88.9% | 42,483,881 | 80.1% |
| | total | 172,515,126 | 152,310,815 | 88.3% | 123,533,120 | 81.1% |
| −DMS | 1 | 49,519,857 | 43,477,224 | 87.8% | 30,058,553 | 69.1% |
| 42° C. | 2 | 46,376,890 | 41,483,875 | 89.5% | 29,681,209 | 71.6% |
| | 3 | 45,514,706 | 40,344,033 | 88.6% | 31,541,691 | 78.2% |
| | total | 141,411,453 | 125,305,132 | 88.6% | 91,281,453 | 72.8% |
| +DMS | 1 | 59,495,970 | 52,715,770 | 88.6% | 40,814,235 | 77.4% |
| 42° C. | 2 | 43,235,010 | 38,268,283 | 88.5% | 28,967,456 | 75.7% |
| | 3 | 57,443,353 | 50,652,383 | 88.2% | 38,257,273 | 75.5% |
| | total | 160,174,333 | 141,636,436 | 88.4% | 108,038,964 | 76.3% |

^{a}High quality (Q > 30) with length over 20 nt which is the minimum required for mapping in this study.
^{b}Total of 630 million high quality reads in all libraries combined.

Heat-Induced RNA Structural Changes in Rice Differ from Known Prokaryotic RNA Temperature-Sensing Mechanisms

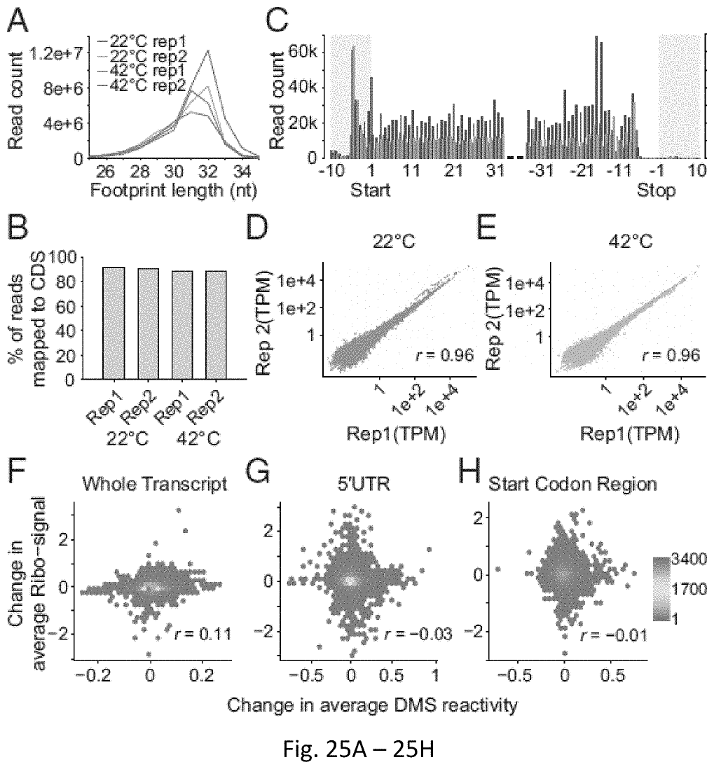

In bacteria, temperature-induced changes in 5′UTR structures of individual RNAs, referred to as RNA thermometers, modulate translation efficiency (15). In rice RNA structuromes, variation in heat induced structural reactivity change was greater in 5′UTRs (FIG. 23A) than in other transcript regions (FIG. 23B and FIG. 23C). A possible relationship between mRNA structure and translation was explored. Ribo-seq translatome profiles were determined after 10 minutes of the same temperature treatments as for Structure-seq libraries (FIG. 19A, FIG. 25A through FIG. 25E and Table 8. However, no correlation was found between the average temperature-induced change in DMS reactivity in the whole transcript, around the start codon, or in the entire 5′UTR, and change in ribosome association between temperatures (FIG. 25F through FIG. 25H). Nor was evidence found in the rice transcriptome or structurome itself for several specific known bacterial RNA thermometers (25)(see Example 4). Thus, heat induced RNA structural changes in rice identified here appear to differ from those described to date in prokaryotes. The data presented herein suggest that application of global in vivo structure probing methods to prokaryotes would reveal temperature-dependent relationships between mRNA structure and mRNA abundance such as those described here.

TABLE 8
Mapping statistics of Ribo-seq libraries

| | | | | | Nuclear- | Chloroplast- | |
|---|---|---|---|---|---|---|---|
| | | | | Genome | encoded rRNA | encoded rRNA | cDNA |
| Sample | | | | mapped reads^{b} | mapped reads^{b} | mapped reads^{b} | mapped reads |
| name | Temp. | Rep. | All reads^{a} | (% of All) | (% of All) | (% of All) | (% of All) |
| C10m^{c} | 22° C. | 1 | 144,214,556 | 133,066,498 | 64,790,999 | 31,472,873 | 55,216,455 |
| | | | | (92.3%) | (44.9%) | (21.8%) | (38.3%) |
| | | 2 | 133,960,640 | 123,943,648 | 60,051,024 | 31,062,270 | 43,932,282 |
| | | | | (92.5%) | (44.8%) | (23.2%) | (32.8%) |
| H10m^{c} | 42° C. | 1 | 134,512,418 | 124,759,176 | 52,210,311 | 17,063,054 | 29,721,738 |
| | | | | (92.7%) | (38.8%) | (12.7%) | (22.1%) |
| | | 2 | 133,690,235 | 124,781,078 | 61,442,088 | 20,380,018 | 37,142,708 |
| | | | | (93.3%) | (46.0%) | (15.2%) | (27.8%) |

^{a}High quality (Q > 30) and adapter trimmed reads.
^{b}Some reads map to both nuclear and chloroplast genomes.
^{c}In the sample name, “C” indicates control, “H” indicates 42° C. × 10 min heat treatment.

Heat-Induced Unfolding Promotes Transcript Degradation

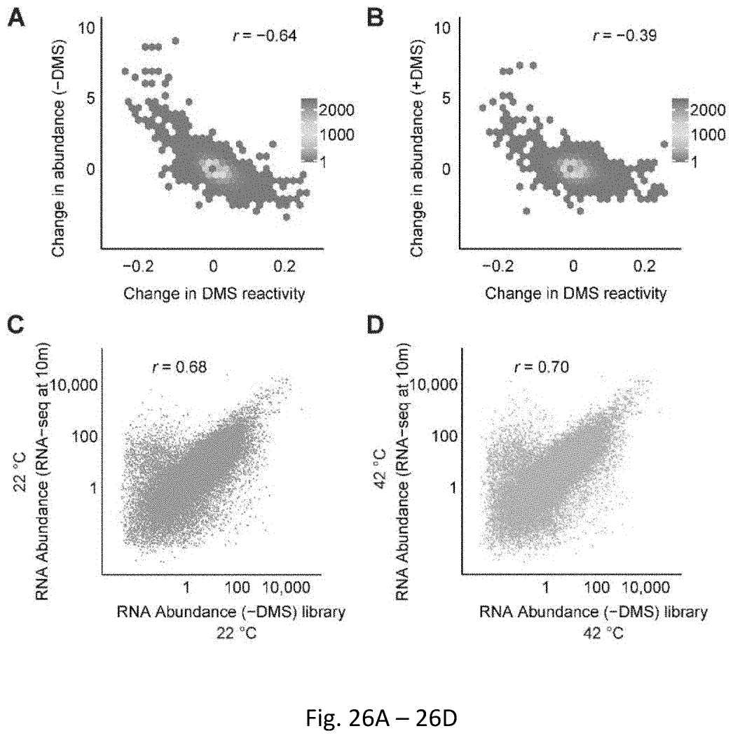

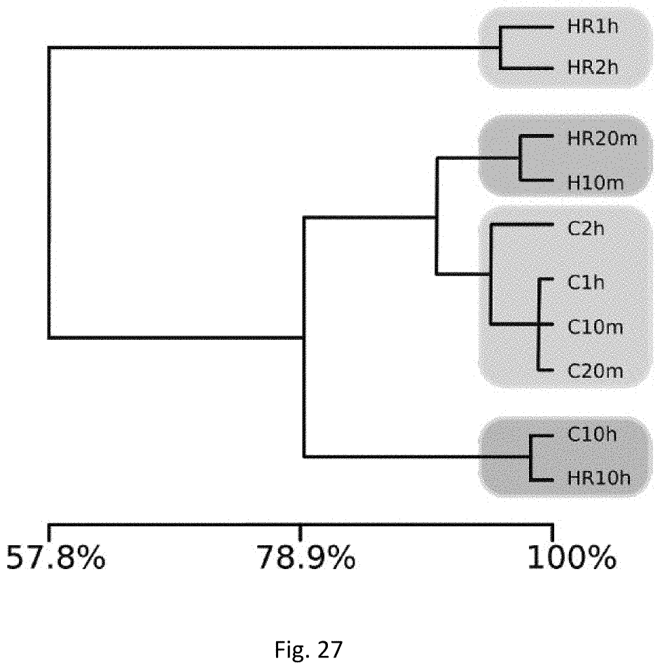

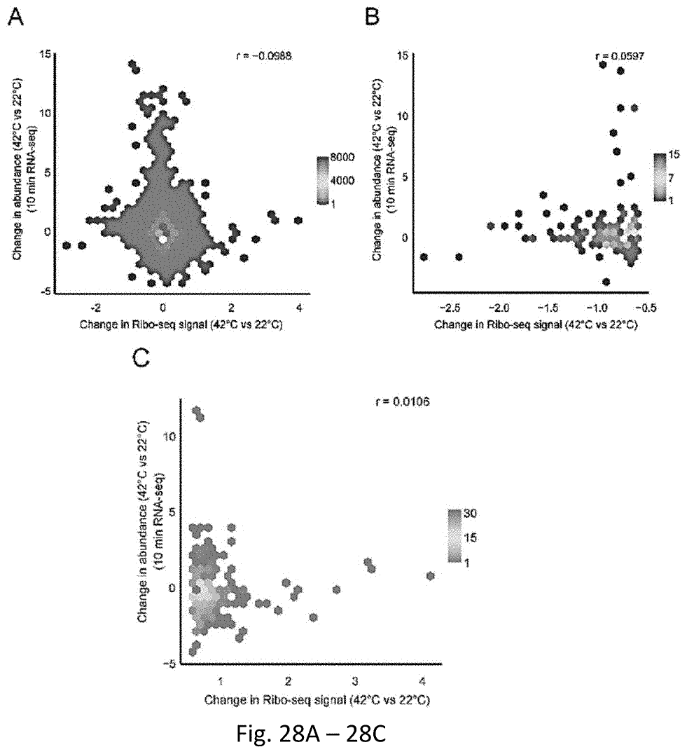

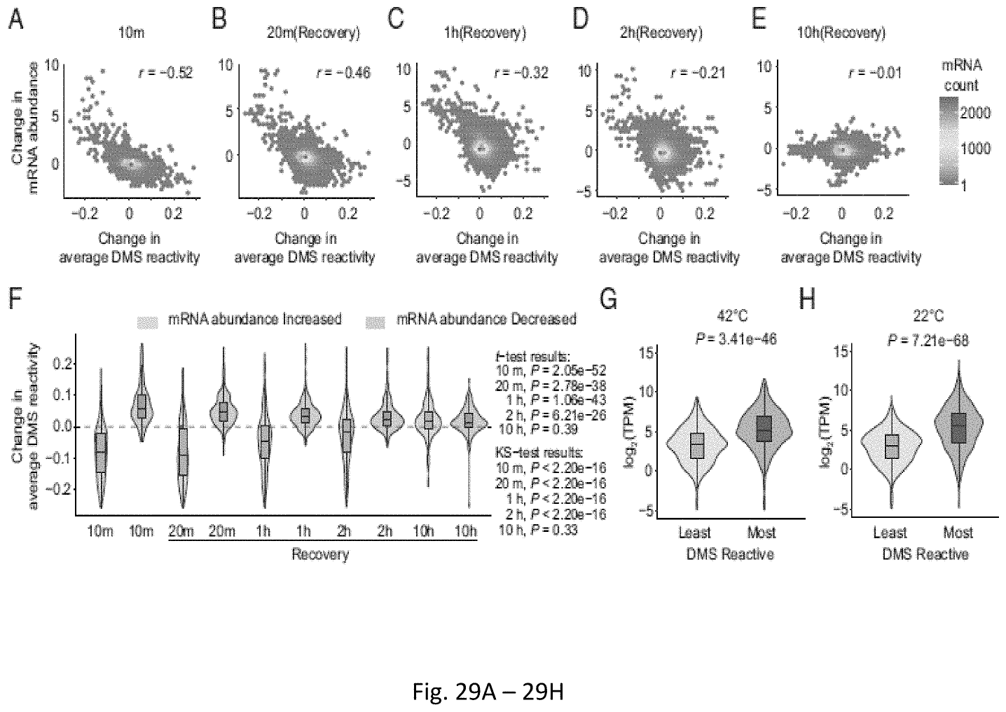

Rapid changes in plant mRNA transcriptomes in response to stimuli have been documented (26). Without being bound by theory, it was anticipated that acute heat shock might result in mRNA abundance changes, and was hypothesized that RNA structure could be regulatory of such changes, indeed, it was found that of the 14,292 transcripts for which there was Structure-seq data at both temperatures, 1,052 (7.4%) showed a statistically significant change in abundance between 42° C. heat shock and 22° C. control samples. A strong inverse correlation was observed between temperature-induced change in DMS reactivity and temperature-induced change in transcript abundance as quantified from −DMS libraries (note that reads from −DMS libraries are analogous to RNA-seq library reads; (FIG. 26A and FIG. 26B). To further evaluate the relationship between RNA structure change and transcript abundance change, classical RNA-seq experiments were performed that quantified transcript abundance change over a longer time course post-heat shock (FIG. 19A) after the same 10 minutes of 42° C. or 22° C. conditions as were employed in the RNA structurome experiments (FIG. 27, FIG. 28 and Table 9). RNAseq data at 10 minutes were highly consistent with the mRNA abundance measurements from −DMS Structure-seq libraries (FIG. 26C and FIG. 26D). The RNA-seq experiments confirmed a significant negative correlation between change in DMS reactivity and change in transcript abundance at 10-20 minutes after heat shock, and even out to 1 hour (FIG. 29A through FIG. 29C). After 2 hours, and especially after 10 hours, the correlation weakened and was eventually lost (FIG. 29D and FIG. 29E), presumably reflecting a mechanism in which the structurome and transcriptome are rapidly affected by heat shock and then slowly recover (FIG. 27). A converse analysis, in which mRNAs with the greatest increase or decrease in abundance between temperatures were first identified and then analyzed for DMS reactivity, confirmed this inverse relationship as well as its time dependence (FIG. 29F). Next, possible mechanistic origins of this effect were investigated. These results suggested that at least part of the inverse relationship between reactivity and abundance arises from preferential degradation of less structured, highly reactive transcripts. The exosome complex is responsible for one of the major pathways of RNA degradation and is largely conserved throughout eukaryotes, it degrades RNA in a 3′-to-5′ direction (27), and only RNAs with a sufficiently long single-stranded 3′ tail can initiate tunneling through the exosome core (28). Thus, exosome mediated degradation of unfolded transcripts would be consistent with the observation of heat-induced DMS reactivity increases in 3′UTRs (FIG. 23E through FIG. 23G). With the notion of 3′ end-initiated degradation of the RNA, the 5% of mRNAs with greatest heat-induced increase or decrease in DMS reactivity were compared and the former set of transcripts was found to have significantly greater U content in the final 10 nt of the 3′UTR (FIG. 24A). This result is consistent with U base-pairing with the adjacent polyA tail that at least partially melts out at 42° C. and facilitates exosome-based degradation.

TABLE 9
Mapping statistics of time course RNA-seq libraries

| Treatment | | | | | Genome | | | |
|---|---|---|---|---|---|---|---|---|
| Temp. & | Recovery | Sample | | Total | mapped reads | % of | Multi- | % of |
| duration | time | Name^{a} | Rep. | reads^{b} | (GMR) | total | mapped reads | GMR |
| 22° C. | 0 min | C10m | 1 | 33,869,785 | 32,978,562 | 97.4% | 1,776,249 | 5.2% |
| for 10 min | | | 2 | 33,531,678 | 32,460,700 | 96.8% | 1,892,381 | 5.6% |
| | 10 min | C20m | 1 | 31,261,223 | 30,760,972 | 98.4% | 1,418,674 | 4.5% |
| | | | 2 | 43,170,666 | 42,285,712 | 98.0% | 2,246,220 | 5.2% |
| | 50 min | C1h | 1 | 35,228,054 | 34,510,009 | 98.0% | 1,387,559 | 3.9% |
| | | | 2 | 36,928,419 | 35,989,953 | 97.5% | 1,791,360 | 4.9% |
| | 1 h | C2h | 1 | 29,090,627 | 28,391,336 | 97.6% | 1,196,994 | 4.1% |
| | 50 min | | 2 | 32,243,175 | 31,408,171 | 97.4% | 1,386,573 | 4.3% |
| | 9 h | C10h | 1 | 41,378,526 | 40,347,435 | 97.5% | 2,057,179 | 5.0% |
| | 50 min | | 2 | 33,635,765 | 32,669,302 | 97.1% | 1,810,499 | 5.4% |
| 42° C. | 0 min | H10m | 1 | 45,314,781 | 44,186,535 | 97.5% | 2,320,600 | 5.1% |
| for 10 min | | | 2 | 33,168,426 | 32,416,819 | 97.7% | 1,534,421 | 4.6% |
| | 10 min | HR20m | 1 | 33,440,984 | 32,159,843 | 96.2% | 3,868,691 | 11.6% |
| | | | 2 | 33,604,361 | 32,695,384 | 97.3% | 1,596,237 | 4.8% |
| | 50 min | HR1h | 1 | 38,936,971 | 37,407,796 | 96.1% | 3,380,004 | 8.7% |
| | | | 2 | 29,188,625 | 28,375,413 | 97.2% | 1,722,330 | 5.9% |
| | 1 h | HR2h | 1 | 32,705,369 | 31,808,867 | 97.3% | 1,580,836 | 4.8% |
| | 50 min | | 2 | 41,806,142 | 40,970,085 | 98.0% | 2,273,954 | 5.4% |
| | 9 h | HR10h | 1 | 33,685,237 | 32,834,538 | 97.5% | 3,069,028 | 9.1% |
| | 50 min | | 2 | 33,292,915 | 31,760,585 | 95.4% | 1,896,497 | 5.7% |

^{a}In the sample name, “C” indicates control, “H” indicates 42° C. × 10 min heat treatment and “HR” means recovery after the 42° C. × 10 min heat treatment. Timepoints are identical to those shown in FIG. 1a.
^{b}Total of 707 million reads in all libraries.

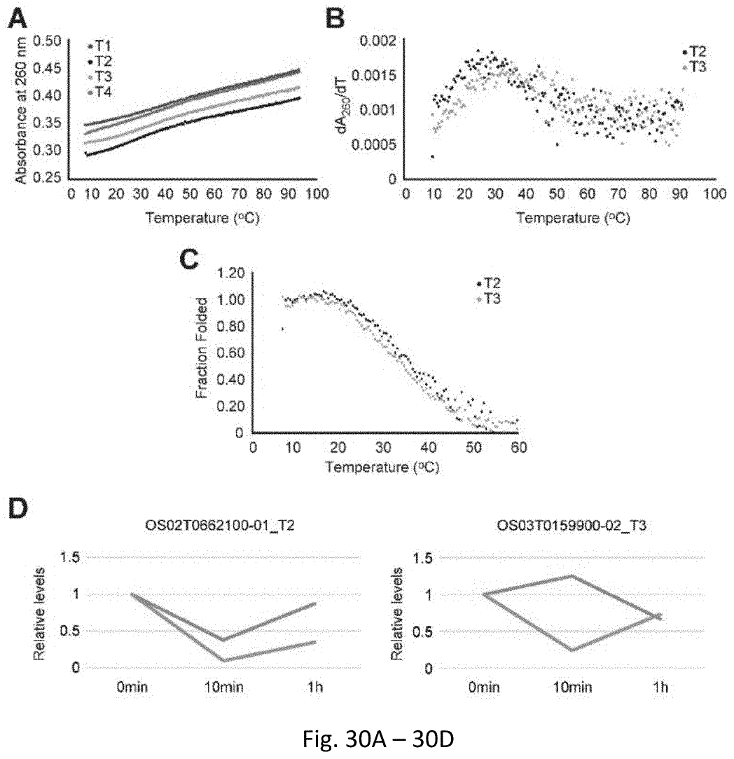

To test the hypothesis that increased reactivity in the 3′UTR arises from heat-induced unfolding of RNA structure, four 3′UTR sequences were selects and RNAs were prepared comprising the last 10 nt of each transcript fused to a 15-nt polyA tail (designated T1-T4). Sequences were chosen from 3′UTR sequences in the top 5% of transcripts with greatest loss in abundance at 42° C. T1-T4 also had predicted maximal gain in single-strandedness between 22° C. and 42° C., as derived from free energy estimations at these temperatures, using standard thermodynamic relationships. The stability of T1-T4 structures was assessed by UV-detected thermal denaturation monitored at 260 nm, using in vivo-like monovalent and divalent ion concentrations. Plots of fraction folded versus temperature (FIG. 30) revealed that T2 and T3 (but not T1 and T4) melt with a sigmoidal transition between ˜20 and 40° C., which are temperatures similar to those used for unstressed and heat-stressed rice, respectively. It is notable that T2 and T3 have the highest U content for the last 10 nt of the transcript, of 6 and 7 Us, respectively, whereas T1 and T4 have lower U content of 2 and 5 Us, respectively. The higher U content in T2 and T3, which comes in two regions of at least two Us each, could drive Watson-Crick base pairing with the polyA tail at 22° C., which then melts out at 42° C. These data demonstrate melting of U-rich 3′UTR sequences by 42° C., which could provide the exosome with access to the 3′ end for degradation.

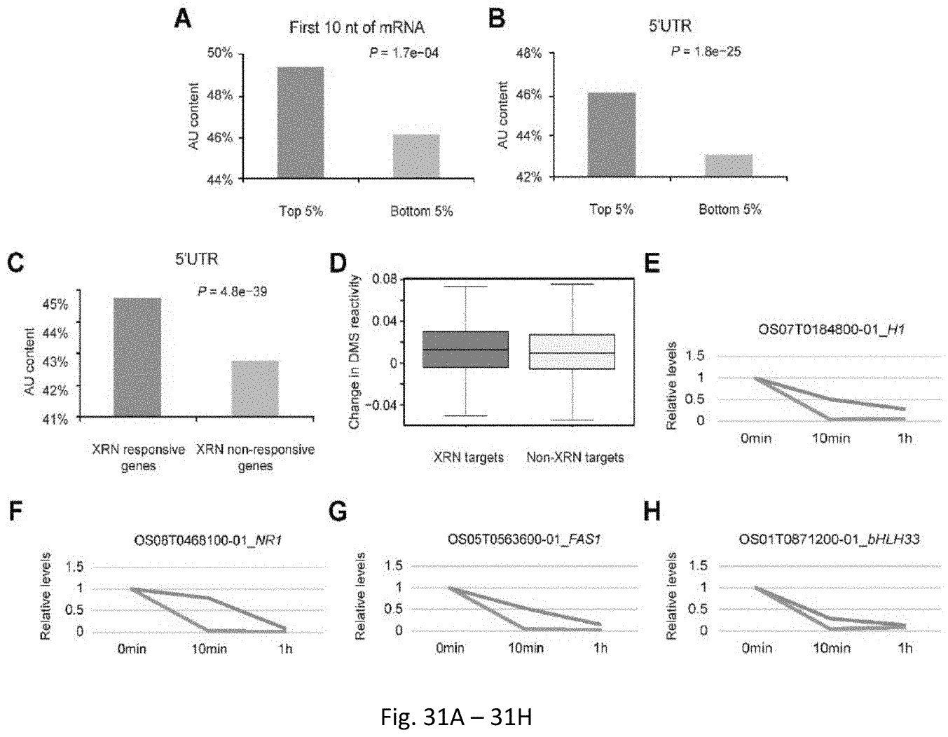

In addition to degradation from the 3′ end. RNA degradation can occur from the 5′end, catalyzed in plants by the plant ortholog of XRN1, XRN4, which is a 5′-to-3′single-stranded exonuclease known to be activated under heat (29). The 5′UTRs of rice orthologs of Arabidopsis XRN4-sensitive transcripts (29) were analyzed and it was found that these transcripts have enriched 5′UTR AU content relative to XRN4-insensitive targets (FIG. 31). The 5% of mRNAs with greatest heat-induced reactivity increase also have enriched AU content at the 5′end, as well as in the entire 5′UTR (FIG. 31), which would facilitate enhanced unfolding, given the weaker base-pairing of AU versus GC, and thus, degradation at higher temperatures.

To further evaluate the hypothesis of a functional relationship between structure changes in the 5′UTR and transcript abundance, the abundance of degradome fragments of the 5% least- and 5% most-reactive mRNAs were compared using data from a rice degradome dataset (GSM1040649; Materials and Methods). [By design, degradome libraries are enriched in uncapped mRNAs subject to 5′-to-3′degradation (30); degradome sequencing thus specifically identifies fragments of degraded mRNA, and so allows an approximate quantification of transcript stability.] At each temperature, the set of transcripts with higher average DMS reactivity were found to have significantly greater abundance of transcript fragments in the degradome (FIG. 29G and FIG. 29H). This finding suggests that high DMS reactivity transcripts are more susceptible to degradation from 5′ ends. Taken together, these results (FIG. 23 and FIG. 29) indicate that melting of both 5′ and 3′ UTRs with heat contributes to mechanisms of selective transcript degradation, and thus transcriptome reprogramming in response to acute heat stress.

Recent technical advances have facilitated the field of RNA structural genomics, allowing studies of RNA structure in vivo and genome-wide (31). Although these tools are powerful, there have been very few studies of in vivo structuromes, let alone in response to stress. The Structure-seq methodology (19) allowed us to probe heat-induced structural changes at single-nucleotide resolution in thousands of transcripts simultaneously (FIG. 19), providing a genome-wide perspective on in vivo temperature modulation of RNA structure, Although other mechanisms undoubtedly contribute, the comprehensive structurome, transcriptome, and translatome results are consistent with a major regulatory role in eukaryotes of temperature-modulated mRNA structures that control mRNA abundance, as opposed to control of mRNA translation as in prokaryotes (32).

In prokaryotes, temperature-induced RNA structural changes around the Shine-Dalgamo sequence exert regulatory roles in protein translation (14). In particular, sequences defined as the ROSE element, four U, and UCCU are prokaryotic 5′UTR RNA thermometers. These motifs sequester the Shine-Dalgamo sequence at low temperatures and melt out at higher temperatures, thus promoting ribosome binding. Only a few of these sequence candidates were found in the 5′UTR dataset, and none exhibited unfolding at 42° C. as would be expected for RNA thermometers. In eukaryotes, the Kozak sequence guides translation initiation. However, only 156 mRNAs containing Kozak sequences were present in both the structurome and Ribo-seq datasets, and these did not exhibit a correlation between DMS reactivity change and heat-induced Ribo-seq signal change in the translatome. These results suggest that RNA-based temperature-sensing mechanisms of eukaryotes differ markedly from those of prokaryotes. These experimental and computational conclusions differ from a previous study in which analysis of a single mRNA, Drosophila melanogaster HSP90, suggested that eukaryotes use prokaryotic-type RNA thermometers (33). This comparison illustrates the value of a genome-wide perspective on in vivo RNA structure.

AU richness was observed in both 3′ and 5′ UTRs that exhibit elevated DMS reactivity at 42° C. (FIG. 24 and FIG. 31), consistent with their melting out, and heat-induced DMS reactivity changes show a strong inverse correlation with heat induced changes in transcript abundance (FIG. 29). These results are suggestive of AU-rich thermometers located in both 5′ and 3′ UTRs, whose melting facilitates RNA degradation; this conclusion is supported by the melts on representative candidates. Consistent with this interpretation, in yeast, mRNAs with a lower in vitro estimated melting temperature declined in abundance under heat shock compared with mRNAs with a higher estimated melting temperature, which was attributed to greater exosome access to unstructured RNA (13).

Evidence for temperature-induced unfolding in 5′UTRs that is associated with mRNA degradation was also observed. A previous study on Arabidopsis reported the down-regulation of several thousand mRNAs after heat shock (29). The majority (85%) of the down-regulated transcripts lost down-regulation in an xm4 mutant (29). Because XRN4 is a single-stranded 5′ to 3′ nuclease, their observation together with the RNA structurome analysis suggest that 5′UTR unfolding facilitates XRN4-mediated degradation, and targeted decay analyses are consistent with this suggestion (FIG. 31).

Protection from DMS reactivity can be afforded by both base pairing and protein binding; thus, the hypothesis that some of the DMS reactivity increases that were observed might be a result of heat-induced loss of RNA-binding proteins in UTR regions was evaluated. Recently, 3′UTR-seq in zebrafish embryos found that AU-rich elements correlated with accelerated degradation after zygotic genome activation (34). In the same study, polyU and UUAG sequences were also associated with delayed degradation of maternal mRNAs early in embryogenesis. In both cases, it was proposed that association with zebrafish mRNA binding proteins, rather than RNA structure, controlled degradation (34). However, a directed analysis of all instances of the UUAG motif in the 3′UTRs of the structurome libraries revealed more instances of no heat-induced change in reactivity (11,861) than either positive (5,157) or negative (3,423) reactivity changes, whereas a change in protein affinity for the binding site should have had a pervasive and uniform signature if protein dissociation was the major causal agent of reactivity changes, 3′UTRs were also assessed in the structurome datasets for the presence of sequences identified as protein-binding mRNA motifs front a PIP-seq analysis in Arabidopsis (35). No enrichment of such motifs was found in regions of the 3′UTR associated with the most increased reactivity on heat exposure, again suggesting that many of the reactivity increases are independent of protein unbinding. Thus, at present, there is no evidence that loss of protein protection has a major contribution to the heat-induced gain in DMS reactivity in rice UTRs.

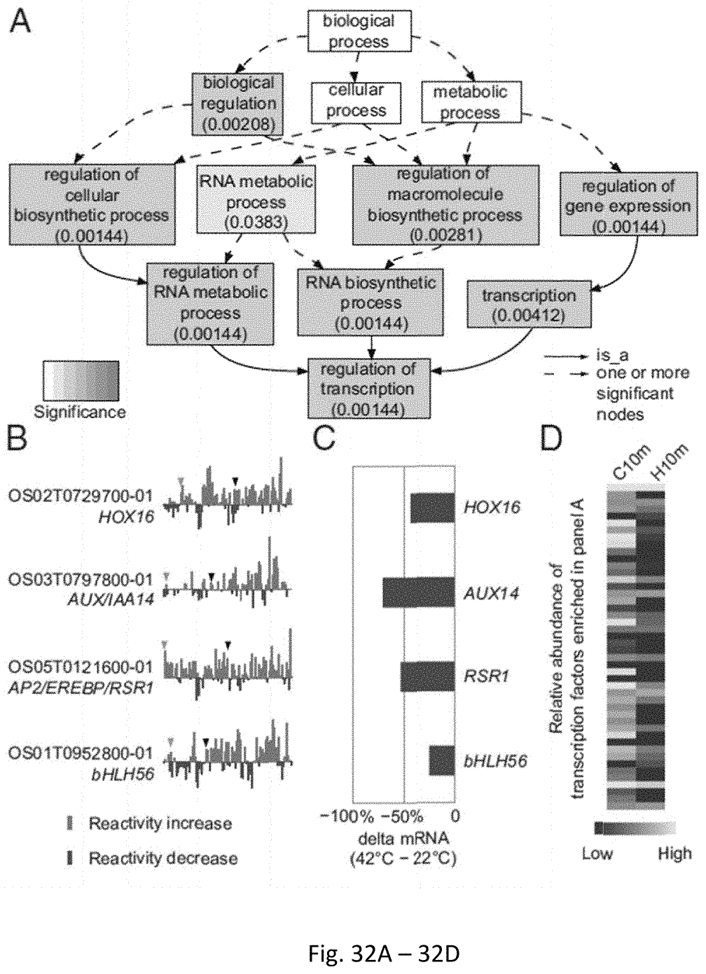

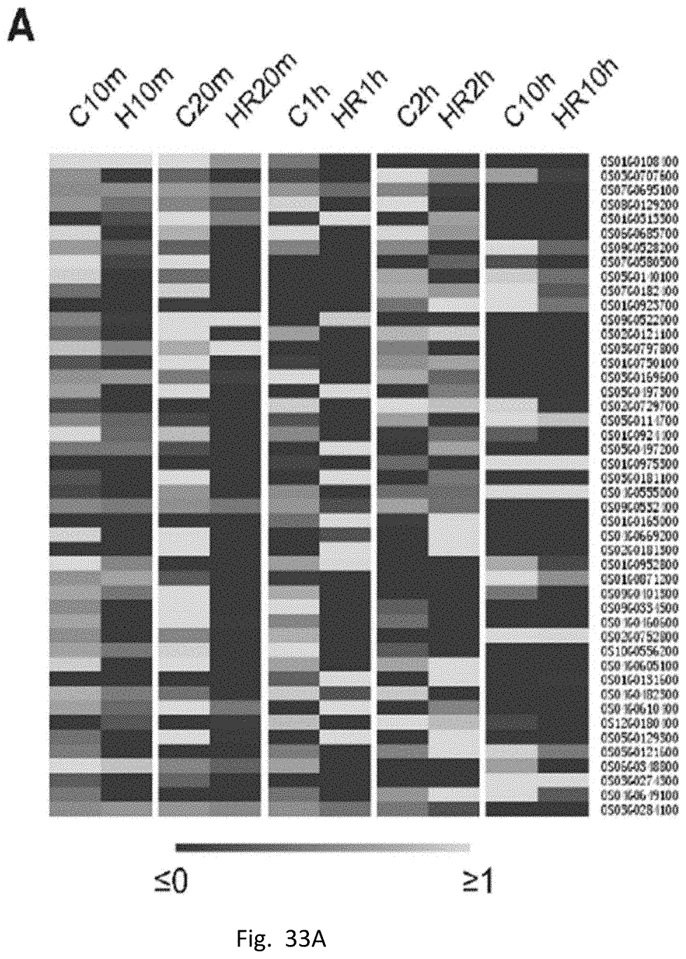

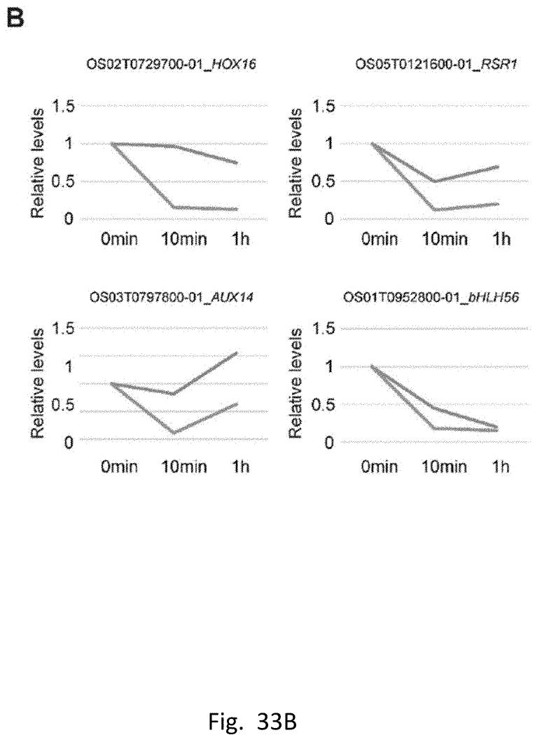

The functional roles of mRNAs with elevated DMS reactivity in response to heat shock (FIG. 32) were evaluated. Gene ontology analysis of the 5% of mRNAs with the greatest heat-induced increase in average DMS reactivity showed a significant overrepresentation of genes that function in transcriptional regulation (FIG. 32A and FIG. 32B). Application of an established assay of mRNA decay (36) confirmed that mRNAs of four transcription factors with dramatic heat-induced DMS reactivity increases showed accelerated decay (FIG. 32C), whereas the RNA-seq analyses broadly confirmed that transcription factors in this category rapidly declined in abundance after heat shock (FIG. 32D and FIG. 33). The functional (FIG. 32) and biochemical (FIG. 30 and FIG. 33) analyses provide a likely mechanistic underpinning to a previous observation that heat stress reduces transcription factor mRNA abundance in rice floral tissues during anthesis (37), a stage of reproductive development in crops that is particularly sensitive to yield losses after heat stress (1), As transcription factors are master regulators of gene expression, the results may imply a type of widespread hierarchical control of transcriptional regulation mediated by RNA structure change in response to temperature, Interestingly, heat shock transcription factors apparently escape this regulatory mechanism, as those in the dataset show only minor DMS reactivity changes after heat shock (Table 10).

TABLE 10
Heat shock transcription factors (HSFs) with coverage
in Structure-seq datasets show diverse changes in average
DMS reactivity at 42° C. as compared to 22° C.

| | | | | Dif- |
|---|---|---|---|---|
| | | Heat | RT | fer- |
| Transcript ID | Description | (42° C.) | (22° C.) | ence |
| OS01T0733200-01 | Similar to Heat shock | 0.17 | 0.18 | 0.01 |
| | transcription factor 29 | | | |
| OS01T0749300-01 | Heat shock transcription | 0.19 | 0.14 | −0.05 |
| | factor | | | |
| OS01T0749300-02 | Heat shock transcription | 0.18 | 0.14 | −0.04 |
| | factor | | | |
| OS02T0527300-01 | Similar to Heat shock | 0.23 | 0.21 | −0.02 |
| | transcription factor 31 | | | |
| OS03T0161900-01 | Similar to Heat shock | 0.24 | 0.06 | −0.18 |
| | transcription factor A-2d | | | |
| OS03T0795900-01 | Similar to Heat shock | 0.23 | 0.18 | −0.05 |
| | transcription factor 31 | | | |
| OS03T0854500-01 | Similar to Heat shock | 0.25 | 0.27 | 0.02 |
| | transcription factor 31 | | | |
| OS03T0854500-02 | Similar to Heat shock | 0.26 | 0.27 | 0.01 |
| | transcription factor 31 | | | |

In summary, given the multifaceted effects of temperature on RNA structure discovered in this in vivo study of RNA structurome modulation by supraoptimal temperatures, it is proposed that much of the eukaryotic transcriptome functions as an environmental thermosensor. It is proposed that in eukaryotes, transcripts are dynamically subject to degradation by a molecular mechanism involving heat-induced secondary structure unfolding in AU-rich 5′- and 3′-UTRs. Given that RNA structure can be regulated independent of encoded protein sequence through variation in UTR sequence and synonymous SNPs (38), these observations suggest mechanisms by which rice and other crops could be engineered to better withstand temperature and other stresses.

### REFERENCES

1. Bita C E. Gerats T (2013) Plant tolerance to high temperature in a changing environment: Scientific fundamentals and production of heat stress-tolerant crops. Front Plant Sci 4:273.
2. Battisti D S. Naylor R L (2009) Historical warnings of future food insecurity with unprecedented seasonal heat. Science 323:240-244.
3. Peng S, et al. (2004) Rice yields decline with higher night temperature from global warming. Proc Nat Acad Sci USA 101:9971-9975.
4. Zhao C, et al. (2017) Temperature increase reduces global yields of major crops in four independent estimates. Proc Natl Acad Sci USA 114:9326-9331.
5. Kosová K, Vitamvis P, Prášil I T, Renaut J (2011) Plant proteome changes under abiotic stress—Contribution of proteomics studies to understanding plant stress response. J Proteomics 74:1301-1322.
6. Obata T, et al. (2015) Metabolite profiles of maize leaves in drought, heat, and combined stress field trials reveal the relationship between metabolism and grain yield. Plant Physiol 169:2665-2683.
7. Kotak S, et al. (2007) Complexity of the heat stress response in plants. Curr Opin Plant Biol 10:310-316.
8. Bevilacqua P C, Ritchey L E, Su Z, Assmann S M (2016) Genome-wide analysis of RNA secondary structure. Annu Rev Genet 50:235-266.
9. Schmitz K M, Mayer C, Postepska A, Grumnt 1(2010) Interaction of noncoding RNA with the rDNA promoter mediates recruitment of DNMT3b and silencing of rRNA genes. Genes Dev 24:2264-2269.
10. Buratti E, Baralle F E (2004) Influence of RNA secondary structure on the pre-mRNA splicing process. Mol Cell Biol 24:10505-10514.
11. Kutchko K M, et al. (2015) Multiple conformations are a conserved and regulatory feature of the RBI 5′ UTR. RNA 21:1274-1285.
12. Toscano C, et al. (2006) A silent mutation (2939G>A, exon 6; CYP2D6*59) leading to impaired expression and function of CYP2D6. Pharmacogenet Genomics 16:767-770.
13. Wan Y, et al. (2012) Genome-wide measurement of RNA folding energies. Mol Cell 48:169-A181.
14. Righetti F, et al. (2016) Temperature-responsive in vitro RNA structurome of Yersinia pseudotuberculosis. Proc Natl Acad Sci USA 113:7237-7242.
15. Kortmann J, Narberhaus F (2012) Bacterial RNA thermometers: Molecular zippers and switches. Nat Rev Microbiol 10:255-265.
16. Ding Y, et al. (2014) In vivo genome-wide profiling of RNA secondary structure reveals novel regulatory features. Nature 505:696-700.
17. Wan Y, et al. (2014) Landscape and variation of RNA secondary structure across the human transcriptome. Nature 505:706-709.
18. Spitale R C, et al. (2015) Structural imprints in vivo decode RNA regulatory mechanisms. Nature 519:486-490.
19. Ritchey L E, et A (2017) Structure-seq2: Sensitive and accurate genome-wide profiling of RNA structure in vivo. Nucleic Acids Res 45:e135.
20. Deng H, et al. (2018) Rice in vivo RNA structurome reveals RNA secondary structure conservation and divergence in plants. Mo) Plant 11:607-622.
21. Leamy K A, Assmann S M, Mathews D H, Bevilacqua P C (2016) Bridging the gap between in vitro and in vivo RNA folding. Q Rev Biophys 49:e10.
22. Schymanski S J, Or D, Zwieniecki M (2013) Stomatal control and leaf thermal and hydraulic capacitances under rapid environmental fluctuations. PLoS One 8:e54231.
23. Tinoco I, Jr, Bustamante C (1999) How RNA folds, J Mol Biol 293:271-281.
24, Wu X, Bartel D P (2017) Widespread influence of 3′-end structures on mammalian mRNA processing and stability. Cell 169:905-917.e11.
25. Krajewski S S, Narberhaus F (2014) Temperature-driven differential gene expression by RNA thermosensors. Biochim Biophys Acta 1839:978-988.
26. McClure B A, Guilfoyle T (1987) Characterization of a class of small auxin-inducible soybean polyadenylated RNAs. Plant Mol Biol 9:611-623.
27. Lykke-Andersen S, Tomecki R. Jensen T H, Dziembowski A (2011.)
The eukaryotic RNA exosome: Same scaffold but variable catalytic subunits. RNA Biol 8:61-66,
28. Bonneau F, Basquin J. Ebert J, Lorentzen E, Conti E (2009) The yeast exosome functions as a macromolecular cage to channel RNA substrates for degradation, Cell 139:547-559.
29. Merret R., et al. (2015) Heat-induced ribosome pausing triggers mRNA co-translational decay in Arabidopsis thaliana. Nucleic Acids Res 43:4121-4132.
30. Addo-Quaye C., Eshoo T W, Bartel D P, Axtell M J (2008) Endogenous siRNA and miRNA targets identified by sequencing of the Arabidopsis degradome. Curr Biol 18:758-762.
31. Bevilacqua P C, Assmann S M (2018) Technique development for probing RNA structure in vivo and genome-wide. Cold Spring Harb Perspect Biol 10:a032250.
32. Mustoe A M, et al. (2018) Pervasive regulatory functions of mRNA structure revealed by high-resolution SHAPE probing. Cell 173:181-195.e18,
33. Ahmed R, Duncan R F (2004) Translational regulation of Hsp90 mRNA. AUG-proximal 5′-uttranslated region elements essential for preferential heat shock translation. J Biol Chem 279:49919-49930.
34. Rabani M, Pieper L. Chew G L, Schier A F (2017) A massively parallel reporter assay of 3′ UTR sequences identifies in vivo rules for mRNA degradation. Mol Cell 68:1083-1094.e5,
35. Gosai S J, et al. (2015) Global analysis of the RNA-protein interaction and RNA secondary structure landscapes of the Arabidopsis nucleus. Mol Cell 57:376-388.
36. Park S H, et at (2012) Posttranscriptional control of photosynthetic mRNA decay under stress conditions requires 3′ and 5′ untranslated regions and correlates with differential polysome association in rice. Plant Physiol 159:1111-1124.
37. Gonzaez-Schain N, et al (2016) Genome-wide transcriptome analysis during anthesis reveals new insights into the molecular basis of heat stress responses in tolerant and sensitive rice varieties. Plant Cell Physiol 57:57-68.
38. Solem A C, HalvorsenM, Ramos S B, Laederach A (2015) The potential of the riboSNitch in personalized medicine. Wiley Interdiscip Rev RNA 6:517-532.
39. Juntawong P, Girke T, Bazin J. Bailey-Serres J (2014) Translational dynamics revealed by genome-wide profiling of ribosome footprints in Arabidopsis. Proc Natl Acad Sci USA 111:E203-E212.

### Example 3: In Vivo RNA Structural Probing of Uracil and Guanine Base Pairing by 1-ethyl-3-3-dimethylaminopropyl carbodiimide (EDC)

Reagents that modify different positions of the nucleotides have been employed in in vivo structure-probing. SHAPE reagents, which react with the ribose sugar, have the advantage of modifying all four nucleotides, and can provide structural information because reactivity is strongly diminished by base pairing (Merino et al, 2005). While the original SHAPE reagents are not strongly membrane-permeant, the SHAPE reagent NAI crosses cell membranes, allowing in vivo application (Spitale et at 2013. Lee et al. 2017). Other reagents modify the Watson-Crick (WC) face of nucleotides such that the presence of reactivity directly indicates that the nucleotide is not engaged in standard base pairing or interaction with proteins. Dimethyl sulfate (DMS) alkylates the N1 of adenines (A) and the N3 of cytosines (C) and was the first reagent used to provide a genome-wide picture of the RNA structurome (Ding et al. 2014; Rouskin et al. 2014). Recently, glyoxal and its hydrophobic derivatives, methylglyoxal and phenylglyoxal, were developed as in vivo probes that block RT through modification of the WC amidine functionality of guanine (G), with significant but lesser reactivity on the amidine faces of A and C (Mitchell et al. 2018). Methyl- and phenylglyoxal proved more effective than glyoxal, likely because their more hydrophobic character allows increased permeation through the lipid bilayer. Finally, the recently-developed LASER reagent nicotinoyl azide (NAz) reacts via a light-triggered nitrene at the C8 position of purines, which is away from the WC face, and induces an RT stop (Feng et al, 2018), This reagent is of special interest because it is sensitive to protein protection and tertiary structure but is not generally influenced by base pairing.

Missing within this arsenal of in vivo structure-probing reagents is one that modifies the WC face of uracils (U), which make unique and important contributions to RNA structure. For instance. A-U pairing in the 3′ UTR is especially important in gene regulation (Wan et at, 2012; Rabani et al. 2017). Moreover, U tends to pair with both A and G, making absence of U base pairing particularly notable. The carbodiimide t-cyclohexyl-3-(2-morpholinoethyl)-carbodiimide methyl-p-toluenesulfonate (CMCT) has been used for many years to probe Us and Gs in vitro (Harris et al. 1995; Ziehler and Engelke 2001), but is not generally amenable to in vivo work. Cellular application of CMCT has been described but requires either sonication, cell lysates, or cell-damaging agents such as DMSO, high concentrations of CaCl₂), or sodium borate (Noller and Chaires 1972; Harris et al. 1995; Balzer and Wagner 1998; Antal et al, 2002; Incarnato et al. 2014). Therefore, currently only As, Cs, and Gs can be probed directly in vivo without cellular damage.

In this work, it is demonstrated that the water-soluble carbodiimide 1-ethyl-3-(3-dimethylaminopropyl)carbodiimide (EDC) can enter intact, non-permeabilized cells and react with the WC face of Us and Gs in RNAs with high specificity. EDC is a common reagent that is often used to catalyze the formation of peptide bonds (Williams and Ibrahim 1981; Nakajima and Ikada 1995; Madison and Carnali 2013). EDC is shown to enter intact plant and bacterial cells without previous disruption of the cell wall or cell membrane and covalently modify accessible Us and Gs on the WC face at neutral pH, marking novel use of this reagent as a valuable in vivo RNA secondary structure probe. Paired with glyoxal, EDC also provides a probe for identifying pKa-perturbed Gs in vivo and genomewide.

The materials and methods used for these experiments are now described

Plant Materials and Growth Conditions.

Standard 100 mm×15 mm petri dishes were inverted and the lids (now on the bottom) were lined with filter paper prior to the addition of ˜30-40 Oryza sativa (rice) seeds per 100 mm dish or ˜50-60 seeds per 150 mm dish. Approximately 100 mL of tap water was added and the seeds were covered with the bottom of the dish. The seeds were incubated in a 30-37° C. greenhouse under light of intensity ˜500 μmol photons m−2 s−1 supplied by natural daylight supplemented with 1000 W metal halide lamps (Philips Lighting Co) for 7-8 days. Seedlings then were transferred to pre-moistened Sunshine LC1 RSi potting soil (SunGro Horticulture) in 15 cm tall pots so that the seeds were ˜1 cm below the soil surface and the radicle or roots were completely buried within the soil. Water was added to an underlying plastic tray to ˜6 cm depth and the level was allowed to drop during the course of the growth incubation, since excessive watering of the seedlings can inhibit growth. A spoonful (˜0.5-1 g) of Sprint 330 powdered iron chelate (BASF) was added to the water to prevent seedling iron deficiency. The seedlings were illuminated with ˜500 μmol photons m−2 s−1 light intensity as above for another 7-8 days until attaining a height of ˜8-12 cm, E. coli growth conditions. E. coli (strain MG1655) was inoculated in liquid LB media and incubated overnight at 37° C. without shaking. The overnight culture was diluted 1:100 into 125 mL side-arm flasks each containing 19 mL of fresh LB media for each reaction condition and incubated at 37° C. in a shaking water bath until attaining a Klett value of 80 (mid-exponential growth phase).

In Vitro EDC Probing of Rice RNA.

All reactions involving 1-Ethyl-3-(3-dimethylaminopropyl)carbodiimide (EDC) were performed in a chemical fume hood. For all in vitro experiments, untreated rice seedlings that were grown for 14-16 days as described above were cut 5-10 mm above the soil line, and total RNA was extracted from these plants using the procedure described below. Reaction buffer was added to 1 μg total RNA to give a final total volume of 5 μL containing 50 mM pH buffer (one of the following: MES for pH 6, HEPES for pH 7-8, or CHES for pH 9.2), 50 mM KCl, and 0.5 mM MgCl2. The reaction was mixed thoroughly and incubated at room temperature for 5 minutes to allow equilibration. EDC stock solution (5.65 M, Sigma-Aldrich: 39391-10ML [listed as N-(3-Dimethylaminopropyl)-N′-ethylcarbodiimide]) was diluted to twice the desired final concentration in deionized water, and 5 μL of this diluted stock was added to the reaction mixture to give the desired final EDC concentration in a final reaction volume of 10 μL. In the control (−EDC) treatment, an equivalent volume of deionized water was added to the reaction mixture in place of EDC. Reactions proceeded for 2 minutes, 5 minutes, or 15 minutes at room temperature (˜22° C. before being quenched by the addition of 3 μL of 1 M sodium acetate (pH 6), 1 μL glycogen, and 35 μL 95% ethanol, followed immediately by freezing on dry ice for 1 hour and subsequent ethanol precipitation of the RNA. For reactions testing a dithiothreitol (DTT) quench, three separate quench solutions were prepared: DL-1.4 dithiothreitol (Acros Organics; 16568_0250) dissolved to 2.5 M in deionized water; 1 g of DTT dissolved in 5 mL of 1 M sodium acetate (pH 5); or 1 M sodium acetate (pH 5). With each quench condition, 201 μl of the quench solution was added either prior to the addition of 5 μL EDC or after a 5 minutes reaction with EDC. In vivo EDC probing of rice. All reactions involving EDC were performed in a chemical fume hood. Rice seedlings grown for 14-16 days as described above were cut 5-10 mm above the soil line. For reactions in a desired EDC concentration, 4-6 excised seedlings were placed in a 50 mL Falcon tube that contained buffer (HEPES, pH 7, HEPES, pH 8, or CHES, pH 9.2). KCl, and MgCl2 such that the addition of EDC diluted in deionized water gave a final total volume of 10 mL containing 50 mM pH buffer, 50 mM KCl, 0.5 mM MgCl2, and EDC of the desired final concentration (110 to 565 mM). In control (−EDC) reactions, equivalent volumes of deionized water were added in place of EDC. For all experimental and control conditions, the reactions occurred for 15 minutes at room temperature with periodic shaking and swirling. For treatments using only a water wash, the reaction buffer was decanted and the seedlings were washed 6 times with ˜20 mL deionized water each wash before immediate drying and freezing in liquid N2, For treatments using a DTT quench, 1 g of DL-1,4 dithiothreitol (Acros Organics; 16568_0250) was added to the tube, which was then shaken vigorously for 2 minutes. Then, the reaction buffer was decanted and the seedlings were washed 3 times with ˜20 mL deionized water for each wash before immediate drying and quick freezing in liquid N2. Frozen seedlings then were subjected to total RNA extraction as described below, with separate mortars and pestles used for each treatment.

In Vivo Phenylglyoxal Probing of Rice

All reactions involving phenylglyoxal were performed in a chemical fume hood. Control and experimental treatments with phenylglyoxal were performed as described previously (Mitchell et al. 2018), For treatments using only a water wash, the reaction buffer was decanted and the seedlings were washed 6 times with ˜20 mL deionized water each wash before immediate drying and freezing in liquid N2. For treatments using a DTT quench, 1 g of DL-1,4 dithiothreitol (Acros Organics; 16568_0250) was added to the tube, which was then shaken vigorously for 2 minutes. Then, the reaction buffer was decanted and the seedlings were washed 3 times with ˜20 mL deionized water each wash before immediate drying and quick freezing in liquid N2. Frozen seedlings then were subjected to total RNA extraction as described above, with separate mortars and pestles used for each treatment.

Total RNA Extraction from Rice,

Untreated or EDC-treated rice seedlings were quickly frozen in liquid nitrogen and stored at −80° C. until use. Frozen tissue was ground to fine powder using a mortar and pestle pre-cleaned with RNase Zap (Ambion). In an Eppendorf tube, 80-100 mg of powder was added to 350 mL of lysis buffer (Macherey-Nagel) and 35 mL of 500 mM dithiothreitol (DTT), then centrifuged for 1 minute at >11.000 rpm. The supernatant was then subjected to total RNA extraction following the protocol described in the NucleoSpin RNA Plant kit (Macherey-Nagel). In vivo EDC probing of E. coli. All reactions involving EDC were performed in a chemical fume hood. EDC diluted in distilled water was added to E. coli cells grown as described above to give final concentrations of EDC ranging from 5.7 to 113 mM in a total volume of 20 mL. The reactions were allowed to proceed for 5 minutes at 37° C. with continuous shaking, followed by the addition of 0.8 g DTT and additional shaking for 2 minutes at 37° C. to quench the reaction. Cell growth was arrested by removing 6 mL of treated cells and adding to 6 mL of a frozen slurry buffer containing 10 mM Tris-1C (pH 7.2), 5 mM MgCl2, 25 mM NaN3, 1.5 mM chloramphenicol, and 12.5% ethanol, followed by incubation on ice for 10 minutes. Cell pellets were washed twice in the same buffer, Total RNA was extracted from the final cell pellets using the RNeasy Mini kit (Qiagen), and the extracted RNA was subjected to phenol chloroform extraction and ethanol precipitation after treatment with Turbo DNase (Ambion).

Gene-Specific Reverse Transcription.

Reverse transcription was performed on in vitro or in vivo total RNA extracted from rice or E. coli as previously described (Mitchell et al. 2018), using 32P-radiolabeled primer targeting rice 5.8S rRNA (5′-GCGTGACGCCCAGGCA-3′ SEQ WD NO:23), rice 28S rRNA (5′-GGACGCCTCTCCAGACTACAATTCG-3′; SEQ ID NO:24), or E. coli 16S rRNA (5′-TTACTCACCCGTCCGCTCACTCG-3′; SEQ ID NO:25).

Gene-Specific Reverse Transcription for E. coli.

E. coli total RNA extracted as described above was combined with 10× First Strand Synthesis buffer (Invitrogen) and nuclease-free water to give 2 μg of total in a 4.5 μL volume. Next, 1 μL of ˜500,000 cpm/μL 32P-radiolabeled primer complementary to 16S rRNA (shown above) was added to the total RNA sample. The solution was incubated at 95° C. for 1 minute then cooled to 35° C. for 1 minute to anneal the primer. Once cooled, 3 μL of reverse transcription reaction buffer was added to a final concentration of 8 mM MgCl2, 10 mM DTT, and 1 mM dNTPs. The solution was heated to 55° C. for 1 minute, 0.5 μL of 200 Units/μL Superscript III reverse transcriptase (Invitrogen) was added to the reaction, and reverse transcription was allowed to proceed at 55° C. for 15 minutes. Next, 1 μL of 1M NaOH was added to the solution, which was then heated to 95° C. for 5 minutes to hydrolyze all contaminating RNAs and to heat denature reverse transcriptase. Lastly, an equal volume (11 μL) of 2× stop solution containing 100% deionized formamide, 20 mM Tris-HCl, 40 mM EDTA, 0.1% xylene cyanol, and 0.025% bromophenol blue was added to the reaction. The mixture was loaded onto a 6% denaturing polyacrylamide gel (83 M Urea) and run at a constant 80 W for ˜90 minutes. The resulting data was analyzed using semi-automated footprinting analysis software (SAFA) (Das et al. 2005).

Calculation of Significant EDC Modification,

Chemical modification was calculated essentially as previously described (Mitchell et al. 2018). Briefly, in all plots constructed from SAFA results, significant EDC modification was calculated in the following manner. The background-corrected band intensity for all residues within the examined nucleotide range-except for Us, Gs, and the largest and smallest values for each reaction condition—were averaged and their standard deviation was calculated. Next, the value for significant EDC modification (S) for a number of reaction conditions n was calculated as the grand average of the averages (Aᵢ) plus three times the standard deviation for each reaction condition (a), as shown below:

$$S = \frac{Σ ( {A}_{i} + 3 {σ}_{i} )}{n}$$

Here, as most reaction conditions give bands of light intensity even in the absence of modification by a reagent, three standard deviations from the mean ensures sufficient separation between such background bands and bands genuinely caused by modified nucleotides.

EDC Reaction Quench:

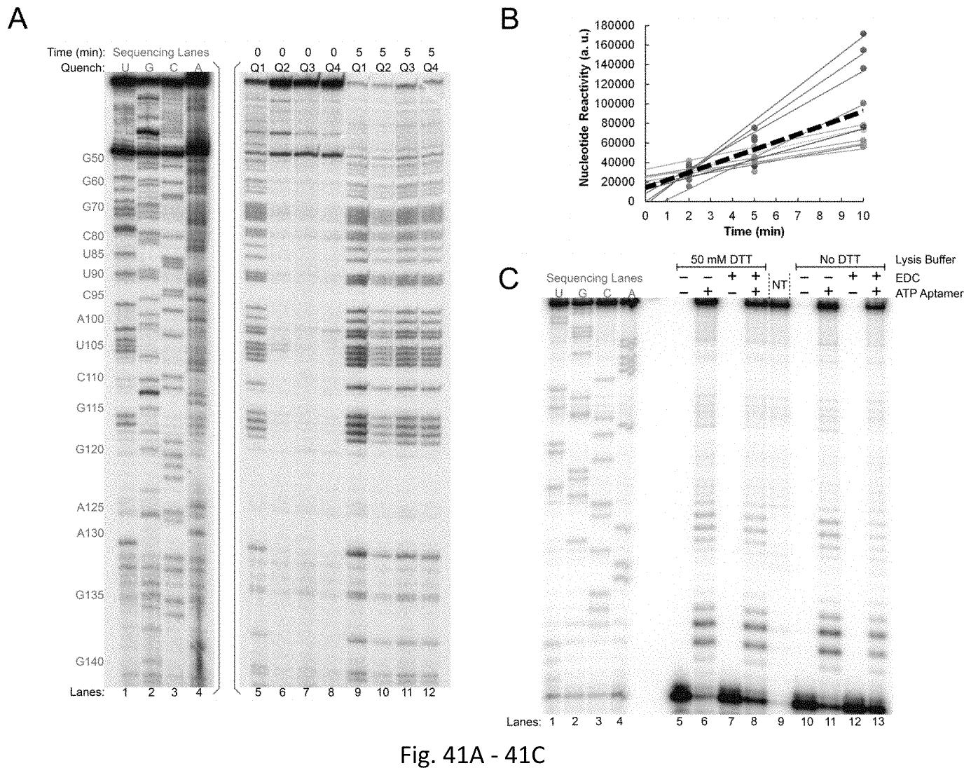

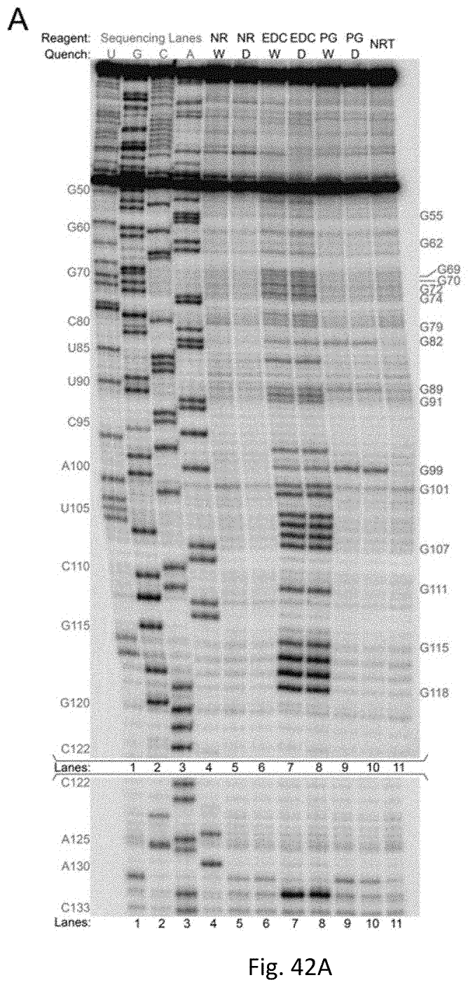

The EDC reaction was quenched by a three-step process. First, 1 g of solid dithiothreitol (DTT) was added prior to three water washes of the plant tissue. Tests showed that DTT prevents EDC from reacting with uracils or guanines in vitro (FIG. 41A). Second, after the water washes, the tissue was quickly frozen in liquid N2. Third, the sample was thawed in a lysis buffer containing additional DTT at 50 mM. The reaction was adequately quenched by this three-step process as revealed by time points for reactivity of various nucleotides that extrapolated back to the origin (FIG. 41B), as well as by a quench control. In the quench control, the 57 nt ATP aptamer RNA, which is not natural to rice and thus contains a sequence not found in total rice RNA, was doped into the lysis buffer used in the RNA extraction. There was no EDC-specific reaction of the ATP aptamer (FIG. 41C), indicating that the EDC had been successfully quenched by the prior treatment. Importantly, RT extension only occurs when the ATP aptamer is present (FIG. 41C, Lanes 5, 7, 10, 12).

The results of the experiments are now described

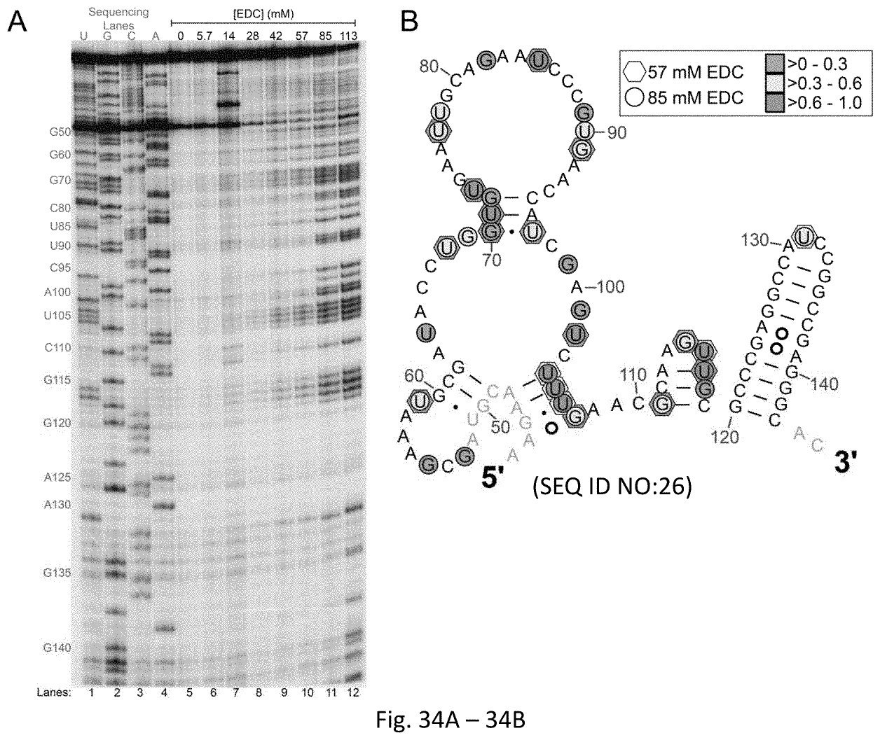

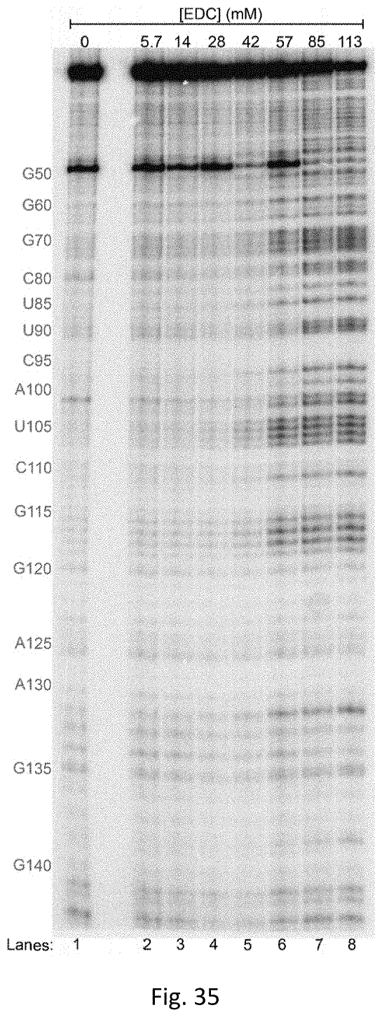

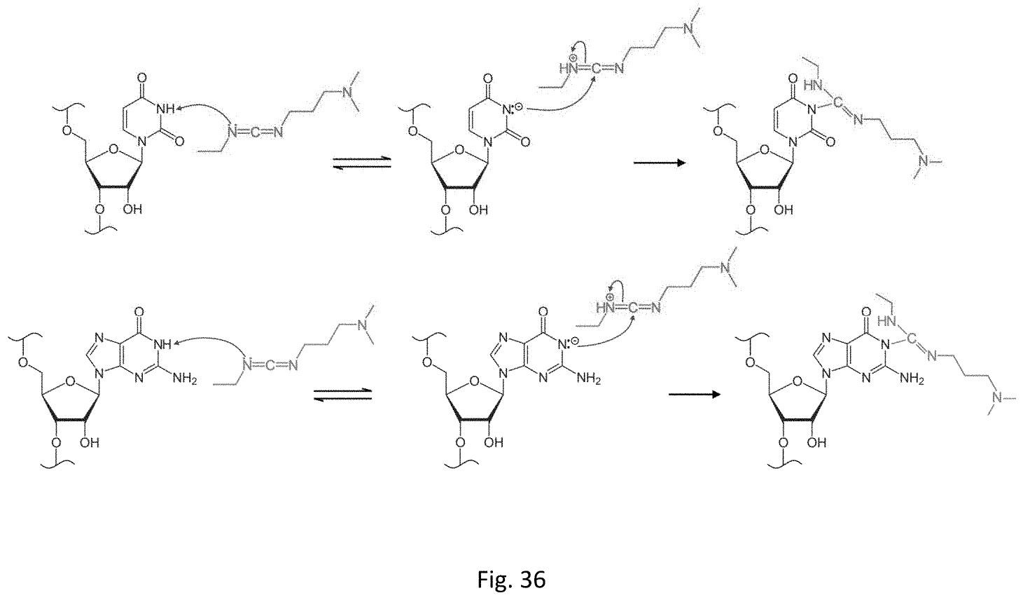

While in vitro reactions with RNA-modifying reagents typically are inapplicable to a biological context, they can often provide valuable information on the efficacy of the reagent and conditions for in vivo probing. The U modification activity of the carbodiimide EDC was determined in vitro, using primer extension and denaturing PAGE of rice 5.8S rRNA. Selected buffers spanned a pH range of 6 to 9.2 and contained 50 mM K⁺ and 0.5 mM Mg²⁺ to mimic typical cytoplasmic cation concentrations (Walker et al. 1996; Karley and White 2009; Gout et al, 2014). In the examined region of G33 to C143, EDC displayed robust and specific modification of Us and Gs to different extents that reflect RNA structure (FIG. 34A and FIG. 35, where the same EDC concentrations are tested for a shorter reaction time). Reactivity of EDC did not modify any As or Cs throughout the examined region, consistent with the known chemistry of carbodiimide reagents (FIG. 36). Increasing the concentration of EDC increased the extent of reaction and resulted in several new sites (FIG. 34B).

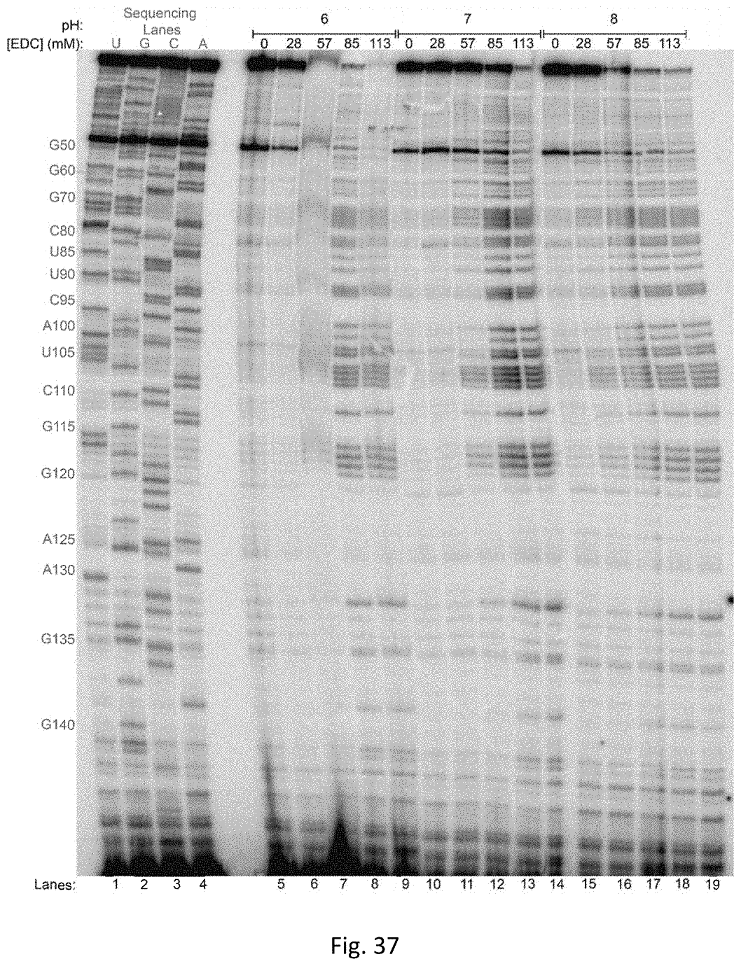

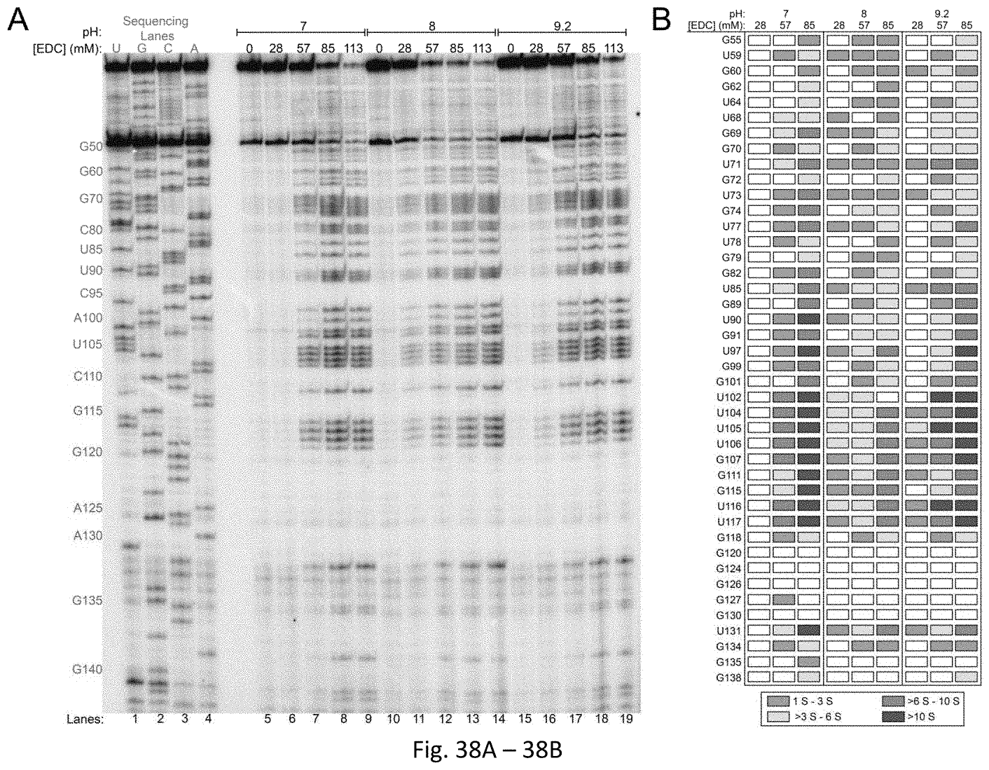

In comparing in vitro studies of EDC to an in vitro study of glyoxal (Mitchell et al. 2018), it was found that ˜10× more EDC was required to achieve observable base modifications in the same timeframe of 5 minutes (2.5 mM for glyoxal, methylglyoxal, and phenylglyoxal vs >28 mM for EDC). Notably, EDC concentrations above 85 mM led to excessive modification of the RNA and resultant loss of single hit kinetics (FIG. 34A). A slight pH-dependence was observed for in vitro EDC reactivity when using low (28 mM) concentrations of EDC; reactions at pi 6 (FIG. 37) and pH 7 (FIG. 38) gave no observed modifications while reactions at pH 8 or pH 9.2 resulted in modifications, which might reflect deprotonation of the carbodiimide EDC (FIG. 38; also see FIG. 37). Notably, increasing the EDC concentration eliminated this pH dependence. Finally, across all of the in vitro conditions tested, while EDC readily modifies both Us and Gs, it appears to favor modification of Us by a factor of ˜16.

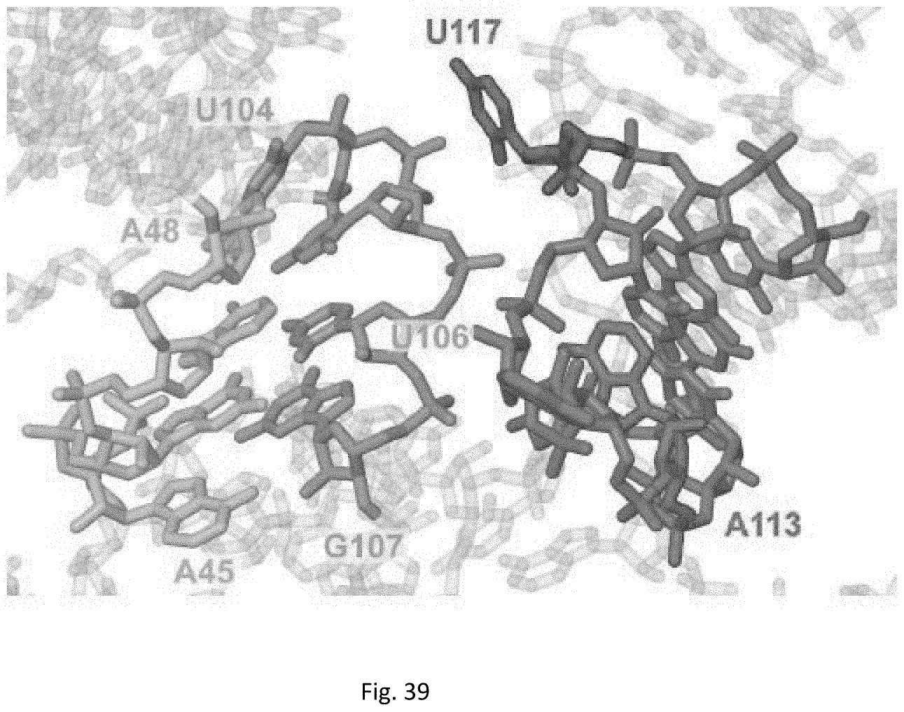

Interestingly, one intense region of EDC reactivity aligns with a long-range phylogenetically predicted four base helical strand containing U104 to G107, and another is found along a local stemloop spanning G111 to G119 (FIG. 34 and FIG. 38). For the long-range pairing, U106 forms a wobble pair with G46, and G107 forms a sheared pair with A45 (Heus and Pardi 1991; SantaLucia and Turner 1993). The sheared G-A pair exposes the WC face of the G to EDC, while the G.U wobble is significantly weaker than WC base pairs (Turner 2000). The two remaining base pairs are A-U pairs, which are relatively weak leading to a high probability of transient unwinding of the helix, which would allow access to EDC. For the local stem-loop of G111 to G119, while U117 is shown paired with A113 in the secondary structure derived from comparative analysis (Cannone et al. 2002; Gutell et at, 2002), it is unpaired and flipped outward in the homologous yeast cryo-EM structure (Schmidt et al 2016) (FIG. 39), This is not unlike the highly reactive G107 being flipped out in its sheared base pair. On the other hand, the 10-bp stem-loop spanning G120 to C143, analogous to the G-C rich 9-bp stem-loop in the yeast cryo-EM structure, did not give any modifications except for a single base in the loop (FIG. 34B), indicating that Gs in strong helices do not react with EDC.

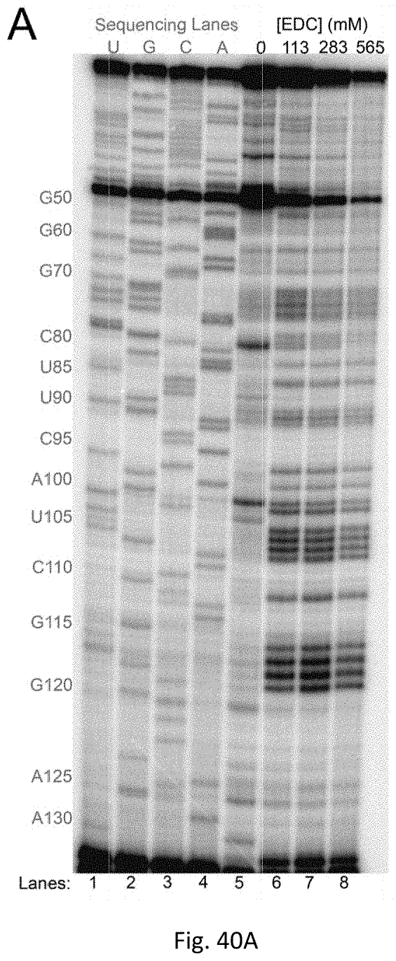

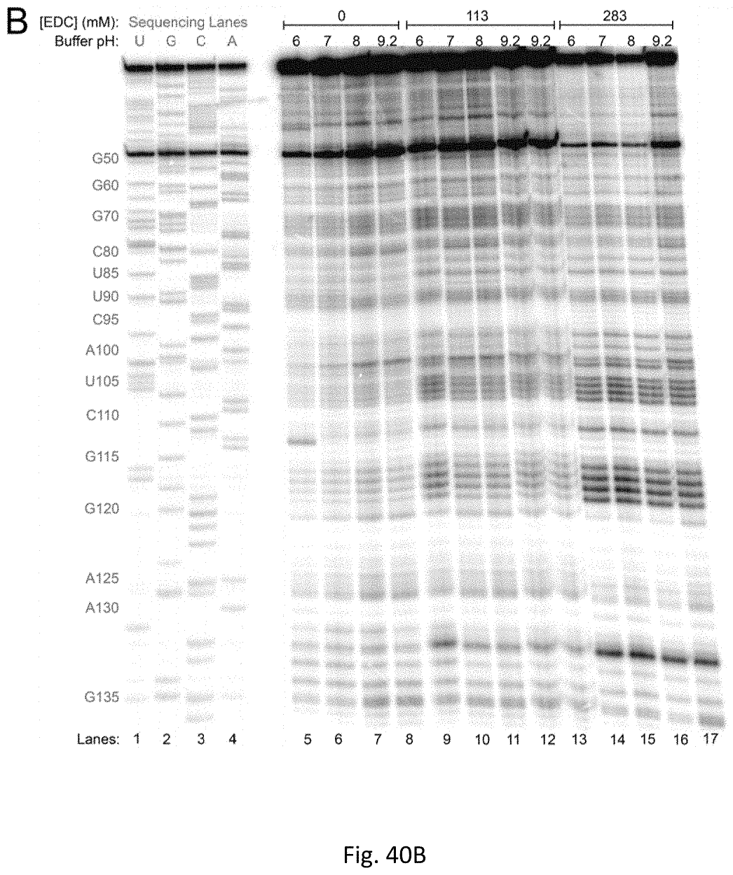

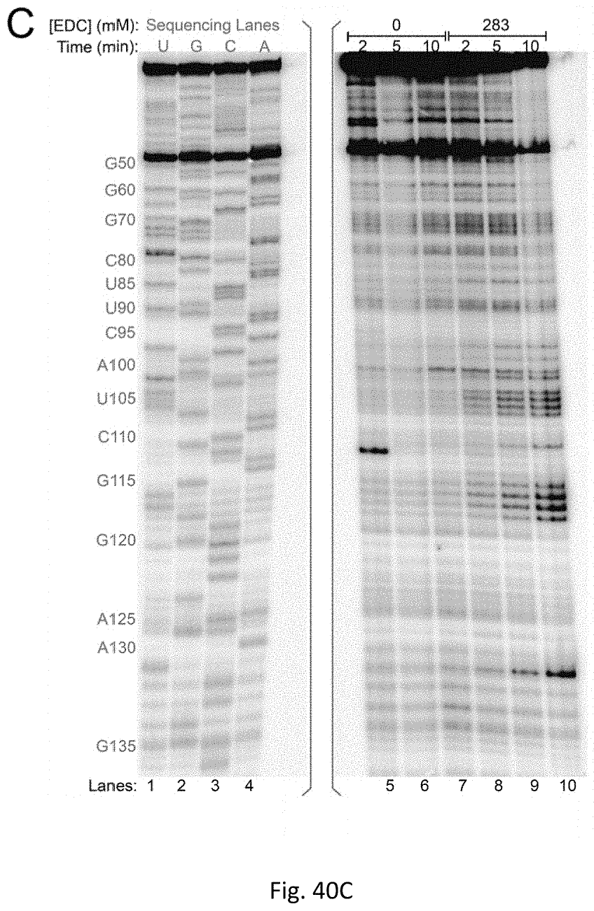

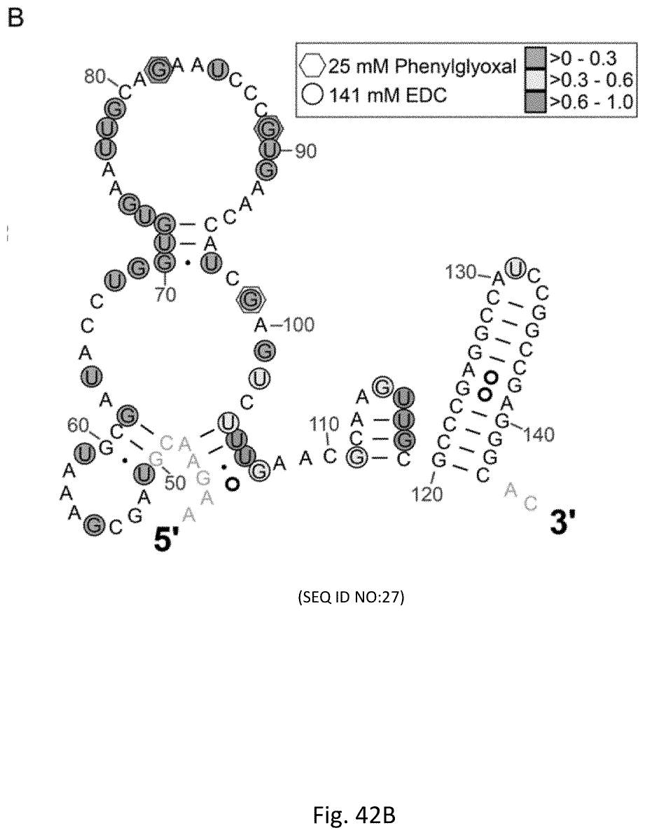

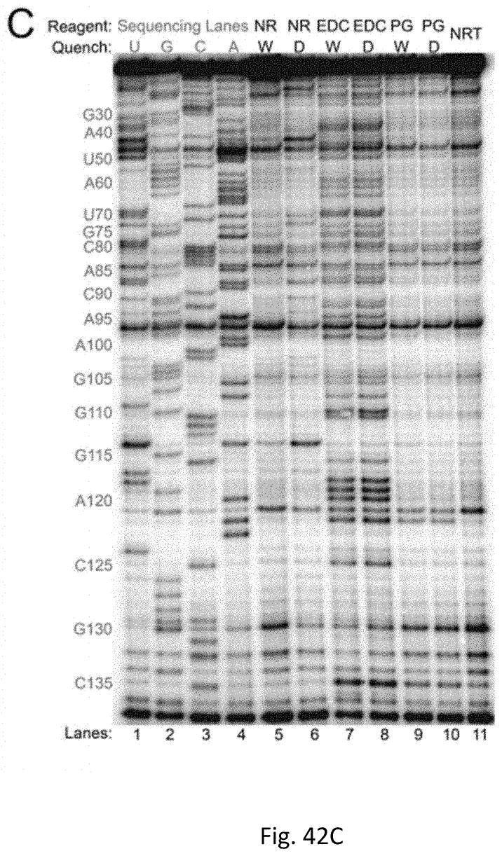

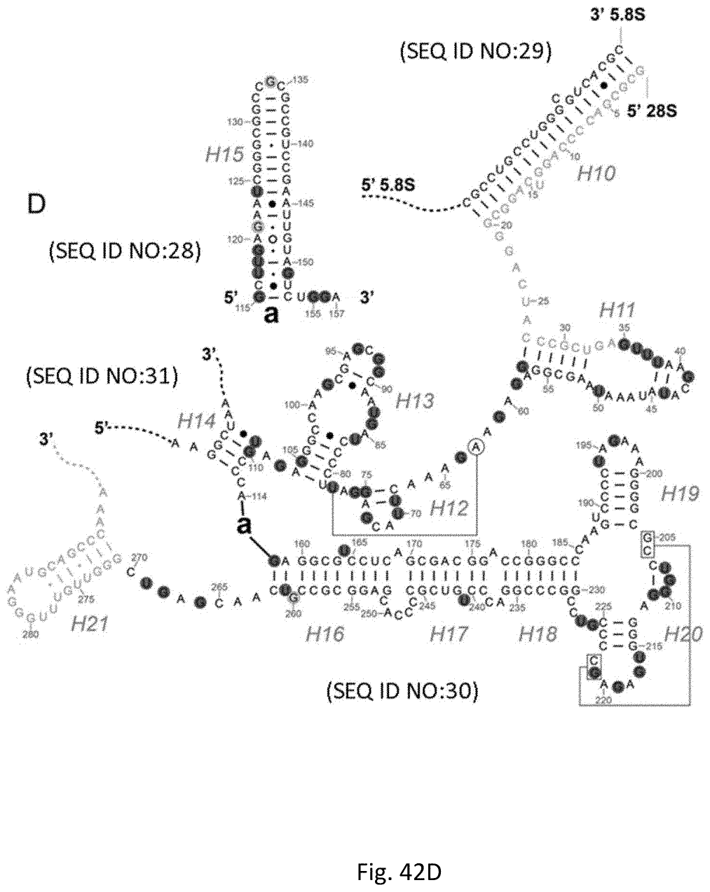

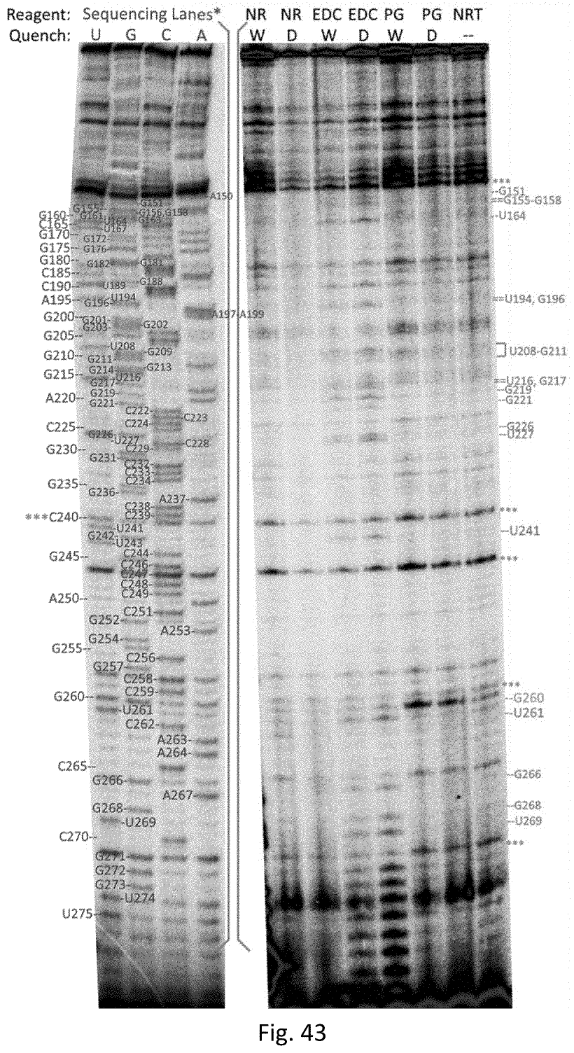

Upon determining that EDC specifically modified Us and Gs in vitro, rice tissue was exposed to EDC to test whether the reagent could probe RNA structure within intact cells without artificially permeabilizing the cell wall or membrane with detergents or other reagents (Holmberg et al. 1994; Incarnato et al 2014). As with glyoxal and its derivatives, the excised shoots of 2-week-old rice seedlings were incubated for 15 minutes in buffers containing 50 mM K⁺, 0.5 mM Mg²⁺, and EDC ranging from 113 to 565 mM. Similar to the aforementioned in vitro results, EDC modified almost all Us and Gs within single-stranded loops and weak helices when probing 5. AS rRNA in vivo (FIG. 4A). No modification is observed at As or Cs, indicating that EDC is base specific in vivo. EDC concentrations above 283 mM led to a sharp decrease in the intensity of the full-length band and of the bands for many of the modified nucleotides (FIG. 40A), indicating excessive modification. As such, all subsequent in vivo experiments in rice used a maximum EDC concentration of 283 mM. Similar to the in vitro conditions tested above, varying the external buffer pH from 6 to 9.2 had no effect on modifications in 113 mM and 283 mM EDC (FIG. 40B). Again. EDC preferably reacted with U over G, with a U-to-G reactivity ratio of 1.4 in vivo, similar to the value of 1.6 found in vitro. Varying the EDC reaction time from 2 minutes to 10 minutes revealed a time dependence for in vivo base modification, with increasing reactivity observed at longer times (FIG. 40C; also see quantitation of reactivity time dependence in FIG. 41B). In vivo probing of both rice 5.8S rRNA (FIG. 42A through FIG. 42B) and 28S rRNA (FIG. 42C through FIG. 42D; also see FIG. 43 for additional data on 28S rRNA) reveals EDC modification of almost all unpaired Us and Gs within loops or within or immediately adjacent to relatively unstable helices, confirming that EDC reports on RNA secondary structure. While some nucleotides are denoted as unmodified as a result of uncertainty owing to natural RT stops, the vast majority of unmodified bases form WC base pairs within stable helices. For example, Gs present within helices H16-H20, which are predicted to be base paired, are not modified by EDC or phenylglyoxal (FIG. 40D). H15 provides a stark illustration of high EDC reactivity within a subregion of an otherwise stable and unreactive helix. Specifically, the subregion G115 to U124 has five non-canonical WC interactions near the base of the stem and is quite reactive with EDC, while the apex of the stem is mostly GC base pairs and is unreactive. FIG. 40 and FIG. 41 confirm by several approaches that the reaction is quenched prior to RNA extraction. Thus, EDC is capable of reporting on RNA secondary structure in vivo.

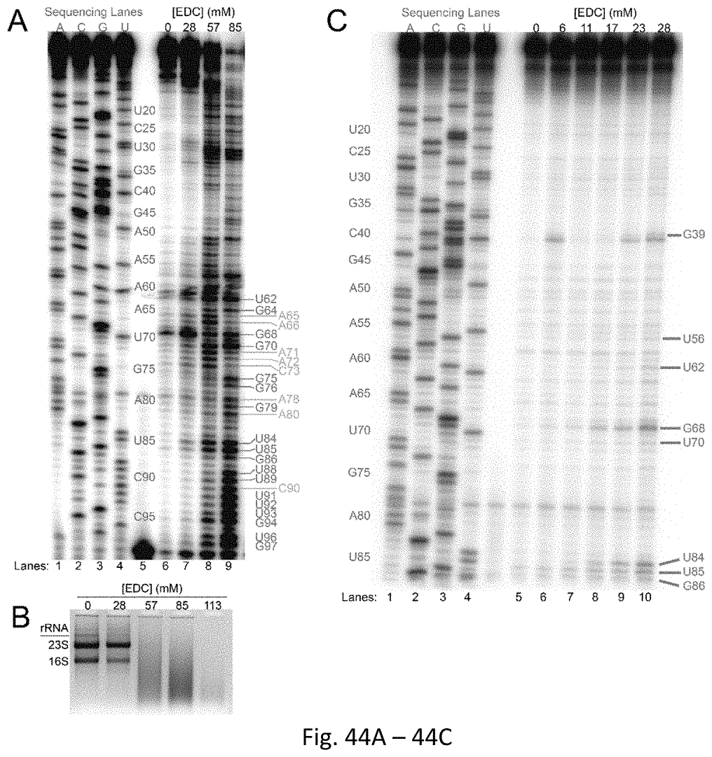

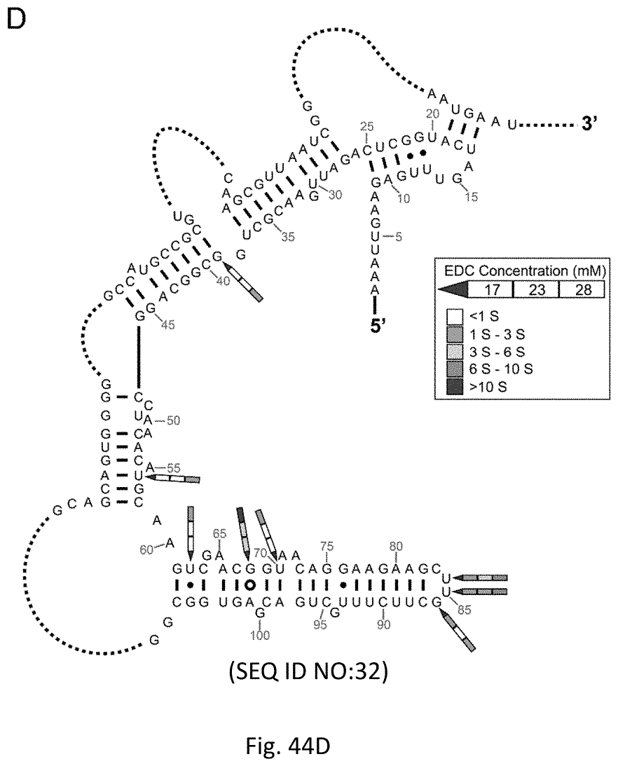

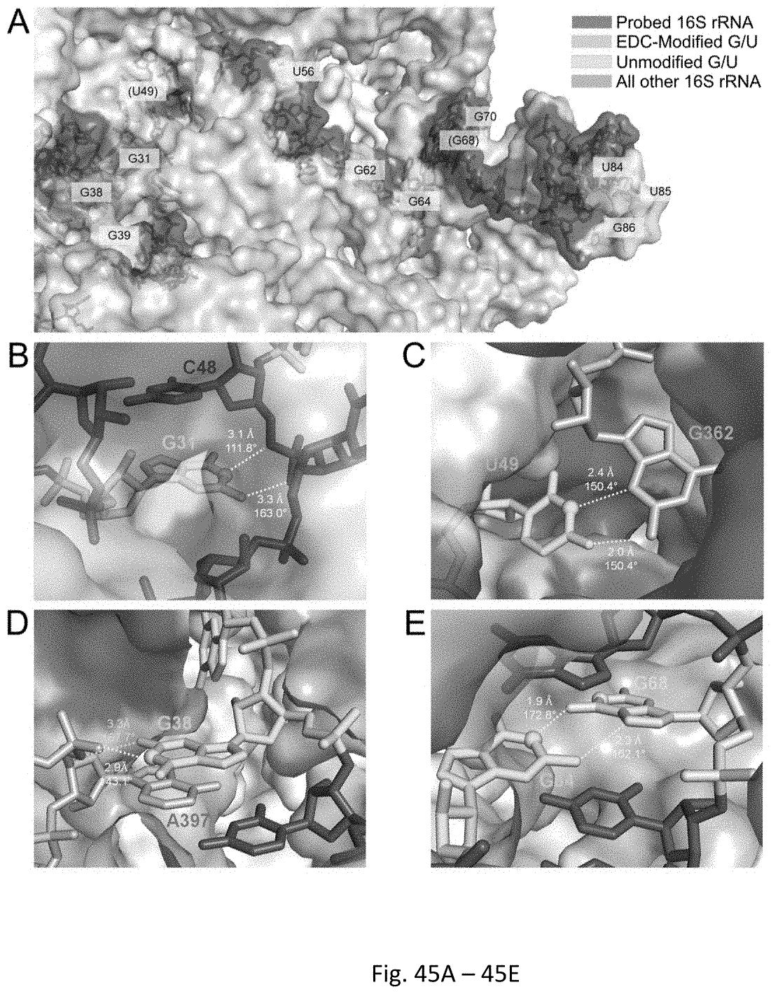

To test whether EDC can probe RNA structure in vivo within multiple domains of life, Gram-negative E. coli strain MG1655 was treated with EDC and probed 16S rRNA. Examining a range of EDC concentrations from 28 mM to 141 mM revealed that EDC successfully entered cells and modified RNA (FIG. 44A), Treatment with ≥57 mM EDC led to an excessive number of bands upon separation of reverse transcription products by denaturing PAGE, including As and Cs that EDC cannot modify, which was attributed to degradation of the RNA. Separation of in vivo EDC-treated total RNA on an agarose get confirmed degradation of the RNA at 57 mM EDC, with the loss of the discrete rRNA bands and the formation of a broad smear (FIG. 44B). Furthermore, treatments with EDC concentrations above 57 mM severely diminished yields from RNA extraction and led to the formation of an unidentified precipitate upon quenching the EDC reaction with DTT. Based on these initial results, in vivo modification of E. coli cells was tested using a range of 6 mM to 28 mM EDC. EDC modification specifically at Gs and Us (FIG. 44C) was detected. At the tested concentration of 28 mM, EDC favored modification of Us in E. coli, giving a U-to-G ratio of 1.5, similar to the in vitro and in vivo ratios with rice. Lower EDC concentrations resulted in ratios <1, with the value skewed by unusually strong EDC modification of G68—a G that forms a sheared pair with A 101 and exposes its WC face in what is apparently a highly reactive conformation, as described above for rice 5.8S rRNA. Upon mapping the modified bases onto the E. coli 16S rRNA secondary structure derived from comparative analysis (Cannone et al. 2002), it was observed that the nucleotides with highest EDC reactivity were the sheared G68 and the hairpin loop nucleotides U84, U85, and 086 (FIG. 44D). All other EDC-modified nucleotides are positioned adjacent to bulges (G39, US6, and U70) or are involved in a G.U wobble pair (G62), presumably providing access to modification. Interestingly, EDC did not modify four Gs and Us (G31, G38, U49, and G64) shown as single-stranded within the 16S rRNA secondary structure (FIG. 44D). Examination of the E. coli 70S ribosome crystal structure revealed that the base of G31 and the entirety of U49 are buried within the interior of the ribosome and thus are solvent inaccessible, consistent with their observed lack of modification (see FIG. 45). Conversely, G38 and G64 are solvent exposed. However, all four unmodified nucleotides exhibit interactions involving the endocyclic N1 of G or N3 of U that would inhibit deprotonation by EDC (see FIG. 45; also see FIG. 36 for EDC reaction scheme). G31 and G38 each are in position to hydrogen bond with the bridging 05′ of C48 and the non-bridging oxygen of A397, respectively, with the bonding distances being ˜3 Å for each pair (see FIG. 45). U49 is further protected by base pairing between its WC face and the sugar edge of G362. A similar interaction exists between the WC face of 064 and the Hoogsteen face of G68 (FIG. 45).

It is of interest to compare the properties of EDC with glyoxal, which also reacts with (s in vivo (Mitchell et al. 2018), In the G50 to C143 region of rice 5.8S rRNA, EDC modified 34 out of 47 possible nucleotides, consisting of 16 out of 29 Gs and 18 out of 18 Us (FIG. 42B). By comparison, phenylglyoxal only modified three nucleotides (G82, G89, G99) within that same region. The larger examined region for 28S rRNA, spanning from G35 in H111 to C270 just upstream of H21, provides another example of this effect. Here, 54 out of 113 Gs and Us are modified by EDC, consisting of 35 out of 80 is and 19 out of 33 Us (FIG. 42D). Conversely, phenylglyoxal only modified three Gs (G121, G134, and G260) within this extended region of 28S rRNA. Only N1-deprotonated anionic Gs can react with glyoxals, since glyoxal is an electrophile, which likely accounts for the lower reactivity of glyoxal compared to EDC. Moreover, Gs typically have a pKa of 9 on the N1, which is further elevated in WC base pairs (Legault and Pardi 1997; Wilcox et al. 2011). Given that the cytosol of most cells is at a pH of ˜7, any sites of glyoxalation may arise from Gs with pKas shifted towards neutrality. When comparing EDC, a nucleophilic reagent that reacts with N1-protonated neutral Gs (FIG. 38), with glyoxal, unpaired Gs with shifted pKas may thus become apparent.

In conclusion, the experiments present a novel application of the water-soluble carbodiimide EDC as an in vivo probe of RNA secondary structure. EDC targets the WC face of unpaired Us and to a lesser extent Gs with high specificity at neutral pH and within intact cells across multiple domains of life, importantly, EDC finally resolves the information gap that has existed for 30 years for in vivo structural probing of base-pairing interactions. The combined application of WC-specific probes in EDC and DMS, along with sugar-reactive SHAPE reagents and the C8-A/G reactive reagent NAz, will provide a once-iuattainable comprehensive picture of in vivo base pairing, backbone flexibility, secondary structure formation, and protein protection for all four RNA bases.

### REFERENCES

Altuvia S, Komitzer D, Teff D, Oppenheim A B. 1989. Alternative mRNA structures of the cIII gene of bacteriophage lambda determine the rate of its translation initiation, J Mol Biol 210: 265-280.
Antal M, Boros E, Solymosy F, Kiss T. 2002. Analysis of the structure of human telomerase RNA in vivo, Nucleic Acids Res 30; 912-920.
Babitzke P. 1997. Regulation of tryptophan biosynthesis: Trp-ing the TRAP or how Bacillus subtilis reinvented the wheel, Mol Microbiol 26: 1-9.
Balzer M, Wagner R. 1998. A chemical modification method for the structural analysis of RNA and RNA protein complexes within living cells. Anal Biochem 256: 240-242.
Barmwal R P, Loh E, Godin K S. Yip J, Lavender H, Tang C M, Varani G. 2016. Structure and mechanism of a molecular rheostat, an RNA thermometer that modulates immune evasion by Neisseria meningitidis. Nucleic Acids Res 44: 9426-9437.
Bevilacqua P C, Assmann S M. 201). Technique development for probing RNA structure in vivo and genome-wide. In Additional Perspectives on RNA Worlds, (ed. T R Cech, J A Steitz., J F Atkins). Cold Spring Harbor Laboratory Press, New York, N.Y. (in press).
Bevilacqua P C, Ritchey L E. Su Z, Assmann S M. 2016. Genome-Wide Analysis of RNA Secondary Structure. Annu Rev Genet 50: 235-266.
Cannone J J, Subramanian S, Schnare M N, Collett J R. D'Souza L M, Du Y, Feng B, Lin N, Madabusi L V, Muller K M et al. 2002. The comparative RNA web (CRW) site: an online database of comparative sequence and structure information for ribosomal, intron, and other RNAs. BMC Bioinformatics 3: 2.
Das R, Laederach A, Pearlman S M, Herschlag D. Altman R B. 2005. SAFA: semi-automated footprinting analysis software for high-throughput quantification of nucleic acid footprinting experiments. RNA 11: 344-354.
Ding Y, Tang Y. Kwok C K, Zhang Y, Bevilacqua P C, Assmann S M. 2014. In vivo genome-wide profiling of RNA secondary structure reveals novel regulatory features. Nature 505: 696-700.
Fedorova O, Zingler N. 2007. Group II introns: structure, folding and splicing mechanism. Biol Chem 388:665-678.
Feng C, Chan D, Joseph 3, Muuronen M, Coldren W H, Dai N, Correa I R, Jr, Furche F, Hadad C M, Spitale R C. 2018. Light-activated chemical probing of nucleobase solvent accessibility inside cells. Nat Chem Biol 14: 276-283.
Gout E. Rebeille F. Douce R., Bligny R. 2014. Interplay of Mg2+, ADP, and ATP in the cytosol and mitochondria: unravelling the role of Mg2+ in cell respiration, Proc Natl Acad Sci USA 111:E4560-4567.
Guerrier-Takada C, Gardiner K, Marsh T, Pace N, Altman S. 1983. The RNA moiety of ribonuclease P is the catalytic subunit of the enzyme. Cell 35: 849-857.
Gitell R R, Lee J C. Cannone J J. 2002. The accuracy of ribosomal RNA comparative structure models. Curr Opin Struct Biol 12: 301-310.
Harris K A, Jr., Crothers D M, Ullu E. 1995. In vivo structural analysis of spliced leader RNAs in Trypanosoma brucei and Leptomonas collosoma: a flexible structure that is independent of cap4 methylations. RNA 1: 351-362.
Heus H A. Pardi A. 1991. Structural features that give rise to the unusual stability of RNA hairpins containing GNRA loops. Science 253: 191-194.
Holmberg L. Melander Y, Nygard O. 1994. Probing the structure of mouse Ehrlich ascites cell 5.85, 185 and 28S ribosomal RNA in situ. Nucleic Acids Res 22: 1374-1382.
Incamato D, Neri F, Anselmi F, Oliviero S. 2014. Genome-wide proftiling of mouse RNA secondary structures reveals key features of the mammalian transcriptome. Genome Biol 15: 491,
Karley A J, White P J. 2009. Moving cationic minerals to edible tissues: potassium, magnesium, calcium. Curr Opin Plant Biol 12: 291-298.
Kortmann J, Sczodrok S, Rinnenthal J, Schwalbe H, Narberhaus F. 2011. Translation on demand by a simple RNA-based thermosensor. Nucleic Acids Res 39: 2855-2868.
Kumari S, Bugaut A, Huppert J L, Balasubramanian S. 2007. An RNA G-quadruplex in the 5′ UTR of the NRAS proto-oncogene modulates translation. Nat Chem Biol 3: 218-221.
Kwok C K, Ding Y, Shahid S, Assmann S M, Bevilacqua P C. 2015a. A stable RNA G-quadruplex within the 5′-UTR of Arabidopsis thaliana ATR mRNA inhibits translation. Biochem 1467: 91-102.
Kwok C K, Tang Y, Assmann S M, Bevilacqua P C. 2015b. The RNA structurome: transcriptome-wide structure probing with next-generation sequencing. Trends Biochem Sci 40: 221-232.
Lee B, Flynn R A., Kadina A, Guo J K, Kool E T, Chang H Y. 2017. Comparison of SHAPE reagents for mapping RNA structures inside living cells. RNA 23: 169-174.
Legault P, Pardi A. 1997. Unusual dynamics and pKa shift at the active site of a lead-dependent ribozyme. J Am Chem Soc 119: 6621-6628.
Madison S A, Camali JO. 2013, pH Optimization of Amidation via CarbodiimidesInd Eng Chem Res 52:13547-13555.
Merino E J. Wilkinson K A, Coughlan J L. Weeks K M. 2005. RNA structure analysis at single nucleotide resolution by selective 2-hydroxyl acylation and primer extension (SHAPE). J Am Chem Soc 127:4223-4231,
Mitchell D, 3rd. Ritchey L E, Park H, Babitzke P. Assmann S M, Bevilacqua P C. 2018. Glyoxals as in vivo RNA structural probes of guanine base-pairing. RNA 24: 114-124.

Mitchell D, 3rd, Russell R. 2014. Folding pathways of the Tetrahymena ribozyme. J Mol Biol 426: 2300-2312.
Nakajima N, Ikada Y. 1995. Mechanism of amide formation by carbodiimide for bioconjugation in aqueous media. Bioconjug Chem 6: 123-130.
Naville M, Gautheret D. 2010. Transcription attenuation in bacteria: theme and variations. Brief Funct Genomics 9: 178-189.
Noller U F, Chaires J B. 1972. Functional modification of 16S ribosomal RNA by kethoxal. Proc Natl Acad Sci USA 69: 3115-3118.
Peselis A. Serganov A. 2014. Themes and variations in riboswitch structure and function. Biochim Biophys Acta 1839: 908-918.
Rabani M, Pieper L, Chew G L., Schier A F. 2017. A Massively Parallel Reporter Assay of 3′ UTR Sequences Identifies In Vivo Rules for mRNA Degradation. Mol Cell 68: 1083-1094 e1085.
Rouskin S, Zubradt M. Washieti S. Keilis M, Weissman J S. 2014. Genome-wide probing of RNA structure reveals active unfolding of mRNA structures in vivo. Nature 505: 701-705.
SantaLucia J, Jr., Turner D H. 1993. Structure of (rGGCGAGCC)2 in solution from NMR and restrained molecular dynamics. Biochemistry 32: 12612-12623.
Schmidt C, Becker T, Heuer A, Braunger K, Shanmuganathan V. Pech M, Berninghausen O, Wilson D N. Beckmann R. 2016. Structure of the hypusinylated eukaryotic translation factor eIF-5A bound to the ribosome. Nucleic Acids Res 44: 1944-1951.
Spitale R C, Crisalli P, Flynn R A. Torre E A, Kool E T, Chang H Y. 2013. RNA SHAPE analysis in living cells. Nat Chem Biol 9: 18-20,
Teixeira A, Tahiri-Alaoui A. West S. Thomas B, Ramadass A, Martianov 1, Dye M, James W, Proudfoot N J, Akoulitchev A. 2004, Autocatalytic RNA cleavage in the human beta-globin pre-mRNA promotes transcription termination. Nature 432: 526-530.
Turner D H. 2000. Conformational Changes. In Nucleic Acids: Structurc, Properties, and Functions, (ed. V A Bloomfield, D M Crothers, I Tinoco. Jr.), pp. 259-334. University Science Books, Sausalito, C A. Walker D J, Leigh R A, Miller A J, 1996. Potassium homeostasis in vacuolaite plant cells. Proc Natl Acad Sci USA 93: 10510-10514.
Wan Y, Qu K, Ouyang Z, Kertesz M, Li J, Tibshirani R, Makino D L. Nutter R C, Segal E, Chang H Y. 2012. Genome-wide measurement of RNA folding energies. Mol Cell 48: 169-181.
Wan Y, Qu K. Zhang Q C, Flynn R A, Manor O. Ouyang Z, Zhang J, Spitale R C, Snyder M P, Segal E et al. 2014. Landscape and variation of RNA secondary structure across the human transcriptome. Nature 505: 706-709.
West S, Gromak N, Proudfoot N J. 2004. Human 5′->3′ exonuclease Xrn2 promotes transcription termination at co-transcriptional cleavage sites. Nature 432: 522-525,
Wilcox J L, Ahluwalia A K. Bevilacqua P C. 2011. Charged nucleobases and their potential for RNA catalysis. Acc Chem Res 44: 1270-1279,
Wiliams A, Ibrahim I T. 1981. Carbodiimide Chemistry: Recent Advances. Chem Rev 81: 589-636,
Winkler W, Nahvi A, Breaker R R. 2002. Thiamine derivatives bind messenger RNAs directly to regulate bacterial gene expression. Nature 419:952-956.
Yanofsky C. 1981. Attenuation in the control of expression of bacterial operons. Nature 289: 751-758.
Zaug A J, Cech T R. 1986. The intervening sequence RNA of Tetrahymena is an enzyme. Science 231: 470-475.
Ziehler W A, Engelke D R. 2001. Probing RNA structure with chemical reagents and enzymes. Curr Protoc Nucleic Acid Chem Chapter 6: Unit 61.

### Example 4: Evaluating the Oryza Sativa RNA Structurome for the Presence of Prokaryotic-Type RNA Thermometers

RNA secondary structures are known to modulate translation initiation in prokaryotes: for example, strong mRNA structure can impede ribosome binding to the Shine-Dalgamo (SD) sequence (AGGA) (19). RNA thermometers (RNATs) in prokaryotes function by temperature-dependent changes in secondary structure that alter accessibility of the SD sequence to the ribosome, thereby controlling translation initiation in a temperature-dependent manner (20, 21). The repression of heat shock gene expression (ROSE) element and four U element are two common types of RNA thermometers found in prokaryotes. These two types of RNATs operate in similar ways: the SD sequence is harbored in a hairpin structure at low temperature and the local hairpin melts at high temperature to expose the SD sequence, allowing ribosome binding. Another type of RNAT, found in Synechocystis sp. PCC6803 (22), is similar to the four U element but has UCCU, rather than four U's, base-pairing with the SD sequence. Two other RNATs are associated with two specific genes in prokaryotes: the prfA RNAT found in the 5′UTR of the prfA gene in Listeria monocytogenes (23) and the cssA RNAT found in the 5′UTR of the cssA gene in Neisseria meningitides (24). These thermometers are characterized by a strong hairpin located upstream and nearby the start codon, and have SD sequences within the hairpin that differ from the standard AGGA sequence. Other types of RNATs in prokaryotes also employ similar mechanisms for controlling translation initiation. Narbenhaus and colleagues (25) identified multiple candidate RNATs in Yersinia pseudotuberculosis from genome-wide in vitro RNA structure data by identifying transcripts with a decreased average PARS score (less RNA structure) at the SD region (located 10 nt±4 nt upstream of the start codon) under elevated temperature (25). A subset of these RNATs were validated by observation of significant protein abundance increase under elevated temperatures in transient reporter assays conducted in E, coli. This study provides the first in vivo genome-wide datasets on temperature regulation of a eukaryotic RNA structurome, affording an opportunity to investigate the possible presence of prokaryotic or other types of RNA-based thermometers. The RNA-seq and Ribo-seq data also allow direct assessment in the organism of interest of possible correlations between temperature-regulated RNA structure and transcript abundance or translation. However, as described herein, there is no evidence for prokaryotic-type RNA thermometers in the datasets.

RNA Thermometers Search Based on SD Sequence

a. ROSE Element

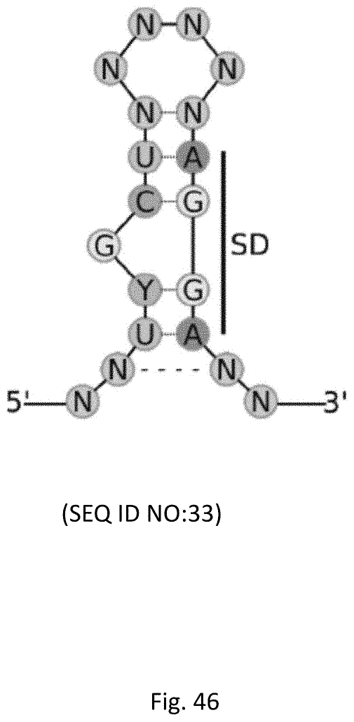

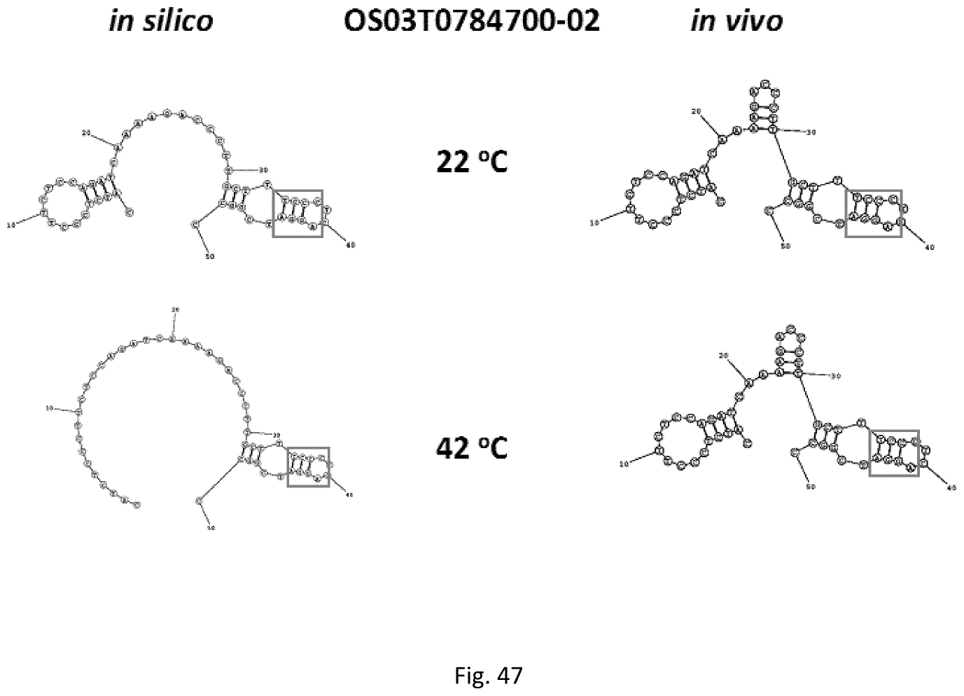

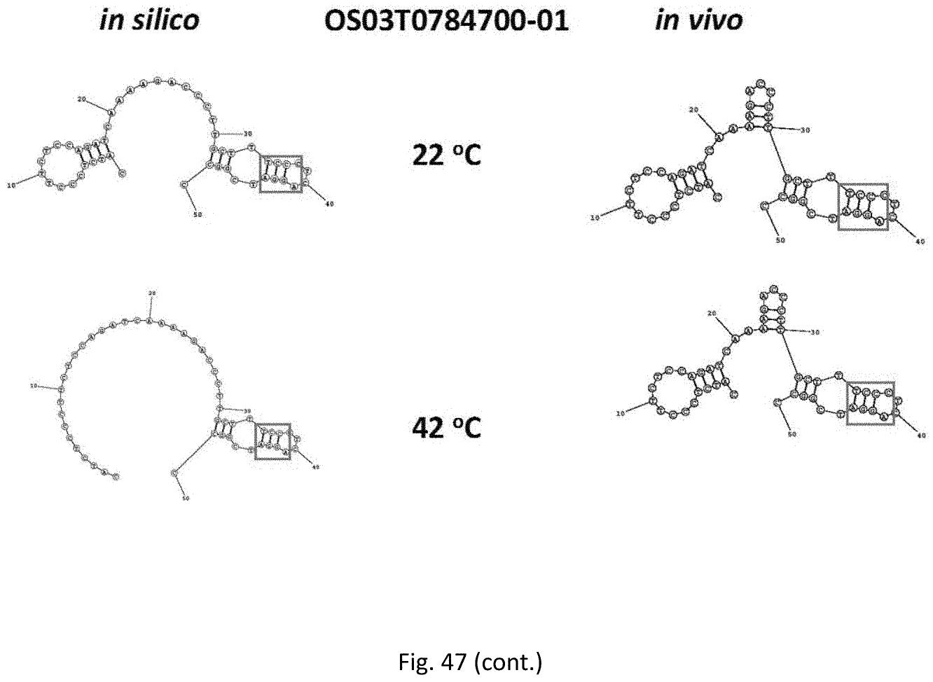

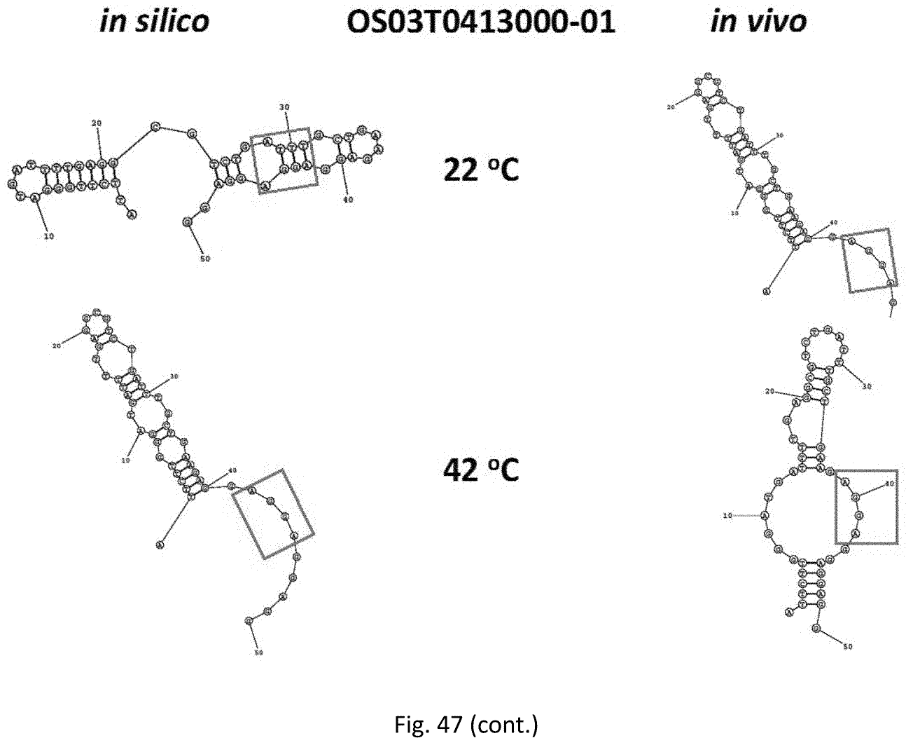

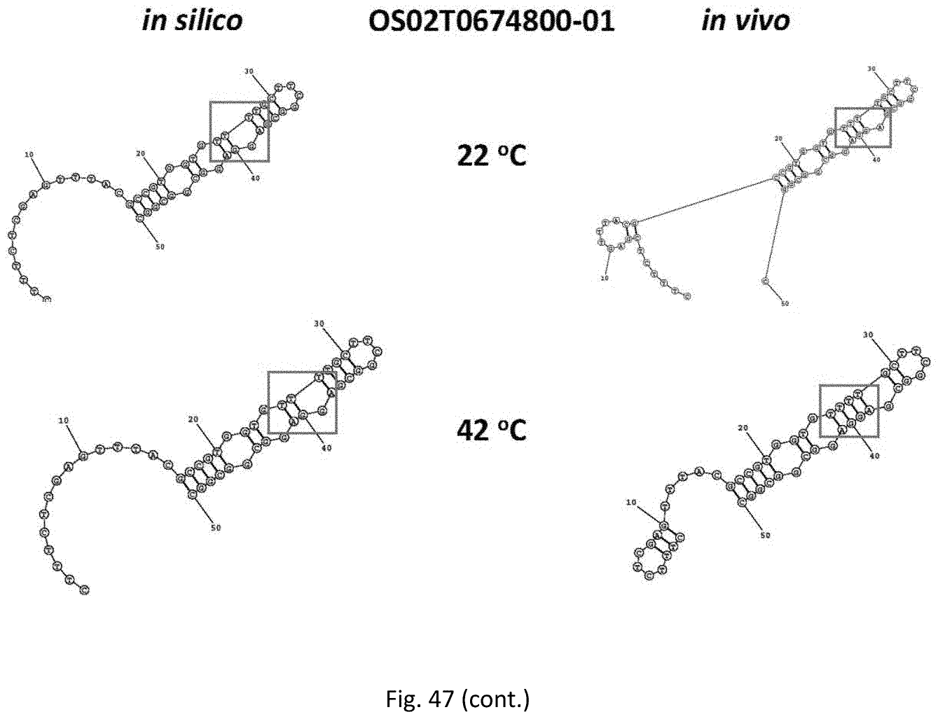

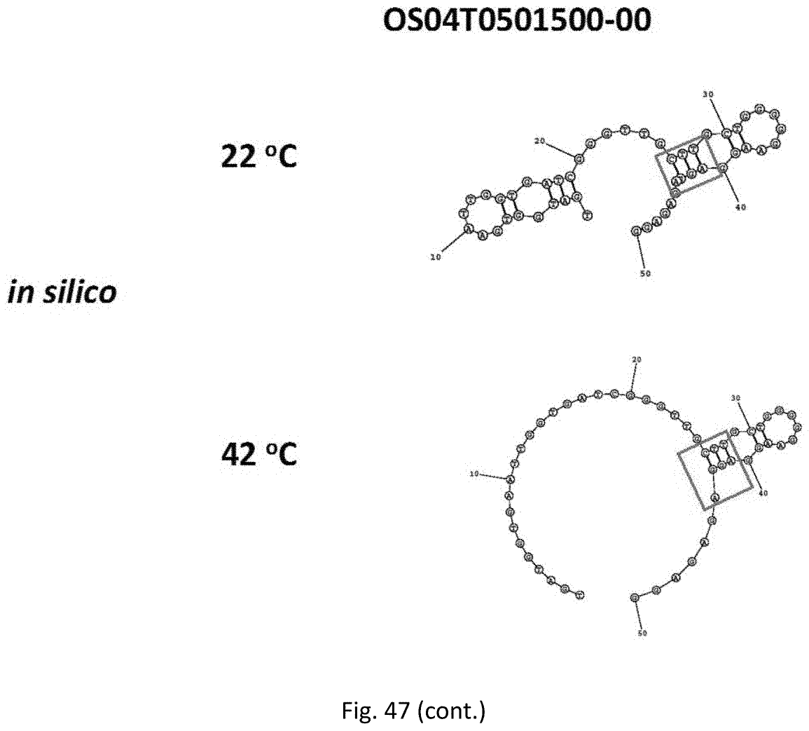

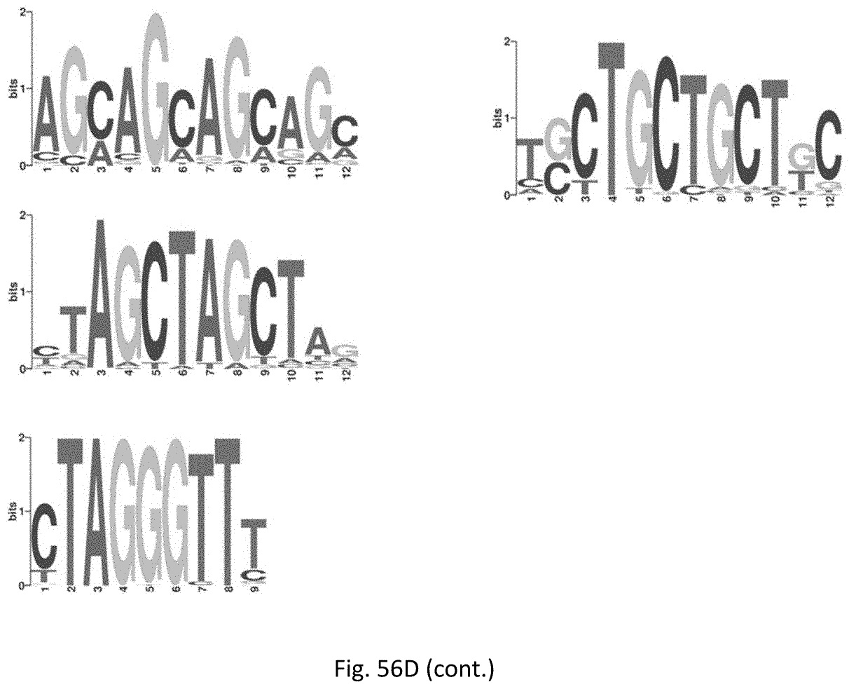

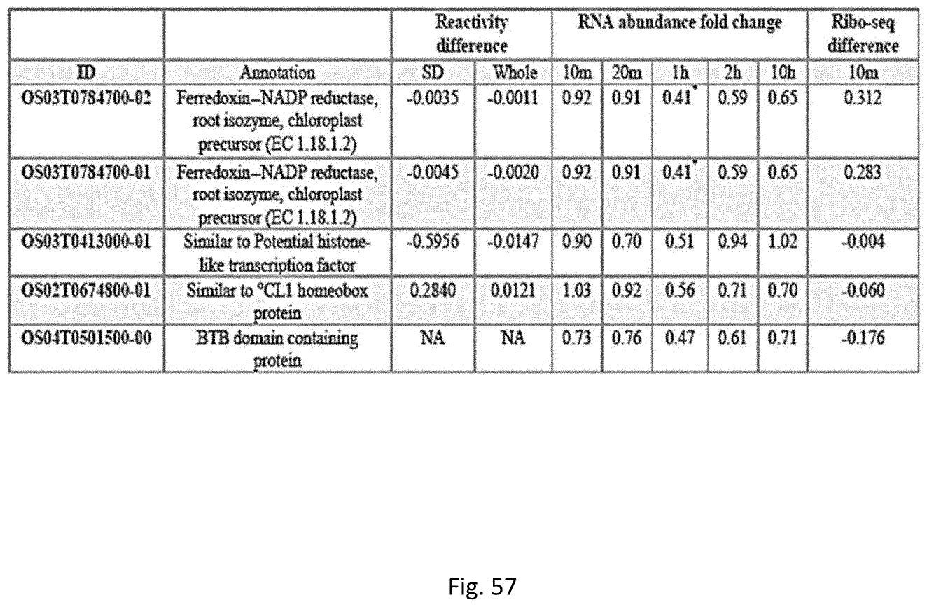

The repression of heat shock gene expression (ROSE) element is an RNA element that regulates translation and is found in the 5′UTRs of some bacterial heat shock genes (26). This element consists of a conserved SD sequence that base pairs with a UYGCU region, where Y represents a pYrimidine (C or U). FIG. 46 shows the RNA structure model of the ROSE element (20.21). A sequence search was performed of the Oryza sativa reference transcriptome for ROSE elements present in the region 50 nt upstream of the start codon in mRNAs that contain a SD sequence located 10 nt±4 nt upstream of the start codon. 1,621 candidates were identified with a SD sequence within this region. Among these, five contained a ROSE element based on sequence identity. Of these, four had sufficient coverage in the RNA structuromes. Structures for these four mRNAs were predicted using RNA structure (27) with and without DMS reactivities as restraints. The fifth mRNA, which does not meet the coverage requirement, was predicted in silico only. However, none of the candidates are predicted to form an RNA secondary structure similar to that of the ROSE element (FIG. 47) at 22° C.; moreover none of these are a heat shock gene. Temperature change has little effect on the predicted RNA structures. None of these candidates exhibit a significant elevation in RNA abundance between 22° C. and 42° C. at any time point (FIG. 57).

FourU Element

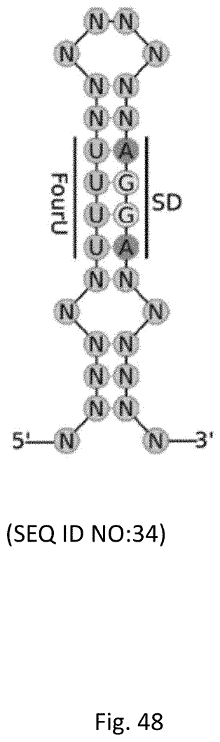

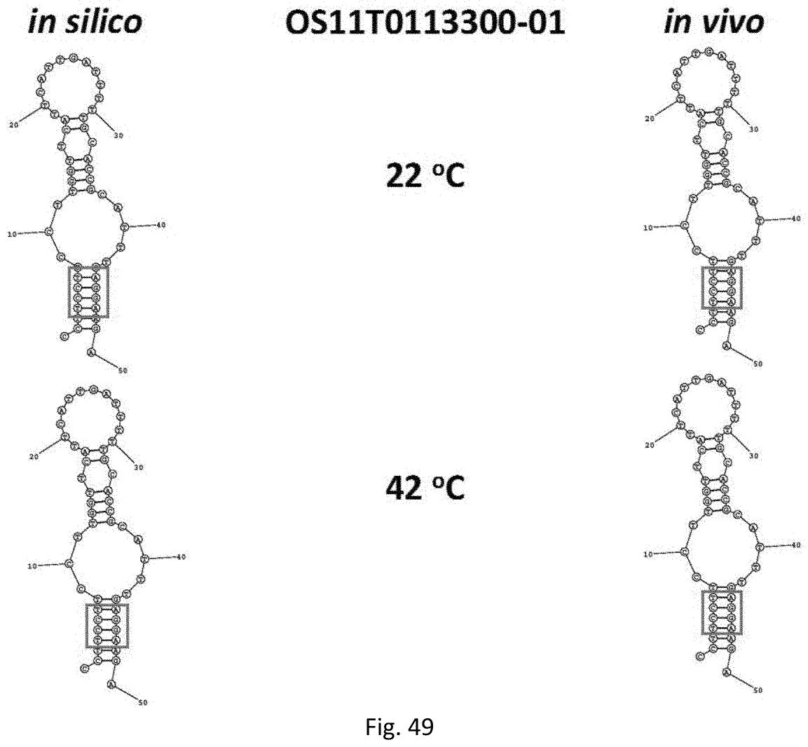

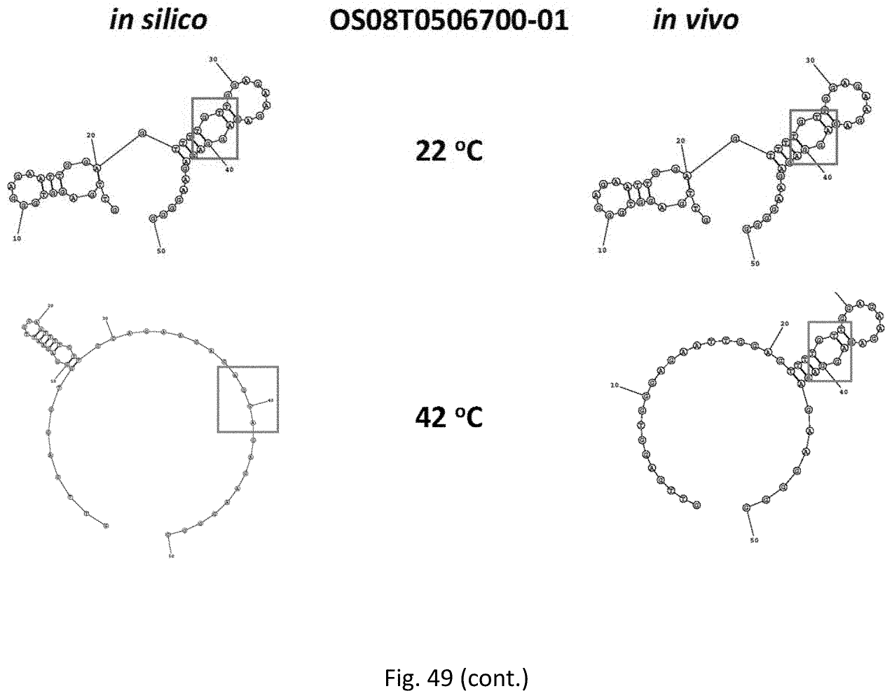

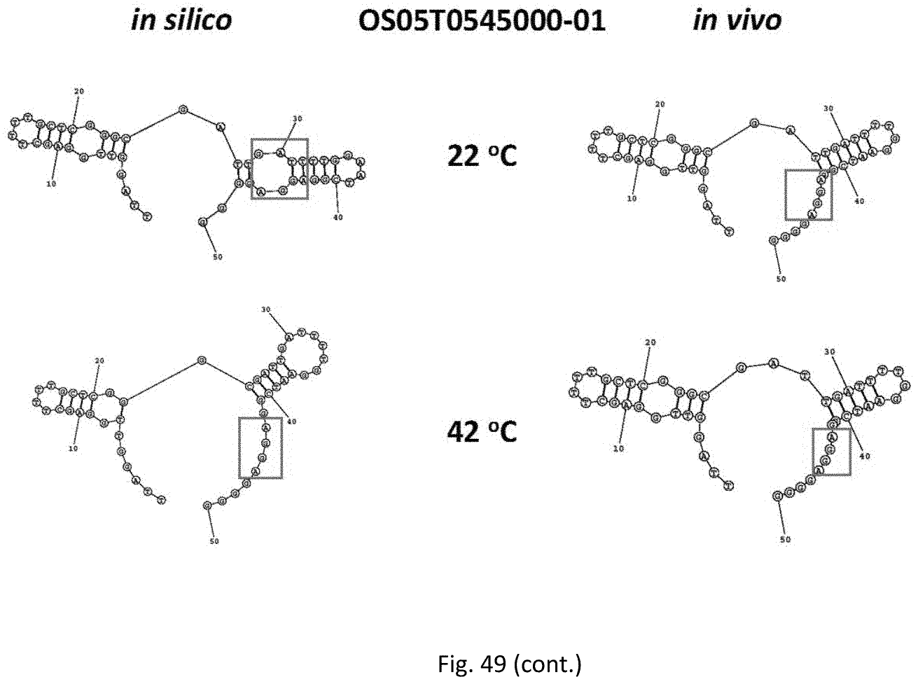

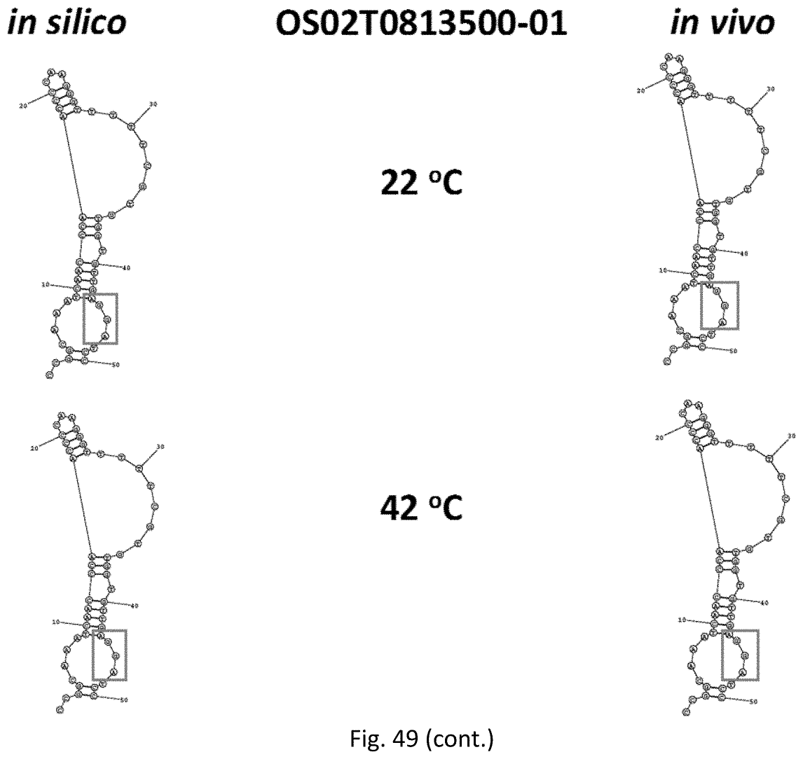

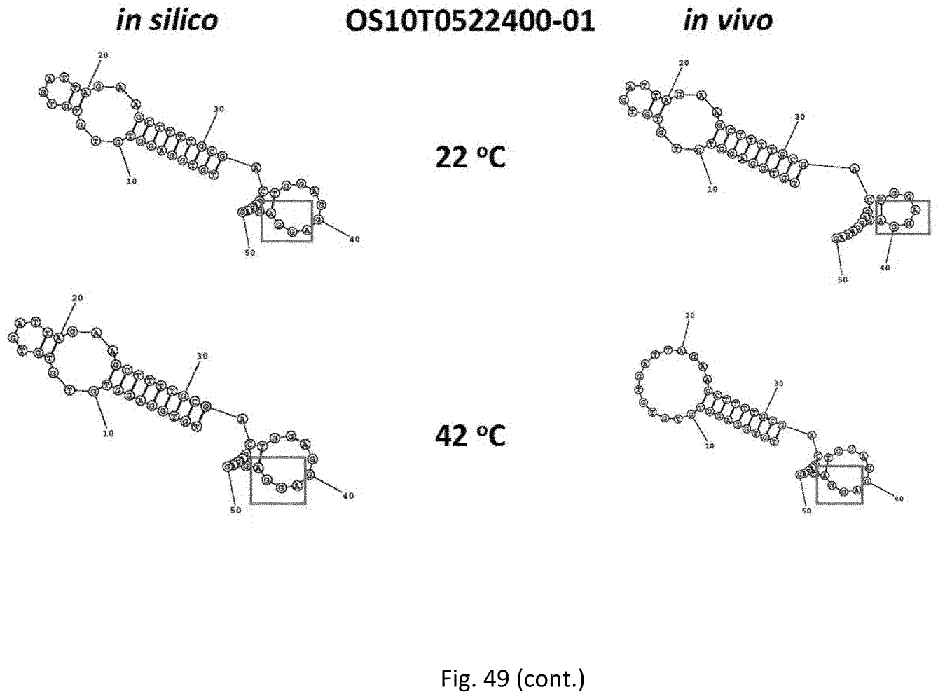

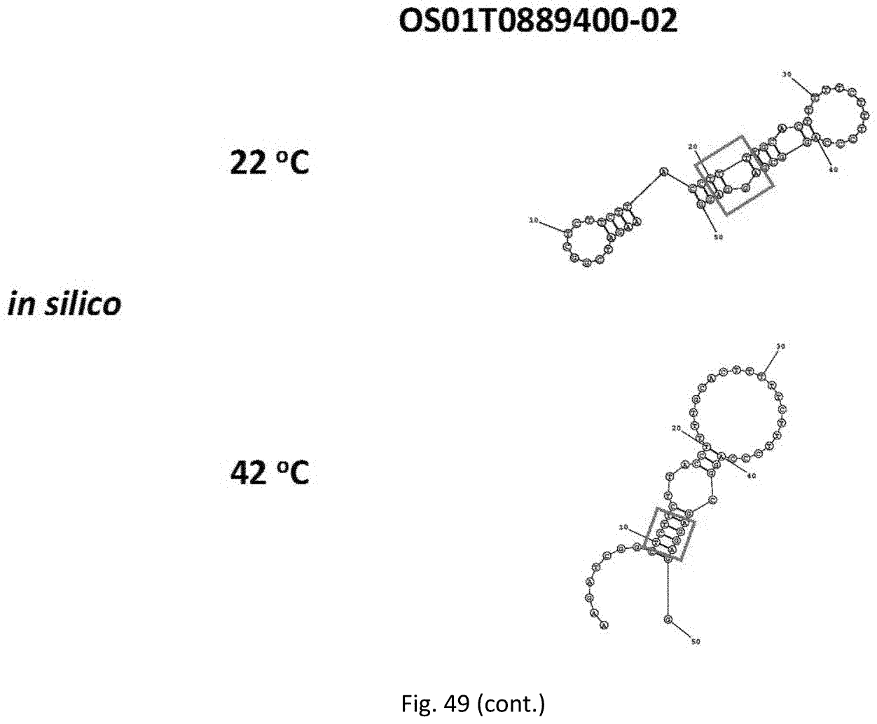

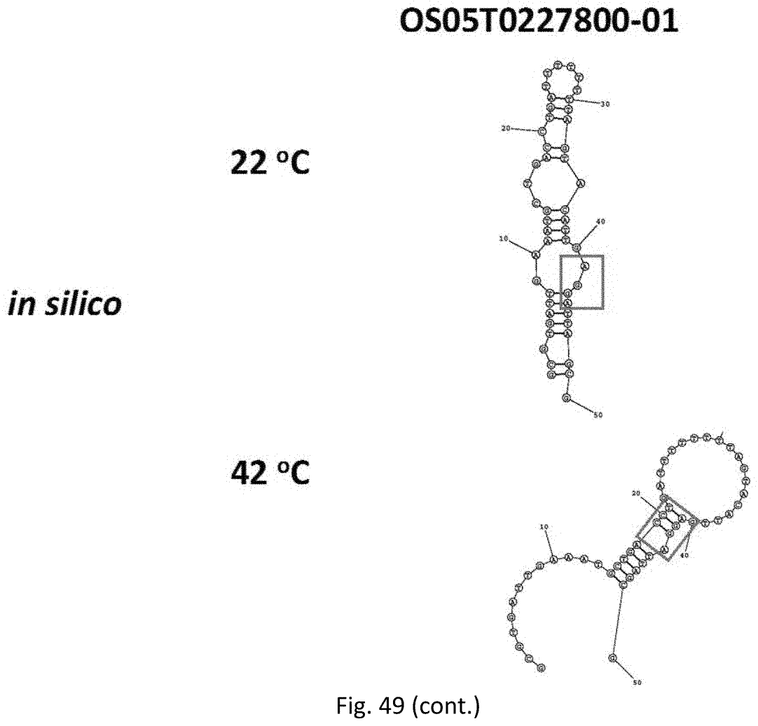

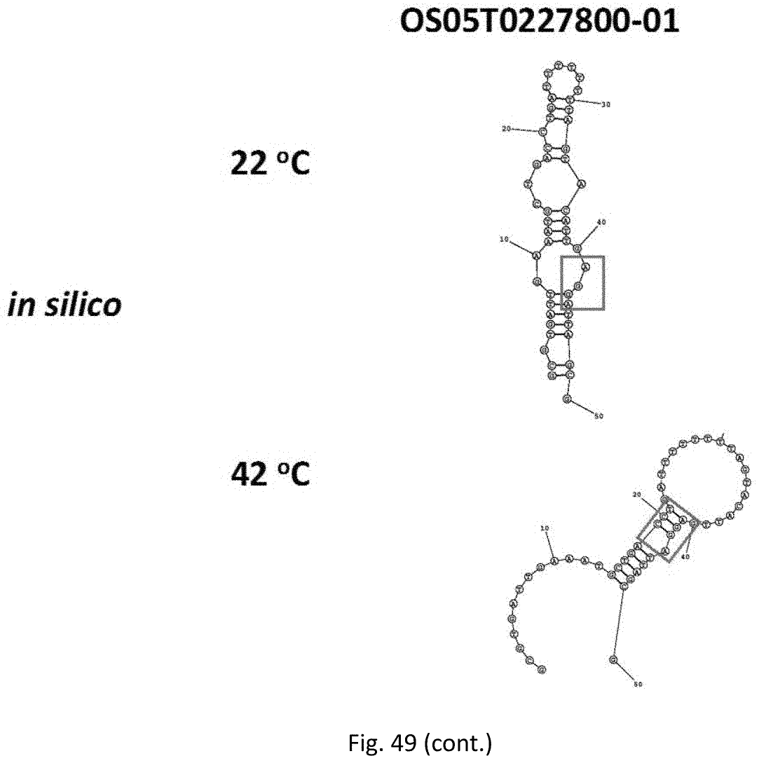

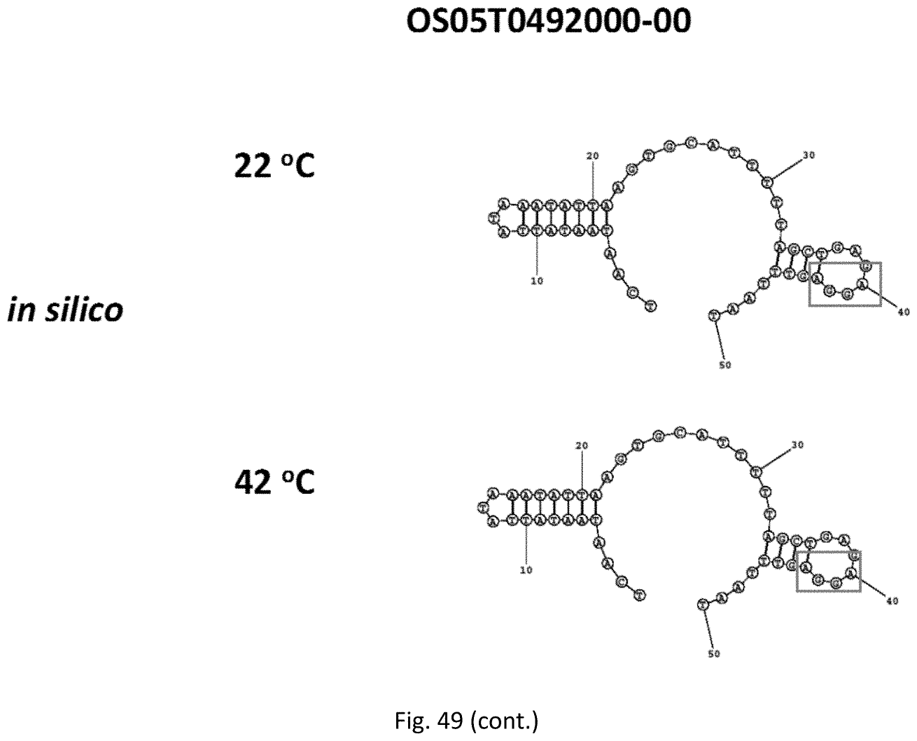

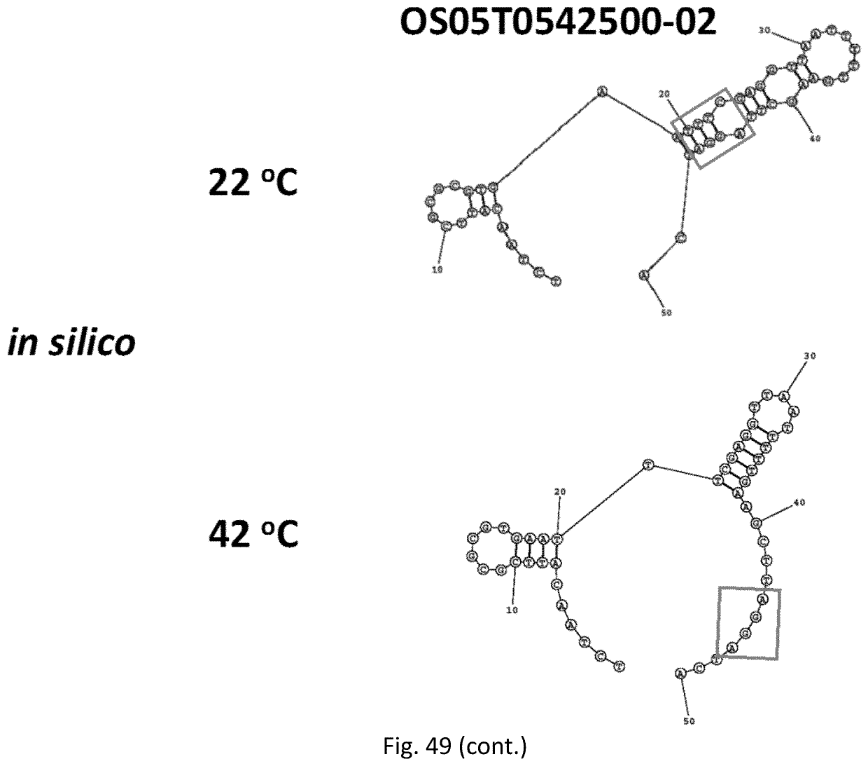

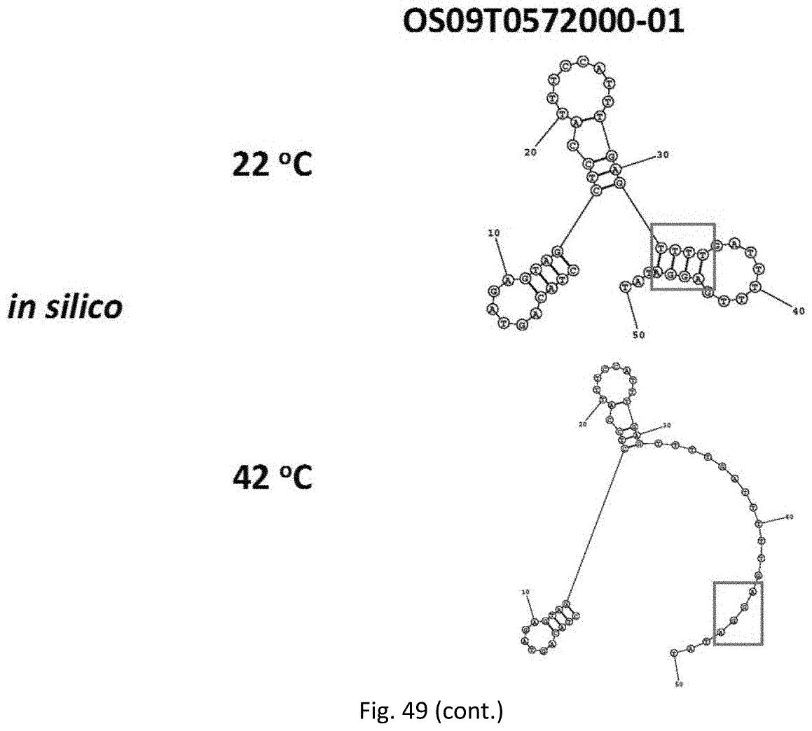

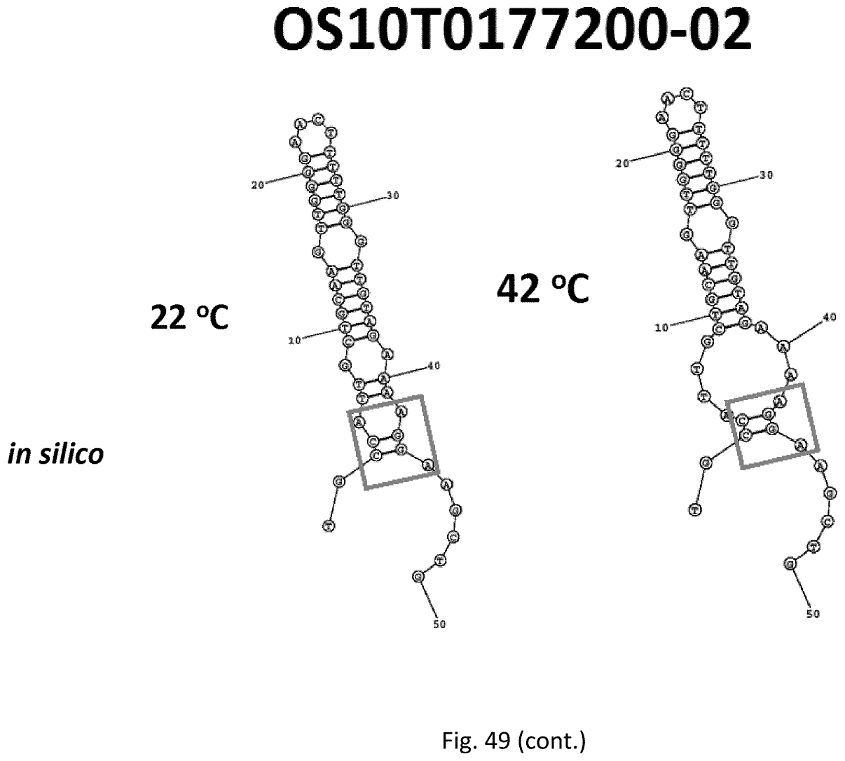

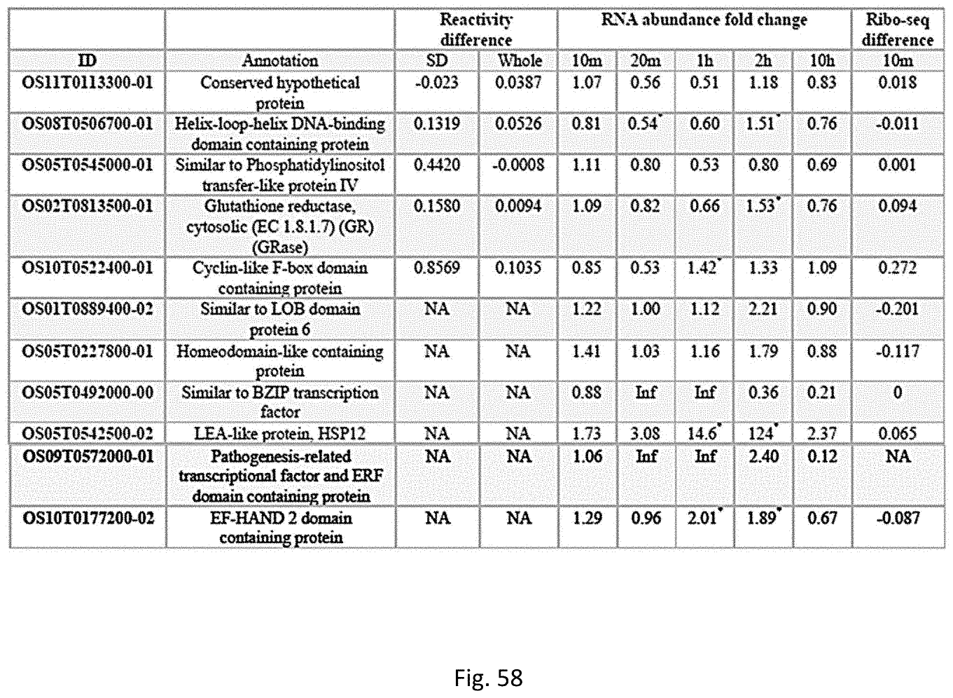

FourU thermometers are a type of RNA thermometer found in Salmonella (28), E. coli (29) and V. cholerae (30). This element consists of a conserved SD sequence that base pairs with a UUUU region. FIG. 48 shows the RNA structure model of the fourU element (20, 21). A sequence search was performed for four U elements in the region 50 nt upstream of the start codon on all the mRNAs with a SD sequence present 10 nt±4 at upstream of the start codon, and identified 11 four U candidates with sequences which match that of the four U element. Of these, five had sufficient coverage in the RNA structuromes for structure prediction. However, only one of these candidates (OS09T0572000-01) forms a predicted RNA secondary structure similar to that of the four U element (FIG. 4) at 22° C. While the SD sequence part of OS09T0572000-01 melts in silico at 42° C., the RNA abundance of OS09T0572000-01 is only 0.07 (TPM), which is too low for RNA structure probing in vivo. Temperature change also has little effect on the remaining 10 RNA structures predicted either with or without DMS reactivities as restraints. One of these candidates (OS05T0542500-02) exhibits a dramatic change in RNA abundance between 22° C. and 42° C. at 1 hr, 2 his and 10 hrs time points (FIG. 58). However, the predicted RNA secondary structure of OS05T0542500-02 is not similar to that of the four U element.

UCCU Element

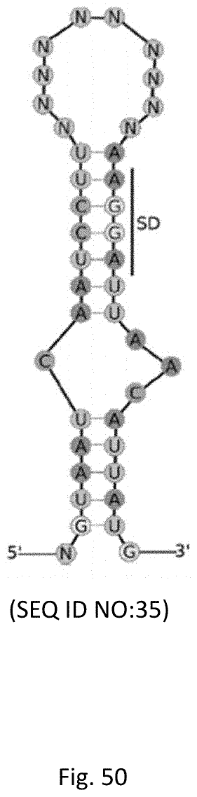

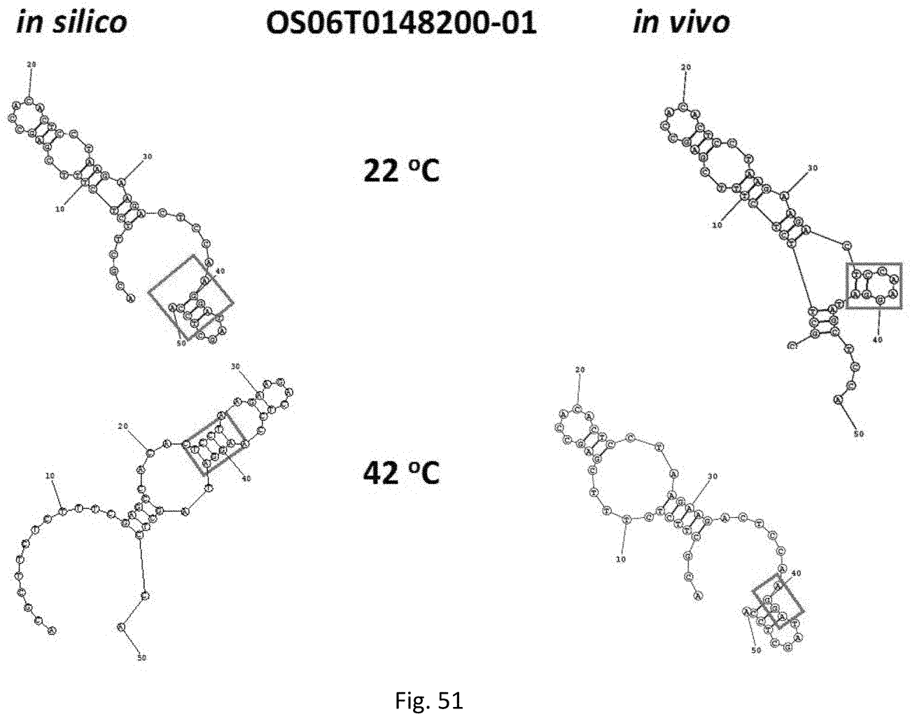

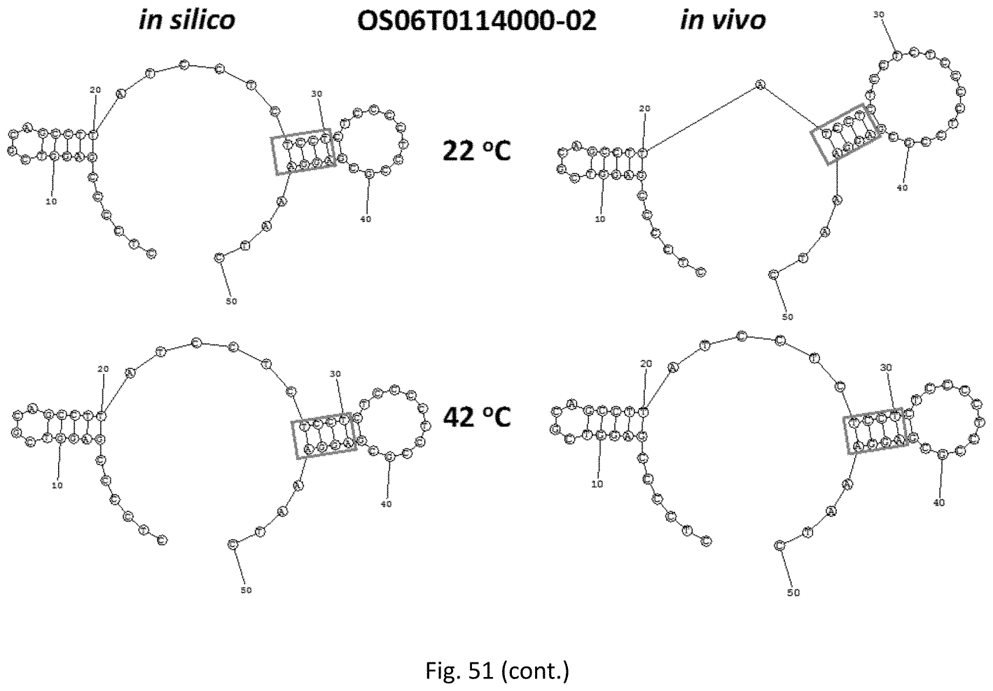

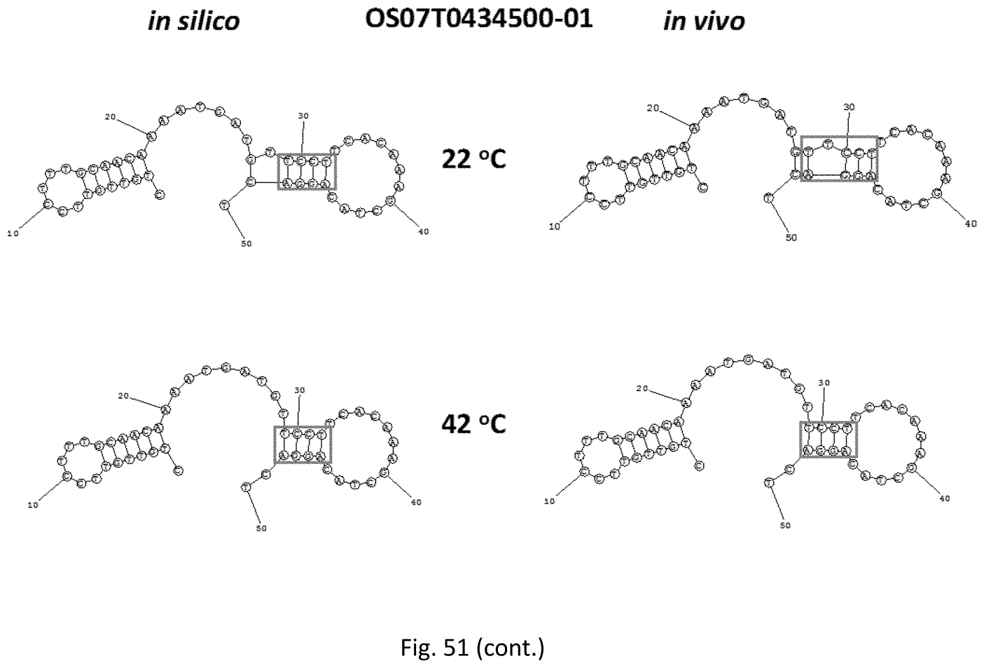

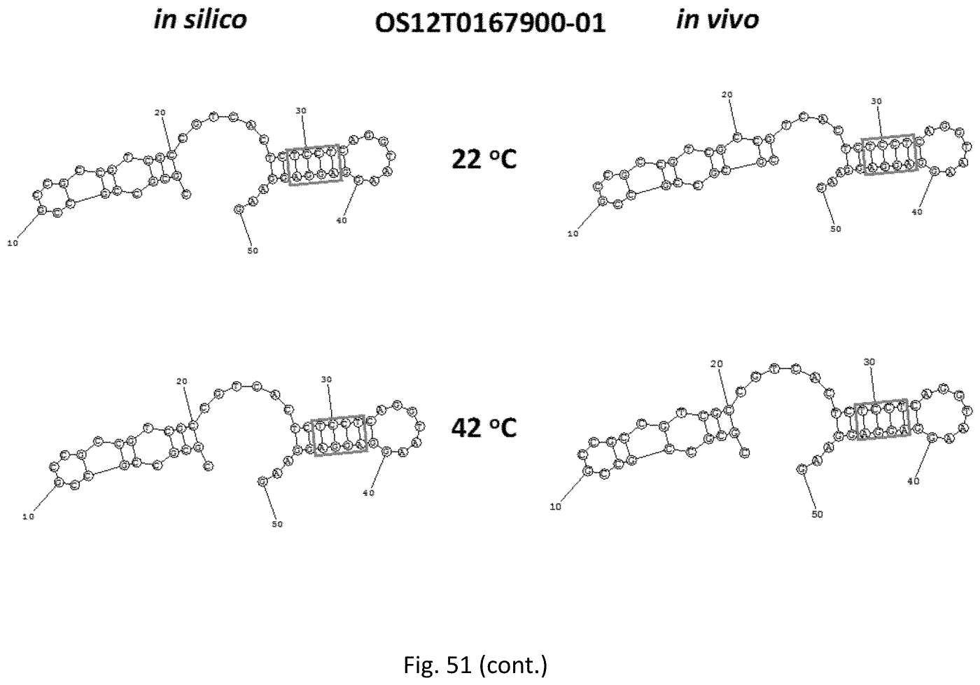

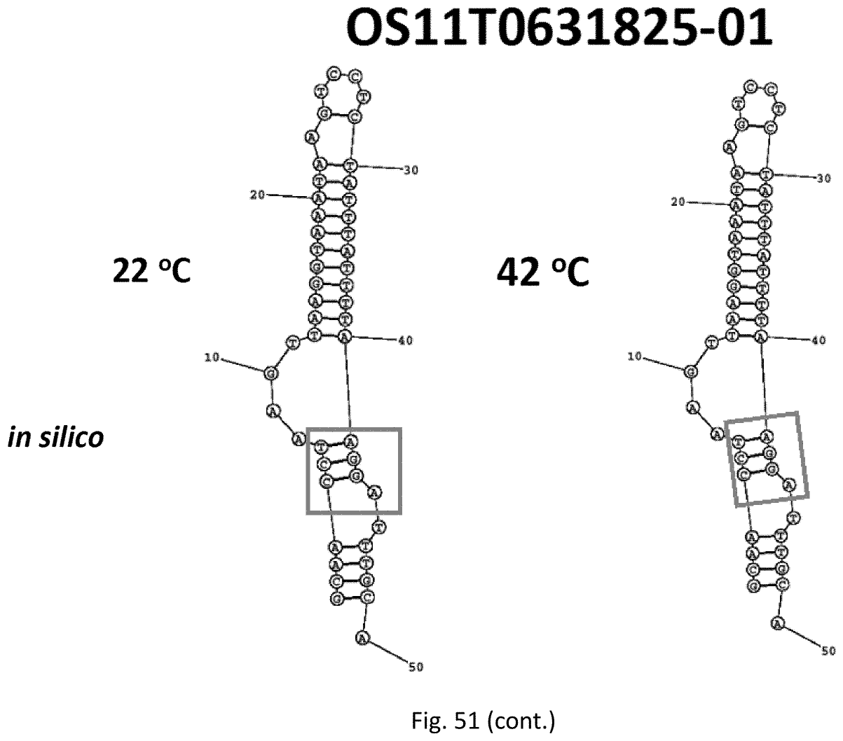

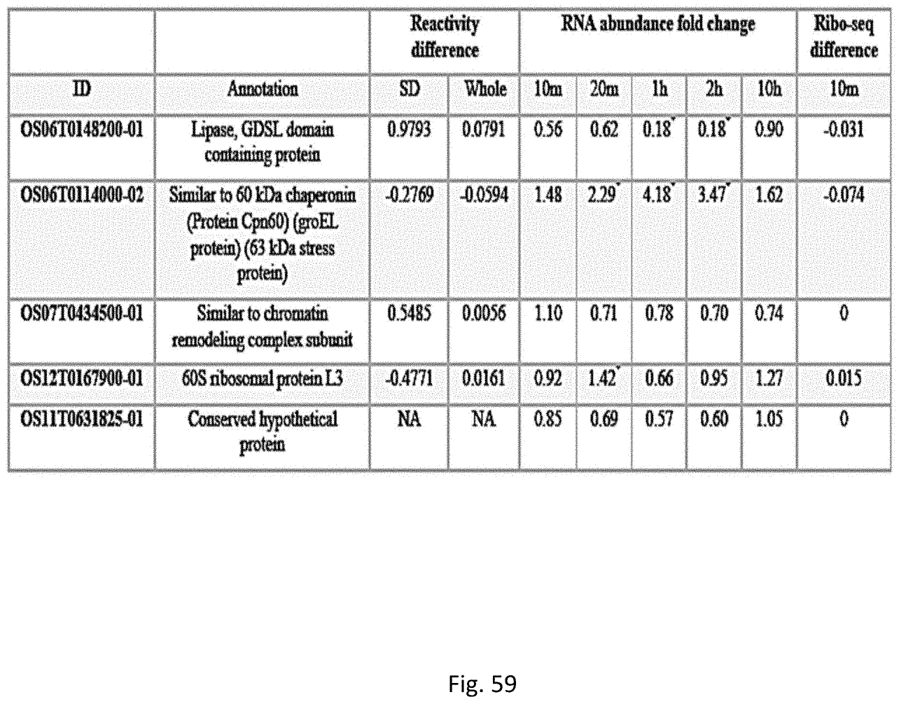

UCCU thermometers are a type of RNA thermometer found in Synechocystis sp. PCC6803 (22). FIG. 50 shows the RNA structure model of the UCCU element (20, 21A sequence search for this type of RNAT was performed in the region 50 nt upstream of the start codon. Among these, five contained a UCCU element based on sequence identity. Of these, four had sufficient coverage in the RNA structuromes. Three of these candidates form a predicted RNA secondary structure similar to the UCCU element both in silico and in vivo at 22° C. (FIG. 51). However, none of these structures melts out at the SD region at 42° C. either in silico or in vivo (FIG. 51). Moreover, unlike the Synechocystis UCCU thermometer, none of these candidates is a heat shock mRNA. OS06T014000-02 has significant elevation of mRNA abundance at 42° C. as compared to 22° C. at 20 min, 1 hr and 2 hrs time points, and OS12T0167900-01 has significant elevation of mRNA abundance at 42° C. as compared to 22° C. at 20 min, however, neither of the candidates shows marked change in Ribo-seq signal between 22° C. and 42° C. (FIG. 59).

Other Types of RNATs in Bacteria

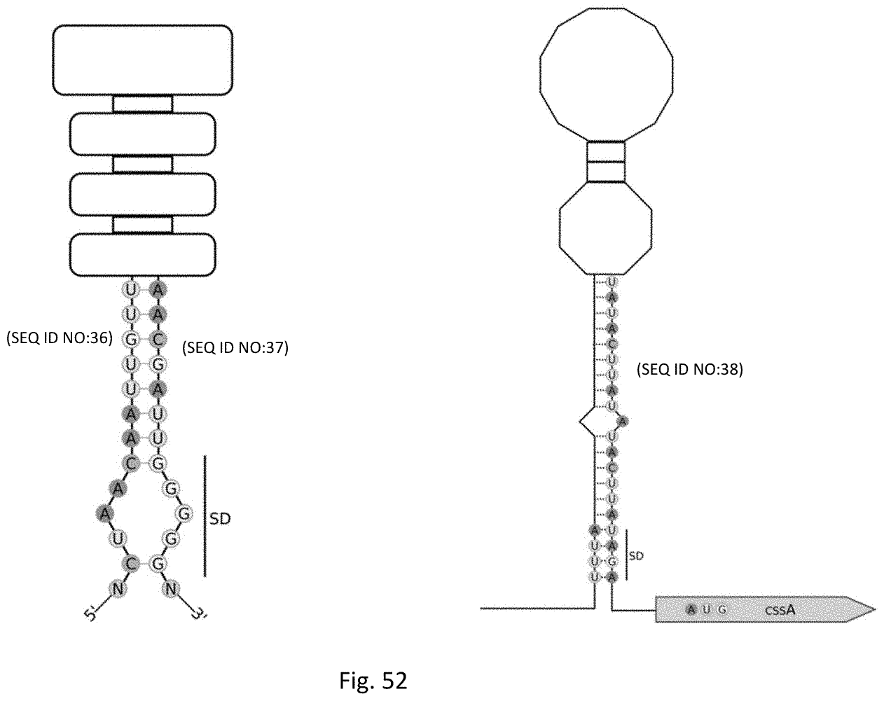

FIG. 52 shows RNA structure models of the prfA 5′UTR RNAT of Listeria monocytogenes (23) and the cssA 5′UTR RNAT of Neisseria meningitidis (24). Exact matches to these sequences were not found in the 5′UTRs of any Oryza sativa mRNAs.

RNA Thermometer Search in Rice Chloroplast Transcriptome

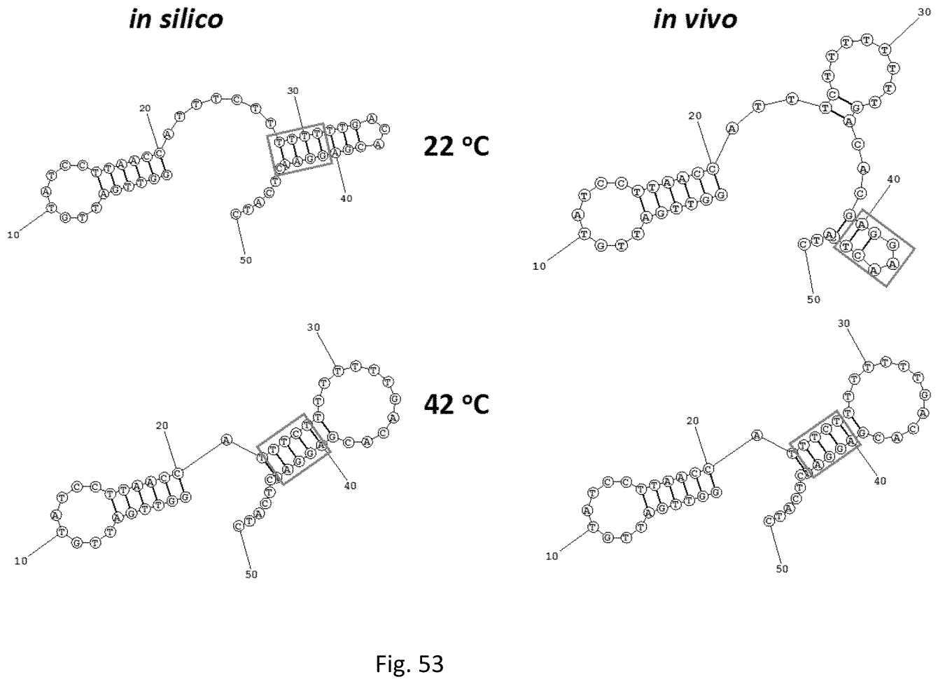

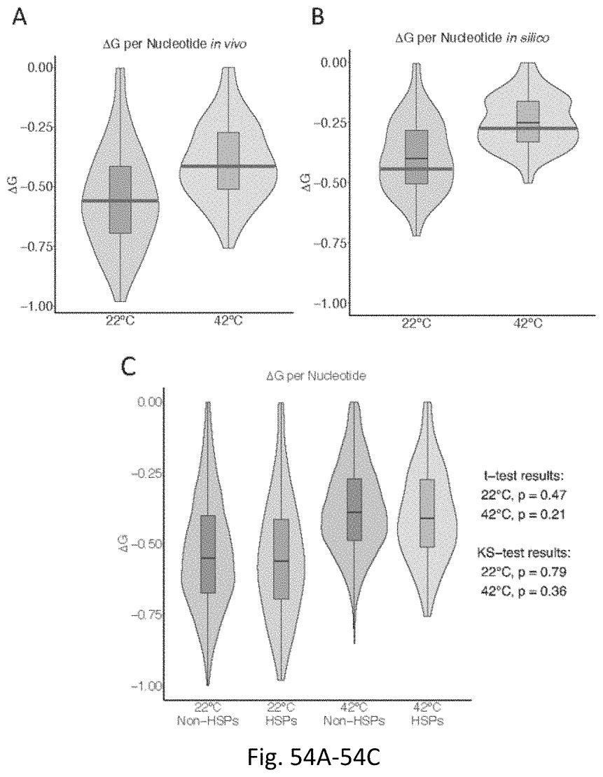

Since chloroplasts are of prokaryotic origin, a search was performed for prokaryotic types of RNA thermometers in the chloroplast transcriptome of rice. No sequence matches to the ROSE element or UCCU element types of RNA thermometers were found within the region 50 nt upstream of the start codon of chloroplast mRNAs. Only one candidate was identified that matches the four U element sequence, located in the region 50 nt upstream of the start codon of the atpH (ATP synthase subunit c) transcript. However, the SD sequence (marked by a square) is not open at 42° C. (FIG. 53) in either the in silico or the in vivo structures of this region, indicating that this candidate is not likely to be an RNA thermometer.

RNA Thermometers in Eukaryotes

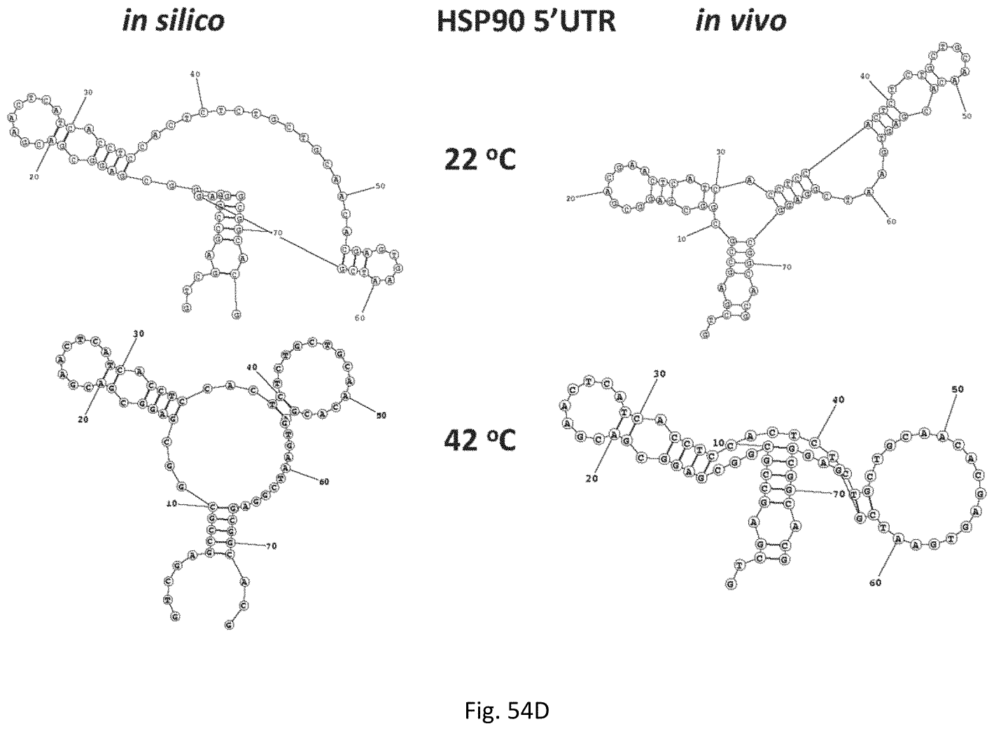

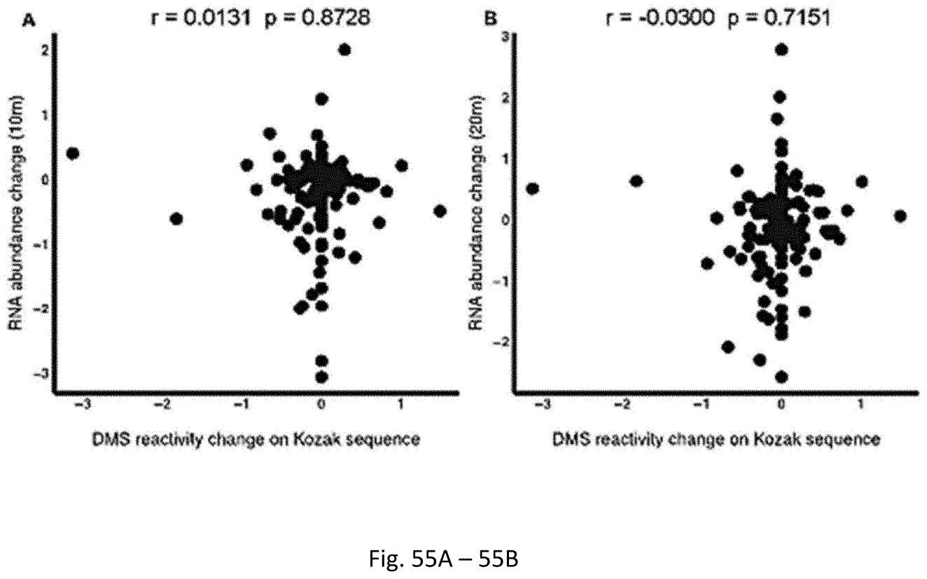

A cis-regulatory element thermometer was proposed for the HSP90 mRNA of the eukaryote, Drosophila melanogaster (31). As for most eukaryotic transcripts, the HSP90 transcript does not contain a SD sequence, but has a ˜3-4 fold increase in protein abundance under heat shock compared to a normal growth temperature. In D. melanogaster the 5′UTR of HSP90 had greater stability (significantly lower free energy per nucleotide) than other HSP mRNAs. In contrast, the ortholog of the HSP90 mRNA was identified in rice (OS06G716700) by sequence alignment and it was found that the free energy per nucleotide of the 5′UTR of the, rice HSP90 mRNA does not differ significantly as compared to other mRNAs that code for HSPs, based on predicted RNA structures in silico or with DMS reactivities as restraints at 22° C. and 42° C. (FIG. 54A and FIG. 54B).

The authors (31) also proposed that unlike the HSP70 and HSP22 mRNA which have minimal 5′UTR RNA secondary structure in D. melanogaster, the Drosophila HSP90 mRNA may adopt a similar mechanism as prokaryotic RNATs, consisting of thermal melting of a stem-containing region near start codon, although no direct evidence was provided. FIG. 54D shows the predicted RNA structure of the 5′UTR of rice HSP90 in silico or with DMS reactivities as restraints at 22° C. and 42° C. Obvious thermal melting of the RNA secondary structure was not observed near the start codon predicted either in silico or with DMS reactivities as restraints at 42° C. In fact, in rice, there is no significant difference in free energy per nucleotide in the 5′UTRs of mRNAs that code for HSPs versus all other mRNAs with sufficient coverage (FIG. 54C). Together, these results provide no evidence that HSP mRNAs in rice function as thermosensors in a similar way to that proposed for the HSP90 cis-regulatory element in D. melanogaster.

Kozak Sequence

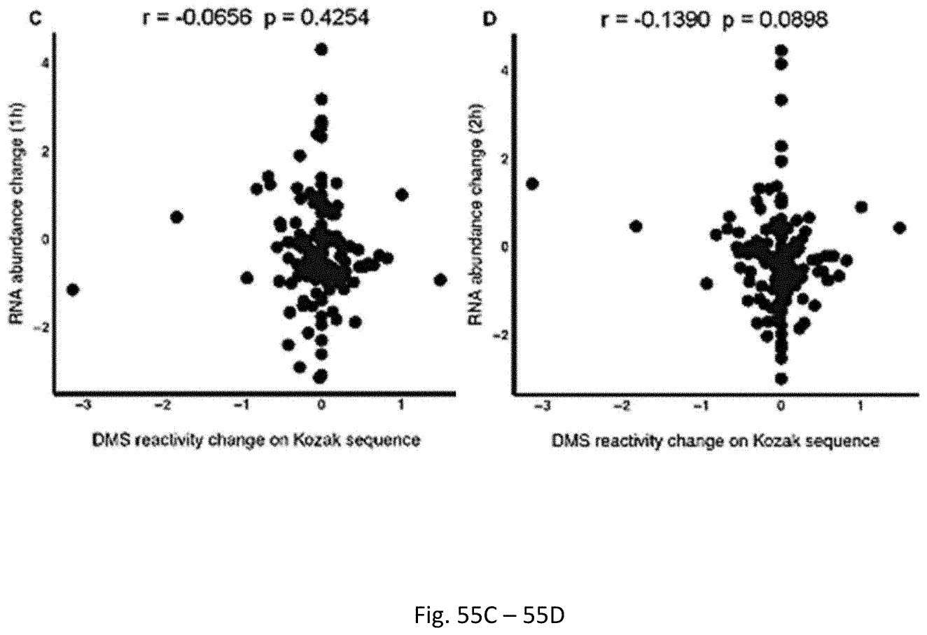

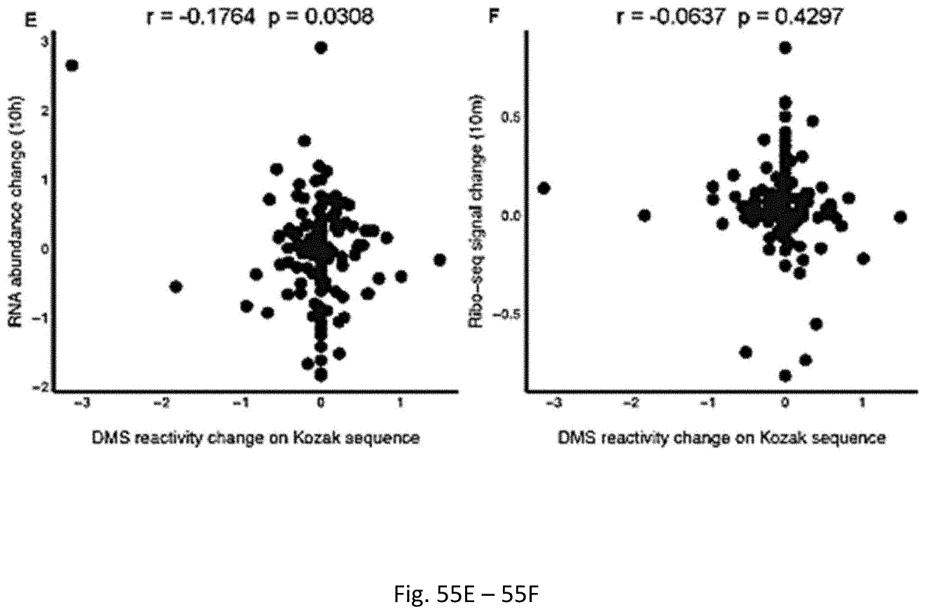

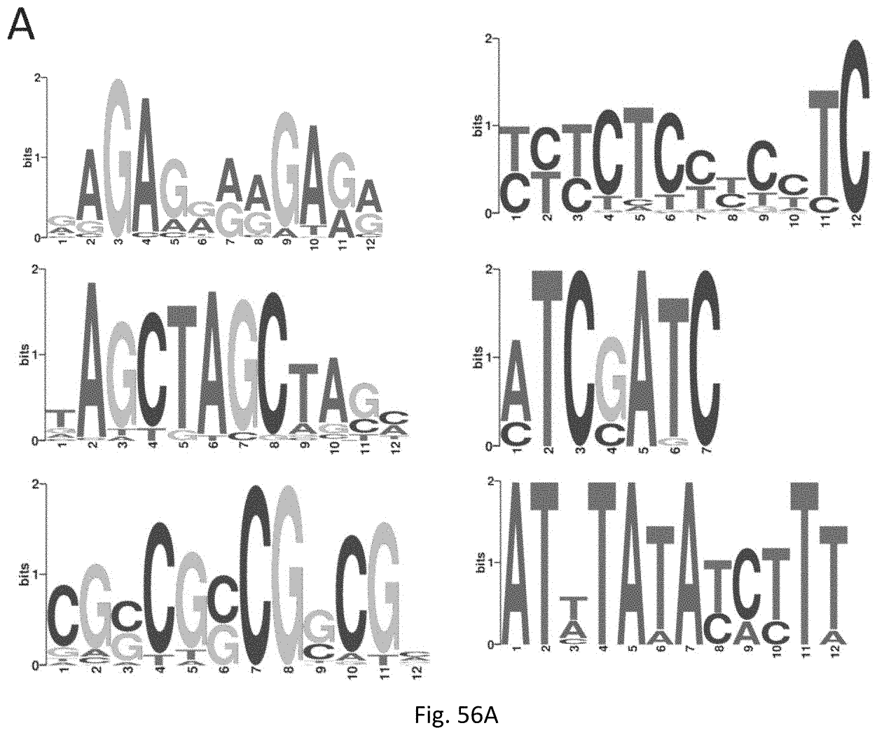

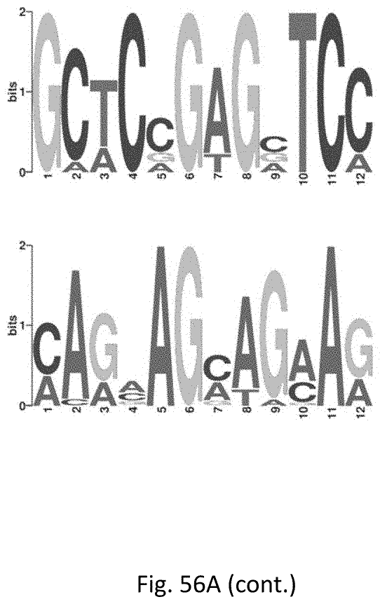

The Kozak consensus sequence is a sequence in eukaryotic mRNAs that plays an important role in translation initiation. Without being bound by theory, it was hypothesized that RNA thermometers in plants may function by temperature-dependent changes in secondary structure that alter accessibility of the Kozak sequence to the ribosome, thus regulating translation. The Kozak sequence in plants is AACA(AUG) as suggested in (32). 158 sequence matches to the Kozak sequence were identified within the set of 14.292 mRNAs with sufficient Structure-seq coverage. The correlation was checked between the average DMS reactivity change on the Kozak sequence between 22° C. and 42° C. of the identified 158 Kozak sequence-containing transcripts and their mRNA abundance fold change (log 2). However, the DMS reactivity change of these mRNAs is not correlated with their abundance fold change (log 2) at any time point (FIG. 55A-FIG. 55E). In addition, the correlation between the average DMS reactivity change on the Kozak sequence between 22° C. and 42° C. of these 158 Kozak sequence-containing transcripts and their Ribo-seq signal change (FIG. 55F) was investigated, but no correlation between average DMS reactivity change and Ribo-seq signal change between 22° C. and 42° C. was observed. These results indicate that the Kozak sequence may not be a target for RNA structure-based regulation of gene expression in Oryza sativa.

RNA Thermometer Search in 5′UTRs within the 50 nt Upstream of the Start Codon in Rice

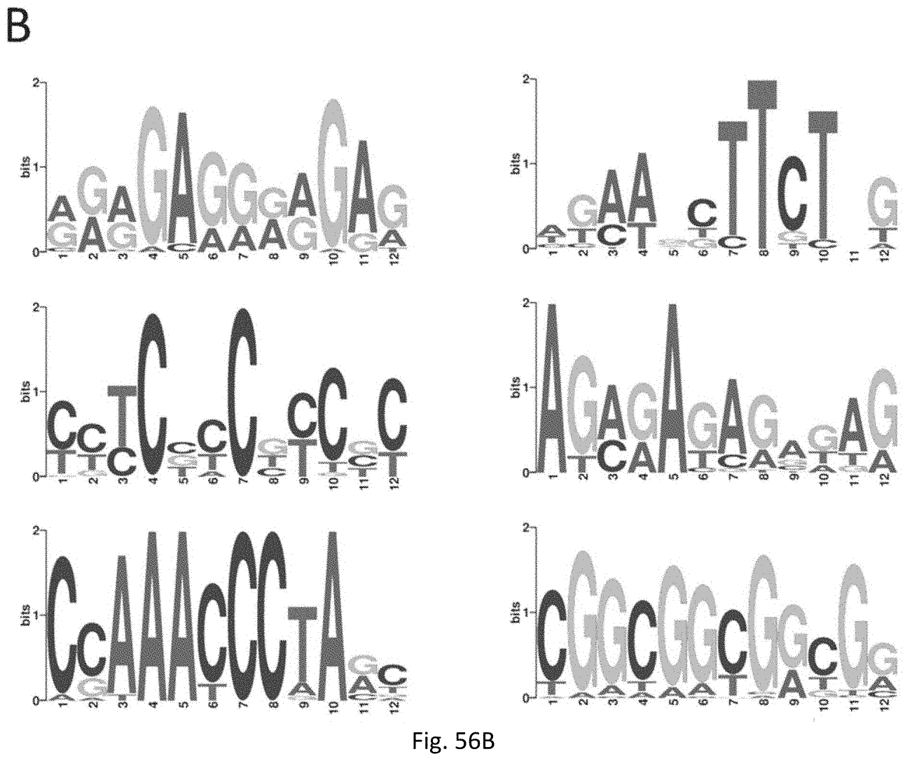

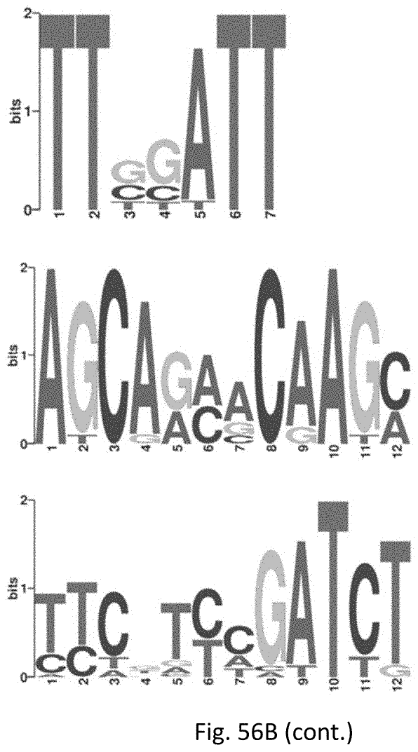

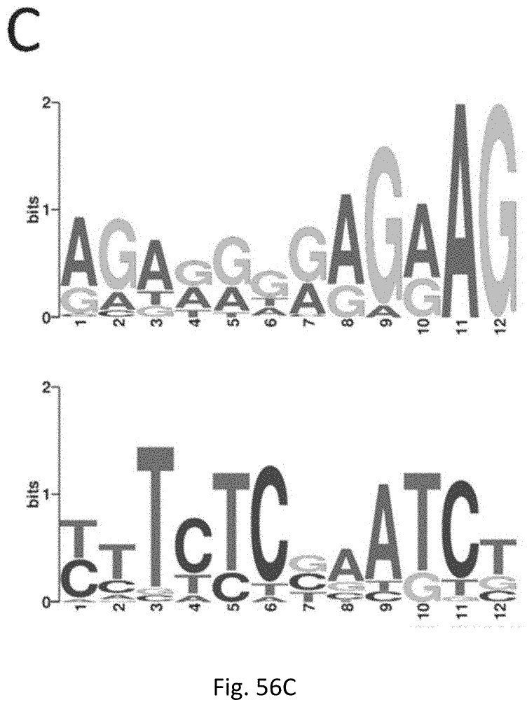

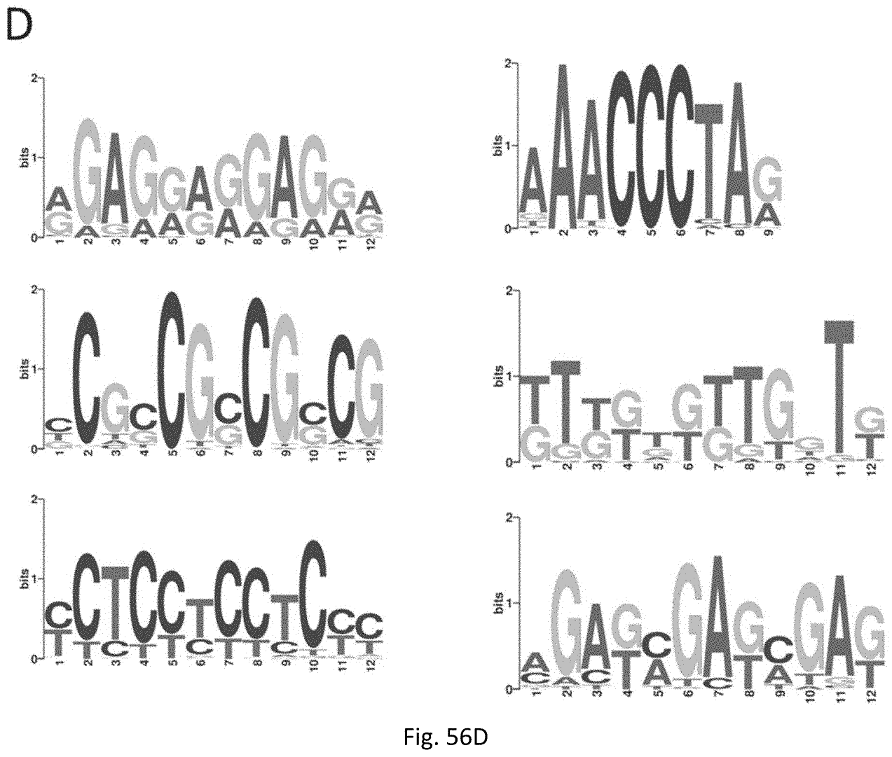

A sequence motif search was performed with the idea that rice might employ a temperature-regulated sequence motif near the start codon that is different from known RNAT translation-related motifs. The motif search was performed using MEME (33) on the 50 nt upstream of the start codon of the “top group” (FIG. 56A) and “bottom group” (FIG. 561) of mRNAs. Here, the top group is the 5% of mRNAs with the most elevated average DMS reactivity at 42° C. as compared to 22° C. and the bottom group is the of 5% mRNAs with the most reduced average reactivity at 42° C. as compared to 22° C. Sequence motif search was also performed on all mRNAs (n=4,308) with elevated Ribo-seq signal at 42° C. and with 5′UTR length y 50 nt (FIG. 56C), and all mRNAs (n=8,739) with sufficient coverage and with 5′UTR length 50 nt in the RNA structuromes (FIG. 56D). A similar AG rich motif was observed as the most overrepresented sequence motif among the four groups. In addition, a strongly overrepresented motif was not observed within each group: motifs within each group are quite different from each other. These results suggest that Oryza sativa may not employ conserved sequence motifs as analogous to the SD sequence in RNATs of bacteria.

Based on the above results, no evidence was found in rice for RNA thermometers of the prokaryotic type. In addition, no evidence was found of any HSP mRNA functioning as a thermosensor in the manner proposed for the HSP90 cis-regulatory element thermometer in Drosophila melanogaster (31), nor was any evidence found for a Kozak sequence acting like the SD sequence of prokaryotic RNA thermometers. In addition, no clear evidence was found for any conserved mRNA sequence motif that functions as a RNA thermometer. In summary, evidence in rice for discrete RNA-based thermometers was not found.

### REFERENCES

19. Laursen B S, Sorensen H P, Mortensen K K, & Sperling-Petersen H L (2005) Initiation of protein synthesis in bacteria. Microbiol. Mol, Biol. Rev, 69(1):101-123.
20. Narberhaus F (2010) Translational control of bacterial heat shock and virulence genes by temperature-sensing mRNAs. RNA Biol. 7(1):84-89.
21. Krajewski S S & Narberhaus F (2014) Temperature-driven differential gene expression by RNA thermosensors. Biochim. Biophys. Acta 1839(10):978-988.
22. Waldminghaus T, Gaubig L C, & Narberhaus F (2007) Genome-wide bioinformatic prediction and experimental evaluation of potential RNA thermometers. Mol, Genet Genomics 278(5):555-564.
23. Johansson J, et al. (2002) An RNA thermosensor controls expression of virulence genes in Listeria monocytogenes. Cell 10(5):551-561.
24. Loh E, et al. (2013) Temperature triggers immune evasion by Neisseria meningitidis. Nature 502(7470):237-240.
25. Righetti F. et at (2016) Temperature-responsive in vitro RNA structurome of Yersinia pseudotuberculosis. Proc. Natl Acad. Sci. USA 113(26):7237-7242.
26. Nocker A, et al. (2001) A mRNA-based thermosensor controls expression of rhizobial heat shock genes. Nucleic Acids Res. 29(23):4800-4807.
27. Reuter J S & Mathews D H (2010) RNAstructure: software for RNA secondary structure prediction and analysis. BMC Bioinformatics 11:129.
28. Waldminghaus T, Heidrich N, Brantl S & Narberhaus F (2007) FourU: a novel type of RNA thermometer in Salmonella. Mol. Microbiol. 65(2):413-424.
29. Klinkert B, e al. (2012) Thermogenetic tools to monitor temperature-dependent gene expression in bacteria. J. Biotechnol. 160(1-2):55-63.
30. Weber G G, Kortmann 3, Narberhaus F, & Klose K E (2014) RNA thermometer controls temperature dependent virulence factor expression in Vibrio cholerae. Proc. Natl Acad. Sci. USA 111(39):14241-14246.

31. Ahmed R & Duncan R F (2004) Translational regulation of Hsp90 mRNA. AUG-proximal 5′-untranslated region elements essential for preferential heat shock translation. J. Biol, Chem. 279(48):49919-49930.
32. Lutcke H A, et al. (1987) Selection of AUG initiation codons differs in plants and animals. EMBO J. 6(1):43-48.
33. Bailey T L, et al. (2009) MEME SUITE: tools for motif discovery and searching. Nucleic. Acids Res. 37(Web Server issue):W202-208.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of obtaining nucleotide-resolution RNA structural information in vivo, the method comprising the ordered steps of:
a) treating an RNA molecule in vivo with an agent which covalently modifies unprotected nucleobases,
b) performing reverse transcription (RT) with a random hexamer-containing primer to generate a cDNA molecule,
c) ligating a hairpin donor molecule to the 3′ end of the cDNA molecule,
d) performing PCR amplification of the ligated construct and
e) sequencing the amplified products.

2. The method of claim 1, wherein the agent is selected from the group consisting of dimethyl sulfate (DMS), glyoxal, methylglyoxal, phenylglyoxal, 1-cyclohexyl-3-(2-morpholinoethyl)-carbodiimide methyl-p-toluenesulfonate (CMCT), nicotinoyl azide (NAz), 1-ethyl-3-(3-dimethylaminopropyl)carbodiimide (EDC), 1M7 (1-methyl-7-nitroisatoic anhydride), 1M6 (1-methyl-6-nitroisatoic anhydride), NMIA (N-methyl-isatoic anhydride), FAI (2-methyl-3-furoic acid imidazolide), NAI (2-methylnicotinic acid imidazolide), and NAI-N3 (2-(azidomethyl)nicotinic acid acyl imidazole).

3. The method of claim 1, wherein the random hexamer-containing primer of step b) comprises a nucleotide sequence of SEQ ID NO:6.

4. The method of claim 1, wherein the ligation in step c) comprises ligating a hairpin donor molecule comprising SEQ ID NO:1 to the 3′ end of the cDNA molecule.

5. The method of claim 3, wherein the ligation is performed using T4 DNA ligase.

6. The method of claim 1, wherein the PCR amplification in step d) comprises contacting the ligated construct with a forward primer having a sequence as set forth in SEQ ID NO:3 and a reverse primer having a sequence as set forth in SEQ ID NO:4.

7. The method of claim 1, wherein the sequencing in step e) is performed using a sequencing primer as set forth in SEQ ID NO:5.

8. The method of claim 1, further comprising at least one purification step.

9. The method of claim 8, wherein the method comprises at least one purification step after step b) and before step c).

10. The method of claim 8, wherein the method comprises at least one purification step after step c) and before step d).

11. The method of claim 8, wherein the method comprises at least one purification step after step d) and before step e).

12. The method of claim 8, wherein at least one purification step comprises polyacrylamide gel (PAGE) purification.

13. The method of claim 8, wherein at least one purification step comprises affinity purification.

14. The method of claim 13, wherein the affinity purification comprises biotin/streptavidin affinity purification.

15. The method of claim 8, wherein the method comprises three purification steps.

16. The method of claim 15, wherein the method comprises a first purification step after step b) and before step c), a second purification step after step c) and before step d), and a third purification step after step d) and before step e).

17. A nucleic acid molecule comprising a sequence selected from the group consisting of SEQ ID NO:3, SEQ ID NO:4, SEQ ID NO:5 and SEQ ID NO:6.

18. A kit comprising a nucleic acid molecule comprising a sequence selected from the group consisting of SEQ ID NO:3, SEQ ID NO:4, SEQ ID NO:5, SEQ ID NO:6 and a combination thereof.


SEQUENCE LISTING (56 sequences)

SEQ ID NO: 1 - DNA, 40 bp, Artificial Sequence
  Chemically synthesized, hairpin donor
  misc_feature (23)..(28): any nucleotide
tgaagagcctagtcgctgttcannnnnnctgcccatagag

SEQ ID NO: 2 - DNA, 25 bp, Artificial Sequence
  chemically synthesized, ssDNA unstructured linker
  misc_feature (1)..(3): any nucleotide
nnnagatcggaagagcgtcgtgtag

SEQ ID NO: 3 - DNA, 80 bp, Artificial Sequence
  Chemically synthesized, Illumina TruSeq forward primer
aatgatacggcgaccaccgagatctacactctttccctacacgacgctcttccgatcttgaacagcgactaggctcttca

SEQ ID NO: 4 - DNA, 63 bp, Artificial Sequence
  Chemically synthesized, Illumina TruSeq reverse primers
  misc_feature (25)..(30): N6-N8 sequencing barcode region
caagcagaagacggcatacgagatnnnnnngtgactggagttcagacgtgtgctcttccgatc

SEQ ID NO: 5 - DNA, 33 bp, Artificial Sequence
  Chemically synthesized, sequencing primer
tcttccgatcttgaacagcgactaggctcttca

SEQ ID NO: 6 - DNA, 27 bp, Artificial Sequence
  Chemically Synthesized, random hexamer primer
  misc_feature (22)..(27): n represents any nucleotide
cagacgtgtgctcttccgatcnnnnnn

SEQ ID NO: 7 - DNA, 162 bp, Oryza sativa
cacacgactctcggcaacggatatctcggctctcgcatcgatgaagaacgtagcgaaatgcgatacctggtgtgaattgcagaatcccgtgaaccatcgagtctttgaacgcaagttgcgcccgaggccatccggccgagggcacgcctgcctgggcgtcac

SEQ ID NO: 8 - RNA, 181 nt, Bacillus subtilis
gguuaaguuagaaagggcgcacgguggaugccuuggcacuaggagccgaugaaggacgggacgaacaccgauaugcuucggggagcuguaagcaagcuuugauccggagauuuccgaauggggaaacccaccacucguaauggagugguauccauaucugaauucauaggauaugagaagg

SEQ ID NO: 9 - RNA, 131 nt, Bacillus subtilis
uuuaucggagaguuugauccuggcucaggacgaacgcuggcggcgugccuaauacaugcaagucgagcggacagaugggagcuugcucccgauguuagcggcggacgggugaguaacacguggguaaccug

SEQ ID NO: 10 - RNA, 118 nt, Bacillus subtilis
  misc_feature (1)..(3): n is a, c, g, or u
nnnuugguggcgauagcgaagaggucacacccguucccauaccgaacacggagauuaagcucuucagcgccgaugguagucggggguuucccccugugagaguaggacgccgccaagc

SEQ ID NO: 11 - RNA, 228 nt, Arabidopsis thaliana
guucgaguugucccagaagaaaggggcgacuaagcguagucaucccauuuugauuggacagagugcugccagauuugggucgagugcaagggauaaccacccacuuguuagguugugaaccacuuaagacgaaguguuacuauccuucucggcuguagcuuccuaguuuuucguugcagcgaucuugcgaaccgacgguguucggucaauagggacaccauugaaaag

SEQ ID NO: 12 - RNA, 229 nt, Oryza sativa
guucgaguugucccagaagaaaggggcgacuaagaguagucaucccauuuugauuggacagagugcugccagauuugggucgagugcaagggauaaccacccacuuguuagguugugaaccacuuaagacgaaguguuacuauccuucucggcuguagcuuccuaguuuuucguugcagcgauacuugcgaaccgacgguguucggucaauagggacaccauugaaaag

SEQ ID NO: 13 - DNA, 196 bp, Arabidopsis thaliana
actcccggatactatgatggacgatactggacaatgtggaagcttccattgttcggatgcaccgactccgctcaagagttgaaggaagttgaagaatgcaagaaggagtaccctggcgccttcattaggatcatcggattcgacaacacccgtcaagtccaatgcatcagtttcattgcctacaagcccccaagct

SEQ ID NO: 14 - DNA, 196 bp, Oryza sativa
tcccccggatactacgatggcaggtactggaccatgtggaagctgcccatgttcgggtgcactgacgccacccaggtgctcaaggagctcgaggaggccaagaaggcgtaccctgatgcattcgtccgtatcatcggcttcgacaacgtcaggcaggtgcagctcatcagcttcatcgcctacaagcccccgggct

SEQ ID NO: 15 - RNA, 17 nt, Homo sapiens
ggcacaaaguucugccc

SEQ ID NO: 16 - RNA, 17 nt, Saccharomyces cerevisiae
ggcacaaaguucugccc

SEQ ID NO: 17 - RNA, 17 nt, Haloarcula marismortui
agcaaaaaguuuugcau

SEQ ID NO: 18 - RNA, 17 nt, Oryza sativa
ggcacaaaguucugccc

SEQ ID NO: 19 - DNA, 16 bp, Oryza sativa
agtgaaatagaacgtg

SEQ ID NO: 20 - DNA, 16 bp, Oryza sativa
tcgcaaaagggggtcg

SEQ ID NO: 21 - RNA, 17 nt, Arabidopsis thaliana
gggagugaaauagaaca

SEQ ID NO: 22 - RNA, 18 nt, Oryza sativa
ggguagugaaauagaacg

SEQ ID NO: 23 - DNA, 16 bp, Oryza sativa
gcgtgacgcccaggca

SEQ ID NO: 24 - DNA, 26 bp, Oryza sativa
ggacgcctctccagactacaattcgg

SEQ ID NO: 25 - DNA, 22 bp, Escherichia coli
ttactcacccgtccgccactcg

SEQ ID NO: 26 - RNA, 102 nt, Oryza sativa
aagaacguagcgaaaugcgauaccuggugugaauugcagaaucccgugaaccaucgagucuuugaacgcaaguugcgcccgaggccauccggccgagggcac

SEQ ID NO: 27 - RNA, 102 nt, Oryza sativa; identical to SEQ ID NO: 26

SEQ ID NO: 28 - RNA, 43 nt, Oryza sativa
gcuugagaaucgggcggccgcgccguccgaauuguagucugga

SEQ ID NO: 29 - RNA, 20 nt, Oryza sativa
cgccugccugggcgucacgc

SEQ ID NO: 30 - RNA, 295 nt, Oryza sativa
gcgcgaccccaggucaggcgggacuacccgcugaguuuaagcauauaaauaagcgggagagaagaaacuuacgaggauuccccuaguaacggcgagcgaaccgggagaugcccagcuugagaaucgggcggccgcgccguccgaauuguagucuggagaggcguccucagcgacggaccgggcccaaguccccuagaaaggggcgccugggagggugagagccccguccggcccggacccugucgccccacgaggcgccgucaacgagucggguuguuugggaaugcagcccaaa

SEQ ID NO: 31 - RNA, 8 nt, Oryza sativa
aaggcuaa

SEQ ID NO: 32 - RNA, 171 nt, Escherichia coli
  misc_feature (109)..(112): n is a, c, g, or u
  misc_feature (125)..(128): n is a, c, g, or u
  misc_feature (141)..(145): n is a, c, g, or u
  misc_feature (160)..(164): n is a, c, g, or u
aaauugaagaguuugaucauggcucagauugaacgcuggcggcaggccuaacacaugcaagucgaacgguaacaggaagaagcuugcuucuuugcugacgaguggcggnnnngcagcaguggggnnnngccaugccgcgunnnnngaagcguuaaucggnnnnnaaugaau

SEQ ID NO: 33 - RNA, 19 nt, Artificial Sequence
  Chemically synthesized, representation of a ROSE element
  misc_feature (1)..(19): n represents any nucleotide, y represents a pyrimidine (C or U)
nnuygcunnnnnnaggann

SEQ ID NO: 34 - RNA, 28 nt, Artificial Sequence
  chemically synthesized, representation of a fourU element
  misc_feature (1)..(28): n represents any nucleotide
nnnnnnuuuunnnnnnnnaggannnnnn

SEQ ID NO: 35 - RNA, 40 nt, Artificial Sequence
  Chemically Synthesized, repesentation of a UCCU element
  misc_feature (1)..(40): n represents any nucleotide
nguaaucaauccuunnnnnnnnnnaaggauuaacauuaug

SEQ ID NO: 36 - RNA, 13 nt, Listeria monocytogenes
  misc_feature (1)..(1): n is a, c, g, or u
ncuaacaauuguu

SEQ ID NO: 37 - RNA, 13 nt, Listeria monocytogenes
  misc_feature (13)..(13): n is a, c, g, or u
aacgauugggggn

SEQ ID NO: 38 - RNA, 20 nt, Neisseria meningitidis
uauacuuauauacuuauaga

SEQ ID NO: 39 - DNA, 80 bp, Artificial Sequence; identical to SEQ ID NO: 3
  Chemically Synthesized, Illumina TruSeq Universal Adapter

SEQ ID NO: 40 - DNA, 63 bp, Artificial Sequence
  Chemically Synthesized, Illumina TruSeq Index Adapter reverse complementary primer with Index 4
caagcagaagacggcatacgagattggtcagtgactggagttcagacgtgtgctcttccgatc

SEQ ID NO: 41 - DNA, 63 bp, Artificial Sequence
  Chemically Synthesized, Illumina TruSeq Index Adapter reverse complementary primer with Index 7
caagcagaagacggcatacgagatgatctggtgactggagttcagacgtgtgctcttccgatc

SEQ ID NO: 42 - DNA, 65 bp, Artificial Sequence
  Chemically Synthesized, Illumina TruSeq Index Adapter reverse complementary primer with Index 21
caagcagaagacggcatacgagattccgaaacgtgactggagttcagacgtgtgctcttccgatc

SEQ ID NO: 43 - DNA, 65 bp, Artificial Sequence
  Chemically Synthesized, Illumina TruSeq Index Adapter reverse complementary primer with Index 25
caagcagaagacggcatacgagatatatcagtgtgactggagttcagacgtgtgctcttccgatc

SEQ ID NO: 44 - DNA, 63 bp, Artificial Sequence
  Chemically Synthesized, Illumina TruSeq Index Adapter reverse complementary primer with Index 2
caagcagaagacggcatacgagatacatcggtgactggagttcagacgtgtgctcttccgatc

SEQ ID NO: 45 - DNA, 65 bp, Artificial Sequence
  Chemically Synthesized, Illumina TruSeq Index Adapter reverse complementary primer with Index 18
caagcagaagacggcatacgagatgtgcggacgtgactggagttcagacgtgtgctcttccgatc

SEQ ID NO: 46 - DNA, 63 bp, Artificial Sequence
  Chemically Synthesized, Illumina TruSeq Index Adapter reverse complementary primer with Index 1
caagcagaagacggcatacgagatcgtgatgtgactggagttcagacgtgtgctcttccgatc

SEQ ID NO: 47 - DNA, 65 bp, Artificial Sequence
  Chemically Synthesized, Illumina TruSeq Index Adapter reverse complementary primer with Index 19
caagcagaagacggcatacgagatcgtttcacgtgactggagttcagacgtgtgctcttccgatc

SEQ ID NO: 48 - DNA, 65 bp, Artificial Sequence
  Chemically Synthesized, Illumina TruSeq Index Adapter reverse complementary primer with Index 20
caagcagaagacggcatacgagataaggccacgtgactggagttcagacgtgtgctcttccgatc

SEQ ID NO: 49 - DNA, 65 bp, Artificial Sequence
  Chemically Synthesized, Illumina TruSeq Index Adapter reverse complementary primer with Index 22
caagcagaagacggcatacgagattacgtacggtgactggagttcagacgtgtgctcttccgatc

SEQ ID NO: 50 - DNA, 65 bp, Artificial Sequence
  Chemically Synthesized, Illumina TruSeq Index Adapter reverse complementary primer with Index 23
caagcagaagacggcatacgagatatccactcgtgactggagttcagacgtgtgctcttccgatc

SEQ ID NO: 51 - DNA, 65 bp, Artificial Sequence
  Chemically Synthesized, Illumina TruSeq Index Adapter reverse complementary primer with Index 27
caagcagaagacggcatacgagataaaggaatgtgactggagttcagacgtgtgctcttccgatc

SEQ ID NO: 52 - DNA, 18 bp, Artificial Sequence
  Chemically Synthesized, Adapter sequence
actgtaggcaccatcaat

SEQ ID NO: 53 - DNA, 25 bp, Artificial Sequence
  Chemically Synthesized, OS06T0105350-00, T1
gcgatgctagaaaaaaaaaaaaaaa

SEQ ID NO: 54 - DNA, 25 bp, Artificial Sequence
  Chemically Synthesized, OS02T0662100-01, T2
ttttagattgaaaaaaaaaaaaaaa

SEQ ID NO: 55 - DNA, 25 bp, Artificial Sequence
  Chemically Synthesized, OS03T0159900-02, T3
attaatttttaaaaaaaaaaaaaaa

SEQ ID NO: 56 - DNA, 25 bp, Artificial Sequence
  Chemically Synthesized, OS02T0769100-01, T4
cacattttataaaaaaaaaaaaaaa